(19) United States
(12) Patent Application Publication
(10) Pub. No.: US 2024/0190842 A1
(43) Pub. Date: Jun. 13, 2024

(54) SUBSTITUTED QUINOLINONE-8-CARBONITRILE DERIVATIVES HAVING ANDROGEN DEGRADATION ACTIVITY AND USES THEREOF

(71) Applicant: Accutar Biotechnology Inc., Cranbury, NJ (US)

(72) Inventors: Yimin Qian, Plainsboro, NJ (US); Robert Z. Luo, New York, NY (US); Ji Liu, Shanghai (CN); Ke Liu, Shanghai (CN); Wei He, Zionsville, IN (US); Jie Fan, New York, NY (US)

(73) Assignee: Accutar Biotechnology Inc., Cranbury, NJ (US)

(21) Appl. No.: 18/504,246

(22) Filed: Nov. 8, 2023

Related U.S. Application Data
(60) Provisional application No. 63/423,981, filed on Nov. 9, 2022.

(51) Int. Cl. C07D 401/14; A61P 35/00; C07D 471/04; C07D 471/08; C07D 487/18

(52) U.S. Cl. CPC C07D 401/14; A61P 35/00; C07D 471/04; C07D 471/08; C07D 487/18

(57) ABSTRACT

The present disclosure relates to novel quinolinone-8-carbonitrile based compounds, pharmaceutical compositions containing such compounds, and their use in prevention and treatment of cancer and related diseases and conditions. The compounds disclosed herein exhibit androgen receptor degradation activity.

This application claims the benefit of priority to U.S. Provisional Patent Application No. 63/423,981, filed Nov. 9, 2022, which is hereby incorporated by reference in its entirety.

### FIELD OF THE DISCLOSURE

The present disclosure relates to novel quinolinone-8-carbonitrile based compounds, pharmaceutical compositions containing such compounds, and their use in prevention and treatment of diseases and conditions, e.g., cancer. The compounds disclosed herein exhibit androgen receptor degradation activity.

### BACKGROUND OF THE DISCLOSURE

Androgens, through binding to the Androgen Receptor (AR), govern a wide range of physiological processes. For example, androgens are required for normal prostate development and function as they are key in the AR signaling pathway. Unfortunately, the AR signaling pathway is also implicated in the development and survival of cancers, such as prostate, breast, and other cancers (see, e.g., “Androgen Receptor in Prostate Cancer”, Endocrine Reviews, 2004, 25(2), 276-308; and “Androgen receptors beyond prostate cancer: an old marker as a new target”, Oncotarget, 2014, 6(2), 592-603).

Traditional methods to treat cancers where AR is implicated, such as prostate cancer, involves AR signaling suppression through, for example, androgen deprivation therapy. Such therapy includes chemical and/or surgical castration. Alternatively, anti-androgen therapy may be pursued, whereby a patient is treated with an AR inhibitor, such as enzalutamide (XTANDI®). Although these treatment methods have resulted in improved prognoses for individuals with androgen receptor positive cancer, cancer progression is eventually observed and occurs through, for example, AR gene amplification and/or development of AR mutations.

Accordingly, there exists a need to treat AR positive cancer that halts progression of the cancer, even if the individual has experienced one or more prior therapies. Disclosed herein are novel quinolinone-8-carbonitrile based compounds that exhibit AR degradative activity. The disclosed compounds are useful for the treatment of cancers, such as prostate cancer. In some instances, the cancer is AR positive.

### SUMMARY OF THE DISCLOSURE

The disclosure provides compounds, compositions and methods for modulating the activity of AR. In some embodiments, the compounds exhibit androgen receptor degradation activity.

Disclosed are compounds of Formula (1A) or Formula (1B), or a tautomer, stereoisomer or a mixture of stereoisomers, a pharmaceutically acceptable salt, hydrate, or deuterated derivative thereof:

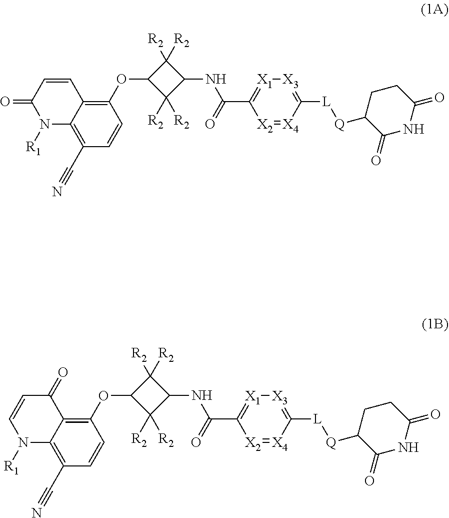

wherein:
R₁ is hydrogen, C₁-C₅ alkyl, C₁-C₅ halogenated alkyl, or C₃-C₅ cycloalkyl;
each R₂ is independently hydrogen, C₁-C₃ alkyl, or C₁-C₃ haloalkyl;
X₁ is CRₐ or N;
X₂ is CR_{b} or N;
X₃ is CR_{c} or N;
X₄ is CR_{d} or N;
each of Rₐ, R_{b}, R_{c}, and R_{d} is independently hydrogen, halogen, C₁-C₃ alkyl, C₁-C₃ haloalkyl, or C₁-C₃ alkoxy;
L is a linker of 1 to 15 carbon atoms in length, wherein one or more carbon atoms are optionally substituted with halogen, oxygen, nitrogen, alkylamino, hydroxyl, sulfur, sulfoxide, sulfone, or amide, and wherein one or more carbon atoms are optionally replaced by cycloalkyl, heterocycle, or heteroaryl, wherein the cycloalkyl, heterocycle, or heteroaryl are each independently substituted with 0, 1, or 2 R₃;
each R₃ is independently hydrogen, halogen, hydroxyl, C₁-C₄alkoxyl, C₁-C₄alkyl, or C₁-C₄haloalkyl;
Q is a 1 to 4 carbon atoms in length, wherein one or more carbon atoms are replaced by a heteroaryl, halogen substituted heteroaryl, alkoxy substituted heteroaryl, aryl, halogen substituted aryl, alkoxy substituted aryl, 6,5-membered fused heterocycle, 6,6-membered fused heterocycle, —C(═O), —C(═O)—NH—, or —C(═O)—NH—(C₁-C₃alkyl), wherein the 6,5-membered fused heterocycle or 6,6-membered fused heterocycle are each independently substituted with 0, 1, or 2 R₄; and
R₄ is independently hydrogen, halogen, hydroxyl, C₁-C₃alkyl, C₁-C₃haloalkyl, C₁-C₃alkoxy or an oxo group.

In some embodiments, L is:

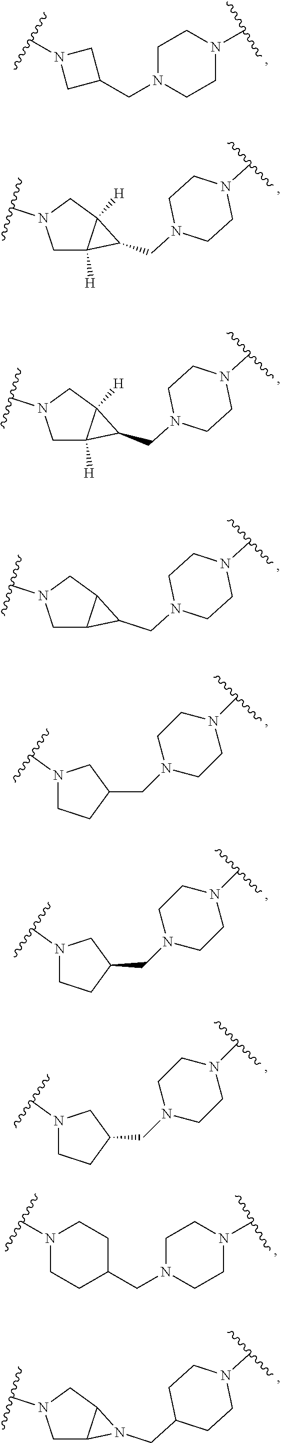

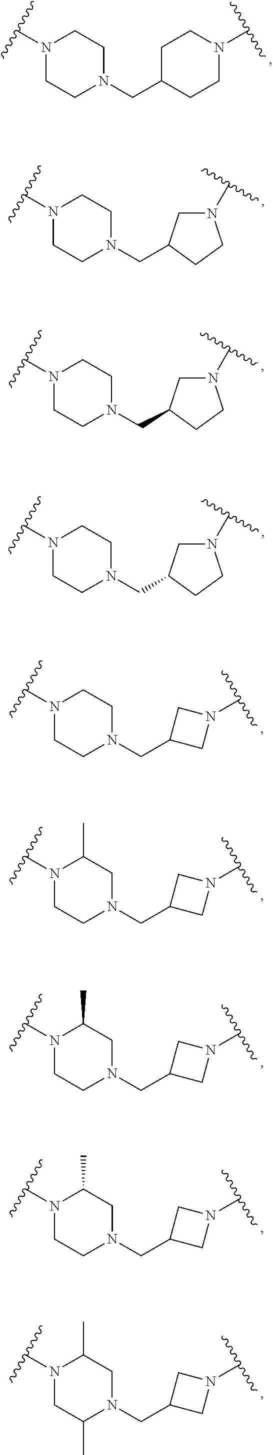

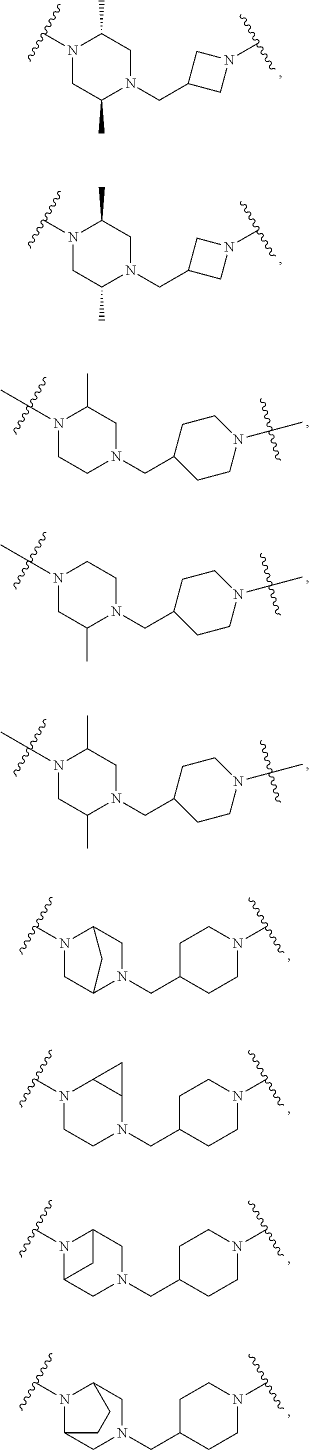

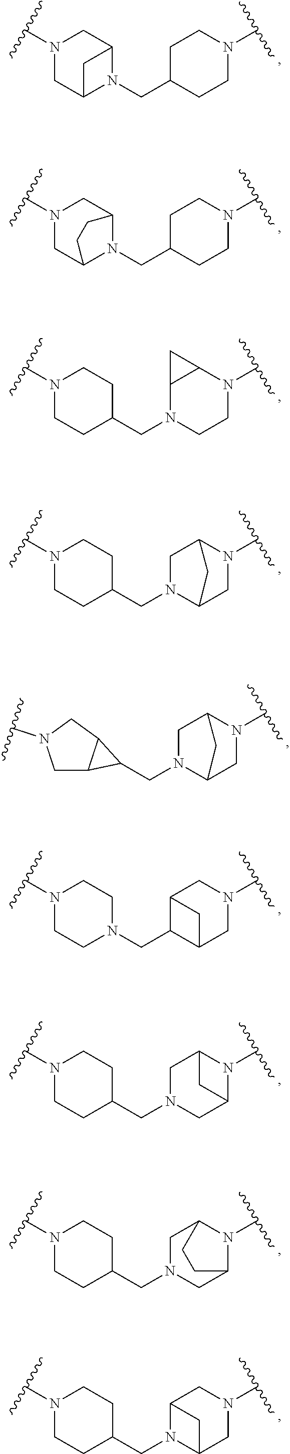

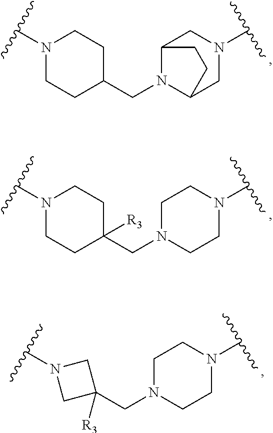

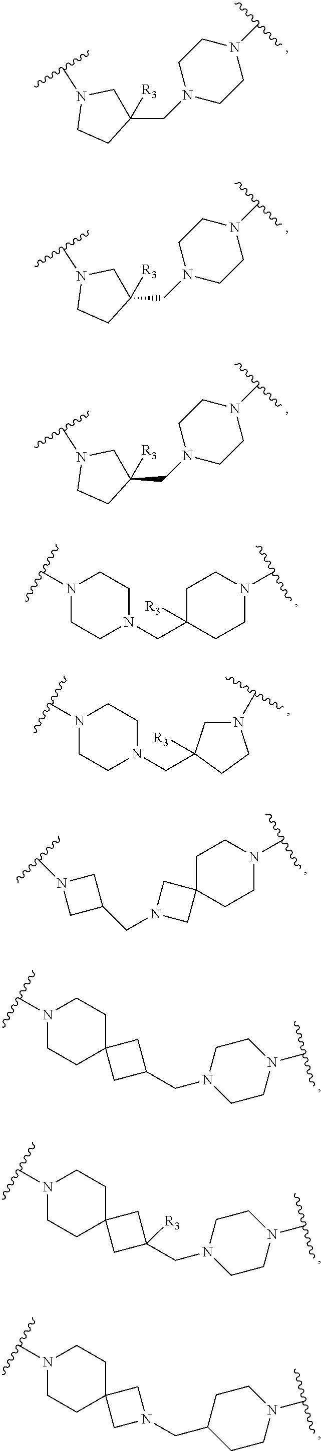

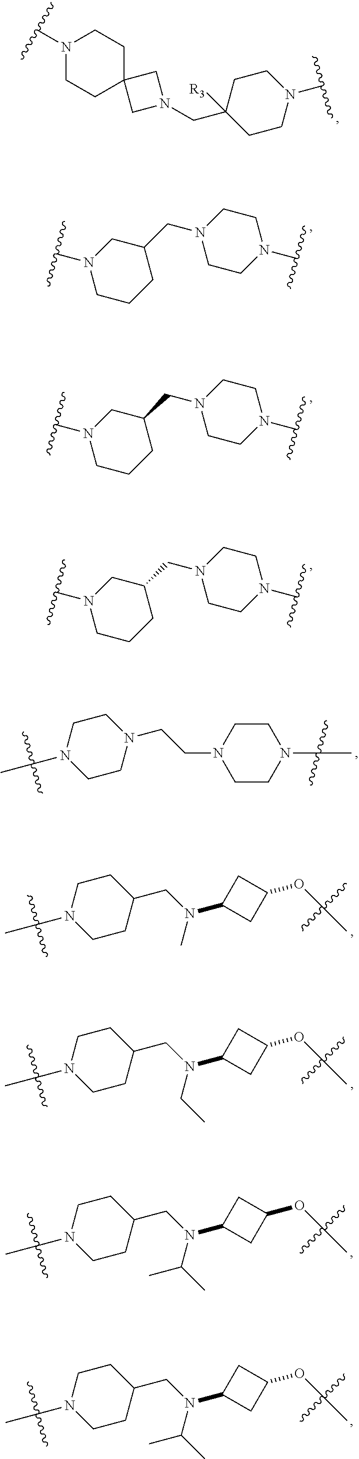

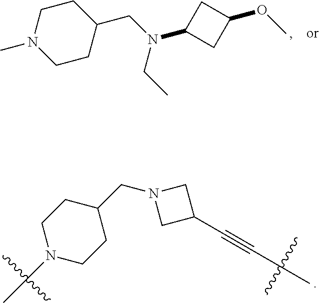

In some embodiments, L is:

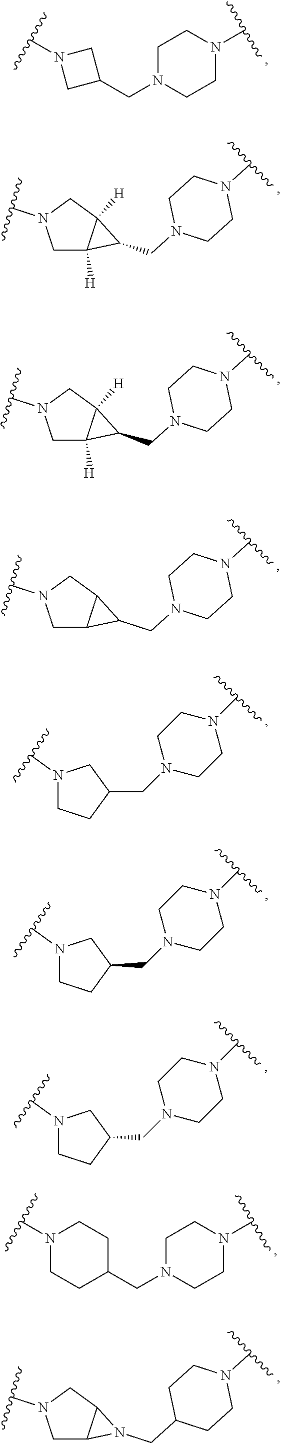

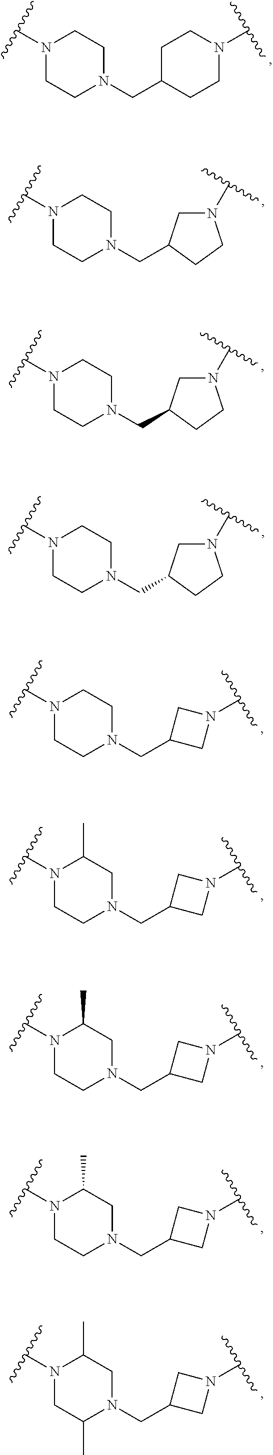

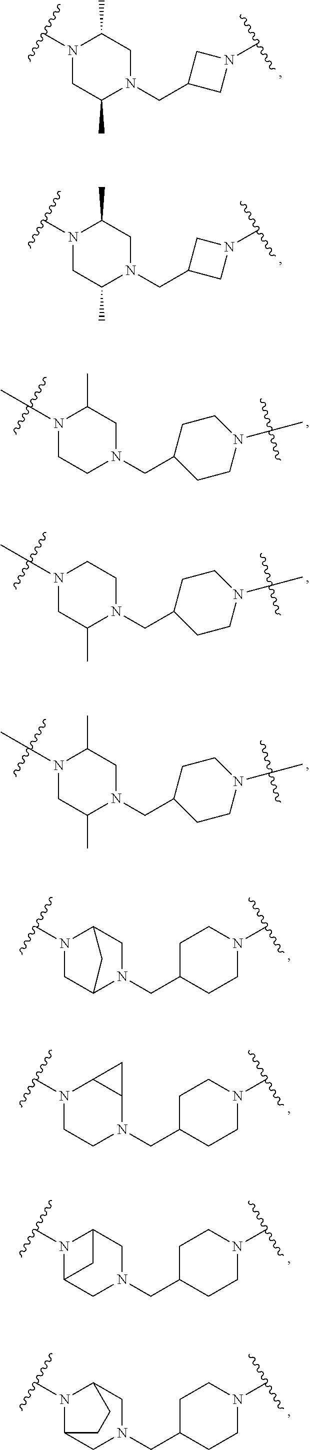

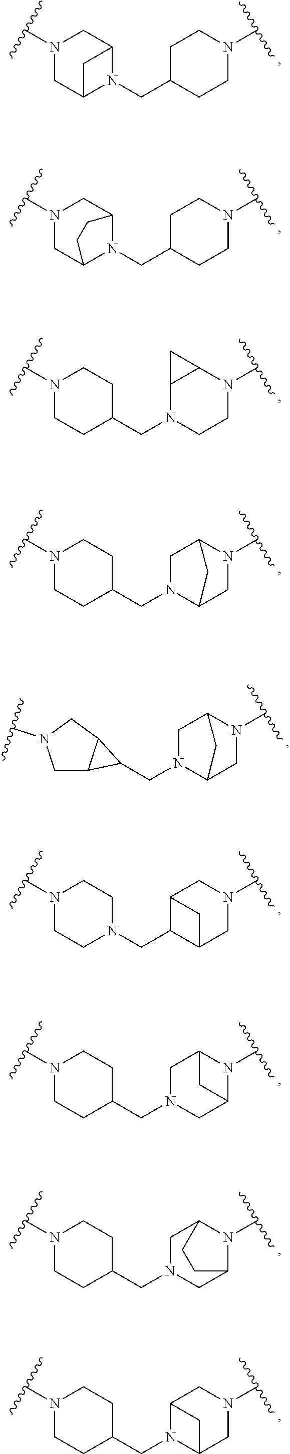

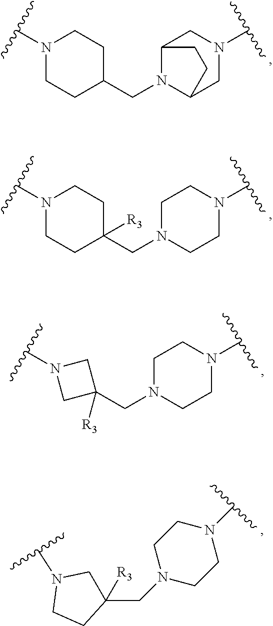

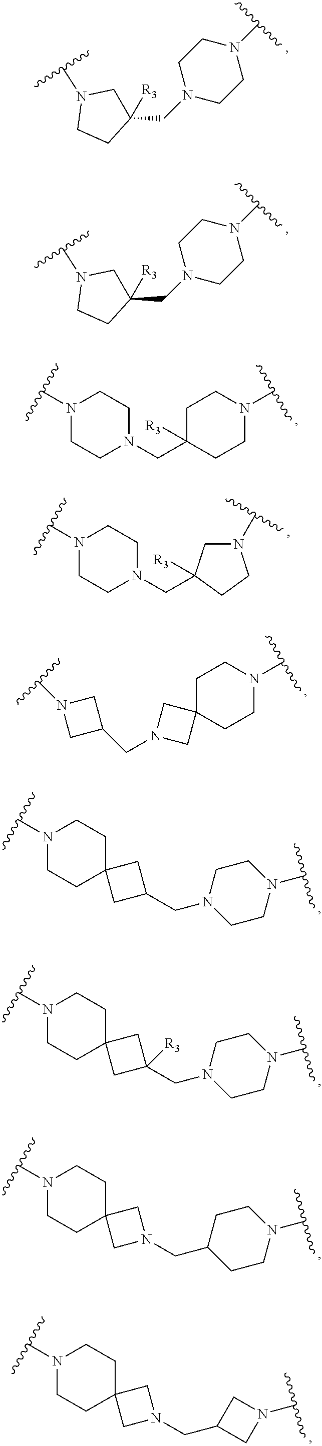

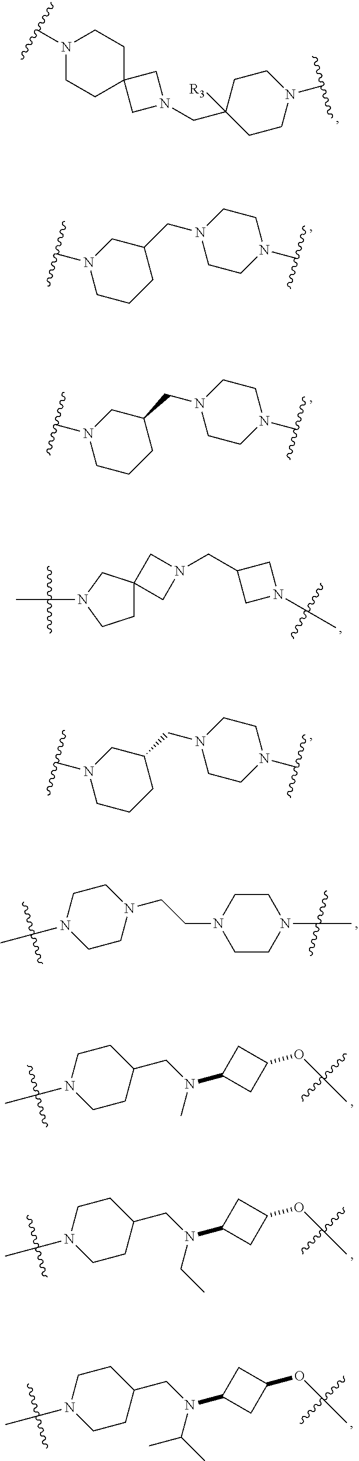

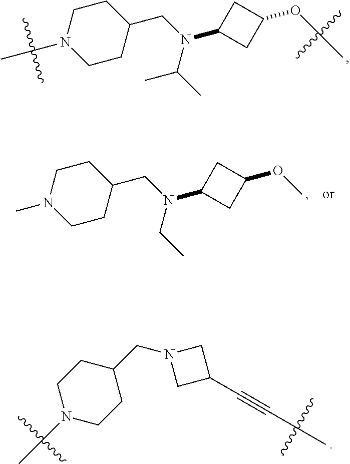

In some embodiments, Q is:

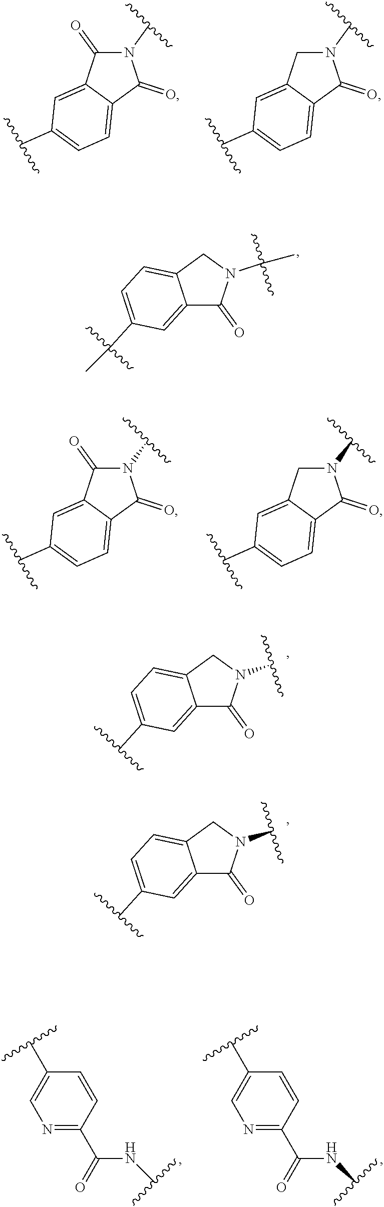

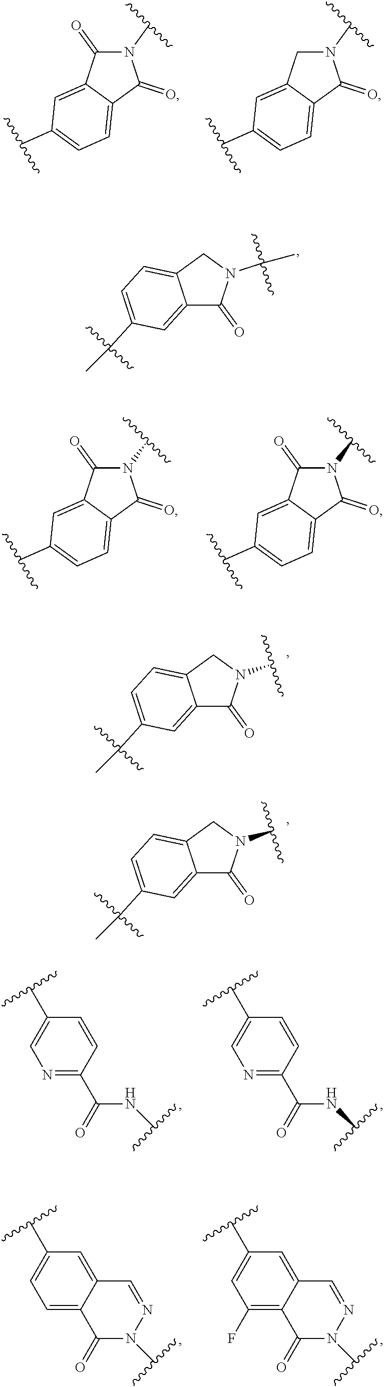

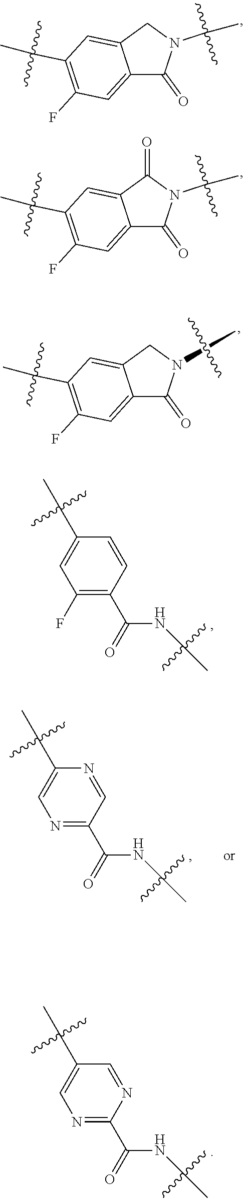

In some embodiments, Q is:

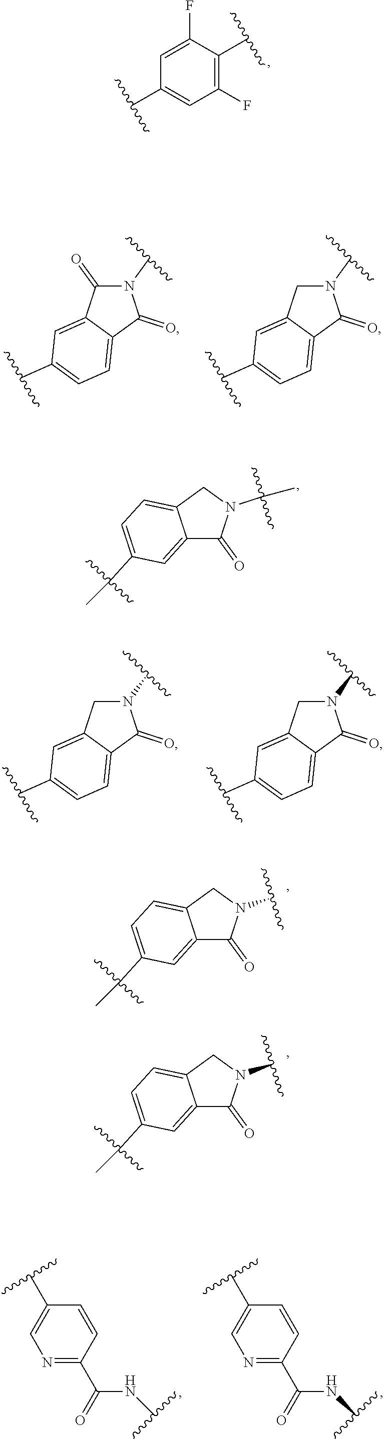

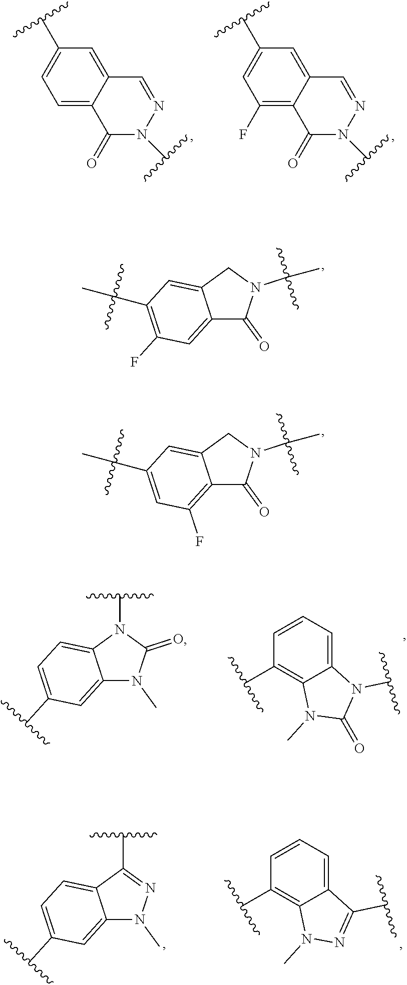

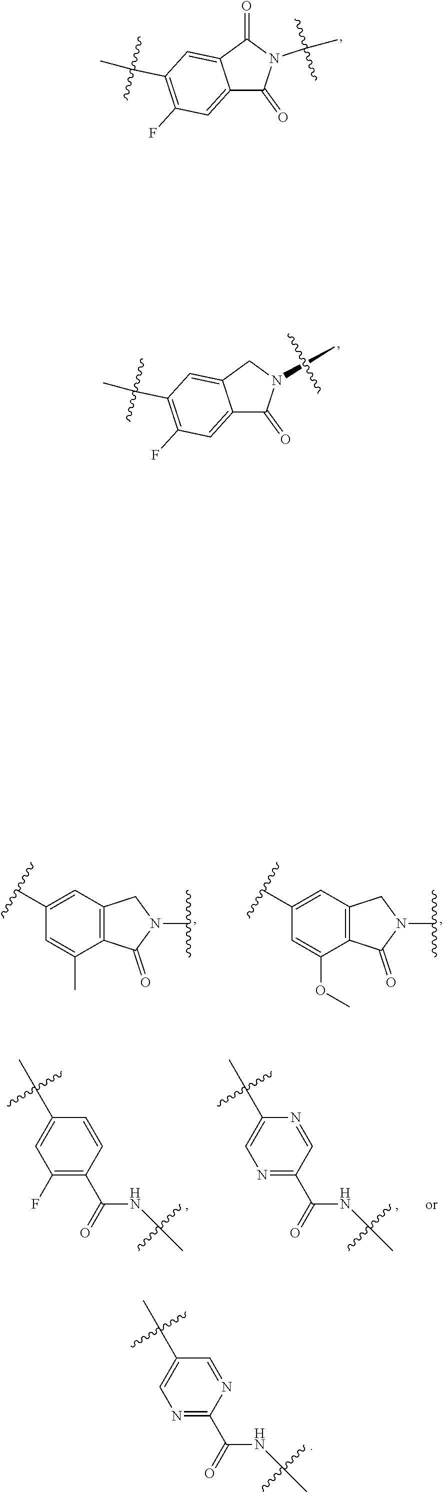

Also disclosed herein is a method of treating cancer, in a subject in need thereof, comprising administering to said subject a compound of Formula (1A) or Formula (1B) or a pharmaceutically acceptable salt thereof, or a pharmaceutical composition comprising a compound of Formula (1A) or Formula (1B) or a pharmaceutically acceptable salt thereof. In at least one embodiment, the pharmaceutical composition of the present disclosure may be for use in (or in the manufacture of medicaments for) the treatment of cancer in the subject in need thereof.

In at least one embodiment, a therapeutically effective amount of a pharmaceutical composition of the present disclosure may be administered to a subject diagnosed with cancer. In some embodiments, the cancer is selected from prostate cancer, head and neck cancer, skin cancer, sarcoma, renal cell carcinoma, adrenocortical carcinoma, bladder cancer, lung cancer, gastric carcinoma, esophageal carcinoma, pancreatic adenocarcinoma, colorectal cancer, connective tissue cancer, glioblastoma multiforme, cervical cancer, uterine cancer, ovarian cancer, and breast cancer.

### BRIEF DESCRIPTION OF THE FIGURES

The accompanying drawings, which are incorporated in and constitute a part of this specification, illustrate the disclosed embodiments and, together with the description, explain the principles of the disclosed embodiments. In the drawings:

FIG. 1A illustrates the androgen receptor (AR) degradative activity of exemplary compound 16 of the present disclosure in VCAP cell lines 24 hours after administration.

FIG. 1B illustrates the androgen receptor (AR) degradative activity of exemplary compound 16 of the present disclosure in LNCAP cell lines 24 hours after administration.

FIG. 1C illustrates the AR degradative activity of exemplary compound 18 of the present disclosure in VCAP cell lines 24 hours after administration.

FIG. 1D illustrates the AR degradative activity of exemplary compound 18 in LNCAP cell lines 24 hours after administration.

FIG. 2A illustrates the AR degradative activity of exemplary compound 16 of the present disclosure in PC3 AR-WT cell lines 24 hours after administration.

FIG. 2B illustrates the AR degradative activity of exemplary compound 29 of the present disclosure in PC3 AR-WT cell lines 24 hours after administration.

### DETAILED DESCRIPTION OF THE DISCLOSURE

### Definitions

As used herein, “cancer” refers to diseases, disorders, and conditions that involve abnormal cell growth with the potential to invade or spread to other parts of the body. Exemplary cancers include, but are not limited to, prostate cancer, head and neck cancer, skin cancer, sarcoma, renal cell carcinoma, adrenocortical carcinoma, bladder cancer, lung cancer, gastric carcinoma, esophageal carcinoma, pancreatic adenocarcinoma, colorectal cancer, connective tissue cancer, glioblastoma multiforme, cervical cancer, uterine cancer, ovarian cancer, and breast cancer.

As used herein, the term “androgen receptor positive” means that androgen receptor is detected by one or more analytical methods, e.g., immunohistochemistry. For example, analysis of a biopsy of a subject's tumor may indicate the presence of androgen receptor. AR status may be tested by circulating cancer cells or circulating tumor DNA in a blood test. In some circumstances an AR test may not be performed.

“Subject” refers to an animal, such as a mammal, that has been or will be the object of treatment, observation, or experiment. The methods described herein may be useful for both human therapy and veterinary applications. In one embodiment, the subject is a human.

As used herein, “treatment” or “treating” refers to an amelioration of a disease or disorder, or at least one discernible symptom thereof. In another embodiment, “treatment” or “treating” refers to an amelioration of at least one measurable physical parameter, not necessarily discernible by the patient. In yet another embodiment, “treatment” or “treating” refers to inhibiting the progression of a disease or disorder, either physically, e.g., stabilization of a discernible symptom, physiologically, e.g., stabilization of a physical parameter, or both. In yet another embodiment, “treatment” or “treating” refers to delaying the onset of a disease or disorder.

A dash (“—”) that is not between two letters or symbols is used to indicate a point of attachment for a substituent. For example, —CN is attached through the carbon atom.

By “optional” or “optionally” it is meant that the subsequently described event or circumstance may or may not occur, and that the description includes instances where the event or circumstance occurs and instances in which is does not. It will be understood by those skilled in the art, with respect to any group containing one or more substituents, that such groups are not intended to introduce any substitution or substitution patterns that are sterically impractical, synthetically non-feasible and/or inherently unstable.

When a range of values is listed, it is intended to encompass each value and sub-range within the range. For example, “C₁-C₆ alkyl” is intended to encompass C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “alkenyl” as used herein refers to an unsaturated, two-carbon group having a carbon-carbon double bond, referred to herein as C₂-alkenyl.

The term “alkoxy” as used herein refers to an alkyl or cycloalkyl covalently bonded to an oxygen atom.

The term “alkyl” as used herein refers to a saturated straight or branched hydrocarbon, such as a straight or branched group of 1-8 carbon atoms, referred to herein as (C₁-C₈)alkyl. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, and octyl. In some embodiments, “alkyl” is a straight-chain hydrocarbon. In some embodiments, “alkyl” is a branched hydrocarbon.

The term “alkynyl” as used herein refers to an unsaturated, two-carbon group having a carbon-carbon triple bond, referred to herein as C₂-alkynyl.

The term “aryl” as used herein refers to a mono-, bi-, or other multi-carbocyclic, aromatic ring system with 5 to 14 ring atoms. The aryl group can optionally be fused to one or more rings selected from aryls, cycloalkyls, heteroaryls, and heterocyclyls. The aryl groups of this present disclosure can be substituted with groups selected from alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide, and thioketone. Exemplary aryl groups include, but are not limited to, phenyl, tolyl, anthracenyl, fluorenyl, indenyl, azulenyl, and naphthyl, as well as benzo-fused carbocyclic moieties such as 5,6,7,8-tetrahydronaphthyl. Exemplary aryl groups also include but are not limited to a monocyclic aromatic ring system, wherein the ring comprises 6 carbon atoms, referred to herein as “C₆-aryl.”

The term “cycloalkyl” as used herein refers to a saturated or unsaturated cyclic, bicyclic, or bridged bicyclic hydrocarbon group of 3-16 carbons, or 3-8 carbons, referred to herein as “(C₃-C₅)cycloalkyl,” derived from a cycloalkane. Exemplary cycloalkyl groups include, but are not limited to, cyclohexanes, cyclohexenes, cyclopentanes, and cyclopentenes. Cycloalkyl groups may be substituted with alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide and thioketone. Cycloalkyl groups can be fused to other cycloalkyl (saturated or partially unsaturated), aryl, or heterocyclyl groups, to form a bicycle, tetracycle, etc. The term “cycloalkyl” also includes bridged and spiro-fused cyclic structures which may or may not contain heteroatoms.

The terms “halo” or “halogen” as used herein refer to —F, —Cl, —Br, and/or —I.

The term “haloalkyl group” as used herein refers to an alkyl group substituted with one or more halogen atoms.

The term “heteroaryl” as used herein refers to a mono-, bi-, or multi-cyclic, aromatic ring system containing one or more heteroatoms, for example 1-4 heteroatoms, such as nitrogen, oxygen, and sulfur. Heteroaryls can be substituted with one or more substituents including alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide and thioketone. Heteroaryls can also be fused to non-aromatic rings. Illustrative examples of heteroaryl groups include, but are not limited to, pyridinyl, pyridazinyl, pyrimidyl, pyrazyl, triazinyl, pyrrolyl, pyrazolyl, imidazolyl, (1,2,3)- and (1,2,4)-triazolyl, pyrazinyl, pyrimidilyl, tetrazolyl, furyl, thienyl, isoxazolyl, thiazolyl, furyl, phenyl, isoxazolyl, and oxazolyl. Exemplary heteroaryl groups include, but are not limited to, a monocyclic aromatic ring, wherein the ring comprises 2-5 carbon atoms and 1-3 heteroatoms, referred to herein as “(C₂-C₅)heteroaryl.” In some embodiments, a heteraryl contains 5 to 10 ring atoms, 1 to 4 of which are heteroatoms selected from N, O, and S. In some embodiments, a heteroaryl contains 5 to 8 ring atoms, 1 to 4 of which are heteroatoms selected from N, O, and S.

The terms “heterocycle,” “heterocyclyl,” or “heterocyclic” as used herein each refer to a saturated or unsaturated 3- to 18-membered ring containing one, two, three, or four heteroatoms independently selected from nitrogen, oxygen, phosphorus, and sulfur. Heterocycles can be aromatic (heteroaryls) or non-aromatic. Heterocycles can be substituted with one or more substituents including alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide and thioketone. Heterocycles also include bicyclic, tricyclic, and tetracyclic groups in which any of the above heterocyclic rings is fused to one or two rings independently selected from aryls, cycloalkyls, and heterocycles. Heterocycles also include bridged and spiro-fused cyclic structures which may or may not contain heteroatoms. Exemplary heterocycles include acridinyl, benzimidazolyl, benzofuryl, benzothiazolyl, benzothienyl, benzoxazolyl, biotinyl, cinnolinyl, dihydrofuryl, dihydroindolyl, dihydropyranyl, dihydrothienyl, dithiazolyl, furyl, homopiperidinyl, imidazolidinyl, imidazolinyl, imidazolyl, indolyl, isoquinolyl, isothiazolidinyl, isothiazolyl, isoxazolidinyl, isoxazolyl, morpholinyl, oxadiazolyl, oxazolidinyl, oxazolyl, piperazinyl, piperidinyl, pyranyl, pyrazolidinyl, pyrazinyl, pyrazolyl, pyrazolinyl, pyridazinyl, pyridyl, pyrimidinyl, pyrimidyl, pyrrolidinyl, pyrrolidin-2-onyl, pyrrolinyl, pyrrolyl, quinolinyl, quinoxaloyl, tetrahydrofuryl, tetrahydroisoquinolyl, tetrahydropyranyl, tetrahydroquinolyl, tetrazolyl, thiadiazolyl, thiazolidinyl, thiazolyl, thienyl, thiomorpholinyl, thiopyranyl, and triazolyl. In some embodiments, a heterocycle contains 5 to 10 ring atoms, 1 to 4 of which are heteroatoms selected from N, O, and S. In some embodiments, a heterocycle contains 5 to 8 ring atoms, 1 to 4 of which are heteroatoms selected from N, O, and S.

The terms “hydroxy” and “hydroxyl” as used herein refer to —OH.

The term “oxo” as used herein refers to a double bond to an oxygen atom (i.e., ═O). For example, when two geminal groups on a carbon atom are “taken together to form an oxo”, then a carbonyl (i.e., C═O) is formed.

The term “pharmaceutically acceptable carrier” as used herein refers to any and all solvents, dispersion media, coatings, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions.

As used herein, the term “pharmaceutically acceptable salt” refers to a salt form of a compound of this disclosure wherein the salt is nontoxic. Pharmaceutically acceptable salts of the compounds of this disclosure include those derived from suitable inorganic and organic acids and bases. A “free base” form of a compound, for example, does not contain an ionically bonded salt.

The phrase “and pharmaceutically acceptable salts and deuterated derivatives thereof” is used interchangeably with “and pharmaceutically acceptable salts thereof and deuterated derivatives of any of the forgoing” in reference to one or more compounds or formulae of the disclosure. These phrases are intended to encompass pharmaceutically acceptable salts of any one of the referenced compounds, deuterated derivatives of any one of the referenced compounds, and pharmaceutically acceptable salts of those deuterated derivatives.

One of ordinary skill in the art would recognize that, when an amount of “a compound or a pharmaceutically acceptable salt thereof” is disclosed, the amount of the pharmaceutically acceptable salt form of the compound is the amount equivalent to the concentration of the free base of the compound. It is noted that the disclosed amounts of the compounds or their pharmaceutically acceptable salts thereof herein are based upon their free base form.

Suitable pharmaceutically acceptable salts are, for example, those disclosed in S. M. Berge, et al. J. Pharmaceutical Sciences, 1977, 66, 1-19. For example, Table 1 of that article provides the following pharmaceutically acceptable salts:

TABLE 1

| Acetate | Iodide | Benzathine |
|---|---|---|
| Benzenesulfonate | Isethionate | Chloroprocaine |
| Benzoate | Lactate | Choline |
| Bicarbonate | Lactobionate | Diethanolamine |
| Bitartrate | Malate | Ethylenediamine |
| Bromide | Maleate | Meglumine |
| Calcium edetate | Mandelate | Procaine |
| Camsylate | Mesylate | Aluminum |
| Carbonate | Methylbromide | Calcium |
| Chloride | Methylnitrate | Lithium |
| Citrate | Methylsulfate | Magnesium |
| Dihydrochloride | Mucate | Potassium |
| Edetate | Napsylate | Sodium |
| Edisylate | Nitrate | Zinc |
| Estolate | Pamoate (Embonate) | |
| Esylate | Pantothenate | |
| Fumarate | Phosphate/diphosphate | |
| Gluceptate | Polygalacturonate | |
| Gluconate | Salicylate | |
| Glutamate | Stearate | |
| Glycollylarsanilate | Subacetate | |
| Hexylresorcinate | Succinate | |
| Hydrabamine | Sulfate | |
| Hydrobromide | Tannate | |
| Hydrochloride | Tartrate | |
| Hydroxynaphthoate | Teociate | |
| | Triethiodide | |

Non-limiting examples of pharmaceutically acceptable acid addition salts include: salts formed with inorganic acids, such as hydrochloric acid, hydrobromic acid, phosphoric acid, sulfuric acid, or perchloric acid; salts formed with organic acids, such as acetic acid, oxalic acid, maleic acid, tartaric acid, citric acid, succinic acid or malonic acid; and salts formed by using other methods used in the art, such as ion exchange. Non-limiting examples of pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, and valerate salts. Pharmaceutically acceptable salts derived from appropriate bases include alkali metal, alkaline earth metal, ammonium, and N⁺(C₁₋₄alkyl)₄ salts. This disclosure also envisions the quaternization of any basic nitrogen-containing groups of the compounds disclosed herein. Suitable non-limiting examples of alkali and alkaline earth metal salts include sodium, lithium, potassium, calcium, and magnesium. Further non-limiting examples of pharmaceutically acceptable salts include ammonium, quaternary ammonium, and amine cations formed using counterions such as halide, hydroxide, carboxylate, sulfate, phosphate, nitrate, lower alkyl sulfonate and aryl sulfonate. Other suitable, non-limiting examples of pharmaceutically acceptable salts include besylate and glucosamine salts.

As used herein, nomenclature for compounds including organic compounds, can be given using common names, IUPAC, IUBMB, or CAS recommendations for nomenclature. One of skill in the art can readily ascertain the structure of a compound if given a name, either by systemic reduction of compound structure using naming conventions, or by commercially available software, such as CHEMDRAW™ (Cambridgesoft Corporation, U.S.A.).

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” depending on the configuration of substituents around the stereogenic carbon atom. The present disclosure encompasses various stereoisomers of these compounds and mixtures thereof. Stereoisomers include enantiomers and diastereomers. Mixtures of enantiomers or diastereomers may be designated “(±)” in nomenclature, but the skilled artisan will recognize that a structure may denote a chiral center implicitly. In some embodiments, an enantiomer or stereoisomer may be provided substantially free of the corresponding enantiomer.

In some embodiments, the compound is a racemic mixture of (S)- and (R)-isomers. In other embodiments, provided herein is a mixture of compounds wherein individual compounds of the mixture exist predominately in an (S)- or (R)-isomeric configuration. For example, the compound mixture has an (S)-enantiomeric excess of greater than about 55%, about 60%, about 65%, about 70%, about 75%, about 80%, about 85%, about 90%, about 95%, about 96%, about 97%, about 98%, about 99%, about 99.5%, or more. In other embodiments, the compound mixture has an (S)-enantiomeric excess of greater than about 55% to about 99.5%, greater than about 60% to about 99.5%, greater than about 65% to about 99.5%, greater than about 70% to about 99.5%, greater than about 75% to about 99.5%, greater than about 80% to about 99.5%, greater than about 85% to about 99.5%, greater than about 90% to about 99.5%, greater than about 95% to about 99.5%, greater than about 96% to about 99.5%, greater than about 97% to about 99.5%, greater than about 98% to greater than about 99.5%, greater than about 99% to about 99.5%, or more. In other embodiments, the compound mixture has an (R)-enantiomeric purity of greater than about 55%, about 60%, about 65%, about 70%, about 75%, about 80%, about 85%, about 90%, about 95%, about 96%, about 97%, about 98%, about 99%, about 99.5% or more. In some other embodiments, the compound mixture has an (R)-enantiomeric excess of greater than about 55% to about 99.5%, greater than about 60% to about 99.5%, greater than about 65% to about 99.5%, greater than about 70% to about 99.5%, greater than about 75% to about 99.5%, greater than about 80% to about 99.5%, greater than about 85% to about 99.5%, greater than about 90% to about 99.5%, greater than about 95% to about 99.5%, greater than about 96% to about 99.5%, greater than about 97% to about 99.5%, greater than about 98% to greater than about 99.5%, greater than about 99% to about 99.5% or more.

Individual stereoisomers of compounds of the present disclosure can be prepared synthetically from commercially available starting materials that contain asymmetric or stereogenic centers, or by preparation of racemic mixtures followed by resolution methods well known to those of ordinary skill in the art. These methods of resolution are exemplified by: (1) attachment of a mixture of enantiomers to a chiral auxiliary, separation of the resulting mixture of diastereomers by recrystallization or chromatography and liberation of the optically pure product from the auxiliary; (2) salt formation employing an optically active resolving agent; or (3) direct separation of the mixture of optical enantiomers on chiral chromatographic columns. Stereoisomeric mixtures can also be resolved into their component stereoisomers by well-known methods, such as chiral-phase gas chromatography, chiral-phase high performance liquid chromatography, crystallizing the compound as a chiral salt complex, or crystallizing the compound in a chiral solvent. Stereoisomers can also be obtained from stereomerically-pure intermediates, reagents, and catalysts by well-known asymmetric synthetic methods.

Geometric isomers can also exist in the compounds of the present disclosure. The present disclosure encompasses the various geometric isomers and mixtures thereof resulting from the arrangement of substituents around a carbon-carbon double bond or arrangement of substituents around a carbocyclic ring. Substituents around a carbon-carbon double bond are designated as being in the “Z” or “E” configuration wherein the terms “Z” and “E” are used in accordance with IUPAC standards. Unless otherwise specified, structures depicting double bonds encompass both the E and Z isomers.

Substituents around a carbon-carbon double bond alternatively can be referred to as “cis” or “trans,” where “cis” represents substituents on the same side of the double bond and “trans” represents substituents on opposite sides of the double bond. The arrangements of substituents around a carbocyclic ring are designated as “cis” or “trans.” The term “cis” represents substituents on the same side of the plane of the ring and the term “trans” represents substituents on opposite sides of the plane of the ring. Mixtures of compounds wherein the substituents are disposed on both the same and opposite sides of plane of the ring are designated “cis/trans.”

The compounds disclosed herein may exist as tautomers and both tautomeric forms are intended to be encompassed by the scope of the present disclosure, even if only one tautomeric structure is depicted.

Additionally, unless otherwise stated, structures described herein are also meant to include compounds that differ only in the presence of one or more isotopically enriched atoms. For example, compounds having the present structures except for the replacement of hydrogen by deuterium (²H) or tritium (³H), or the replacement of a carbon by a ¹³C- or ¹⁴C-carbon atom are within the scope of this disclosure. Such compounds may be useful as, for example, analytical tools, probes in biological assays, or therapeutic agents.

### Compounds

In some embodiments, the present disclosure is directed to a compound of Formula (1A) or Formula (1B), or a tautomer, stereoisomer or a mixture of stereoisomers, a pharmaceutically acceptable salt, hydrate, or deuterated derivative thereof:

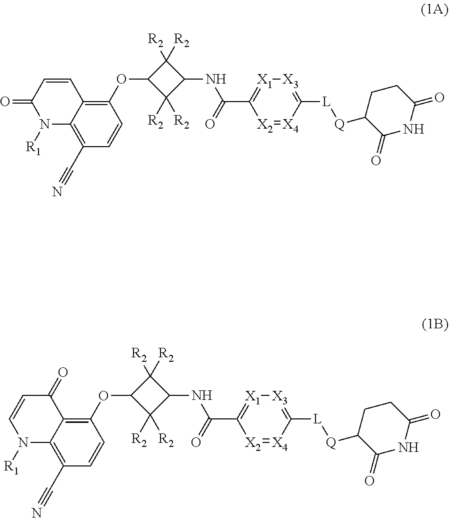

wherein:
R₁ is hydrogen, C₁-C₅ alkyl, C₁-C₅ halogenated alkyl, or C₃-C₅ cycloalkyl;
each R₂ is independently hydrogen, C₁-C₃ alkyl, or C₁-C₃ haloalkyl;
X₁ is CRₐ or N;
X₂ is CR_{b} or N;
X₃ is CR_{c} or N;
X₄ is CR_{d} or N;
each of Rₐ, R_{b}, R_{c}, and R_{d} is independently hydrogen, halogen, C₁-C₃ alkyl, C₁-C₃ haloalkyl, or C₁-C₃ alkoxy;
L is a linker of 1 to 15 carbon atoms in length, wherein one or more carbon atoms are optionally substituted with halogen, oxygen, nitrogen, alkylamino, hydroxyl, sulfur, sulfoxide, sulfone, or amide, and wherein one or more carbon atoms are optionally replaced by cycloalkyl, heterocycle, or heteroaryl, wherein the cycloalkyl, heterocycle, or heteroaryl are each independently substituted with 0, 1, or 2 R₃;
each R₃ is independently hydrogen, halogen, hydroxyl, C₁-C₄alkoxyl, C₁-C₄alkyl, or C₁-C₄haloalkyl;
Q is a 1 to 4 carbon atoms in length, wherein one or more carbon atoms are replaced by a heteroaryl, halogen substituted heteroaryl, alkoxy substituted heteroaryl, aryl, halogen substituted aryl, alkoxy substituted aryl, 6,5-membered fused heterocycle, 6,6-membered fused heterocycle, —C(═O), —C(═O)—NH—, or —C(═O)—NH—(C₁-C₃alkyl), wherein the 6,5-membered fused heterocycle or 6,6-membered fused heterocycle are each independently substituted with 0, 1, or 2 R₄; and
R₄ is independently hydrogen, halogen, hydroxyl, C₁-C₃alkyl, C₁-C₃haloalkyl, C₁-C₃alkoxy or an oxo group.

In some embodiments, X₄ is N. In some embodiments, X₃ and X₄ are each N. In some embodiments, X₁ and X₄ are each N. In some embodiments, X₂ and X₄ are each N. In some embodiments, X₁ is CRₐ X₂ is CR_{b}, X₃ is CR_{c}, and X₄ is CR_{d}. In some embodiments, Rₐ, R_{b}, R_{c}, and R_{d} are each independently H, Cl, Br, F, or I. In some embodiments, Rₐ, R_{b}, R_{c}, and R_{d} are each independently H or F. In some embodiments, Rₐ, R_{b}, R_{c}, and R_{d} are each H. In some embodiments, Rₐ is F. In some embodiments, R_{b} is F. In some embodiments, R_{c} is F. In some embodiments, R_{d} is F. In some embodiments, R₁ is H. In some embodiments, R₁ is —CH₃, C₁-C₅ alkyl, or a deuterated C₁-C₅ alkyl. In some embodiments, R₁ is —CD₃. In some embodiments, R₁ is —CH₂—CF₃, —CH₂CH₃, or —CH(CH₃)₂. In some embodiments, R₁ is —CH₂—CH₃. In some embodiments, R₁ is

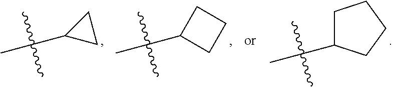

In some embodiments, each R₂ is independently H, —CH₃, or —CH₂—CH₃. In some embodiments, each R₂ is independently H or —CH₃. In some embodiments, R₂ is —CH₃. In some embodiments, each R₂ is identical. In some embodiments, each R₂ is different.

In some embodiments, L is a linker of 1 to 10 carbon atoms in length, wherein one or more carbon atoms are optionally replaced by heterocycle, wherein the heterocycle is a monocyclic heterocycle, a fused heterocycle, a spiro heterocycle, or a bridged heterocycle, and wherein the monocyclic heterocycle, the fused heterocycle, the spiro heterocycle, or the bridged heterocycle are each independently substituted with 0 or 1 R₃.

In some embodiments, L is:

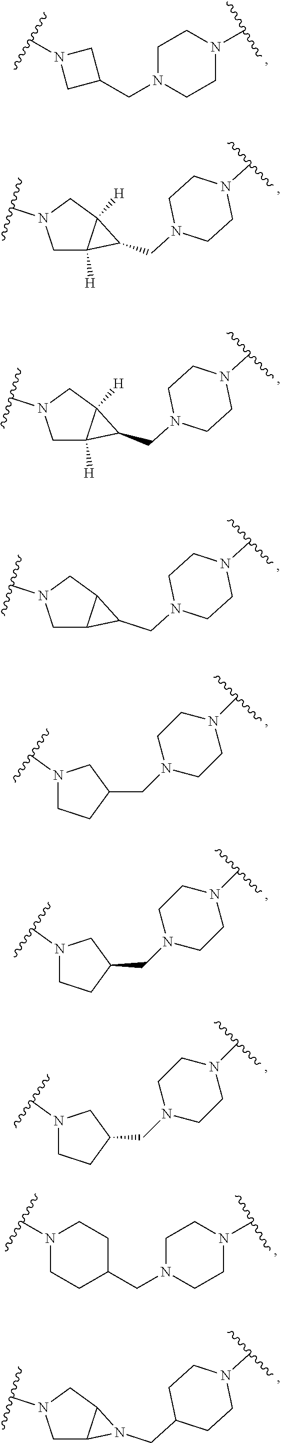

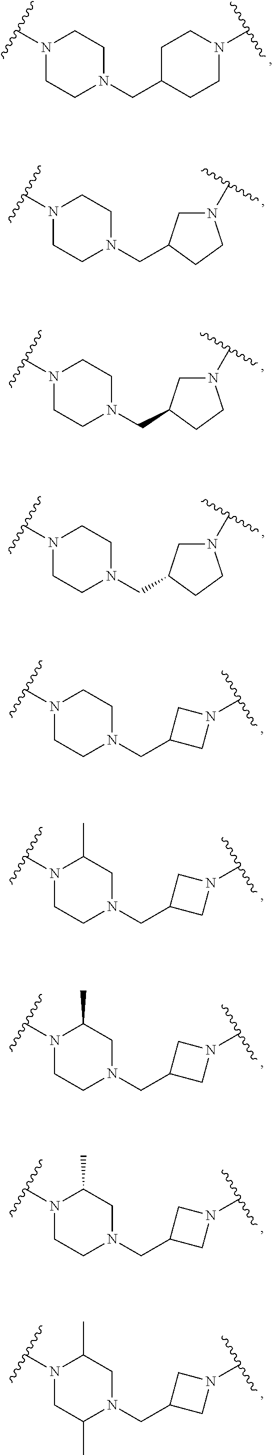

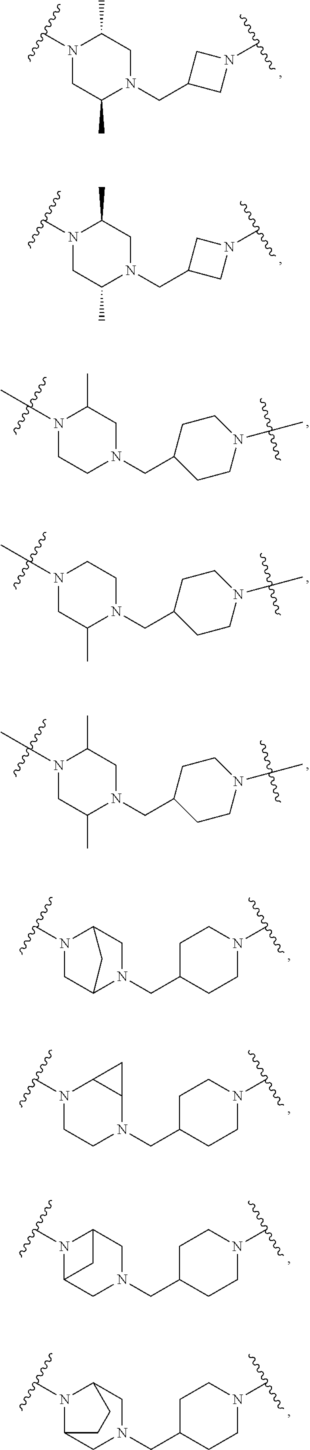

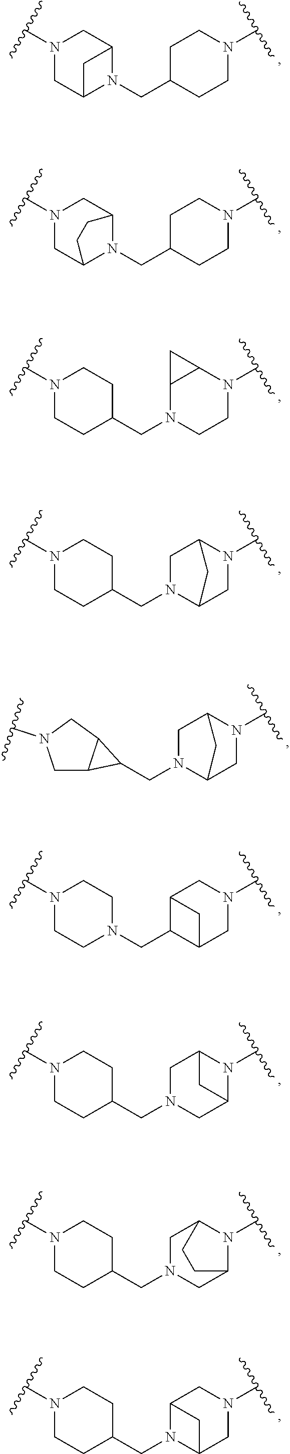

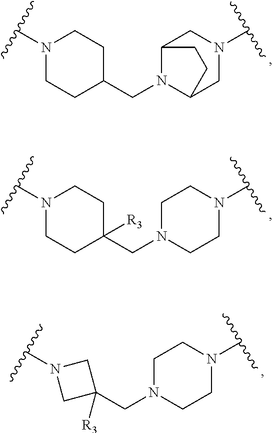

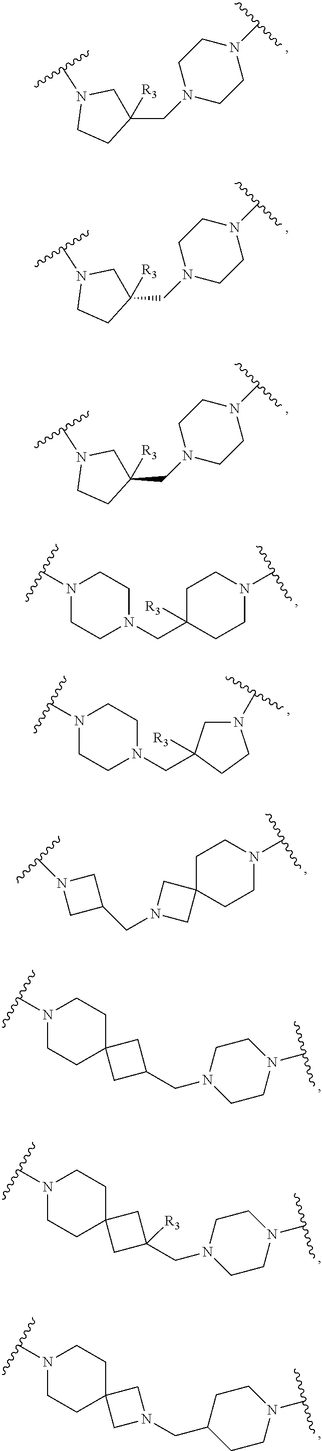

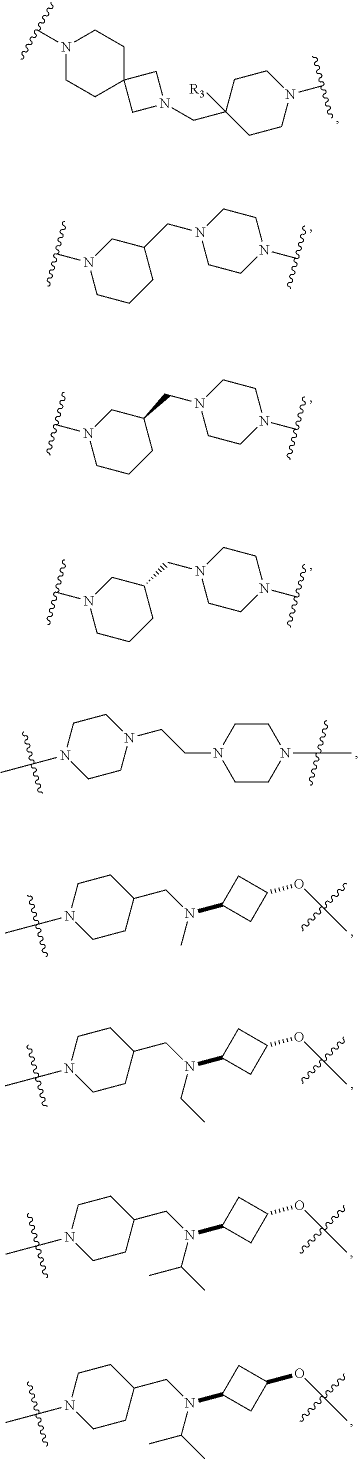

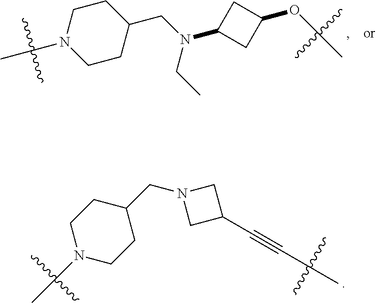 [SMILES: *O[C@H]1C[C@@H](N(CC)CC2CCN(*)CC2)C1 ; *C#CC1CN(CC2CCN(*)CC2)C1]

In some embodiments, L is:

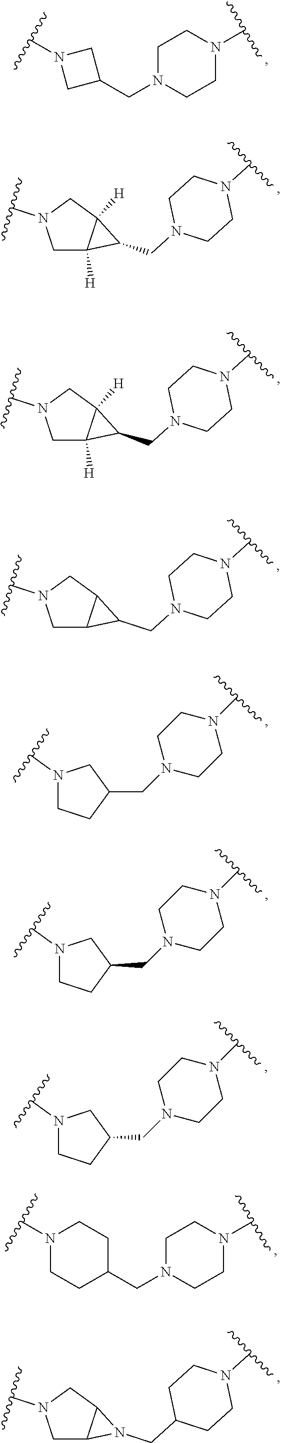

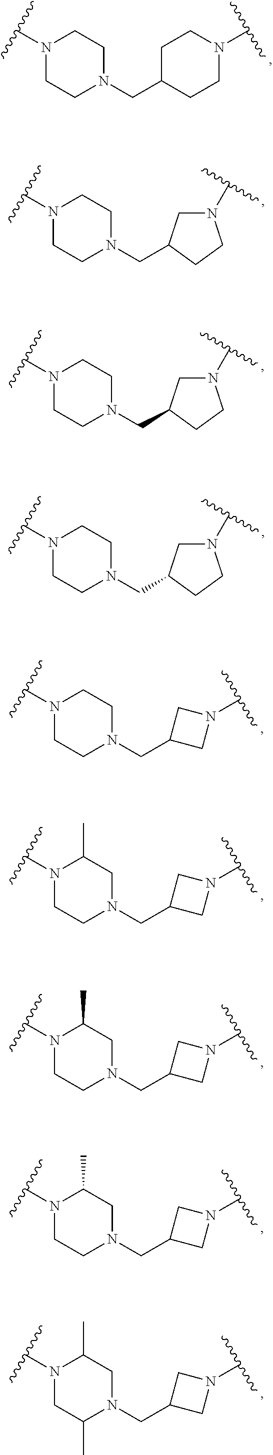

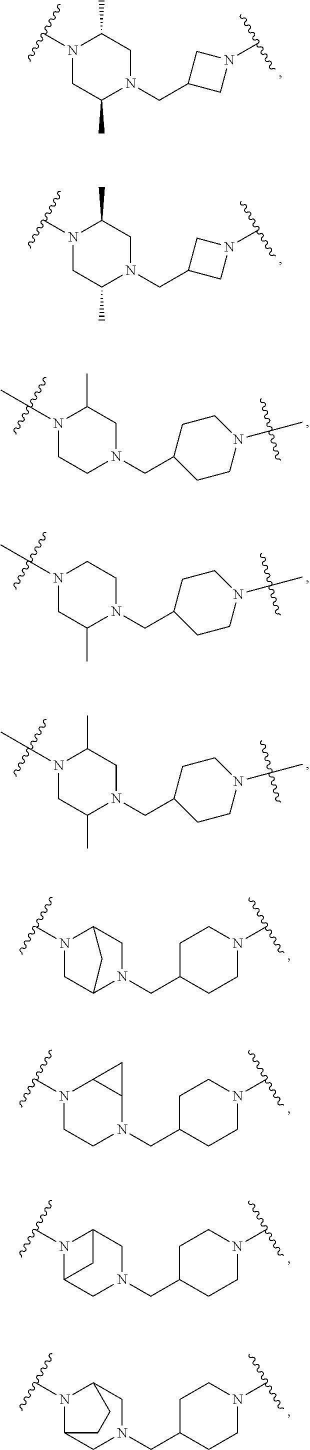

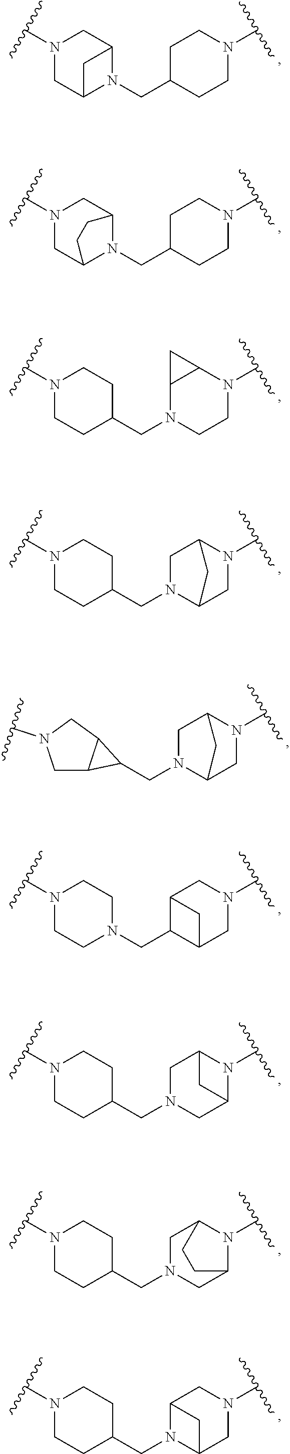

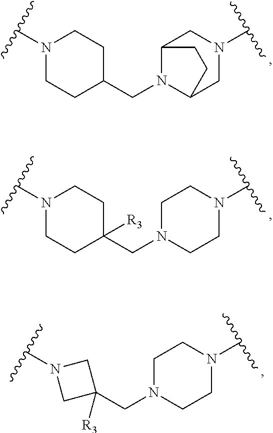

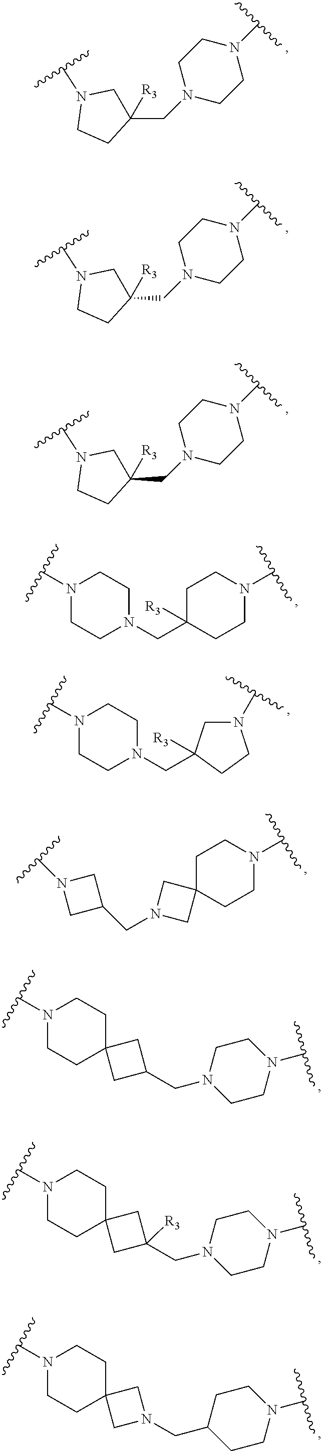

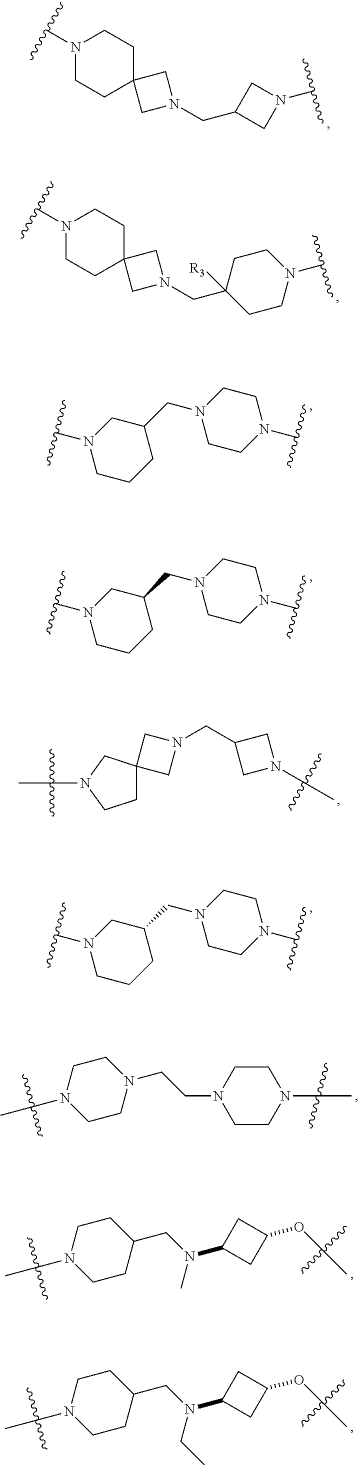

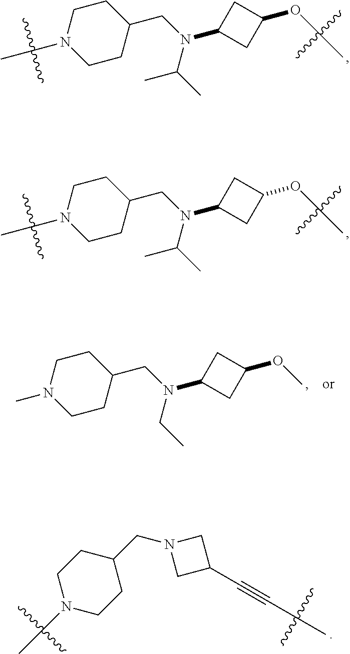

In some embodiments, R₃ is independently hydrogen, halogen, hydroxyl, or C₁-C₃ alkoxyl. In some embodiments, R₃ is

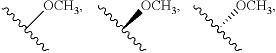

OH, or F. In some embodiments, R₃ is F.

In some embodiments, Q is:

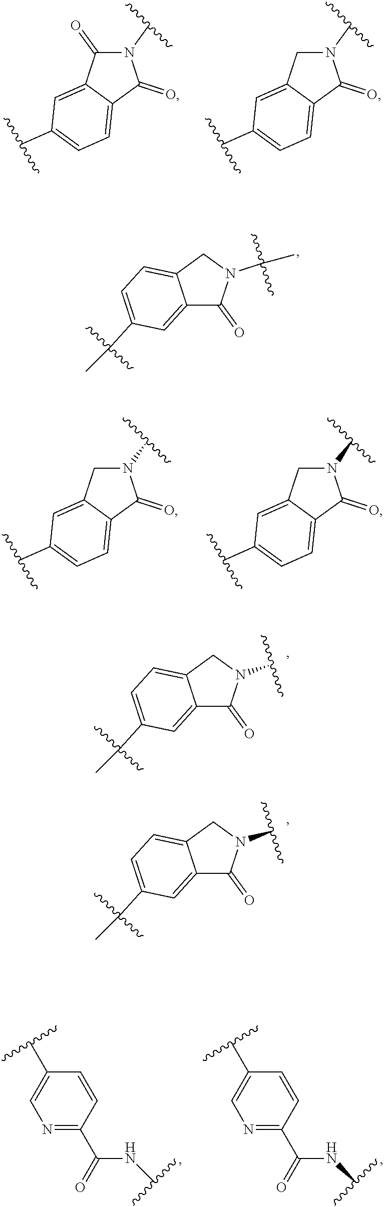

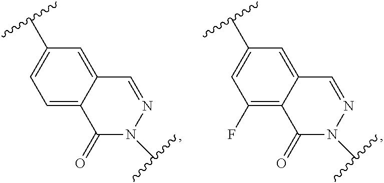 [SMILES: *c1ccc2c(=O)n(*)ncc2c1 ; *c1cc(F)c2c(=O)n(*)ncc2c1]

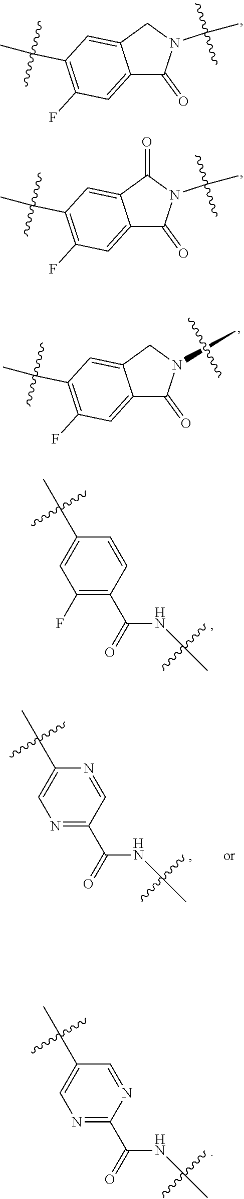

In some embodiments, Q is:

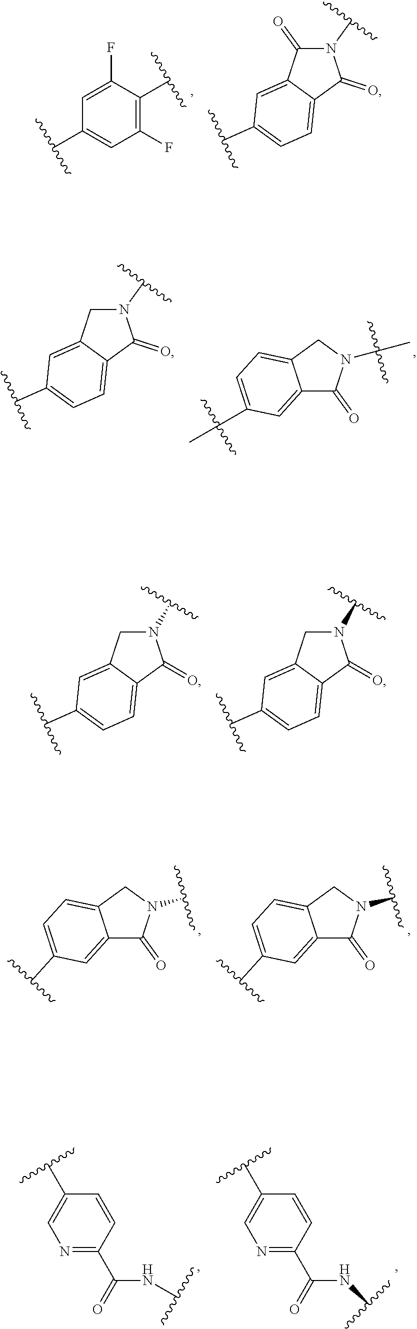

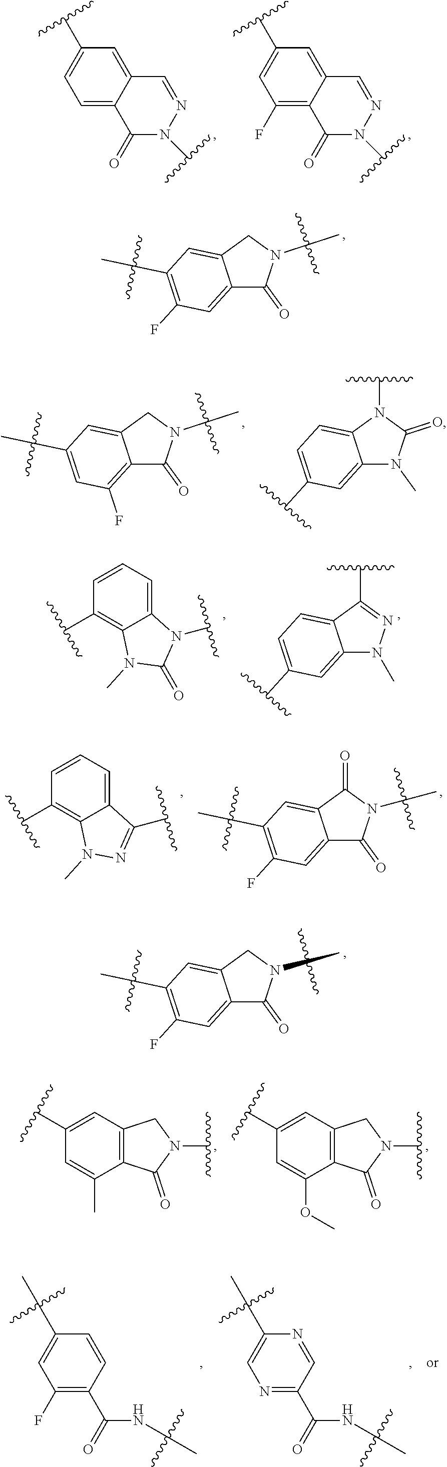

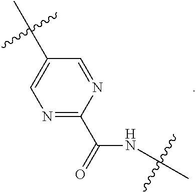 [SMILES: *NC(=O)c1ncc(*)cn1]

In some embodiments, provided herein is a pharmaceutically acceptable salt of a compound of Formula (1A) or (1B). In some embodiments, provided herein is a deuterated derivative of a pharmaceutically acceptable salt of a compound of Formula (1A) or (1B). In some embodiments, provided herein is a compound of Formula (1A) or (1B).

In some embodiments, provided herein is a compound chosen from the compounds listed in Table 2 or a tautomer, stereoisomer or a mixture of stereoisomers, or pharmaceutically acceptable salt, or hydrate, or deuterated derivative thereof.

TABLE 2
Exemplary Compounds of the Present Disclosure

| Compound # | Structure | Name |
|---|---|---|
| 1 | 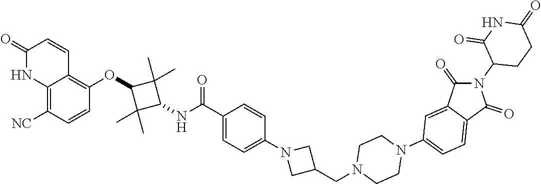 | N-((1,3-trans)-3-((8-cyano-2-oxo- 1,2-dihydroquinolin-5-yl)oxy)- 2,2,4,4-tetramethylcyclobutyl)-4- (3-((4-(2-(2,6-dioxopiperidin-3-yl)- 1,3-dioxoisoindolin-5-yl)piperazin- 1-yl)methyl)azetidin-1- yl)benzamide |
| 2 | 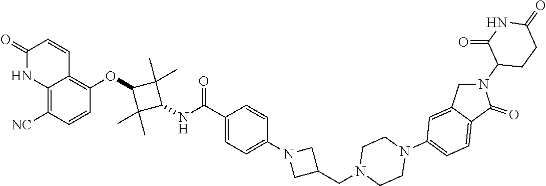 | N-((1,3-trans)-3-((8-cyano-2-oxo- 1,2-dihydroquinolin-5-yl)oxy)- 2,2,4,4-tetramethylcyclobutyl)-4- (3-((4-(2-(2,6-dioxopiperidin-3-yl)- 1-oxoisoindolin-5-yl)piperazin-1- yl)methyl)azetidin-1-yl)benzamide |
| 3 | 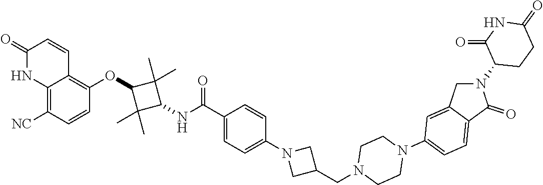 | N-((1,3-trans)-3-((8-cyano-2-oxo- 1,2-dihydroquinolin-5-yl)oxy)- 2,2,4,4-tetramethylcyclobutyl)-4- (3-((4-(2-((S)-2,6-dioxopiperidin- 3-yl)-1-oxoisoindolin-5- yl)piperazin-1-yl)methyl)azetidin- 1-yl)benzamide |
| 4 | 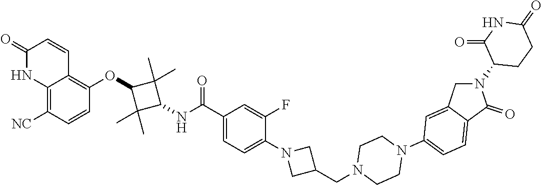 | N-((1,3-trans)-3-((8-cyano-2-oxo- 1,2-dihydroquinolin-5-yl)oxy)- 2,2,4,4-tetramethylcyclobutyl)-4- (3-((4-(2-((S)-2,6-dioxopiperidin- 3-yl)-1-oxoisoindolin-5- yl)piperazin-1-yl)methyl)azetidin- 1-yl)-3-fluorobenzamide |
| 5 | 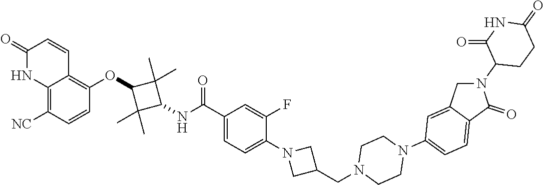 | N-((1,3-trans)-3-((8-cyano-2-oxo- 1,2-dihydroquinolin-5-yl)oxy)- 2,2,4,4-tetramethylcyclobutyl)-4- (3-((4-(2-(2,6-dioxopiperidin-3-yl)- 1-oxoisoindolin-5-yl)piperazin-1- yl)methyl)azetidin-1-yl)-3- fluorobenzamide |
| 6 | 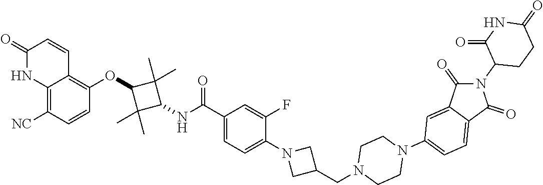 | N-((1,3-trans)-3-((8-cyano-2-oxo- 1,2-dihydroquinolin-5-yl)oxy)- 2,2,4,4-tetramethylcyclobutyl)-4- (3-((4-(2-(2,6-dioxopiperidin-3-yl)- 1,3-dioxoisoindolin-5-yl)piperazin- 1-yl)methyl)azetidin-1-yl)-3- fluorobenzamide |
| 7 | 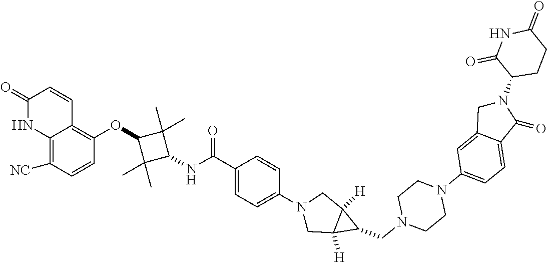 | N-((1,3-trans)-3-((8-cyano-2-oxo- 1,2-dihydroquinolin-5-yl)oxy)- 2,2,4,4-tetramethylcyclobutyl)-4- ((1α,5α,6α)-6-((4-(2-((S)-2,6- dioxopiperidin-3-yl)-1- oxoisoindolin-5-yl)piperazin-1- yl)methyl)-3- azabicyclo[3.1.0]hexan-3- yl)benzamide |
| 8 | 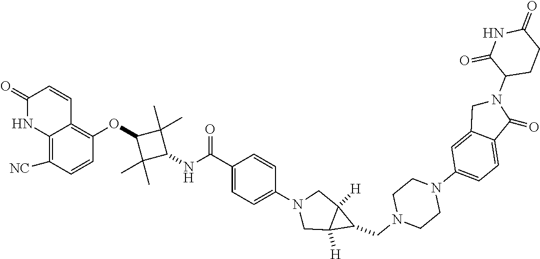 | N-((1,3-trans)-3-((8-cyano-2-oxo- 1,2-dihydroquinolin-5-yl)oxy)- 2,2,4,4-tetramethylcyclobutyl)-4- ((1α,5α,6α)-6-((4-(2-(2,6- dioxopiperidin-3-yl)-1- oxoisoindolin-5-yl)piperazin-1- yl)methyl)-3- azabicyclo[3.1.0]hexan-3- yl)benzamide |
| 9 | 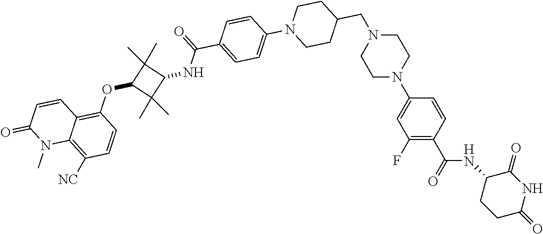 [SMILES: Cn1c(=O)ccc2c(O[C@H]3C(C)(C)[C@H](NC(=O)c4ccc(N5CCC(CN6CCN(c7ccc(C(=O)N[C@H]8CCC(=O)NC8=O)c(F)c7)CC6)CC5)cc4)C3(C)C)ccc(C#N)c21] | N-((1,3-trans)-3-((8-cyano-2-oxo- 1,2-dihydroquinolin-5-yl)oxy)- 2,2,4,4-tetramethylcyclobutyl)-4- ((1α,5α,6α)-6-((4-(2-(2,6- dioxopiperidin-3-yl)-1,3- dioxoisoindolin-5-yl)piperazin-1- yl)methyl)-3- azabicyclo[3.1.0]hexan-3- yl)benzamide |
| 10 | 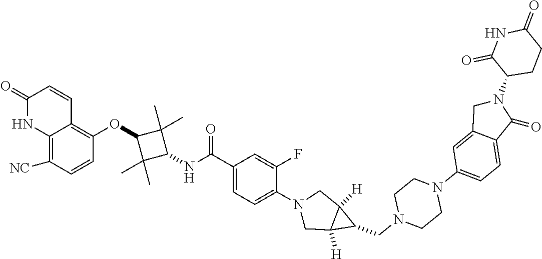 | N-((1,3-trans)-3-((8-cyano-2-oxo- 1,2-dihydroquinolin-5-yl)oxy)- 2,2,4,4-tetramethylcyclobutyl)-4- ((1α,5α,6α)-6-((4-(2-((S)-2,6- dioxopiperidin-3-yl)-1- oxoisoindolin-5-yl)piperazin-1- yl)methyl)-3- azabicyclo[3.1.0]hexan-3-yl)-3- fluorobenzamide |
| 11 | 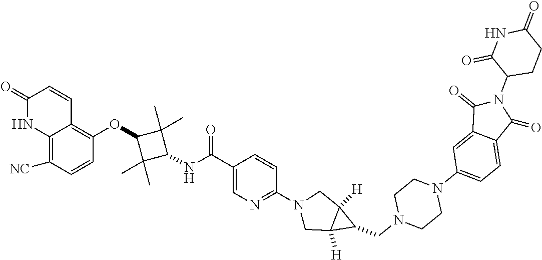 | N-((1,3-trans)-3-((8-cyano-2-oxo- 1,2-dihydroquinolin-5-yl)oxy)- 2,2,4,4-tetramethylcyclobutyl)-6- ((1α,5α,6α)-6-((4-(2-(2,6- dioxopiperidin-3-yl)-1,3- dioxoisoindolin-5-yl)piperazin-1- yl)methyl)-3- azabicyclo[3.1.0]hexan-3- yl)nicotinamide |
| 12 | 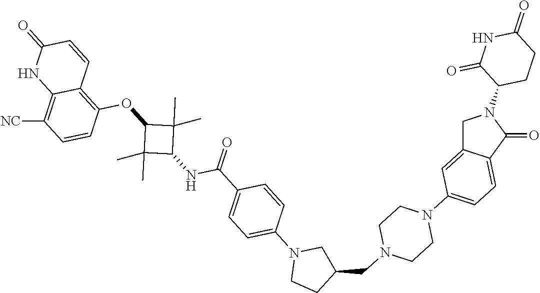 | N-((1,3-trans)-3-((8-cyano-2-oxo- 1,2-dihydroquinolin-5-yl)oxy)- 2,2,4,4-tetramethylcyclobutyl)-4- ((R)-3-((4-(2-((S)-2,6- dioxopiperidin-3-yl)-1- oxoisoindolin-5-yl)piperazin-1- yl)methyl)pyrrolidin-1- yl)benzamide |
| 13 | 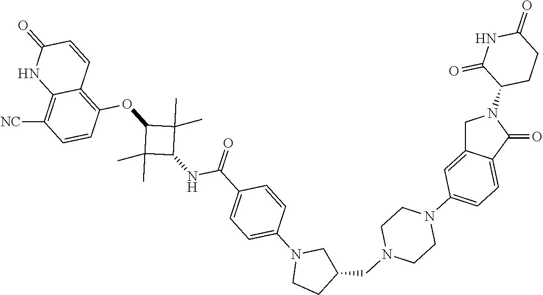 | N-((1,3-trans)-3-((8-cyano-2-oxo- 1,2-dihydroquinolin-5-yl)oxy)- 2,2,4,4-tetramethylcyclobutyl)-4- ((S)-3-((4-(2-((S)-2,6- dioxopiperidin-3-yl)-1- oxoisoindolin-5-yl)piperazin-1- yl)methyl)pyrrolidin-1- yl)benzamide |
| 14 | 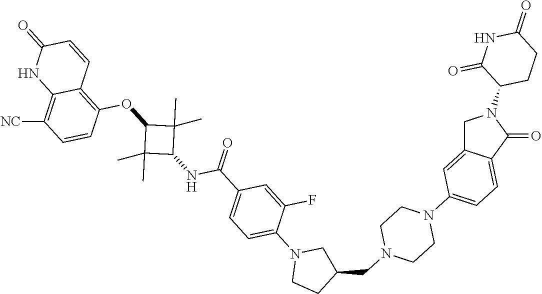 | N-((1,3-trans)-3-((8-cyano-2-oxo- 1,2-dihydroquinolin-5-yl)oxy)- 2,2,4,4-tetramethylcyclobutyl)-4- ((R)-3-((4-(2-((S)-2,6- dioxopiperidin-3-yl)-1- oxoisoindolin-5-yl)piperazin-1- yl)methyl)pyrrolidin-1-yl)-3- fluorobenzamide |
| 15 | 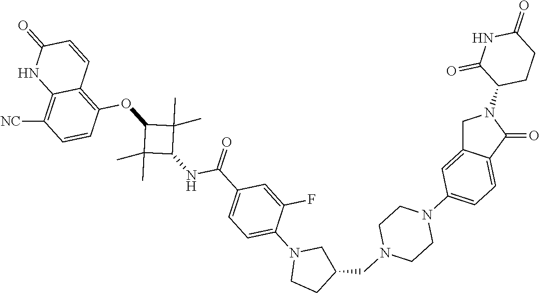 | N-((1r,3S)-3-((8-cyano-2-oxo-1,2- dihydroquinolin-5-yl)oxy)-2,2,4,4- tetramethylcyclobutyl)-4-((S)-3- ((4-(2-((S)-2,6-dioxopiperidin-3- yl)-1-oxoisoindolin-5-yl)piperazin- 1-yl)methyl)pyrrolidin-1-yl)-3- fluorobenzamide |
| 16 | 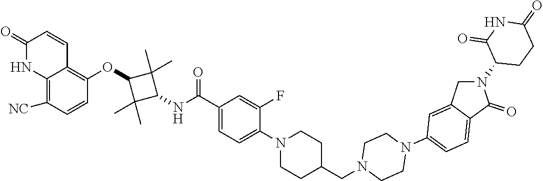 | N-((1,3-trans)-3-((8-cyano-2-oxo- 1,2-dihydroquinolin-5-yl)oxy)- 2,2,4,4-tetramethylcyclobutyl)-4- (4-((4-(2-((S)-2,6-dioxopiperidin- 3-yl)-1-oxoisoindolin-5- yl)piperazin-1-yl)methyl)piperidin- 1-yl)-3-fluorobenzamide |
| 17 | 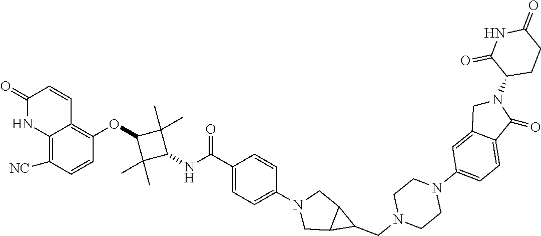 | N-((1,3-trans)-3-((8-cyano-2-oxo- 1,2-dihydroquinolin-5-yl)oxy)- 2,2,4,4-tetramethylcyclobutyl)-4- (6-((4-(2-((S)-2,6-dioxopiperidin- 3-yl)-1-oxoisoindolin-5- yl)piperazin-1-yl)methyl)-3- azabicyclo[3.1.0]hexan-3- yl)benzamide |
| 18 | 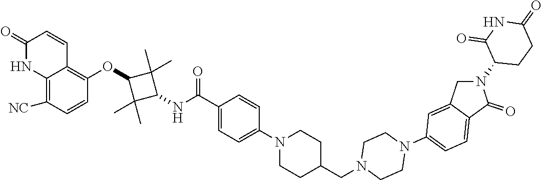 | N-((1,3-trans)-3-((8-cyano-2-oxo- 1,2-dihydroquinolin-5-yl)oxy)- 2,2,4,4-tetramethylcyclobutyl)-4- (4-((4-(2-((S)-2,6-dioxopiperidin- 3-yl)-1-oxoisoindolin-5- yl)piperazin-1-yl)methyl)piperidin- 1-yl)benzamide |
| 19 | 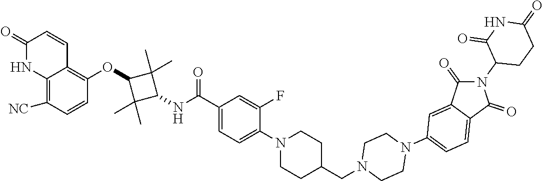 | N-((1,3-trans)-3-((8-cyano-2-oxo- 1,2-dihydroquinolin-5-yl)oxy)- 2,2,4,4-tetramethylcyclobutyl)-4- (4-((4-(2-(2,6-dioxopiperidin-3-yl)- 1,3-dioxoisoindolin-5-yl)piperazin- 1-yl)methyl)piperidin-1-yl)-3- fluorobenzamide |
| 20 | 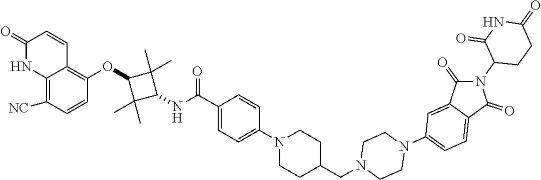 | N-((1,3-trans)-3-((8-cyano-2-oxo- 1,2-dihydroquinolin-5-yl)oxy)- 2,2,4,4-tetramethylcyclobutyl)-4- (4-((4-(2-(2,6-dioxopiperidin-3-yl)- 1,3-dioxoisoindolin-5-yl)piperazin- 1-yl)methyl)piperidin-1- yl)benzamide |
| 21 | 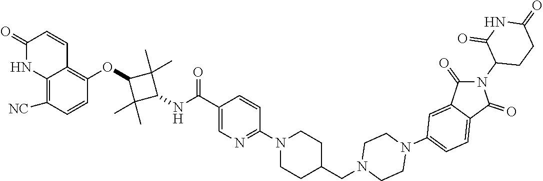 | N-((1,3-trans)-3-((8-cyano-2-oxo- 1,2-dihydroquinolin-5-yl)oxy)- 2,2,4,4-tetramethylcyclobutyl)-6- (4-((4-(2-(2,6-dioxopiperidin-3-yl)- 1,3-dioxoisoindolin-5-yl)piperazin- 1-yl)methyl)piperidin-1- yl)nicotinamide |
| 22 | 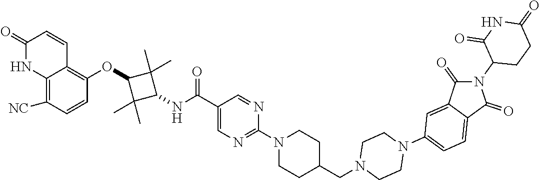 | N-((1,3-trans)-3-((8-cyano-2-oxo- 1,2-dihydroquinolin-5-yl)oxy)- 2,2,4,4-tetramethylcyclobutyl)-2- (4-((4-(2-(2,6-dioxopiperidin-3-yl)- 1,3-dioxoisoindolin-5-yl)piperazin- 1-yl)methyl)piperidin-1- yl)pyrimidine-5-carboxamide |
| 23 | 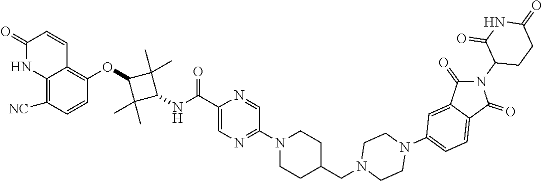 | N-((1,3-trans)-3-((8-cyano-2-oxo- 1,2-dihydroquinolin-5-yl)oxy)- 2,2,4,4-tetramethylcyclobutyl)-5- (4-((4-(2-(2,6-dioxopiperidin-3-yl)- 1,3-dioxoisoindolin-5-yl)piperazin- 1-yl)methyl)piperidin-1- yl)pyrazine-2-carboxamide |
| 24 | 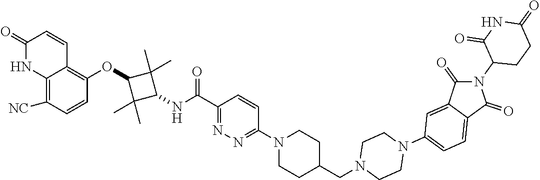 | N-((1,3-trans)-3-((8-cyano-2-oxo- 1,2-dihydroquinolin-5-yl)oxy)- 2,2,4,4-tetramethylcyclobutyl)-6- (4-((4-(2-(2,6-dioxopiperidin-3-yl)- 1,3-dioxoisoindolin-5-yl)piperazin- 1-yl)methyl)piperidin-1- yl)pyridazine-3-carboxamide |
| 25 | 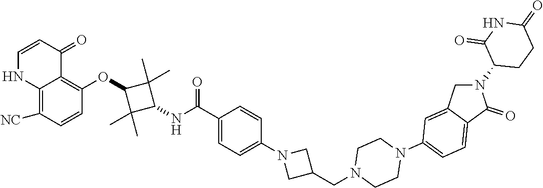 | N-((1,3-trans)-3-((8-cyano-4-oxo- 1,4-dihydroquinolin-5-yl)oxy)- 2,2,4,4-tetramethylcyclobutyl)-4- (3-((4-(2-((S)-2,6-dioxopiperidin- 3-yl)-1-oxoisoindolin-5- yl)piperazin-1-yl)methyl)azetidin- 1-yl)benzamide |
| 26 | 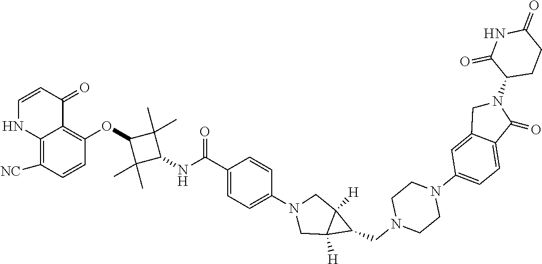 | N-((1,3-trans)-3-((8-cyano-4-oxo- 1,4-dihydroquinolin-5-yl)oxy)- 2,2,4,4-tetramethylcyclobutyl)-4- ((1α,5α,6α)-6-((4-(2-((S)-2,6- dioxopiperidin-3-yl)-1- oxoisoindolin-5-yl)piperazin-1- yl)methyl)-3- azabicyclo[3.1.0]hexan-3- yl)benzamide |
| 27 | 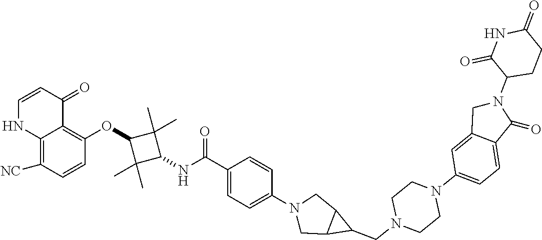 | N-((1,3-trans)-3-((8-cyano-4-oxo- 1,4-dihydroquinolin-5-yl)oxy)- 2,2,4,4-tetramethylcyclobutyl)-4- (6-((4-(2-(2,6-dioxopiperidin-3-yl)- 1-oxoisoindolin-5-yl)piperazin-1- yl)methyl)-3- azabicyclo[3.1.0]hexan-3- yl)benzamide |
| 28 | 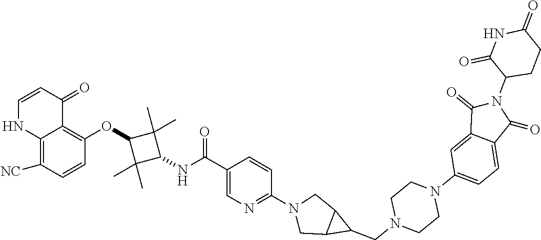 | N-((1,3-trans)-3-((8-cyano-4-oxo- 1,4-dihydroquinolin-5-yl)oxy)- 2,2,4,4-tetramethylcyclobutyl)-6- (6-((4-(2-(2,6-dioxopiperidin-3-yl)- 1,3-dioxoisoindolin-5-yl)piperazin- 1-yl)methyl)-3- azabicyclo[3.1.0]hexan-3- yl)nicotinamide |
| 29 | 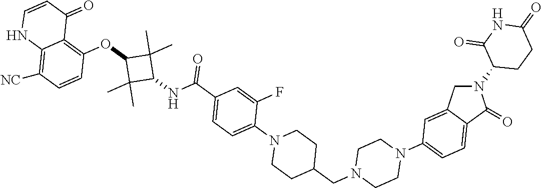 | N-((1,3-trans)-3-((8-cyano-4-oxo- 1,4-dihydroquinolin-5-yl)oxy)- 2,2,4,4-tetramethylcyclobutyl)-4- (4-((4-(2-((S)-2,6-dioxopiperidin- 3-yl)-1-oxoisoindolin-5- yl)piperazin-1-yl)methyl)piperidin- 1-yl)-3-fluorobenzamide |
| 30 | 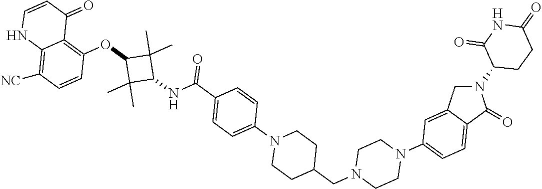 | N-((1,3-trans)-3-((8-cyano-4-oxo- 1,4-dihydroquinolin-5-yl)oxy)- 2,2,4,4-tetramethylcyclobutyl)-4- (4-((4-(2-((S)-2,6-dioxopiperidin- 3-yl)-1-oxoisoindolin-5- yl)piperazin-1-yl)methyl)piperidin- 1-yl)benzamide |
| 31 | 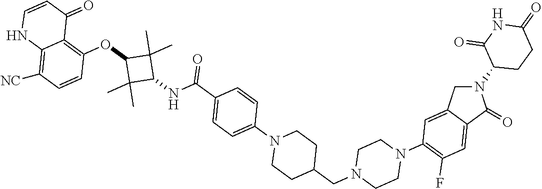 | N-((1,3-trans)-3-((8-cyano-4-oxo- 1,4-dihydroquinolin-5-yl)oxy)- 2,2,4,4-tetramethylcyclobutyl)-4- (4-((4-(2-((S)-2,6-dioxopiperidin- 3-yl)-6-fluoro-1-oxoisoindolin-5- yl)piperazin-1-yl)methyl)piperidin- 1-yl)benzamide |
| 32 | 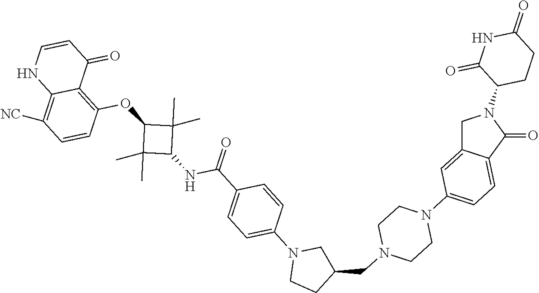 | N-((1,3-trans)-3-((8-cyano-4-oxo- 1,4-dihydroquinolin-5-yl)oxy)- 2,2,4,4-tetramethylcyclobutyl)-4- ((R)-3-((4-(2-((S)-2,6- dioxopiperidin-3-yl)-1- oxoisoindolin-5-yl)piperazin-1- yl)methyl)pyrrolidin-1- yl)benzamide |
| 33 | 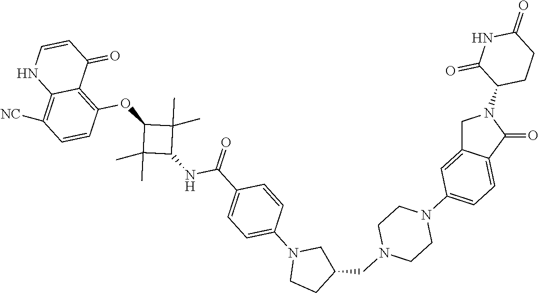 | N-((1,3-trans)-3-((8-cyano-4-oxo- 1,4-dihydroquinolin-5-yl)oxy)- 2,2,4,4-tetramethylcyclobutyl)-4- ((S)-3-((4-(2-((S)-2,6- dioxopiperidin-3-yl)-1- oxoisoindolin-5-yl)piperazin-1- yl)methyl)pyrrolidin-1- yl)benzamide |
| 34 | 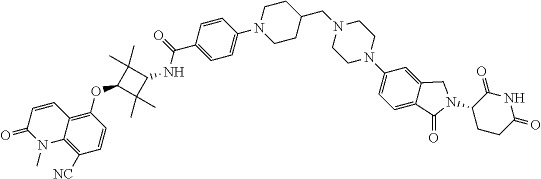 | N-((1,3-trans)-3-((8-cyano-1- methyl-2-oxo-1,2-dihydroquinolin- 5-yl)oxy)-2,2,4,4- tetramethylcyclobutyl)-4-(4-((4-((2- ((S)-2,6-dioxopiperidin-3-yl)-1- oxoisoindolin-5-yl)piperazin-1- yl)methyl)piperidin-1- yl)benzamide |
| 35 | 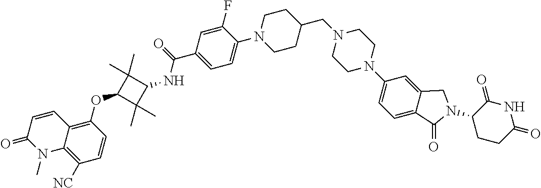 | N-((1,3-trans)-3-((8-cyano-1- methyl-2-oxo-1,2-dihydroquinolin- 5-yl)oxy)-2,2,4,4- tetramethylcyclobutyl)-4-(4-((4-(2- ((S)-2,6-dioxopiperidin-3-yl)-1- oxoisoindolin-5-yl)piperazin-1- yl)methyl)piperidin-1-yl)-3- fluorobenzamide |
| 36 | 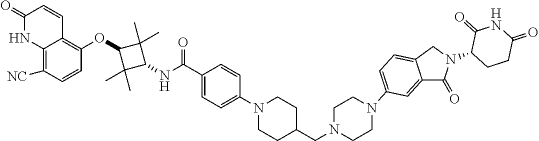 | N-((1,3-trans)-3-((8-cyano-2-oxo- 1,2-dihydroquinolin-5-yl)oxy)- 2,2,4,4-tetramethylcyclobutyl)-4- (4-((4-(2-((S)-2,6-dioxopiperidin- 3-yl)-3-oxoisoindolin-5- yl)piperazin-1-yl)methyl)piperidin- 1-yl)benzamide |
| 37 | 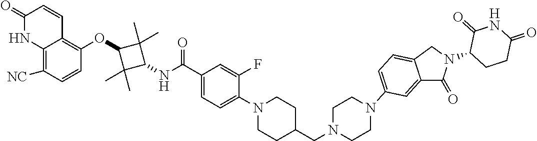 | N-((1,3-trans)-3-((8-cyano-2-oxo- 1,2-dihydroquinolin-5-yl)oxy)- 2,2,4,4-tetramethylcyclobutyl)-4- (4-((4-(2-((S)-2,6-dioxopiperidin- 3-yl)-3-oxoisoindolin-5- yl)piperazin-1-yl)methyl)piperidin- 1-yl)-3-fluorobenzamide |
| 38 | 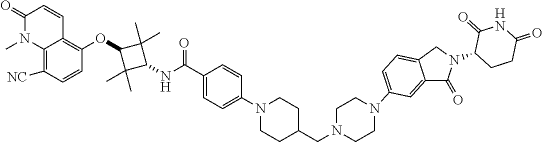 | N-((1,3-trans)-3-((8-cyano-1- methyl-2-oxo-1,2-dihydroquinolin- 5-yl)oxy)-2,2,4,4- tetramethylcyclobutyl)-4-(4-((4-(2- ((S)-2,6-dioxopiperidin-3-yl)-3- oxoisoindolin-5-yl)piperazin-1- yl)methyl)piperidin-1- yl)benzamide |
| 39 | 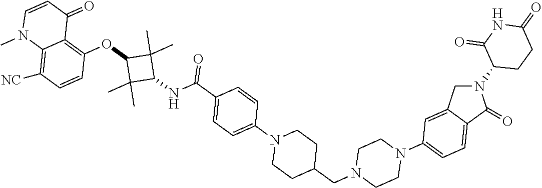 | N-((1,3-trans)-3-((8-cyano-1- methyl-4-oxo-1,4-dihydroquinolin- 5-yl)oxy)-2,2,4,4- tetramethylcyclobutyl)-4-(4-((4-(2- ((S)-2,6-dioxopiperidin-3-yl)-1- oxoisoindolin-5-yl)piperazin-1- yl)methyl)piperidin-1- yl)benzamide |
| 40 | 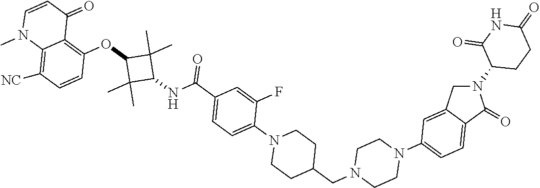 | N-((1,3-trans)-3-((8-cyano-1- methyl-4-oxo-1,4-dihydroquinolin- 5-yl)oxy)-2,2,4,4- tetramethylcyclobutyl)-4-(4-((4-(2- ((S)-2,6-dioxopiperidin-3-yl)-1- oxoisoindolin-5-yl)piperazin-1- yl)methyl)piperidin-1-yl)-3- fluorobenzamide |
| 41 | 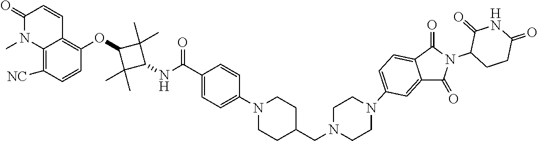 | N-((1,3-trans)-3-((8-cyano-1- methyl-2-oxo-1,2-dihydroquinolin- 5-yl)oxy)-2,2,4,4- tetramethylcyclobutyl)-4-(4-((4-(2- (2,6-dioxopiperidin-3-yl)-1,3- dioxoisoindolin-5-yl)piperazin-1- yl)methyl)piperidin-1- yl)benzamide |
| 42 | 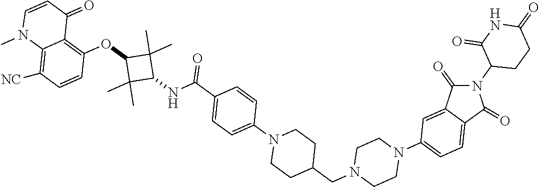 | N-((1,3-trans)-3-((8-cyano-1- methyl-4-oxo-1,4-dihydroquinolin- 5-yl)oxy)-2,2,4,4- tetramethylcyclobutyl)-4-(4-((4-(2- (2,6-dioxopiperidin-3-yl)-1,3- dioxoisoindolin-5-yl)piperazin-1- yl)methyl)piperidin-1- yl)benzamide |
| 43 | 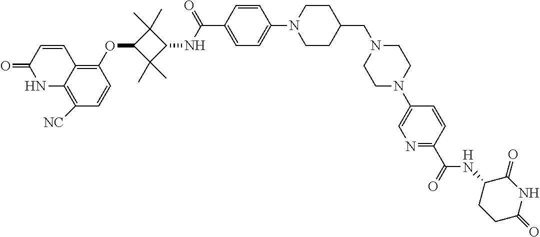 | 5-(4-((1-(4-(((1,3-trans)-3-((8- cyano-2-oxo-1,2-dihydroquinolin- 5-yl)oxy)-2,2,4,4- tetramethylcyclobutyl)carbamoyl) phenyl)piperidin-4- yl)methyl)piperazin-1-yl)-N-((S)- 2,6-dioxopiperidin-3- yl)picolinamide |
| 44 | 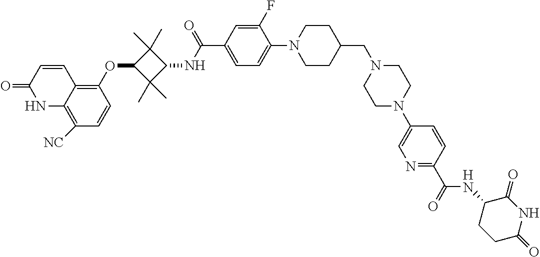 | 5-(4-((1-(4-(((1,3-trans)-3-((8- cyano-2-oxo-1,2-dihydroquinolin- 5-yl)oxy)-2,2,4,4- tetramethylcyclobutyl)carbamoyl)- 2-fluorophenyl)piperidin-4- yl)methyl)piperazin-1-yl)-N-((S)- 2,6-dioxopiperidin-3- yl)picolinamide |
| 45 | 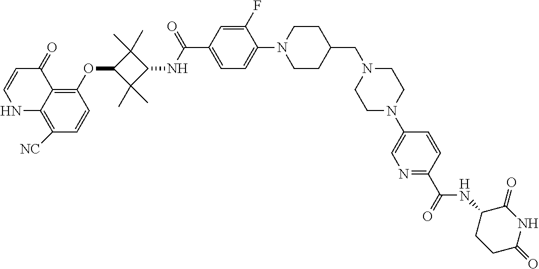 | 5-(4-((1-(4-(((1,3-trans)-3-((8- cyano-4-oxo-1,4-dihydroquinolin- 5-yl)oxy)-2,2,4,4- tetramethylcyclobutyl)carbamoyl)- 2-fluorophenyl)piperidin-4- yl)methyl)piperazin-1-yl)-N-((S)- 2,6-dioxopiperidin-3- yl)picolinamide |
| 46 | 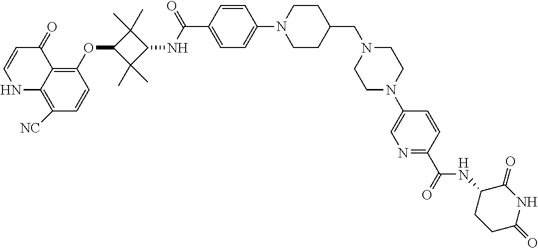 | 5-(4-((1-(4-(((1,3-trans)-3-((8- cyano-4-oxo-1,4-dihydroquinolin- 5-yl)oxy)-2,2,4,4- tetramethylcyclobutyl)carbamoyl) phenyl)piperidin-4- yl)methyl)piperazin-1-yl)-N-((S)- 2,6-dioxopiperidin-3- yl)picolinamide |
| 47 | 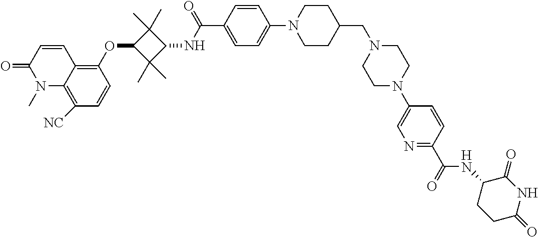 | 5-(4-((1-(4-(((1,3-trans)-3-((8- cyano-1-methyl-2-oxo-1,2- dihydroquinolin-5-yl)oxy)-2,2,4,4- tetramethylcyclobutyl)carbamoyl) phenyl)piperidin-4- yl)methyl)piperazin-1-yl)-N-((S)- 2,6-dioxopiperidin-3- yl)picolinamide |
| 48 | 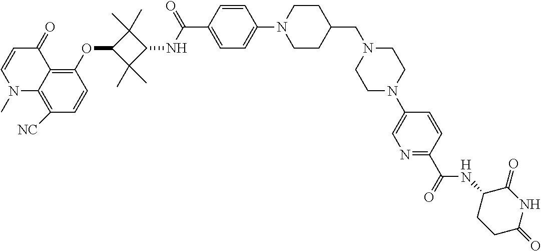 | 5-(4-((1-(4-(((1,3-trans)-3-((8- cyano-1-methyl-4-oxo-1,4- dihydroquinolin-5-yl)oxy)-2,2,4,4- tetramethylcyclobutyl)carbamoyl) phenyl)piperidin-4- yl)methyl)piperazin-1-yl)-N-((S)- 2,6-dioxopiperidin-3- yl)picolinamide |
| 49 | 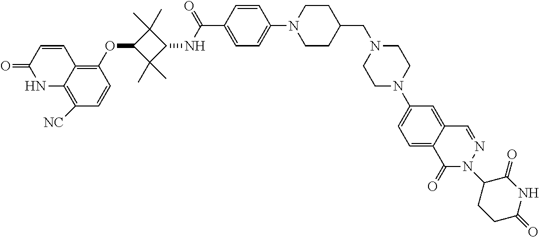 | N-((1,3-trans)-3-((8-cyano-2-oxo- 1,2-dihydroquinolin-5-yl)oxy)- 2,2,4,4-tetramethylcyclobutyl)-4- (4-((4-(2-(2,6-dioxopiperidin-3-yl)- 1-oxo-1,2-dihydrophthalazin-6- yl)piperazin-1-yl)methyl)piperidin- 1-yl)benzamide |
| 50 | 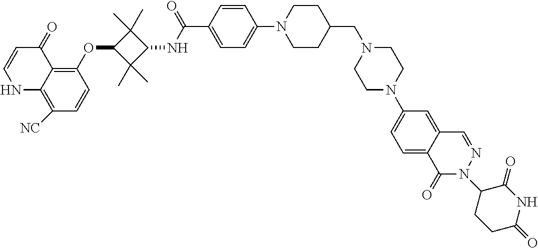 | N-((1,3-trans)-3-((8-cyano-4-oxo- 1,4-dihydroquinolin-5-yl)oxy)- 2,2,4,4-tetramethylcyclobutyl)-4- (4-((4-(2-(2,6-dioxopiperidin-3-yl)- 1-oxo-1,2-dihydrophthalazin-6- yl)piperazin-1-yl)methyl)piperidin- 1-yl)benzamide |
| 51 | 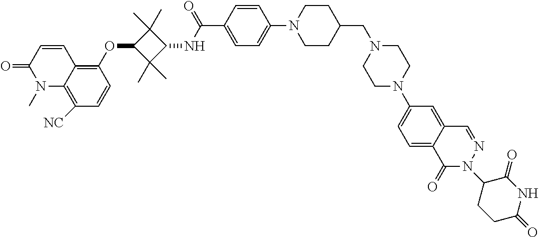 | N-((1,3-trans)-3-((8-cyano-1- methyl-2-oxo-1,2-dihydroquinolin- 5-yl)oxy)-2,2,4,4- tetramethylcyclobutyl)-4-(4-((4-(2- (2,6-dioxopiperidin-3-yl)-1-oxo- 1,2-dihydrophthalazin-6- yl)piperazin-1-yl)methyl)piperidin- 1-yl)benzamide |
| 52 | 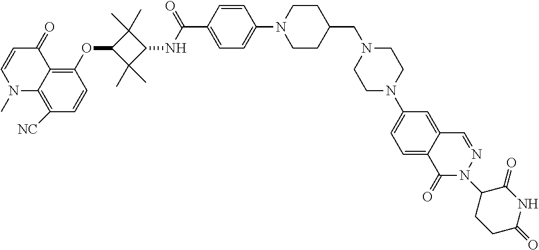 | N-((1,3-trans)-3-((8-cyano-1- methyl-4-oxo-1,4-dihydroquinolin- 5-yl)oxy)-2,2,4,4- tetramethylcyclobutyl)-4-(4-((4-(2- (2,6-dioxopiperidin-3-yl)-1-oxo- 1,2-dihydrophthalazin-6- yl)piperazin-1-yl)methyl)piperidin- 1-yl)benzamide |
| 53 | 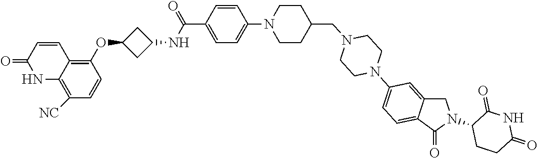 | N-((1,3-trans)-3-((8-cyano-2-oxo- 1,2-dihydroquinolin-5- yl)oxy)cyclobutyl)-4-(4-((4-(2-((S)- 2,6-dioxopiperidin-3-yl)-1- oxoisoindolin-5-yl)piperazin-1- yl)methyl)piperidin-1- yl)benzamide |
| 54 | 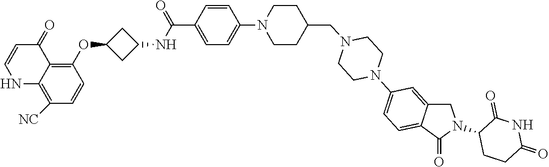 | N-((1,3-trans)-3-((8-cyano-4-oxo- 1,4-dihydroquinolin-5- yl)oxy)cyclobutyl)-4-(4-((4-(2-((S)- 2,6-dioxopiperidin-3-yl)-1- oxoisoindolin-5-yl)piperazin-1- yl)methyl)piperidin-1- yl)benzamide |
| 55 | 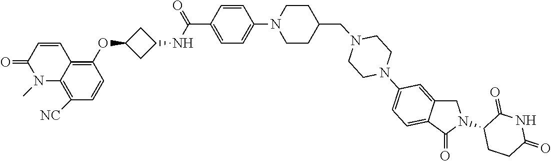 | N-((1,3-trans)-3-((8-cyano-1- methyl-2-oxo-1,2-dihydroquinolin- 5-yl)oxy)cyclobutyl)-4-(4-((4-(2- ((S)-2,6-dioxopiperidin-3-yl)-1- oxoisoindolin-5-yl)piperazin-1- yl)methyl)piperidin-1- yl)benzamide |
| 56 | 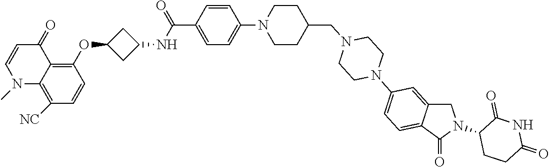 | N-((1,3-trans)-3-((8-cyano-1- methyl-4-oxo-1,4-dihydroquinolin- 5-yl)oxy)cyclobutyl)-4-(4-((4-(2- ((S)-2,6-dioxopiperidin-3-yl)-1- oxoisoindolin-5-yl)piperazin-1- yl)methyl)piperidin-1- yl)benzamide |
| 57 | 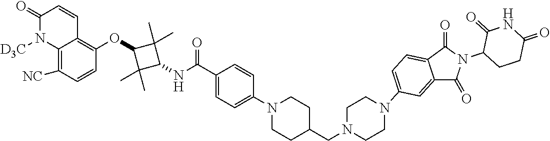 | N-((1,3-trans)-3-((8-cyano-1- (methyl-d3)-2-oxo-1,2- dihydroquinolin-5-yl)oxy)-2,2,4,4- tetramethylcyclobutyl)-4-(4-((4-(2- (2,6-dioxopiperidin-3-yl)-1,3- dioxoisoindolin-5-yl)piperazin-1- yl)methyl)piperidin-1- yl)benzamide |
| 58 | 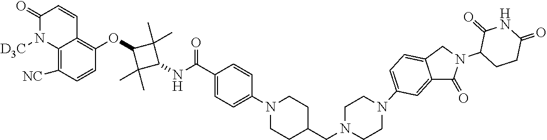 | N-((1,3-trans)-3-((8-cyano-1- (methyl-d3)-2-oxo-1,2- dihydroquinolin-5-yl)oxy)-2,2,4,4- tetramethylcyclobutyl)-4-(4-((4-(2- (2,6-dioxopiperidin-3-yl)-3- oxoisoindolin-5-yl)piperazin-1- yl)methyl)piperidin-1- yl)benzamide |
| 59 | 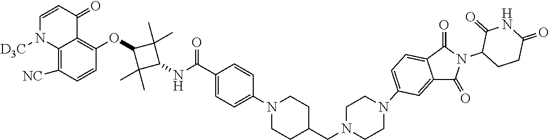 | N-((1,3-trans)-3-((8-cyano-1- (methyl-d3)-4-oxo-1,4- dihydroquinolin-5-yl)oxy)-2,2,4,4- tetramethylcyclobutyl)-4-(4-((4-(2- (2,6-dioxopiperidin-3-yl)-1,3- dioxoisoindolin-5-yl)piperazin-1- yl)methyl)piperidin-1- yl)benzamide |
| 60 | 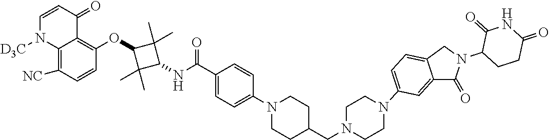 | N-((1,3-trans)-3-((8-cyano-1- (methyl-d3)-4-oxo-1,4- dihydroquinolin-5-yl)oxy)-2,2,4,4- tetramethylcyclobutyl)-4-(4-((4-(2- (2,6-dioxopiperidin-3-yl)-3- oxoisoindolin-5-yl)piperazin-1- yl)methyl)piperidin-1- yl)benzamide |
| 61 | 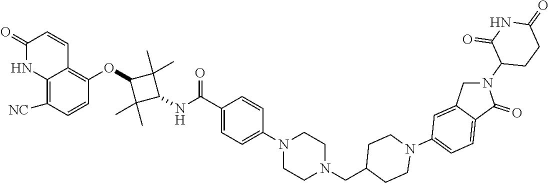 | N-((1,3-trans)-3-((8-cyano-2-oxo- 1,2-dihydroquinolin-5-yl)oxy)- 2,2,4,4-tetramethylcyclobutyl)-4- (4-((1-(2-(2,6-dioxopiperidin-3-yl)- 1-oxoisoindolin-5-yl)piperidin-4- yl)methyl)piperazin-1- yl)benzamide |
| 62 | 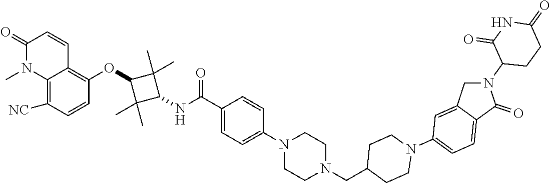 | N-((1,3-trans)-3-((8-cyano-1- methyl-2-oxo-1,2-dihydroquinolin- 5-yl)oxy)-2,2,4,4- tetramethylcyclobutyl)-4-(4-((1-(2- (2,6-dioxopiperidin-3-yl)-1- oxoisoindolin-5-yl)piperidin-4- yl)methyl)piperazin-1- yl)benzamide |
| 63 | 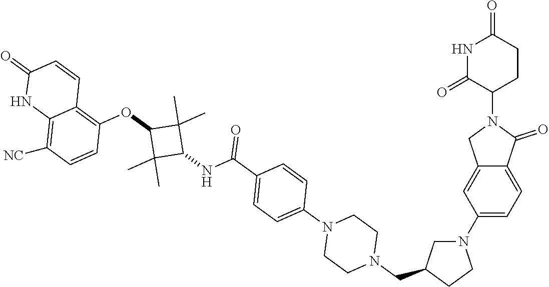 | N-((1,3-trans)-3-((8-cyano-2-oxo- 1,2-dihydroquinolin-5-yl)oxy)- 2,2,4,4-tetramethylcyclobutyl)-4- (4-(((3S)-1-(2-(2,6-dioxopiperidin- 3-yl)-1-oxoisoindolin-5- yl)pyrrolidin-3-yl)methyl) piperazin-1-yl)benzamide |
| 64 | 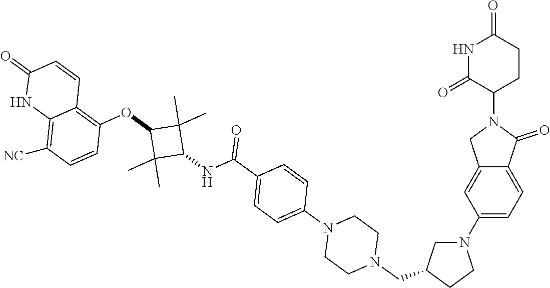 | N-((1,3-trans)-3-((8-cyano-2-oxo- 1,2-dihydroquinolin-5-yl)oxy)- 2,2,4,4-tetramethylcyclobutyl)-4- (4-(((3R)-1-(2-(2,6-dioxopiperidin- 3-yl)-1-oxoisoindolin-5- yl)pyrrolidin-3-yl)methyl) piperazin-1-yl)benzamide |
| 65 | 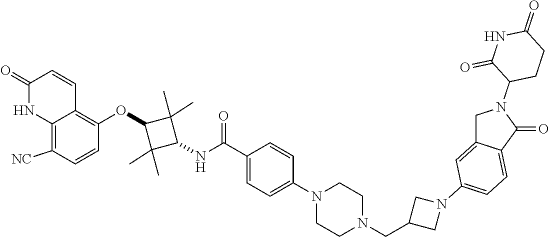 | N-((1,3-trans)-3-((8-cyano-2-oxo- 1,2-dihydroquinolin-5-yl)oxy)- 2,2,4,4-tetramethylcyclobutyl)-4- (4-((1-(2-(2,6-dioxopiperidin-3-yl)- 1-oxoisoindolin-5-yl)azetidin-3- yl)methyl)piperazin-1- yl)benzamide |
| 66 | 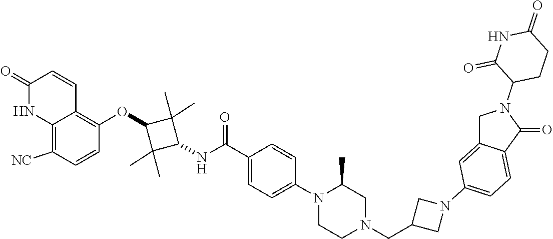 | N-((1,3-trans)-3-((8-cyano-2-oxo- 1,2-dihydroquinolin-5-yl)oxy)- 2,2,4,4-tetramethylcyclobutyl)-4- ((2S)-4-((1-(2-(2,6-dioxopiperidin- 3-yl)-1-oxoisoindolin-5-yl)azetidin- 3-yl)methyl)-2-methylpiperazin-1- yl)benzamide |
| 67 | 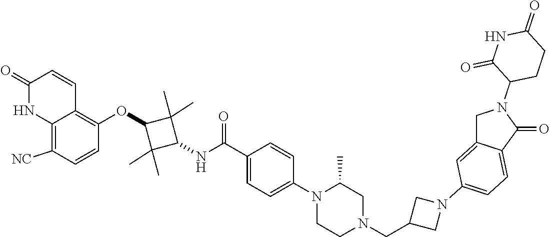 | N-((1,3-trans)-3-((8-cyano-2-oxo- 1,2-dihydroquinolin-5-yl)oxy)- 2,2,4,4-tetramethylcyclobutyl)-4- ((2R)-4-((1-(2-(2,6-dioxopiperidin- 3-yl)-1-oxoisoindolin-5-yl)azetidin- 3-yl)methyl)-2-methylpiperazin-1- yl)benzamide |
| 68 | 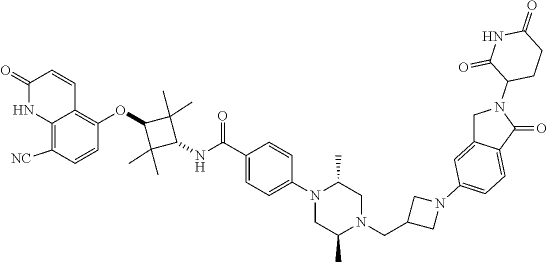 | N-((1,3-trans)-3-((8-cyano-2-oxo- 1,2-dihydroquinolin-5-yl)oxy)- 2,2,4,4-tetramethylcyclobutyl)-4- ((2R,5S)-4-((1-(2-(2,6- dioxopiperidin-3-yl)-1- oxoisoindolin-5-yl)azetidin-3- yl)methyl)-2,5-dimethylpiperazin- 1-yl)benzamide |
| 69 | 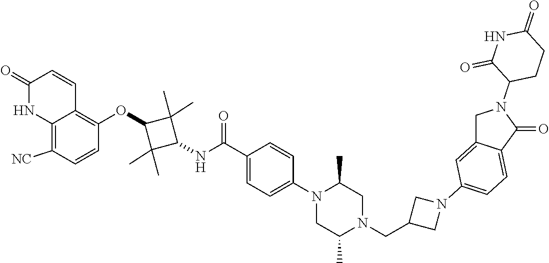 | N-((1,3-trans)-3-((8-cyano-2-oxo- 1,2-dihydroquinolin-5-yl)oxy)- 2,2,4,4-tetramethylcyclobutyl)-4- ((2S,5R)-4-((1-(2-(2,6- dioxopiperidin-3-yl)-1- oxoisoindolin-5-yl)azetidin-3- yl)methyl)-2,5-dimethylpiperazin- 1-yl)benzamide |
| 70 | 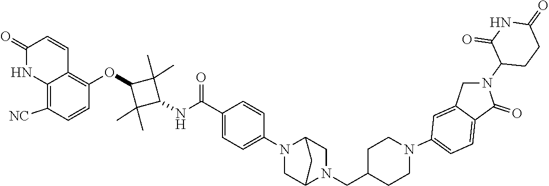 | N-((1,3-trans)-3-((8-cyano-2-oxo- 1,2-dihydroquinolin-5-yl)oxy)- 2,2,4,4-tetramethylcyclobutyl)-4- (5-((1-(2-(2,6-dioxopiperidin-3-yl)- 1-oxoisoindolin-5-yl)piperidin-4- yl)methyl)-2,5- diazabicyclo[2.2.1]heptan-2- yl)benzamide |
| 71 | 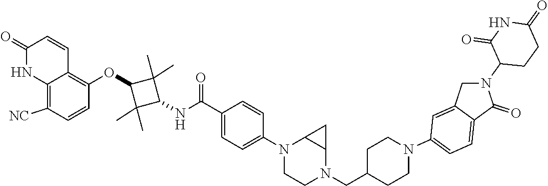 | N-((1,3-trans)-3-((8-cyano-2-oxo- 1,2-dihydroquinolin-5-yl)oxy)- 2,2,4,4-tetramethylcyclobutyl)-4- (5-((1-(2-(2,6-dioxopiperidin-3-yl)- 1-oxoisoindolin-5-yl)piperidin-4- yl)methyl)-2,5- diazabicyclo[4.1.0]heptan-2- yl)benzamide |
| 72 | 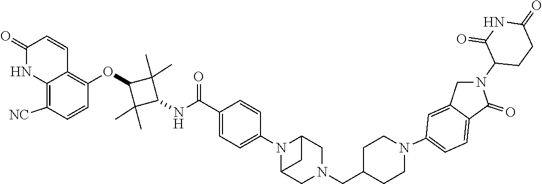 | N-((1,3-trans)-3-((8-cyano-2-oxo- 1,2-dihydroquinolin-5-yl)oxy)- 2,2,4,4-tetramethylcyclobutyl)-4- (3-((1-(2-(2,6-dioxopiperidin-3-yl)- 1-oxoisoindolin-5-yl)piperidin-4- yl)methyl)-3,6- diazabicyclo[3.1.1]heptan-6- yl)benzamide |
| 73 | 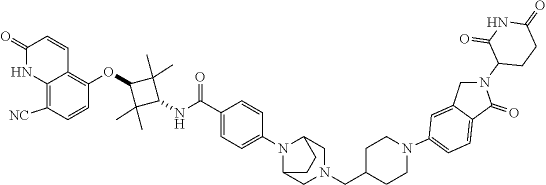 | N-((1,3-trans)-3-((8-cyano-2-oxo- 1,2-dihydroquinolin-5-yl)oxy)- 2,2,4,4-tetramethylcyclobutyl)-4- (3-((1-(2-(2,6-dioxopiperidin-3-yl)- 1-oxoisoindolin-5-yl)piperidin-4- yl)methyl)-3,8- diazabicyclo[3.2.1]octan-8- yl)benzamide |
| 74 | 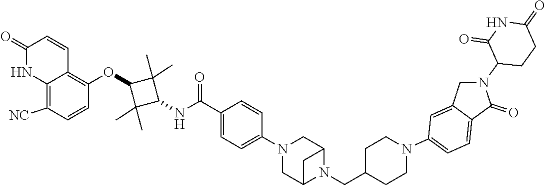 | N-((1,3-trans)-3-((8-cyano-2-oxo- 1,2-dihydroquinolin-5-yl)oxy)- 2,2,4,4-tetramethylcyclobutyl)-4- (6-((1-(2-(2,6-dioxopiperidin-3-yl)- 1-oxoisoindolin-5-yl)piperidin-4- yl)methyl)-3,6- diazabicyclo[3.1.1]heptan-3- yl)benzamide |
| 75 | 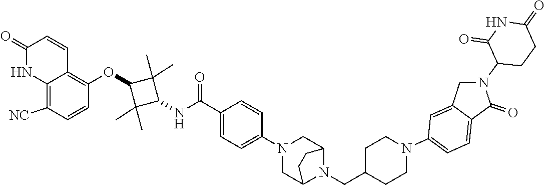 | N-((1,3-trans)-3-((8-cyano-2-oxo- 1,2-dihydroquinolin-5-yl)oxy)- 2,2,4,4-tetramethylcyclobutyl)-4- (8-((1-(2-(2,6-dioxopiperidin-3-yl)- 1-oxoisoindolin-5-yl)piperidin-4- yl)methyl)-3,8- diazabicyclo[3.2.1]octan-3- yl)benzamide |
| 76 | 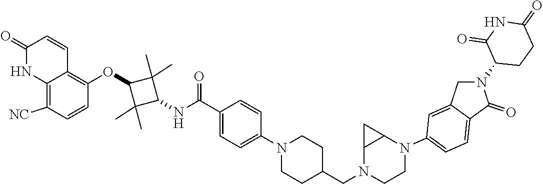 | N-((1,3-trans)-3-((8-cyano-2-oxo- 1,2-dihydroquinolin-5-yl)oxy)- 2,2,4,4-tetramethylcyclobutyl)-4- (4-((5-(2-((S)-2,6-dioxopiperidin- 3-yl)-1-oxoisoindolin-5-yl)-2,5- diazabicyclo[4.1.0]heptan-2- yl)methyl)piperidin-1- yl)benzamide |
| 77 | 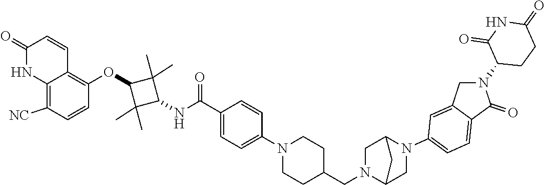 | N-((1,3-trans)-3-((8-cyano-2-oxo- 1,2-dihydroquinolin-5-yl)oxy)- 2,2,4,4-tetramethylcyclobutyl)-4- (4-((5-(2-((S)-2,6-dioxopiperidin- 3-yl)-1-oxoisoindolin-5-yl)-2,5- diazabicyclo[2.2.1]heptan-2- yl)methyl)piperidin-1- yl)benzamide |
| 78 | 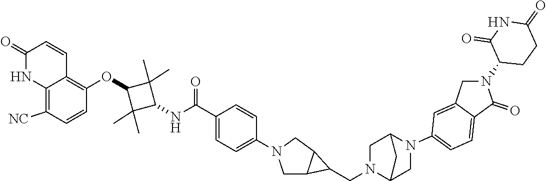 | N-((1,3-trans)-3-((8-cyano-2-oxo- 1,2-dihydroquinolin-5-yl)oxy)- 2,2,4,4-tetramethylcyclobutyl)-4- (6-((5-(2-((S)-2,6-dioxopiperidin- 3-yl)-1-oxoisoindolin-5-yl)-2,5- diazabicyclo[2.2.1]heptan-2- yl)methyl)-3- azabicyclo[3.1.0]hexan-3- yl)benzamide |
| 79 | 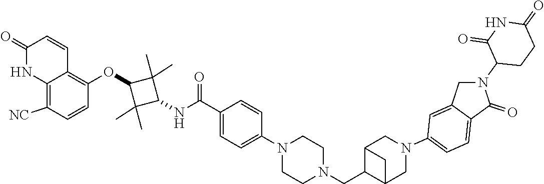 | N-((1,3-trans)-3-((8-cyano-2-oxo- 1,2-dihydroquinolin-5-yl)oxy)- 2,2,4,4-tetramethylcyclobutyl)-4- (4-((3-(2-(2,6-dioxopiperidin-3-yl)- 1-oxoisoindolin-5-yl)-3- azabicyclo[3.1.1]heptan-6- yl)methyl)piperazin-1- yl)benzamide |
| 80 | 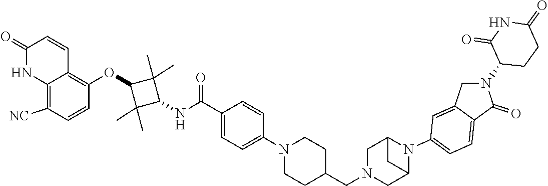 | N-((1,3-trans)-3-((8-cyano-2-oxo- 1,2-dihydroquinolin-5-yl)oxy)- 2,2,4,4-tetramethylcyclobutyl)-4- (4-((6-(2-((S)-2,6-dioxopiperidin- 3-yl)-1-oxoisoindolin-5-yl)-3,6- diazabicyclo[3.1.1]heptan-3- yl)methyl)piperidin-1- yl)benzamide |
| 81 | 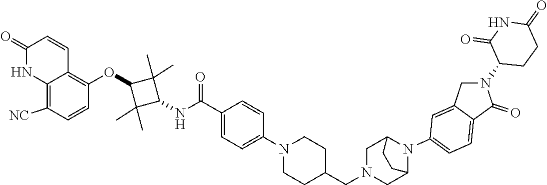 | N-((1,3-trans)-3-((8-cyano-2-oxo- 1,2-dihydroquinolin-5-yl)oxy)- 2,2,4,4-tetramethylcyclobutyl)-4- (4-((8-(2-((S)-2,6-dioxopiperidin- 3-yl)-1-oxoisoindolin-5-yl)-3,8- diazabicyclo[3.2.1]octan-3- yl)methyl)piperidin-1- yl)benzamide |
| 82 | 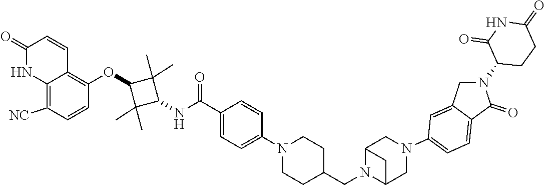 | N-((1,3-trans)-3-((8-cyano-2-oxo- 1,2-dihydroquinolin-5-yl)oxy)- 2,2,4,4-tetramethylcyclobutyl)-4- (4-((3-(2-((S)-2,6-dioxopiperidin- 3-yl)-1-oxoisoindolin-5-yl)-3,6- diazabicyclo[3.1.1]heptan-6- yl)methyl)piperidin-1- yl)benzamide |
| 83 | 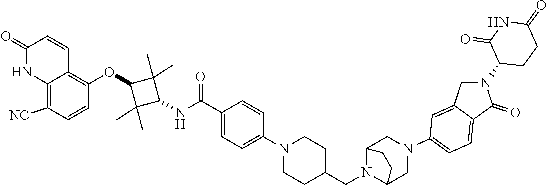 | N-((1,3-trans)-3-((8-cyano-2-oxo- 1,2-dihydroquinolin-5-yl)oxy)- 2,2,4,4-tetramethylcyclobutyl)-4- (4-((3-(2-((S)-2,6-dioxopiperidin- 3-yl)-1-oxoisoindolin-5-yl)-3,8- diazabicyclo[3.2.1]octan-8- yl)methyl)piperidin-1- yl)benzamide |
| 84 | 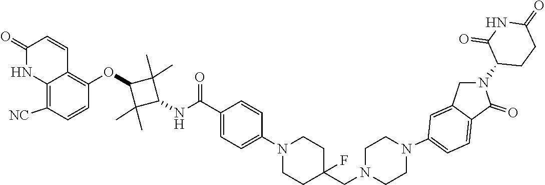 | N-((1,3-trans)-3-((8-cyano-2-oxo- 1,2-dihydroquinolin-5-yl)oxy)- 2,2,4,4-tetramethylcyclobutyl)-4- (4-((4-(2-((S)-2,6-dioxopiperidin- 3-yl)-1-oxoisoindolin-5- yl)piperazin-1-yl)methyl)-4- fluoropiperidin-1-yl)benzamide |
| 85 | 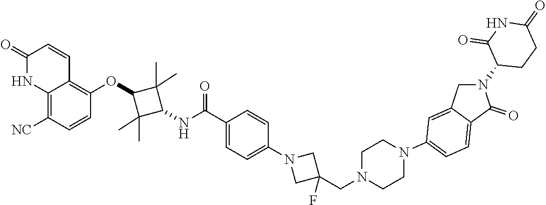 | N-((1,3-trans)-3-((8-cyano-2-oxo- 1,2-dihydroquinolin-5-yl)oxy)- 2,2,4,4-tetramethylcyclobutyl)-4- (3-((4-(2-((S)-2,6-dioxopiperidin- 3-yl)-1-oxoisoindolin-5- yl)piperazin-1-yl)methyl)-3- fluoroazetidin-1-yl)benzamide |
| 86 | 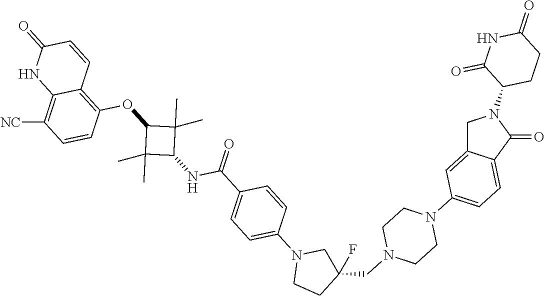 | N-((1,3-trans)-3-((8-cyano-2-oxo- 1,2-dihydroquinolin-5-yl)oxy)- 2,2,4,4-tetramethylcyclobutyl)-4- ((R)-3-((4-(2-((S)-2,6- dioxopiperidin-3-yl)-1- oxoisoindolin-5-yl)piperazin-1- yl)methyl)-3-fluoropyrrolidin-1- yl)benzamide |
| 87 | 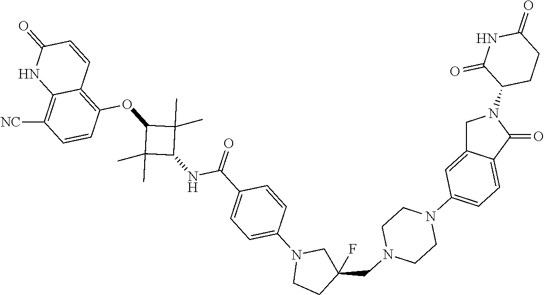 | N-((1,3-trans)-3-((8-cyano-2-oxo- 1,2-dihydroquinolin-5-yl)oxy)- 2,2,4,4-tetramethylcyclobutyl)-4- ((S)-3-((4-(2-((S)-2,6- dioxopiperidin-3-yl)-1- oxoisoindolin-5-yl)piperazin-1- yl)methyl)-3-fluoropyrrolidin-1- yl)benzamide |
| 88 | 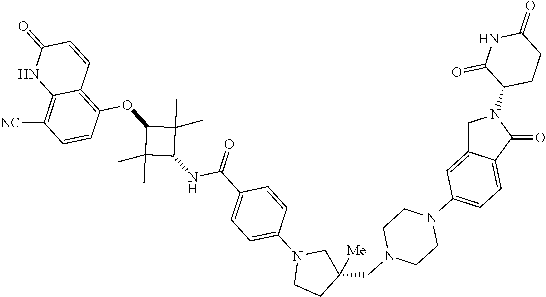 | N-((1,3-trans)-3-((8-cyano-2-oxo- 1,2-dihydroquinolin-5-yl)oxy)- 2,2,4,4-tetramethylcyclobutyl)-4- ((S)-3-((4-(2-((S)-2,6- dioxopiperidin-3-yl)-1- oxoisoindolin-5-yl)piperazin-1- yl)methyl)-3-methylpyrrolidin-1- yl)benzamide |
| 89 | 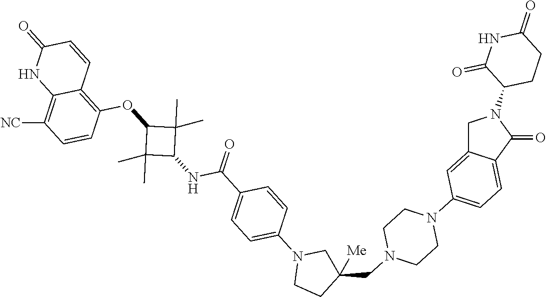 | N-((1,3-trans)-3-((8-cyano-2-oxo- 1,2-dihydroquinolin-5-yl)oxy)- 2,2,4,4-tetramethylcyclobutyl)-4- ((R)-3-((4-(2-((S)-2,6- dioxopiperidin-3-yl)-1- oxoisoindolin-5-yl)piperazin-1- yl)methyl)-3-methylpyrrolidin-1- yl)benzamide |
| 90 | 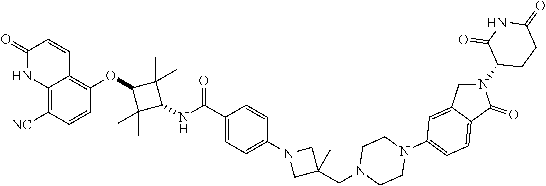 | N-((1,3-trans)-3-((8-cyano-2-oxo- 1,2-dihydroquinolin-5-yl)oxy)- 2,2,4,4-tetramethylcyclobutyl)-5- (3-((4-(2-((S)-2,6-dioxopiperidin- 3-yl)-1-oxoisoindolin-5- yl)piperazin-1-yl)methyl)-3- methylazetidin-1-yl)benzamide |
| 91 | 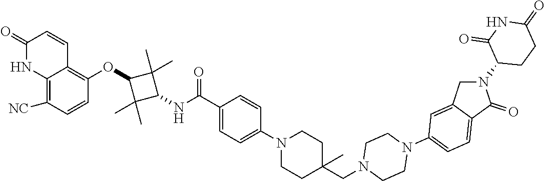 | N-((1,3-trans)-3-((8-cyano-2-oxo- 1,2-dihydroquinolin-5-yl)oxy)- 2,2,4,4-tetramethylcyclobutyl)-4- (4-((4-(2-((S)-2,6-dioxopiperidin- 3-yl)-1-oxoisoindolin-5- yl)piperazin-1-yl)methyl)-4- methylpiperidin-1-yl)benzamide |
| 92 | 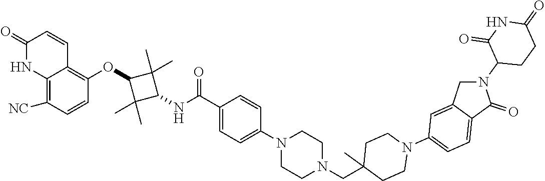 | N-((1,3-trans)-3-((8-cyano-2-oxo- 1,2-dihydroquinolin-5-yl)oxy)- 2,2,4,4-tetramethylcyclobutyl)-4- (4-((1-(2-(2,6-dioxopiperidin-3-yl)- 1-oxoisoindolin-5-yl)-4- methylpiperidin-4- yl)methyl)piperazin-1- yl)benzamide |
| 93 | 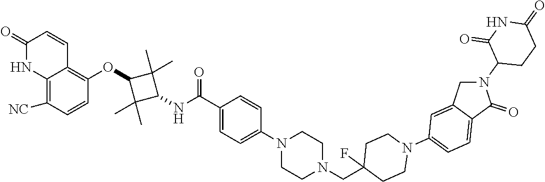 | N-((1,3-trans)-3-((8-cyano-2-oxo- 1,2-dihydroquinolin-5-yl)oxy)- 2,2,4,4-tetramethylcyclobutyl)-4- (4-((1-(2-(2,6-dioxopiperidin-3-yl)- 1-oxoisoindolin-5-yl)-4- fluoropiperidin-4- yl)methyl)piperazin-1- yl)benzamide |
| 94 | 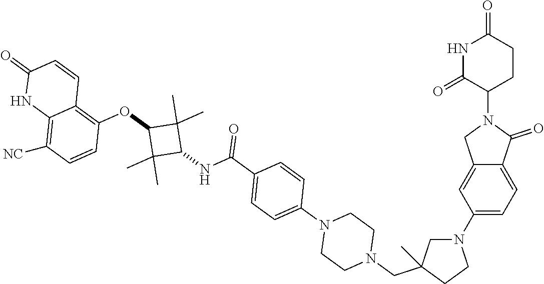 | N-((1,3-trans)-3-((8-cyano-2-oxo- 1,2-dihydroquinolin-5-yl)oxy)- 2,2,4,4-tetramethylcyclobutyl)-4- (4-((1-(2-(2,6-dioxopiperidin-3-yl)- 1-oxoisoindolin-5-yl)-3- methylpyrrolidin-3- yl)methyl)piperazin-1- yl)benzamide |
| 95 | 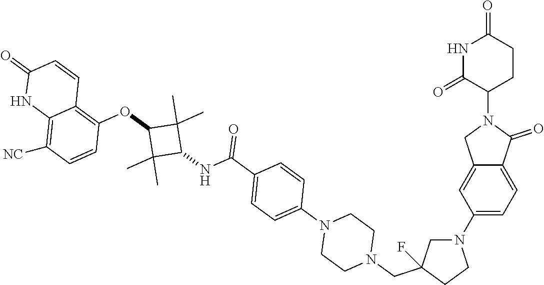 | N-((1,3-trans)-3-((8-cyano-2-oxo- 1,2-dihydroquinolin-5-yl)oxy)- 2,2,4,4-tetramethylcyclobutyl)-4- (4-((1-(2-(2,6-dioxopiperidin-3-yl)- 1-oxoisoindolin-5-yl)-3- fluoropyrrolidin-3- yl)methyl)piperazin-1- yl)benzamide |
| 96 | 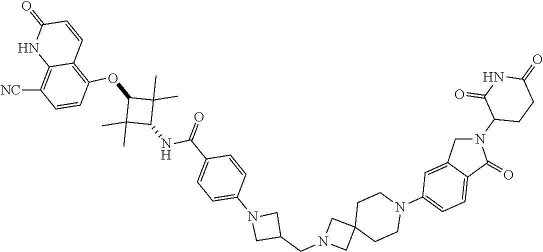 | N-((1,3-trans)-3-((8-cyano-2-oxo- 1,2-dihydroquinolin-5-yl)oxy)- 2,2,4,4-tetramethylcyclobutyl)-4- (3-((7-(2-(2,6-dioxopiperidin-3-yl)- 1-oxoisoindolin-5-yl)-2,7- diazaspiro[3.5]nonan-2- yl)methyl)azetidin-1-yl)benzamide |
| 97 | 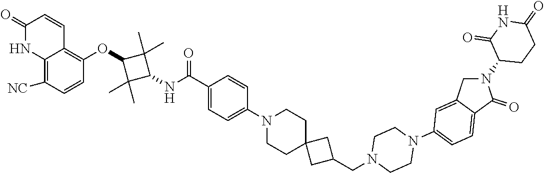 | N-((1,3-trans)-3-((8-cyano-2-oxo- 1,2-dihydroquinolin-5-yl)oxy)- 2,2,4,4-tetramethylcyclobutyl)-4- (2-((4-(2-((S)-2,6-dioxopiperidin- 3-yl)-1-oxoisoindolin-5- yl)piperazin-1-yl)methyl)-7- azaspiro[3.5]nonan-7- yl)benzamide |
| 98 | 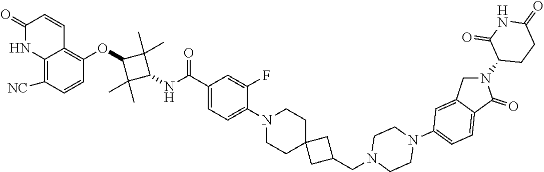 | N-((1,3-trans)-3-((8-cyano-2-oxo- 1,2-dihydroquinolin-5-yl)oxy)- 2,2,4,4-tetramethylcyclobutyl)-4- (2-((4-(2-((S)-2,6-dioxopiperidin- 3-yl)-1-oxoisoindolin-5- yl)piperazin-1-yl)methyl)-7- azaspiro[3.5]nonan-7-yl)-3- fluorobenzamide |
| 99 | 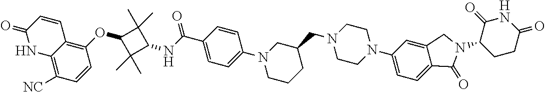 | N-((1,3-trans)-3-((8-cyano-2-oxo- 1,2-dihydroquinolin-5-yl)oxy)- 2,2,4,4-tetramethylcyclobutyl)-4- ((R)-3-((4-(2-((S)-2,6- dioxopiperidin-3-yl)-1- oxoisoindolin-5-yl)piperazin-1- |
| | | yl)methyl)piperidin-1- |
| | | yl)benzamide |
| 100 | 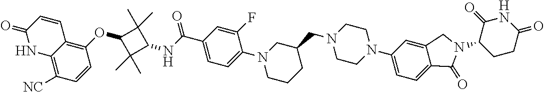 | N-((1,3-trans)-3-((8-cyano-2-oxo- 1,2-dihydroquinolin-5-yl)oxy)- 2,2,4,4-tetramethylcyclobutyl)-4- ((R)-3-((4-(2-((S)-2,6- dioxopiperidin-3-yl)-1- oxoisoindolin-5-yl)piperazin-1- |
| | | yl)methyl)piperidin-1-yl)-3- |
| | | fluorobenzamide |
| 101 | 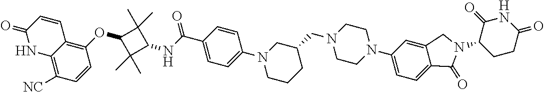 | N-((1,3-trans)-3-((8-cyano-2-oxo- 1,2-dihydroquinolin-5-yl)oxy)- 2,2,4,4-tetramethylcyclobutyl)-4- ((S)-3-((4-((2-((S)-2,6- dioxopiperidin-3-yl)-1- oxoisoindolin-5-yl)piperazin-1- |
| | | yl)methyl)piperidin-1- |
| | | yl)benzamide |
| 102 | 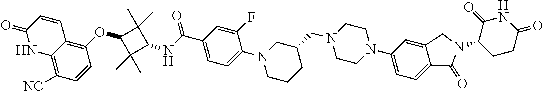 | N-((1,3-trans)-3-((8-cyano-2-oxo- 1,2-dihydroquinolin-5-yl)oxy)- 2,2,4,4-tetramethylcyclobutyl)-4- ((S)-3-((4-(2-((S)-2,6- dioxopiperidin-3-yl)-1- oxoisoindolin-5-yl)piperazin-1- |
| | | yl)methyl)piperidin-1-yl)-3- |
| | | fluorobenzamide |
| 103 | 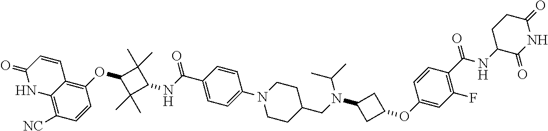 | 4-((1,3-trans)-3-(((1-(4-(((1,3- trans)-3-((8-cyano-2-oxo-1,2- dihydroquinolin-5-yl)oxy)-2,2,4,4- tetramethylcyclobutyl)carbamoyl) phenyl)piperidin-4- yl)methyl)(isopropyl)amino) cyclobutoxy)-N-(2,6-dioxo- piperidin-3-yl)-2-fluoro- benzamide |
| 104 | 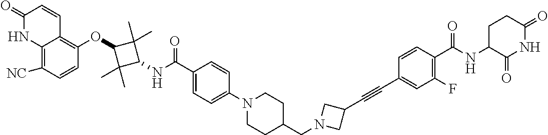 | 4-((1-((1-(4-(((1,3-trans)-3-((8- cyano-2-oxo-1,2-dihydroquinolin- 5-yl)oxy)-2,2,4,4- tetramethylcyclobutyl)carbamoyl) phenyl)piperidin-4- yl)methyl)azetidin-3-yl)ethynyl)-N- (2,6-dioxopiperidin-3-yl)-2- fluorobenzamide |
| 105 | 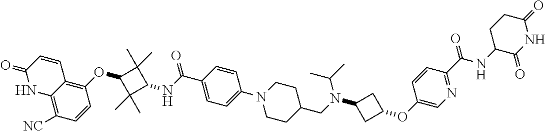 | 5-((1,3-trans)-3-(((1-(4-(((1,3- trans)-3-((8-cyano-2-oxo-1,2- dihydroquinolin-5-yl)oxy)-2,2,4,4- tetramethylcyclobutyl)carbamoyl) phenyl)piperidin-4- yl)methyl)(isopropyl)amino)cyclo- butoxy)-N-(2,6-dioxopiperidin-3- yl)picolinamide |
| 106 | 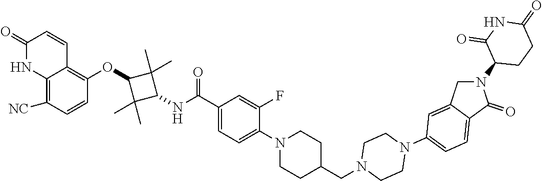 | N-((1,3-trans)-3-((8-cyano-2-oxo- 1,2-dihydroquinolin-5-yl)oxy)- 2,2,4,4-tetramethylcyclobutyl)-4- (4-((4-(2-((R)-2,6-dioxopiperidin- 3-yl)-1-oxoisoindolin-5- yl)piperazin-1-yl)methyl)piperidin- 1-yl)-3-fluorobenzamide |
| 107 | 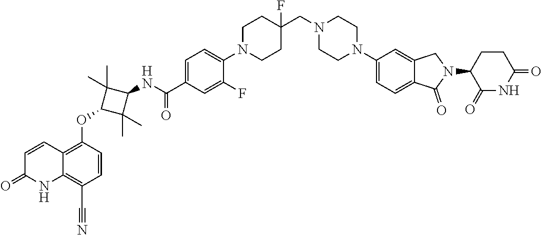 [SMILES: CC1(C)[C@H](NC(=O)c2ccc(N3CCC(F)(CN4CCN(c5ccc6c(c5)CN([C@H]5CCC(=O)NC5=O)C6=O)CC4)CC3)c(F)c2)C(C)(C)[C@H]1Oc1ccc(C#N)c2[nH]c(=O)ccc12] | N-((1,3-trans)-3-((8-cyano-2-oxo- 1,2-dihydroquinolin-5-yl)oxy)- 2,2,4,4-tetramethylcyclobutyl)-4- (4-((4-(2-((S)-2,6-dioxopiperidin- 3-yl)-1-oxoisoindolin-5- yl)piperazin-1-yl)methyl)-4- fluoropiperidin-1-yl)-3- fluorobenzamide |
| 108 | 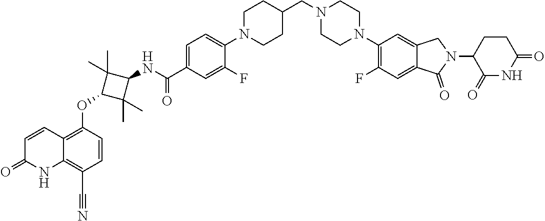 [SMILES: CC1(C)[C@H](NC(=O)c2ccc(N3CCC(CN4CCN(c5cc6c(cc5F)C(=O)N(C5CCC(=O)NC5=O)C6)CC4)CC3)c(F)c2)C(C)(C)[C@H]1Oc1ccc(C#N)c2[nH]c(=O)ccc12] | N-((1,3-trans)-3-((8-cyano-2-oxo- 1,2-dihydroquinolin-5-yl)oxy)- 2,2,4,4-tetramethylcyclobutyl)-4- (4-((4-(2-(2,6-dioxopiperidin-3-yl)- 6-fluoro-1-oxoisoindolin-5- yl)piperidin-1-yl)methyl)piperidin- 1-yl)-3-fluorobenzamide |
| 109 | 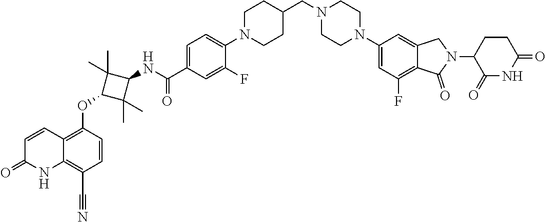 [SMILES: CC1(C)[C@H](NC(=O)c2ccc(N3CCC(CN4CCN(c5cc(F)c6c(c5)CN(C5CCC(=O)NC5=O)C6=O)CC4)CC3)c(F)c2)C(C)(C)[C@H]1Oc1ccc(C#N)c2[nH]c(=O)ccc12] | N-((1,3-trans)-3-((8-cyano-2-oxo- 1,2-dihydroquinolin-5-yl)oxy)- 2,2,4,4-tetramethylcyclobutyl)-4- (4-((4-(2-(2,6-dioxopiperidin-3-yl)- 7-fluoro-1-oxoisoindolin-5- yl)piperazin-1-yl)methyl)piperidin- 1-yl)-3-fluorobenzamide |
| 110 | 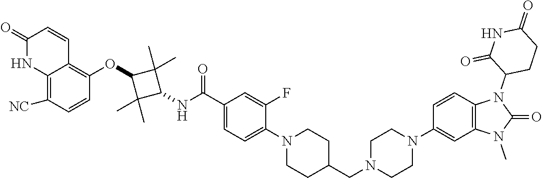 | N-((1,3-trans)-3-((8-cyano-2-oxo- 1,2-dihydroquinolin-5-yl)oxy)- 2,2,4,4-tetramethylcyclobutyl)-4- (4-((4-(1-(2,6-dioxopiperidin-3-yl)- 3-methyl-2-oxo-2,3-dihydro-1H- benzo[d]imidazol-5-yl)piperazin-1- yl)methyl)piperidin-1-yl)-3- fluorobenzamide |
| 111 | 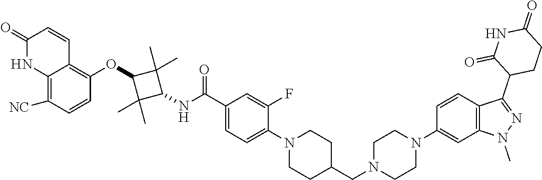 | N-((1,3-trans)-3-((8-cyano-2-oxo- 1,2-dihydroquinolin-5-yl)oxy)- 2,2,4,4-tetramethylcyclobutyl)-4- (4-((4-(3-(2,6-dioxopiperidin-3-yl)- 1-methyl-1H-indazol-6- yl)piperazin-1-yl)methyl)piperidin- 1-yl)-3-fluorobenzamide |
| 112 | 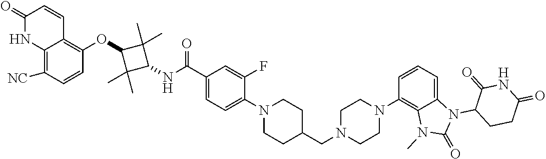 | N-((1,3-trans)-3-((8-cyano-2-oxo- 1,2-dihydroquinolin-5-yl)oxy)- 2,2,4,4-tetramethylcyclobutyl)-4- (4-((4-(1-(2,6-dioxopiperidin-3-yl)- 3-methyl-2-oxo-2,3-dihydro-1H- benzo[d]imidazol-4-yl)piperazin-1- yl)methyl)piperidin-1-yl)-3- fluorobenzamide |
| 113 | 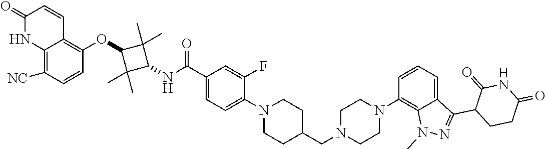 | N-((1,3-trans)-3-((8-cyano-2-oxo- 1,2-dihydroquinolin-5-yl)oxy)- 2,2,4,4-tetramethylcyclobutyl)-4- (4-((4-(3-(2,6-dioxopiperidin-3-yl)- 1-methyl-1H-indazol-7- yl)piperazin-1-yl)methyl)piperidin- 1-yl)-3-fluorobenzamide |
| 114 | 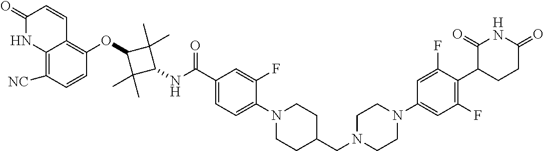 | N-((1,3-trans)-3-((8-cyano-2-oxo- 1,2-dihydroquinolin-5-yl)oxy)- 2,2,4,4-tetramethylcyclobutyl)-4- (4-((4-(4-(2,6-dioxopiperidin-3-yl)- 3,5-difluorophenyl)piperazin-1- yl)methyl)piperidin-1-yl)-3- fluorobenzamide |
| 115 | 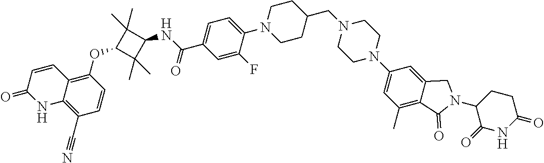 [SMILES: Cc1cc(N2CCN(CC3CCN(c4ccc(C(=O)N[C@H]5C(C)(C)[C@H](Oc6ccc(C#N)c7[nH]c(=O)ccc67)C5(C)C)cc4F)CC3)CC2)cc2c1C(=O)N(C1CCC(=O)NC1=O)C2] | N-((1,3-trans)-3-((8-cyano-2-oxo- 1,2-dihydroquinolin-5-yl)oxy)- 2,2,4,4-tetramethylcyclobutyl)-4- (4-((4-(2-(2,6-dioxopiperidin-3-yl)- 7-methyl-1-oxoisoindolin-5- yl)piperazin-1-yl)methyl)piperidin- 1-yl)-3-fluorobenzamide |
| 116 | 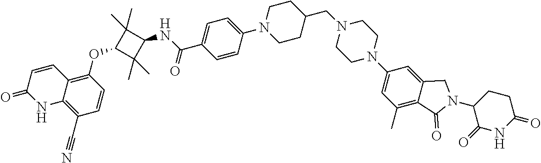 [SMILES: Cc1cc(N2CCN(CC3CCN(c4ccc(C(=O)N[C@H]5C(C)(C)[C@H](Oc6ccc(C#N)c7[nH]c(=O)ccc67)C5(C)C)cc4)CC3)CC2)cc2c1C(=O)N(C1CCC(=O)NC1=O)C2] | N-((1,3-trans)-3-((8-cyano-2-oxo- 1,2-dihydroquinolin-5-yl)oxy)- 2,2,4,4-tetramethylcyclobutyl)-4- (4-((4-(2-(2,6-dioxopiperidin-3-yl)- 7-methyl-1-oxoisoindolin-5- yl)piperazin-1-yl)methyl)piperidin- 1-yl)benzamide |
| 117 | 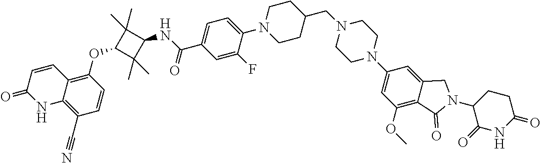 [SMILES: COc1cc(N2CCN(CC3CCN(c4ccc(C(=O)N[C@H]5C(C)(C)[C@H](Oc6ccc(C#N)c7[nH]c(=O)ccc67)C5(C)C)cc4F)CC3)CC2)cc2c1C(=O)N(C1CCC(=O)NC1=O)C2] | N-((1,3-trans)-3-((8-cyano-2-oxo- 1,2-dihydroquinolin-5-yl)oxy)- 2,2,4,4-tetramethylcyclobutyl)-4- (4-((4-(2-(2,6-dioxopiperidin-3-yl)- 7-methoxy-1-oxoisoindolin-5- yl)piperazin-1-yl)methyl)piperidin- 1-yl)-3-fluorobenzamide |
| 118 | 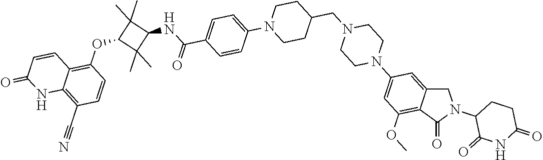 [SMILES: COc1cc(N2CCN(CC3CCN(c4ccc(C(=O)N[C@H]5C(C)(C)[C@H](Oc6ccc(C#N)c7[nH]c(=O)ccc67)C5(C)C)cc4)CC3)CC2)cc2c1C(=O)N(C1CCC(=O)NC1=O)C2] | N-((1,3-trans)-3-((8-cyano-2-oxo- 1,2-dihydroquinolin-5-yl)oxy)- 2,2,4,4-tetramethylcyclobutyl)-4- (4-((4-(2-(2,6-dioxopiperidin-3-yl)- 7-methoxy-1-oxoisoindolin-5- yl)piperazin-1-yl)methyl)piperidin- 1-yl)benzamide |
| 119 | 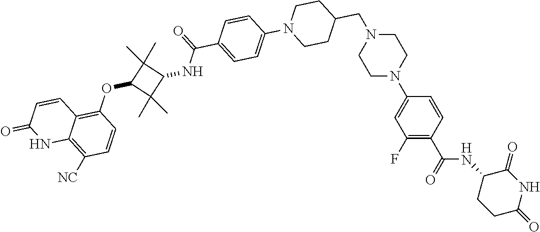 [SMILES: CC1(C)[C@H](NC(=O)c2ccc(N3CCC(CN4CCN(c5ccc(C(=O)N[C@H]6CCC(=O)NC6=O)c(F)c5)CC4)CC3)cc2)C(C)(C)[C@H]1Oc1ccc(C#N)c2[nH]c(=O)ccc12] | 4-(4-((1-(4-(((1,3-trans)-3-((8- cyano-2-oxo-1,2-dihydroquinolin- 5-yl)oxy)-2,2,4,4- tetramethylcyclobutyl)carbamoyl) phenyl)piperidin-4- yl)methyl)piperazin-1-yl)-N-((S)- 2,6-dioxopiperidin-3-yl)-2- fluorobenzamide |
| 120 | 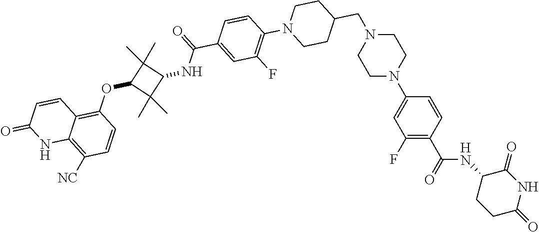 [SMILES: CC1(C)[C@H](NC(=O)c2ccc(N3CCC(CN4CCN(c5ccc(C(=O)N[C@H]6CCC(=O)NC6=O)c(F)c5)CC4)CC3)c(F)c2)C(C)(C)[C@H]1Oc1ccc(C#N)c2[nH]c(=O)ccc12] | 4-(4-((1-(4-(((1,3-trans)-3-((8- cyano-2-oxo-1,2-dihydroquinolin- 5-yl)oxy)-2,2,4,4- tetramethylcyclobutyl)carbamoyl)- 2-fluorophenyl)piperidin-4- yl)methyl)piperazin-1-yl)-N-((S)- 2,6-dioxopiperidin-3-yl)-2- fluorobenzamide |
| 121 | 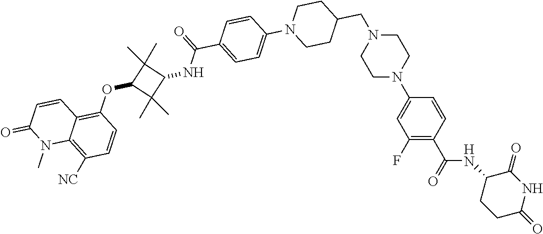 [SMILES: Cn1c(=O)ccc2c(O[C@H]3C(C)(C)[C@H](NC(=O)c4ccc(N5CCC(CN6CCN(c7ccc(C(=O)N[C@H]8CCC(=O)NC8=O)c(F)c7)CC6)CC5)cc4)C3(C)C)ccc(C#N)c21] | 4-(4-((1-(4-(((1,3-trans)-3-((8- cyano-1-methyl-2-oxo-1,2- dihydroquinolin-5-yl)oxy)-2,2,4,4- tetramethylcyclobutyl)carbamoyl) phenyl)piperidin-4- yl)methyl)piperazin-1-yl)-N-((S)- 2,6-dioxopiperidin-3-yl)-2- fluorobenzamide |
| 122 | 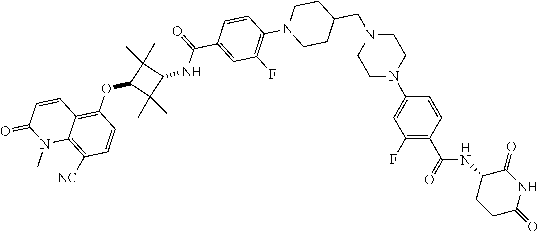 [SMILES: Cn1c(=O)ccc2c(O[C@H]3C(C)(C)[C@H](NC(=O)c4ccc(N5CCC(CN6CCN(c7ccc(C(=O)N[C@H]8CCC(=O)NC8=O)c(F)c7)CC6)CC5)c(F)c4)C3(C)C)ccc(C#N)c21] | 4-(4-((1-(4-(((1,3-trans)-3-((8- cyano-1-methyl-2-oxo-1,2- dihydroquinolin-5-yl)oxy)-2,2,4,4- tetramethylcyclobutyl)carbamoyl)- 2-fluorophenyl)piperidin-4- yl)methyl)piperazin-1-yl)-N-((S)- 2,6-dioxopiperidin-3-yl)-2- fluorobenzamide |
| 123 | 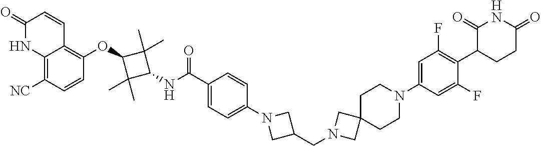 [SMILES: CC1(C)[C@H](NC(=O)c2ccc(N3CC(CN4CC5(CCN(c6cc(F)c(C7CCC(=O)NC7=O)c(F)c6)CC5)C4)C3)cc2)C(C)(C)[C@H]1Oc1ccc(C#N)c2[nH]c(=O)ccc12] | N-((1,3-trans)-3-((8-cyano-2-oxo- 1,2-dihydroquinolin-5-yl)oxy)- 2,2,4,4-tetramethylcyclobutyl)-4- (3-((7-(4-(2,6-dioxopiperidin-3-yl)- 3,5-difluorophenyl)-2,7- diazaspiro[3.5]nonan-2- yl)methyl)azetidin-1-yl)benzamide |
| 124 | 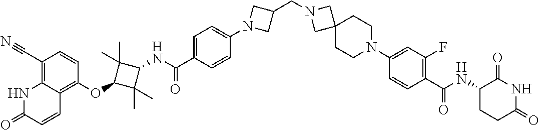 [SMILES: CC1(C)[C@H](NC(=O)c2ccc(N3CC(CN4CC5(CCN(c6ccc(C(=O)N[C@H]7CCC(=O)NC7=O)c(F)c6)CC5)C4)C3)cc2)C(C)(C)[C@H]1Oc1ccc(C#N)c2[nH]c(=O)ccc12] | 4-(2-((1-(4-(((1,3-trans)-3-((8- cyano-2-oxo-1,2-dihydroquinolin- 5-yl)oxy)-2,2,4,4- tetramethylcyclobutyl)carbamoyl) phenyl)azetidin-3-yl)methyl)-2,7- diazaspiro[3.5]nonan-7-yl)-N-((S)- 2,6-dioxopiperidin-3-yl)-2- fluorobenzamide |
| 125 | 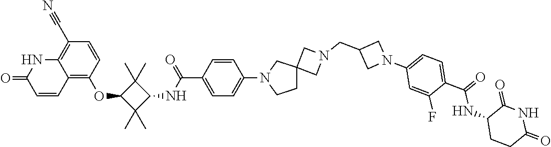 [SMILES: CC1(C)[C@H](NC(=O)c2ccc(N3CCC4(CN(CC5CN(c6ccc(C(=O)N[C@H]7CCC(=O)NC7=O)c(F)c6)C5)C4)C3)cc2)C(C)(C)[C@H]1Oc1ccc(C#N)c2[nH]c(=O)ccc12] | 4-(3-((6-(4-(((1,3-trans)-3-((8- cyano-2-oxo-1,2-dihydroquinolin- 5-yl)oxy)-2,2,4,4- tetramethylcyclobutyl)carbamoyl) phenyl)-2,6-diazaspiro[3.4]octan- 2-yl)methyl)azetidin-1-yl)-N-((S)- 2,6-dioxopiperidin-3-yl)-2- fluorobenzamide |
| 126 | 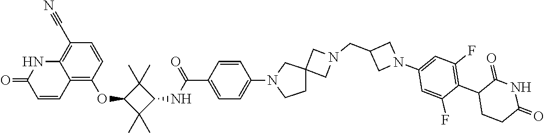 [SMILES: CC1(C)[C@H](NC(=O)c2ccc(N3CCC4(CN(CC5CN(c6cc(F)c(C7CCC(=O)NC7=O)c(F)c6)C5)C4)C3)cc2)C(C)(C)[C@H]1Oc1ccc(C#N)c2[nH]c(=O)ccc12] | N-((1,3-trans)-3-((8-cyano-2-oxo- 1,2-dihydroquinolin-5-yl)oxy)- 2,2,4,4-tetramethylcyclobutyl)-4- (2-((1-(4-(2,6-dioxopiperidin-3-yl)- 3,5-difluorophenyl)azetidin-3- yl)methyl)-2,6- diazaspiro[3.4]octan-6- yl)benzamide |
| 127 | 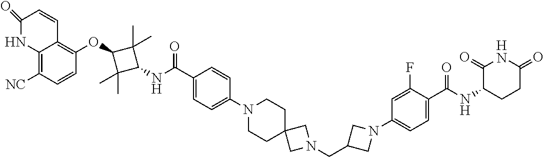 [SMILES: CC1(C)[C@H](NC(=O)c2ccc(N3CCC4(CC3)CN(CC3CN(c5ccc(C(=O)N[C@H]6CCC(=O)NC6=O)c(F)c5)C3)C4)cc2)C(C)(C)[C@H]1Oc1ccc(C#N)c2[nH]c(=O)ccc12] | 4-(3-((7-(4-(((1,3-trans)-3-((8- cyano-2-oxo-1,2-dihydroquinolin- 5-yl)oxy)-2,2,4,4- tetramethylcyclobutyl)carbamoyl) phenyl)-2,7-diazaspiro[3.5]nonan- 2-yl)methyl)azetidin-1-yl)-N-((S)- 2,6-dioxopiperidin-3-yl)-2- fluorobenzamide |
| 128 | 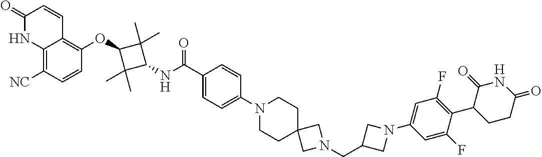 [SMILES: CC1(C)[C@H](NC(=O)c2ccc(N3CCC4(CC3)CN(CC3CN(c5cc(F)c(C6CCC(=O)NC6=O)c(F)c5)C3)C4)cc2)C(C)(C)[C@H]1Oc1ccc(C#N)c2[nH]c(=O)ccc12] | N-((1,3-trans)-3-((8-cyano-2-oxo- 1,2-dihydroquinolin-5-yl)oxy)- 2,2,4,4-tetramethylcyclobutyl)-4- (2-((1-(4-(2,6-dioxopiperidin-3-yl)- 3,5-difluorophenyl)azetidin-3- yl)methyl)-2,7- diazaspiro[3.5]nonan-7- yl)benzamide |

### Pharmaceutical Compositions

Pharmaceutical compositions of the present disclosure comprise at least one compound of Formula (1A) or (1B), or a tautomer, stereoisomer or a mixture of stereoisomers, or pharmaceutically acceptable salt, or hydrate, or deuterated derivative thereof, with a pharmaceutically acceptable carrier. These formulations include those suitable for oral, rectal, topical, buccal and parenteral (e.g., subcutaneous, intramuscular, intradermal, or intravenous) administration. The most suitable form of administration in any given case will depend on the degree and severity of the condition being treated and on the nature of the particular compound being used.

Formulations suitable for oral administration may be presented in discrete units, such as capsules, cachets, lozenges, or tablets, each containing a predetermined amount of a compound of the present disclosure as powder or granules; as a solution or a suspension in an aqueous or non-aqueous liquid; or as an oil-in-water or water-in-oil emulsion. As indicated, such formulations may be prepared by any suitable method of pharmacy which includes the step of bringing into association at least one compound of the present disclosure as the active compound and a carrier or excipient (which may constitute one or more accessory ingredients). The carrier must be acceptable in the sense of being compatible with the other ingredients of the formulation and must not be deleterious to the recipient. The carrier may be a solid or a liquid, or both, and may be formulated with at least one compound described herein as the active compound in a unit-dose formulation, for example, a tablet, which may contain from about 0.05% to about 95% by weight of the at least one active compound. Other pharmacologically active substances may also be present including other compounds. The formulations of the present disclosure may be prepared by any of the well-known techniques of pharmacy consisting essentially of admixing the components.

For solid compositions, conventional nontoxic solid carriers include, for example, pharmaceutical grades of mannitol, lactose, starch, magnesium stearate, sodium saccharin, talc, cellulose, glucose, sucrose, magnesium carbonate, and the like. Liquid pharmacologically administrable compositions can, for example, be prepared by, for example, dissolving or dispersing, at least one active compound of the present disclosure as described herein and optional pharmaceutical adjuvants in an excipient, such as, for example, water, saline, aqueous dextrose, glycerol, ethanol, and the like, to thereby form a solution or suspension. In general, suitable formulations may be prepared by uniformly and intimately admixing the at least one active compound of the present disclosure with a liquid or finely divided solid carrier, or both, and then, if necessary, shaping the product. For example, a tablet may be prepared by compressing or molding a powder or granules of at least one compound of the present disclosure, which may be optionally combined with one or more accessory ingredients. Compressed tablets may be prepared by compressing, in a suitable machine, at least one compound of the present disclosure in a free-flowing form, such as a powder or granules, which may be optionally mixed with a binder, lubricant, inert diluent and/or surface active/dispersing agent(s). Molded tablets may be made by molding, in a suitable machine, where the powdered form of at least one compound of the present disclosure is moistened with an inert liquid diluent.

Formulations suitable for buccal (sub-lingual) administration include lozenges comprising at least one compound of the present disclosure in a flavored base, usually sucrose and acacia or tragacanth, and pastilles comprising the at least one compound in an inert base such as gelatin and glycerin or sucrose and acacia.

Formulations of the present disclosure suitable for parenteral administration comprise sterile aqueous preparations of at least one compound of Formula (1A) or (1B), or a tautomer, stereoisomer or mixture of stereoisomers, or pharmaceutically acceptable salt, or hydrate, or deuterated derivative thereof, which are approximately isotonic with the blood of the intended recipient. These preparations are administered intravenously, although administration may also be affected by means of subcutaneous, intramuscular, or intradermal injection. Such preparations may conveniently be prepared by admixing at least one compound described herein with water and rendering the resulting solution sterile and isotonic with the blood. Injectable compositions according to the present disclosure may contain from about 0.1 to about 5% w/w of the active compound.

Formulations suitable for rectal administration are presented as unit-dose suppositories. These may be prepared by admixing at least one compound as described herein with one or more conventional solid carriers, for example, cocoa butter, and then shaping the resulting mixture.

Formulations suitable for topical application to the skin may take the form of an ointment, cream, lotion, paste, gel, spray, aerosol, or oil. Carriers and excipients which may be used include Vaseline, lanoline, polyethylene glycols, alcohols, and combinations of two or more thereof. The active compound (i.e., at least one compound of Formula (1A) or (1B), or a tautomer, stereoisomer or mixture of stereoisomers, or pharmaceutically acceptable salt, or hydrate, or deuterated derivative thereof) is generally present at a concentration of from about 0.1% to about 15% w/w of the composition, for example, from about 0.5 to about 2%.

The amount of active compound administered may be dependent on the subject being treated, the subject's weight, the manner of administration and the judgment of the prescribing physician. For example, a dosing schedule may involve the daily or semi-daily administration of the encapsulated compound at a perceived dosage of about 1 μg to about 1000 mg. In another embodiment, intermittent administration, such as on a monthly or yearly basis, of a dose of the encapsulated compound may be employed. Encapsulation facilitates access to the site of action and allows the administration of the active ingredients simultaneously, in theory producing a synergistic effect. In accordance with standard dosing regimens, physicians will readily determine optimum dosages and will be able to readily modify administration to achieve such dosages.

A therapeutically effective amount of a compound or composition disclosed herein can be measured by the therapeutic effectiveness of the compound. The dosages, however, may be varied depending upon the requirements of the patient, the severity of the condition being treated, and the compound being used. In one embodiment, the therapeutically effective amount of a disclosed compound is sufficient to establish a maximal plasma concentration. Preliminary doses as, for example, determined according to animal tests, and the scaling of dosages for human administration is performed according to art-accepted practices.

Toxicity and therapeutic efficacy can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compositions that exhibit large therapeutic indices are preferable.

Data obtained from the cell culture assays or animal studies can be used in formulating a range of dosage for use in humans. Therapeutically effective dosages achieved in one animal model may be converted for use in another animal, including humans, using conversion factors known in the art (see, e.g., Freireich et al., Cancer Chemother. Reports 50(4):219-244 (1966) and the following table (Table 3) for Equivalent Surface Area Dosage Factors).

TABLE 3
Equivalent Surface Area Dosage Factors.

To:

| | Mouse | Rat | Monkey | Dog | Human |
|---|---|---|---|---|---|
| From: | (20 g) | (150 g) | (3.5 kg) | (8 kg) | (60 kg) |
| Mouse | 1 | 1/2 | 1/4 | 1/6 | 1/12 |
| Rat | 2 | 1 | 1/2 | 1/4 | 1/7 |
| Monkey | 4 | 2 | 1 | 3/5 | 1/3 |
| Dog | 6 | 4 | 3/5 | 1 | 1/2 |
| Human | 12 | 7 | 3 | 2 | 1 |

The dosage of such compounds lies preferably within a range of circulating concentrations that include the ED₅₀ with little or no toxicity. The dosage may vary within this range depending upon the dosage form employed and the route of administration utilized. Generally, a therapeutically effective amount may vary with the subject's age, condition, and gender, as well as the severity of the medical condition in the subject. The dosage may be determined by a physician and adjusted, as necessary, to suit observed effects of the treatment.

### Methods of Treatment

In some embodiments, a compound of Formula (1A) or (1B), or a tautomer, stereoisomer or mixture of stereoisomers, pharmaceutically acceptable salt, hydrate, or deuterated derivative thereof, is administered to treat cancer in a subject in need thereof. In some embodiments, the cancer is chosen from prostate cancer, head and neck cancer, skin cancer, sarcoma, renal cell carcinoma, adrenocortical carcinoma, bladder cancer, lung cancer, gastric carcinoma, esophageal carcinoma, pancreatic adenocarcinoma, colorectal cancer, connective tissue cancer, glioblastoma multiforme, cervical cancer, uterine cancer, ovarian cancer, and breast cancer. In some embodiments, the cancer is prostate cancer. In some embodiments, the cancer is head and neck cancer. In some embodiments, the cancer is skin cancer. In some embodiments, the cancer is sarcoma. In some embodiments, the cancer is renal cell carcinoma. In some embodiments, the cancer is adrenocortical carcinoma. In some embodiments, the cancer is bladder cancer. In some embodiments, the cancer is lung cancer. In some embodiments, the cancer is gastric carcinoma. In some embodiments, the cancer is esophageal carcinoma. In some embodiments, the cancer is pancreatic adenocarcinoma. In some embodiments, the cancer is colorectal cancer. In some embodiments, the cancer is connective tissue cancer. In some embodiments, the cancer is glioblastoma multiforme. In some embodiments, the cancer is cervical cancer. In some embodiments, the cancer is uterine cancer. In some embodiments, the cancer is ovarian cancer. In some embodiments, the cancer is breast cancer.

In some embodiments, the cancer is androgen receptor positive.

In some embodiments, a compound of Formula (1A) or (1B), or a tautomer, stereoisomer or mixture of stereoisomers, pharmaceutically acceptable salt, hydrate, or deuterated derivative thereof, is administered as a pharmaceutical composition.

In some embodiments, the subject has been previously treated with an anti-cancer agent. In some embodiments, the anti-cancer agent is enzalutamide, apalutamide, bicalutamide, darolutamide, flutamide, abiratarone, or a combination of any of the foregoing. In some embodiments, the anti-cancer agent is enzalutamide.

In some embodiments, provided herein is a use of a compound of Formula (1A) or (1B), or a tautomer, stereoisomer or mixture of stereoisomers, pharmaceutically acceptable salt, hydrate, or deuterated derivative thereof, for treating cancer. In some embodiments, the cancer is selected from prostate cancer, head and neck cancer, skin cancer, sarcoma, renal cell carcinoma, adrenocortical carcinoma, bladder cancer, lung cancer, gastric carcinoma, esophageal carcinoma, pancreatic adenocarcinoma, colorectal cancer, connective tissue cancer, glioblastoma multiforme, cervical cancer, uterine cancer, ovarian cancer, and breast cancer. In some embodiments, the cancer is prostate cancer. In some embodiments, the cancer is head and neck cancer. In some embodiments, the cancer is skin cancer. In some embodiments, the cancer is sarcoma. In some embodiments, the cancer is renal cell carcinoma. In some embodiments, the cancer is adrenocortical carcinoma. In some embodiments, the cancer is bladder cancer. In some embodiments, the cancer is lung cancer. In some embodiments, the cancer is gastric carcinoma. In some embodiments, the cancer is esophageal carcinoma. In some embodiments, the cancer is pancreatic adenocarcinoma. In some embodiments, the cancer is colorectal cancer. In some embodiments, the cancer is connective tissue cancer. In some embodiments, the cancer is glioblastoma multiforme. In some embodiments, the cancer is cervical cancer. In some embodiments, the cancer is uterine cancer. In some embodiments, the cancer is ovarian cancer. In some embodiments, the cancer is breast cancer. In some embodiments, the cancer is androgen receptor positive.

In some embodiments, provided herein is a use of a compound of Formula (1A) or (1B), or a tautomer, stereoisomer or mixture of stereoisomers, pharmaceutically acceptable salt, hydrate, or deuterated derivative thereof, in the preparation of a medicament. In some embodiments, the medicament is for the treatment of cancer. In some embodiments, the cancer is selected from prostate cancer, head and neck cancer, skin cancer, sarcoma, renal cell carcinoma, adrenocortical carcinoma, bladder cancer, lung cancer, gastric carcinoma, esophageal carcinoma, pancreatic adenocarcinoma, colorectal cancer, connective tissue cancer, glioblastoma multiforme, cervical cancer, uterine cancer, ovarian cancer, and breast cancer. In some embodiments, the cancer is prostate cancer. In some embodiments, the cancer is head and neck cancer. In some embodiments, the cancer is skin cancer. In some embodiments, the cancer is sarcoma. In some embodiments, the cancer is renal cell carcinoma. In some embodiments, the cancer is adrenocortical carcinoma. In some embodiments, the cancer is bladder cancer. In some embodiments, the cancer is lung cancer. In some embodiments, the cancer is gastric carcinoma. In some embodiments, the cancer is esophageal carcinoma. In some embodiments, the cancer is pancreatic adenocarcinoma. In some embodiments, the cancer is colorectal cancer. In some embodiments, the cancer is connective tissue cancer. In some embodiments, the cancer is glioblastoma multiforme. In some embodiments, the cancer is cervical cancer. In some embodiments, the cancer is uterine cancer. In some embodiments, the cancer is ovarian cancer. In some embodiments, the cancer is breast cancer. In some embodiments, the cancer is androgen receptor positive.

In some embodiments, provided herein is a method of inhibiting cell growth comprising contacting a cell with a compound of Formula (1A) or (1B), or a tautomer, stereoisomer or mixture of stereoisomers, pharmaceutically acceptable salt, hydrate, or deuterated derivative thereof. In some embodiments, the cell is a cancer cell. In some embodiments, the cancer cell is a prostate cancer cell. In some embodiments, the cell is androgen receptor positive.

In one embodiment, a compound of Formula (1A) or (1B), or a tautomer, stereoisomer or mixture of stereoisomers, pharmaceutically acceptable salt, hydrate, or deuterated derivative thereof, may be administered in combination with another therapeutic agent. The other therapeutic agent can provide additive or synergistic value relative to the administration of a compound of the present disclosure alone. The therapeutic agent can be selected from, for example, hormones and hormonal analogues; signal transduction pathway inhibitors; topoisomerase I inhibitors; topoisomerase II inhibitors; antimetabolite neoplastic agents; antibiotic neoplastic agents; alkylating agents; anti-microtubule agents; platinum coordination complexes; aromatase inhibitors; and anti-mitotic agents.

In some embodiments, the therapeutic agent may be a hormone or hormonal analogue. In some embodiments, the therapeutic agent may be a signal transduction pathway inhibitor. In some embodiments, the therapeutic agent may be a topoisomerase I inhibitor. In some embodiments, the therapeutic agent may be a topoisomerase II inhibitor. In some embodiments, the therapeutic agent may be an antimetabolite neoplastic agent. In some embodiments, the therapeutic agent may be an antibiotic neoplastic agent. In some embodiments, the therapeutic agent may be an alkylating agent. In some embodiments, the therapeutic agent may be an anti-microtubule agent. In some embodiments, the therapeutic agent may be a platinum coordination complex. In some embodiments, the therapeutic agent may be an aromatase inhibitor. In some embodiments, the therapeutic agent may be an anti-mitotic agent.

In some embodiments, the aromatase inhibitor may be selected from anastrazole, letrozole, vorozole, fadrozole, exemestane, and formestane. In some embodiments, the aromatase inhibitor is anastrazole. In some embodiments, the aromatase inhibitor may be letrozole. In some embodiments, the aromatase inhibitor may be vorozole. In some embodiments, the aromatase inhibitor may be fadrozole. In some embodiments, the aromatase inhibitor may be exemestane. In some embodiments, the aromatase inhibitor may be formestane.

In some embodiments, the anti-mitotic agent may be selected from paclitaxel, docetaxel, and Abraxane. In some embodiments, the anti-mitotic agent may be paclitaxel. In some embodiments, the anti-mitotic agent may be docetaxel. In some embodiments, the anti-mitotic agent may be Abraxane.

In some embodiments, a compound of Formula (1A) or (1B), or a tautomer, stereoisomer or mixture of stereoisomers, pharmaceutically acceptable salt, hydrate, or deuterated derivative thereof, may be administered in combination with a hormone or hormonal analog. In some embodiments, a compound of Formula (1A) or (1B), or a tautomer, stereoisomer or mixture of stereoisomers, pharmaceutically acceptable salt, hydrate, or deuterated derivative thereof, may be administered in combination with a signal transduction pathway inhibitor. In some embodiments, a compound of Formula (1A) or (1B), or a tautomer, stereoisomer or mixture of stereoisomers, pharmaceutically acceptable salt, hydrate, or deuterated derivative thereof, may be administered in combination with an antimetabolite neoplastic agent. In some embodiments, a compound of Formula (1A) or (1B), or a tautomer, stereoisomer or mixture of stereoisomers, pharmaceutically acceptable salt, hydrate, or deuterated derivative thereof, may be administered in combination with a topoisomerase I inhibitor. In some embodiments, a compound of Formula (1A) or (1B), or a tautomer, stereoisomer or mixture of stereoisomers, pharmaceutically acceptable salt, hydrate, or deuterated derivative thereof, may be administered in combination with a topoisomerase II inhibitor. In some embodiments, a compound of Formula (1A) or (1B), or a tautomer, stereoisomer or mixture of stereoisomers, pharmaceutically acceptable salt, hydrate, or deuterated derivative thereof, may be administered in combination with an aromatase inhibitor.

### EXAMPLES

The examples and preparations provided below further illustrate and exemplify the compounds as disclosed herein and methods of preparing such compounds. It is to be understood that the scope of the present disclosure is not limited in any way by the scope of the following examples and preparations.

The chemical entities described herein can be synthesized according to one or more illustrative schemes herein and/or techniques well known in the art. Unless specified to the contrary, the reactions described herein take place at atmospheric pressure, generally within a temperature range from about −10° C. to about 200° C. Further, except as otherwise specified, reaction times and conditions are intended to be approximate, e.g., taking place at about atmospheric pressure within a temperature range of about −10° C. to about 200° C. over a period that can be, for example, about 1 to about 24 hours; reactions left to run overnight in some embodiments can average a period of about 16 hours.

Isolation and purification of the chemical entities and intermediates described herein can be affected, if desired, by any suitable separation or purification procedure such as, for example, filtration, extraction, crystallization, column chromatography, thin-layer chromatography or thick-layer chromatography, or a combination of these procedures. See, e.g., Carey et al. Advanced Organic Chemistry, 3^{rd} Ed., 1990 New York: Plenum Press; Mundy et al., Name Reaction and Reagents in Organic Synthesis, 2^{nd} Ed., 2005 Hoboken, NJ: J. Wiley & Sons. Specific illustrations of suitable separation and isolation procedures are given by reference to the examples hereinbelow. However, other equivalent separation or isolation procedures can also be used.

In all of the methods, it is well understood that protecting groups for sensitive or reactive groups may be employed where necessary, in accordance with general principles of chemistry. Protecting groups are manipulated according to standard methods of organic synthesis (T. W. Greene and P. G. M. Wuts (1999) Protective Groups in Organic Synthesis, 3^{rd} Ed., John Wiley & Sons). These groups may be removed at a convenient stage of the compound synthesis using methods that are readily apparent to those skilled in the art.

When desired, the (R)- and (S)-isomers of the nonlimiting exemplary compounds, if present, can be resolved by methods known to those skilled in the art, for example, by formation of diastereoisomeric salts or complexes which can be separated, e.g., by crystallization; via formation of diastereoisomeric derivatives which can be separated, e.g., by crystallization, gas-liquid or liquid chromatography; selective reaction of one enantiomer with an enantiomer-specific reagent, e.g., enzymatic oxidation or reduction, followed by separation of the modified and unmodified enantiomers; or gas-liquid or liquid chromatography in a chiral environment, e.g., on a chiral support, such as silica with a bound chiral ligand or in the presence of a chiral solvent. Alternatively, a specific enantiomer can be synthesized by asymmetric synthesis using optically active reagents, substrates, catalysts or solvents, or by converting one enantiomer to the other by asymmetric transformation.

The compounds described herein can be optionally contacted with a pharmaceutically acceptable acid to form the corresponding acid addition salts. Also, the compounds described herein can be optionally contacted with a pharmaceutically acceptable base to form the corresponding basic addition salts.

In some embodiments, disclosed compounds can generally be synthesized by an appropriate combination of generally well-known synthetic methods. Techniques useful in synthesizing these chemical entities are both readily apparent and accessible to those of skill in the relevant art, based on the instant disclosure. Many of the optionally substituted starting compounds and other reactants are commercially available, e.g., from Millipore Sigma or can be readily prepared by those skilled in the art using commonly employed synthetic methodology.

The discussion below is offered to illustrate certain of the diverse methods available for use in making the disclosed compounds and is not intended to limit the scope of reactions or reaction sequences that can be used in preparing the compounds provided herein. The skilled artisan will understand that standard atom valencies apply to all compounds disclosed herein in genus or named compound for unless otherwise specified.

All final compounds of the experimental described herein were checked for purity by HPLC on a Shimadzu LC-2010A and compounds were detected at the wavelength of 214 nM and 254 nM. Purities for all final compounds were over 95% based on HPLC peaks (214 nM and 254 nM wavelength). Liquid chromatography condition: Column, XBRIDGE C18, 3.6 micron, 2.1×50 mm: Mobile phase, water (0.05% TFA) and acetonitrile (0.05% TFA), linear gradient from 10% acetonitrile to 100% acetonitrile over 7 min; Oven temperature 45° C.; Flow rate, 0.8 mL/mL. H-NMR was obtained on Bruker 400 MHz NMR spectrometer.

### General Synthetic Schemes

Compounds of Formula (1A) and (1B) can be prepared according to the following schemes. The following schemes represent the general methods used in preparing these compounds. However, the synthesis of these compounds is not limited to these representative methods, as they can also be prepared by various other methods those skilled in the art of synthetic chemistry, for example, in a stepwise or modular fashion.

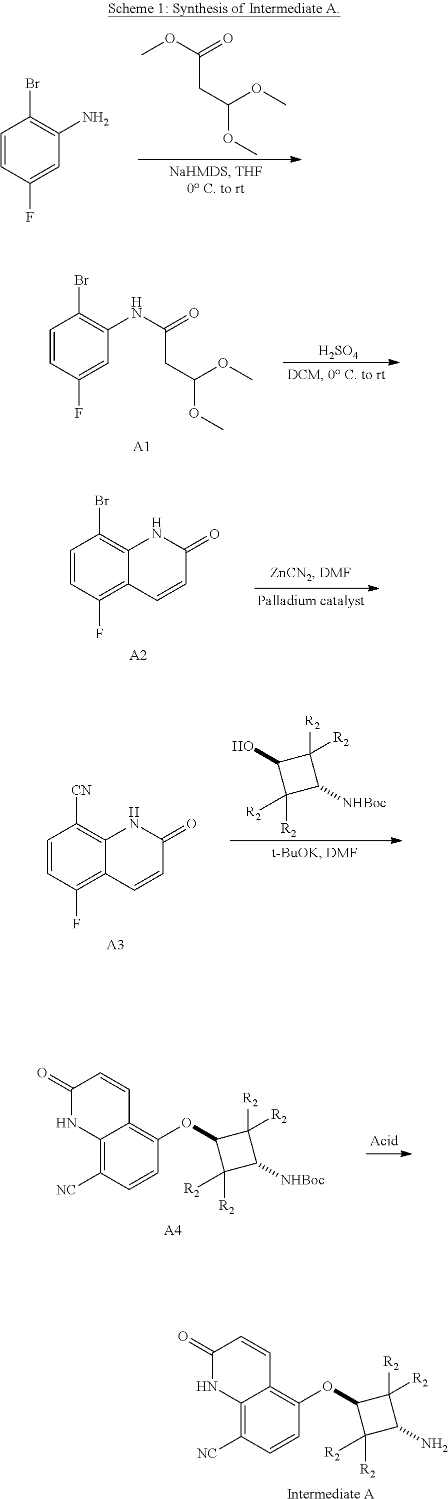

The reaction of 2-bromo-5-fluoroaniline with methyl-3,3-dimethoxypropanoate under basic conditions can form amide A1. The deprotection of acetal group under acidic conditions can generate an aldehyde which can cyclize to form quinolinone A2. The conversion of bromo to nitrile group in the presence of palladium catalyst and zinc cyanide can generate 8-cyano-5-fluoroquinolinone A3. The displacement of 5-F by substituted trans-3-tert-butoxycarbonylaminocyclobutanol under basic condition can generate the aryl ether A4, which can be deprotected under acidic condition to provide Intermediate A.

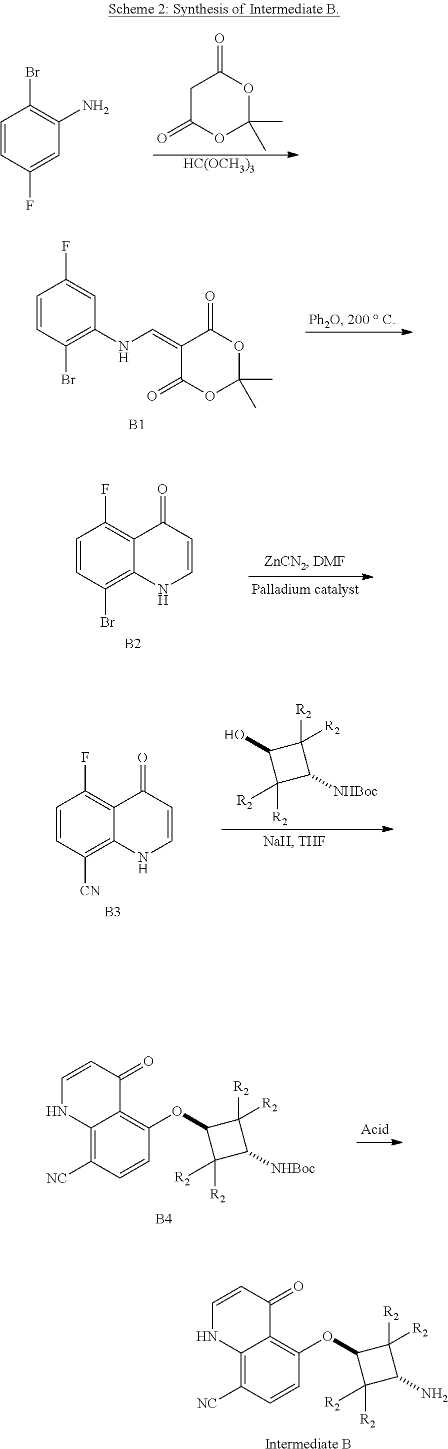

The Intermediate B can be prepared according to Scheme 2. Reaction of 2-bromo-5-fluoroaniline with Meldrum's acid and trimethyl orthoformate can generate B1, which can be converted to 4-oxo-dihydroquinoline B2. The transformation of bromo group in B2 to nitrile group in B3 and the subsequent displacement of 5-F in B3 to generate the aryl ether B4 can followed the similar method as described in Scheme 1. Deprotection of N-Boc in B4 under acidic conditions will provide Intermediate B.

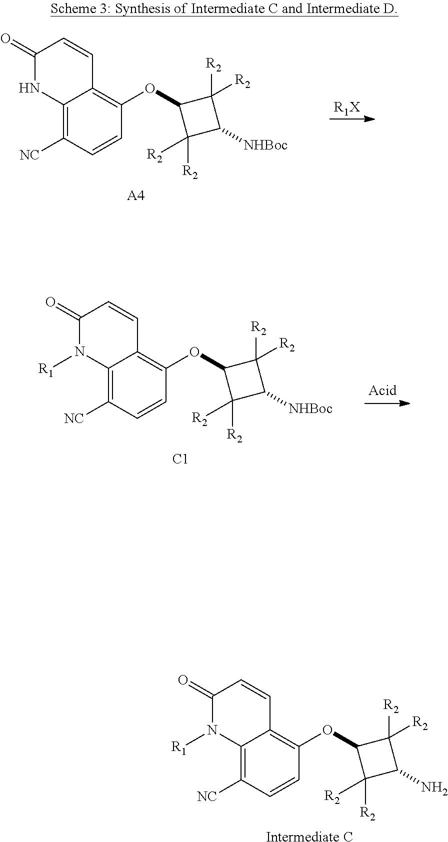

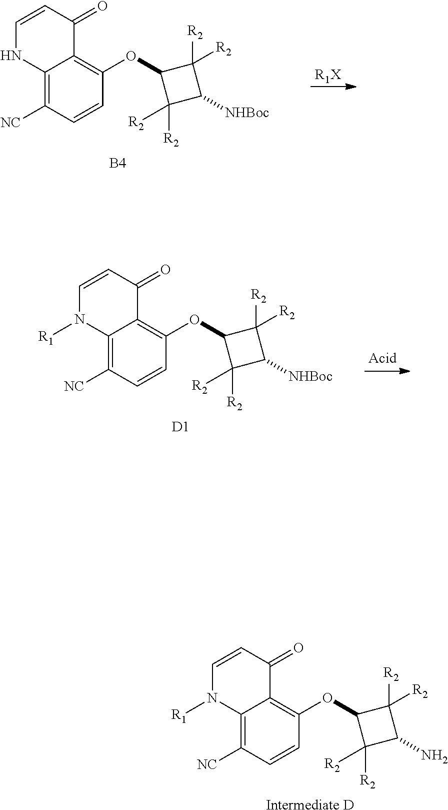

As described in Scheme 3, Intermediate C and Intermediate D can be produced from A4 and B4 via two reaction steps. The N-alkylation can be carried out under basic conditions such as potassium carbonate or sodium hydride to generate C1 and D1, which can be deprotected under acidic conditions to provide Intermediate C and Intermediate D.

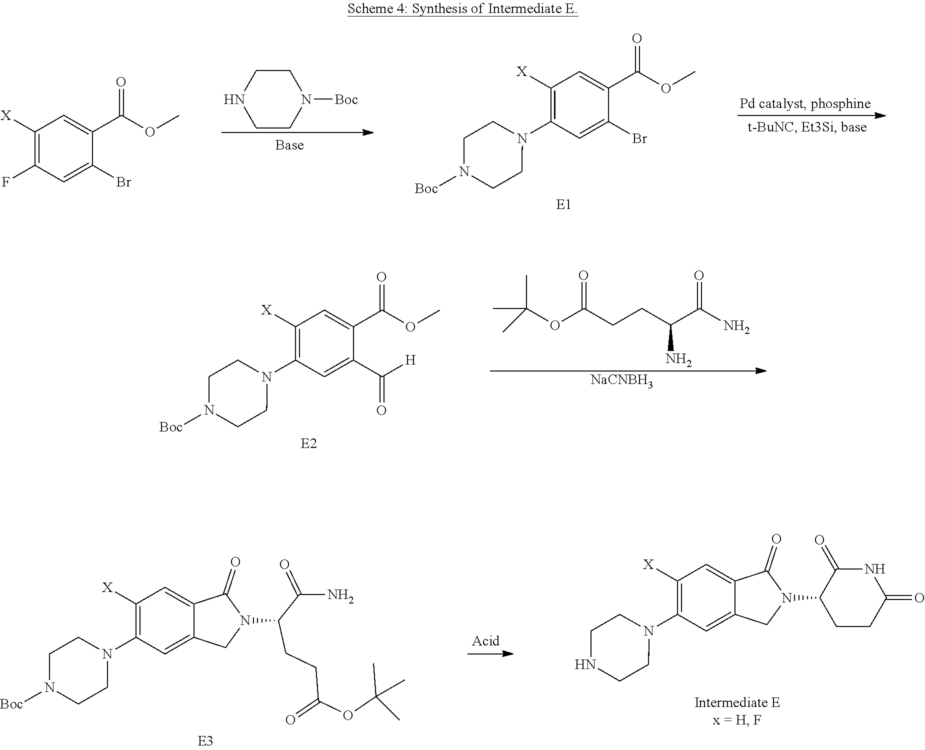

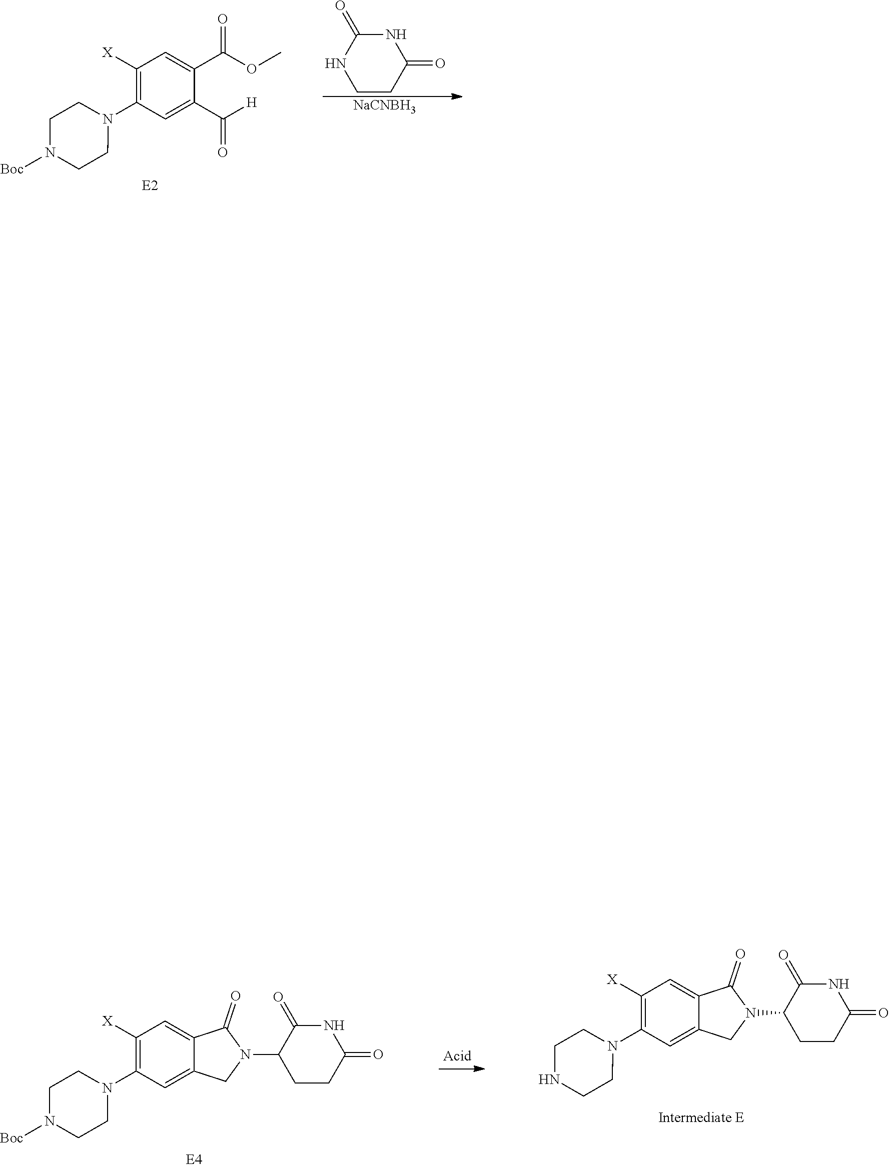

The required chiral and racemate intermediate E can be prepared according to Scheme 4. The fluorine substituted benzoic acid methyl ester can react with N-Boc-piperazine under basic conditions to generate E1. The conversion of the Br to aldehyde group can be achieved under the conditions like what was reported in the literature (Organic Letters 16 (13), 3492-3495, 2014). The reductive amination between E2 and H-Glu(OBu^{t})—NH₂ can generate E3 which can be cyclized under acidic condition such as benzenesulfonic acid to generate Intermediate E. Similarly, the reductive amination between E2 and 2-aminoglutarimide can form racemate E4 which can be deprotected under acidic conditions to provide racemic Intermediate E.

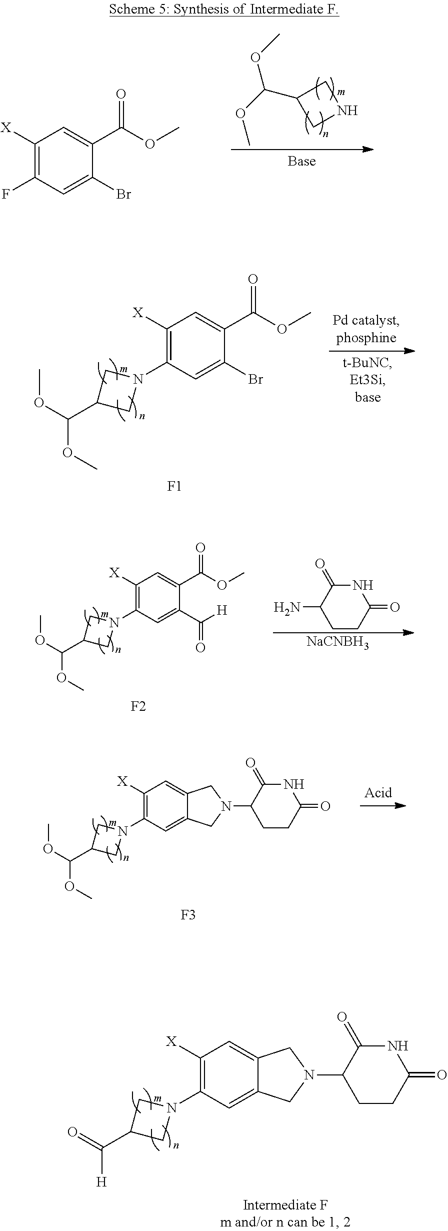

The required aldehyde Intermediate F can be prepared according to Scheme 5 following a similar synthetic sequence as described for Intermediate E.

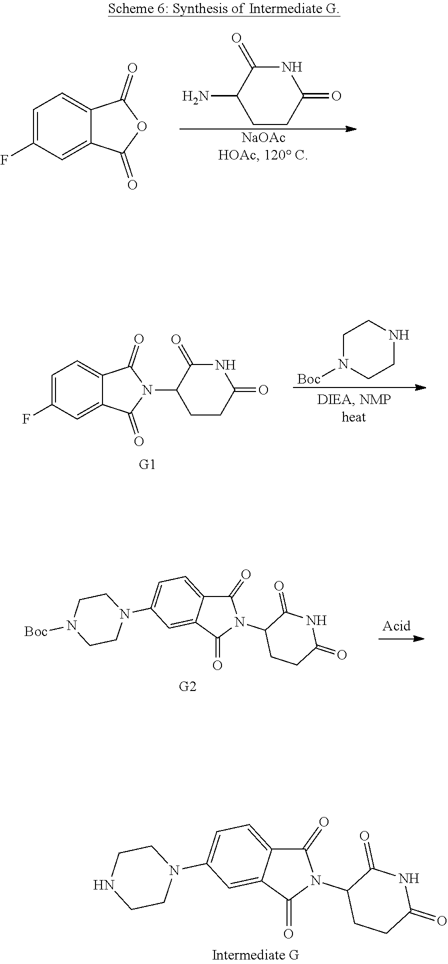

Intermediate G can be prepared from 4-fluorophthalic acid anhydride and 2-aminoglutarimide as shown in Scheme 6.

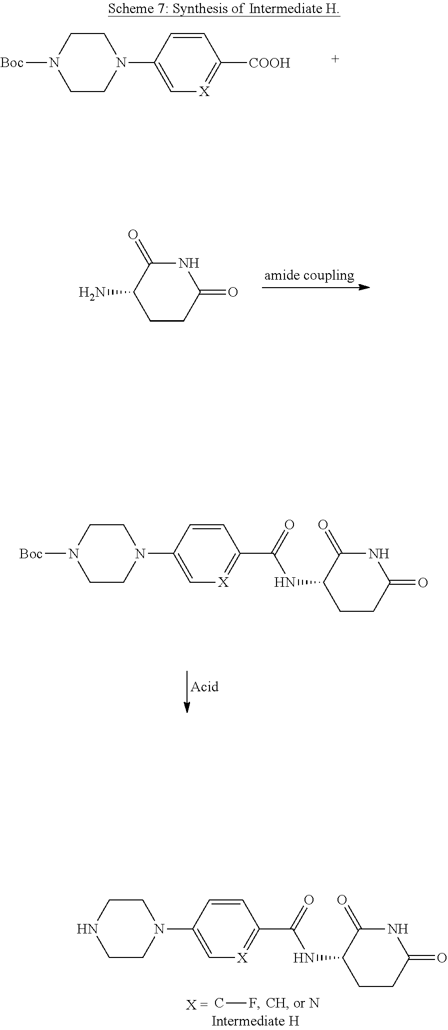

Intermediate H can be prepared through amide coupling as descried in Scheme 7.

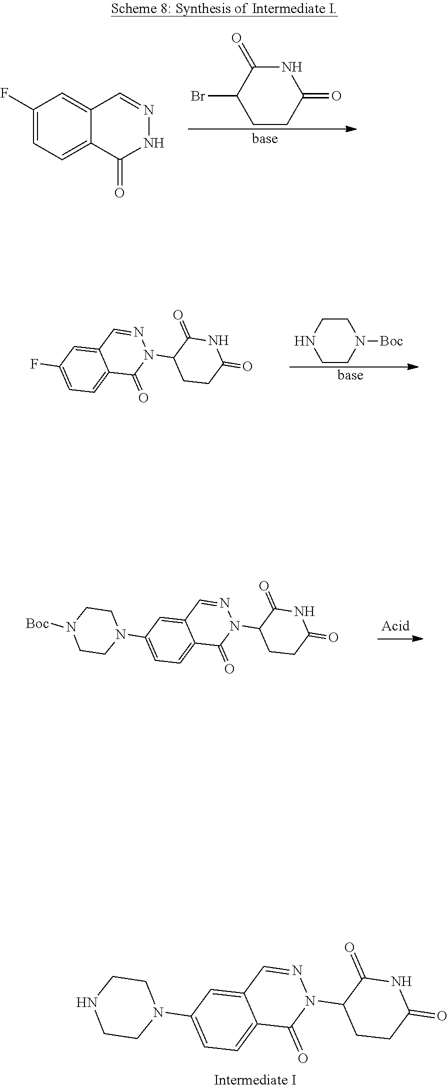

Intermediate I can be prepared from the available 6-fluorophthalazinone as described in Scheme 8.

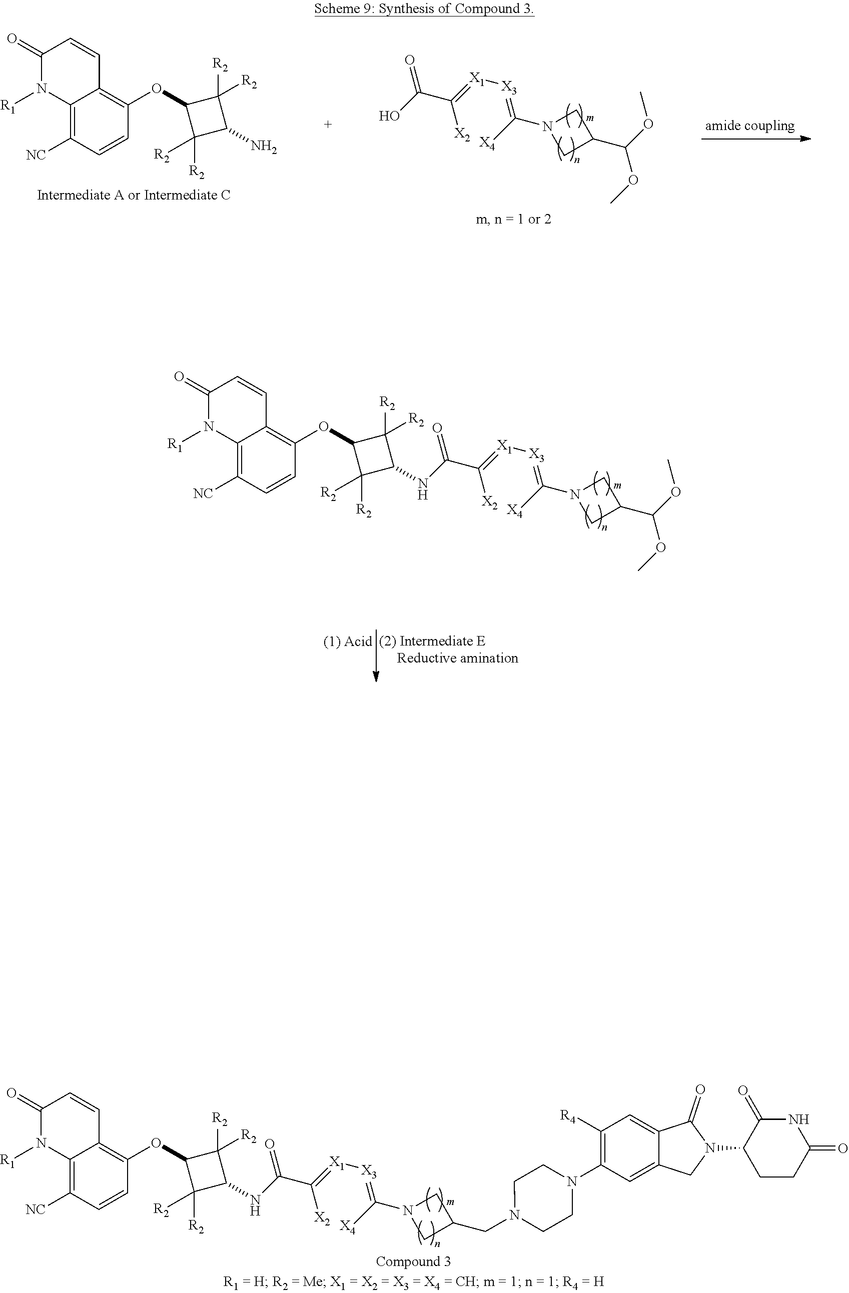

The following compounds can be synthesized using similar synthetic method described in Scheme 9: 2, 4, 5, 7, 8, 10, 12, 13, 14, 15, 16, 17, 18, 25, 26, 27, 29, 30, 31, 32, 33, 34, 35, 36, 37, 38, 39, 40, 53, 54, 55, 56, 58, and 60.

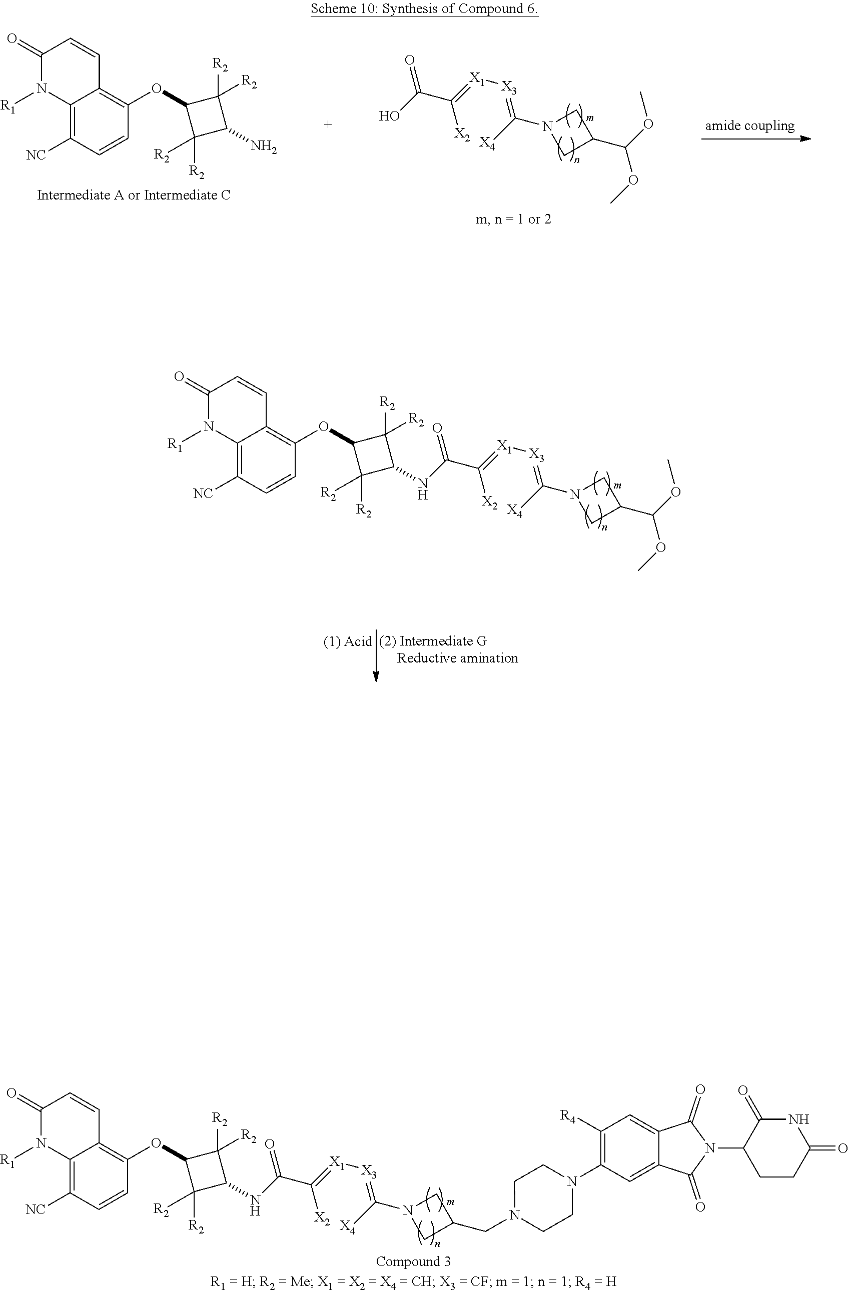

Compound 6 can be synthesized according to the synthetic method described in Scheme 10. Using the similar method as described for the synthesis of compound 6, the following compounds can be prepared: 1, 9, 11, 19, 20, 21, 22, 23, 24, 28, 41, 42, 57, and 59.

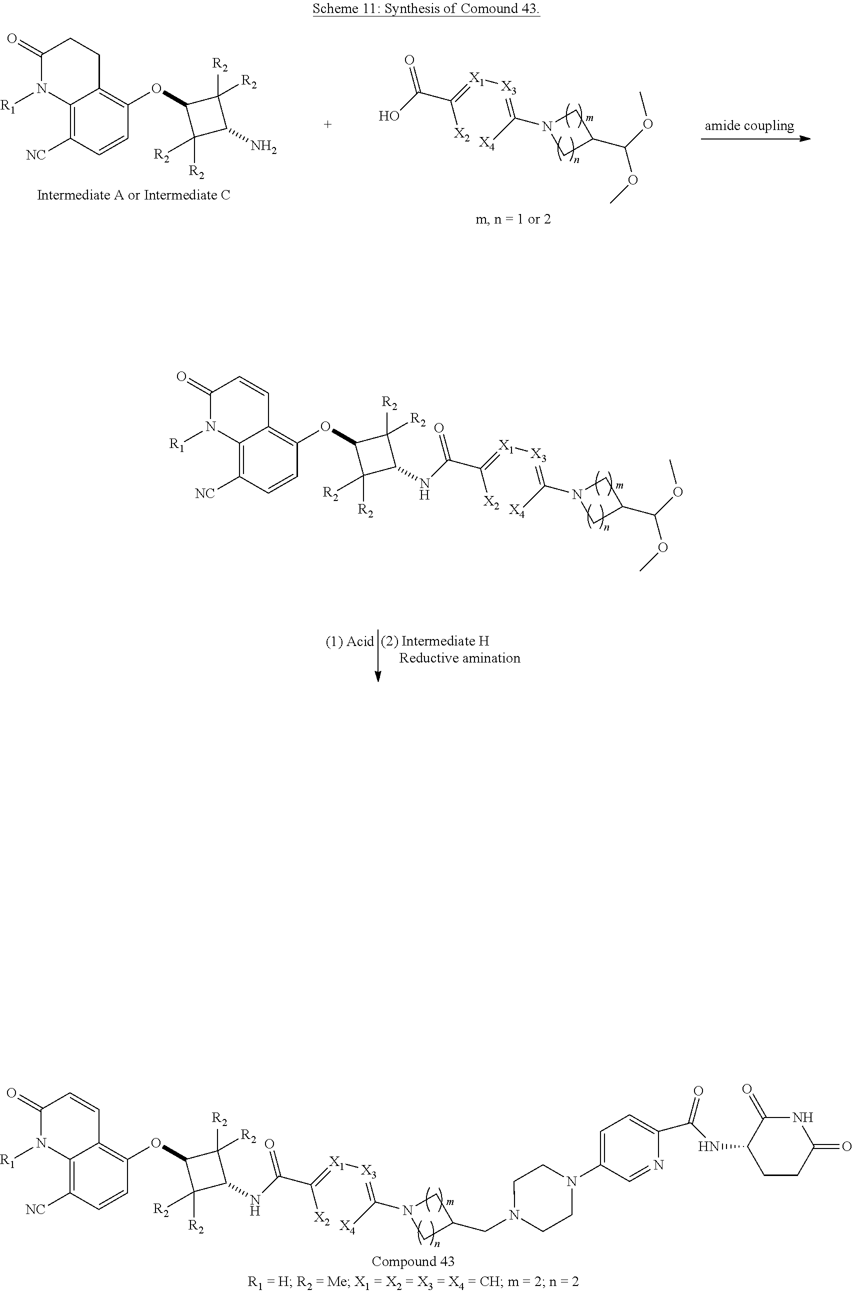

Using the similar method as described for the synthesis of compound 43, the following compounds can be prepared: 44, 45, 46, 47, and 48.

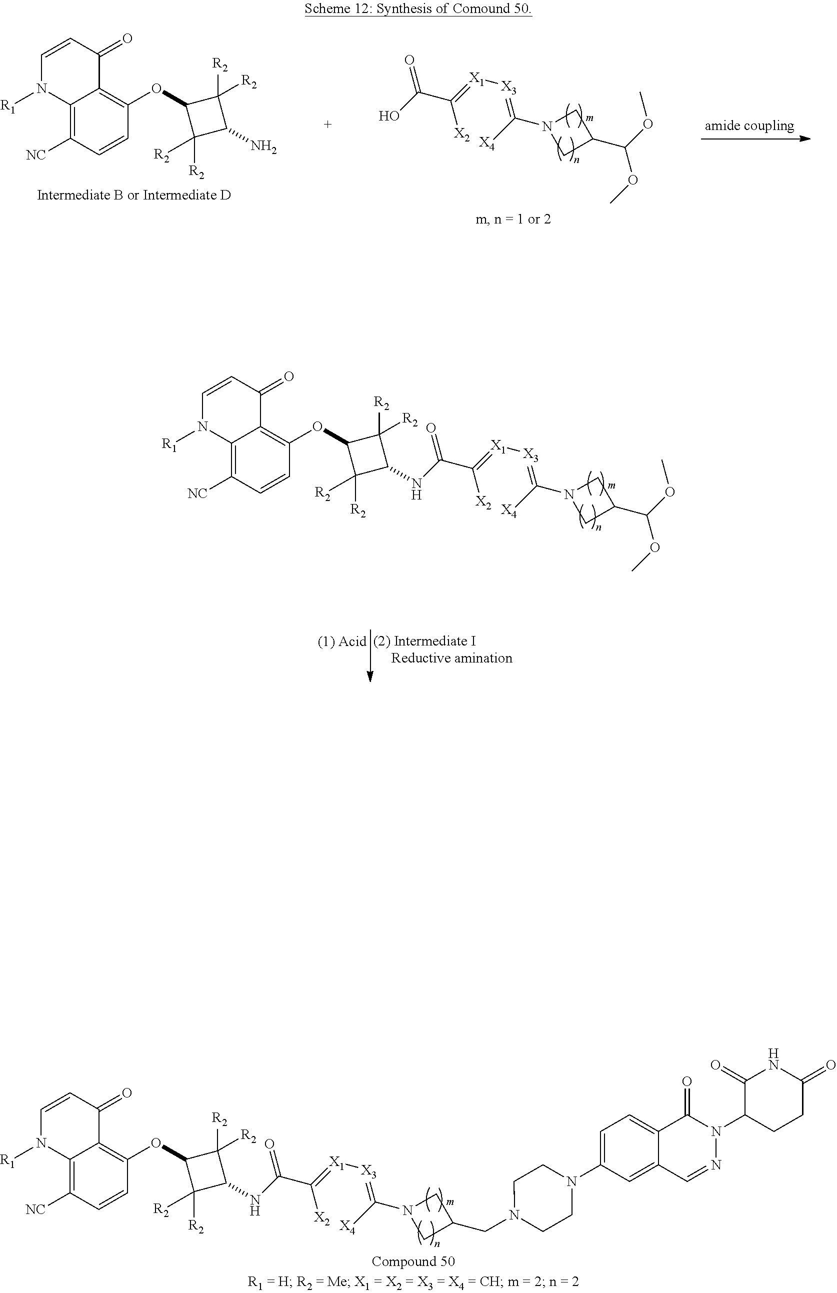

Using the similar method as described for the synthesis of compound 50, the following compounds can be prepared: 49, 51, and 52.

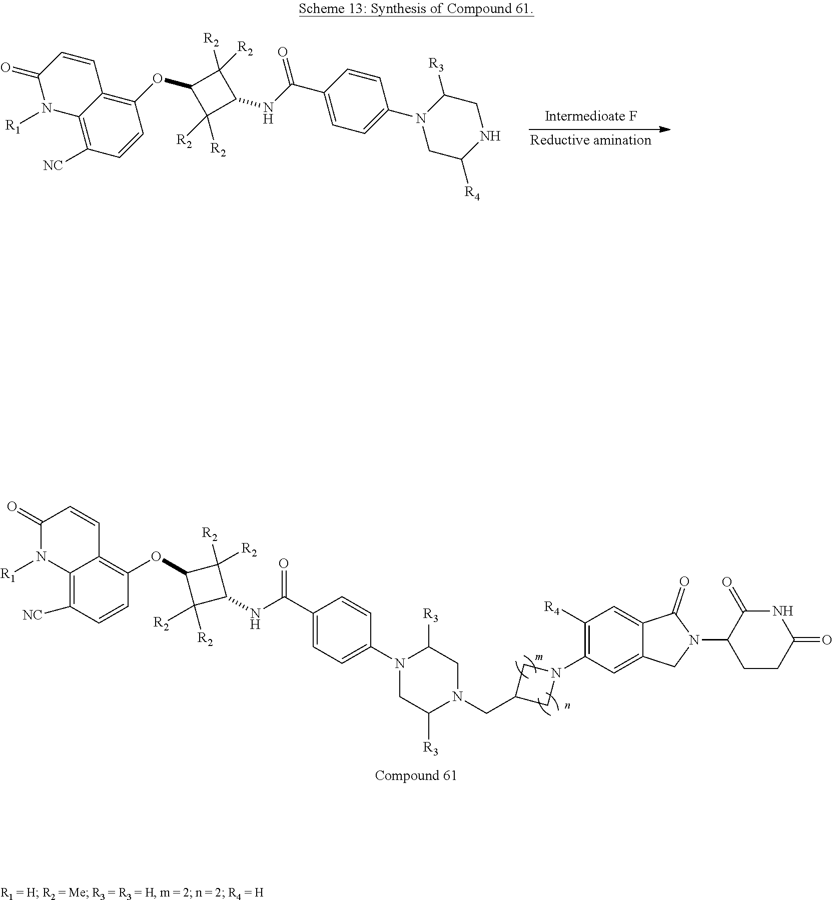

Using the similar method as described for the synthesis of compound 61, the following compounds can be prepared: 62, 63, 64, 65, 66, 67, 68, 69, 70, 71, 72, 73, 74 and 75.

### ABBREVIATIONS

The following abbreviations have the meanings set forth below:
ACN: Acetonitrile
BINAP: (2,2′-bis(diphenylphosphino)-1,1′-binaphthyl
Boc: tert-Butyloxycarbonyl protecting group
Cs₂CO₃: Cesium carbonate
DCM: Dichloromethane
DIEA: N, N-Diisopropylethylamine
DMF: Dimethylformamide
DMSO: Dimethyl sulfoxide
EA: Ethyl acetate
EDCl: 1-Ethyl-3-(3-dimethylaminopropyl)carbodiimide
Et₃Si: Triethylsilane
HATU: Hexafluorophosphate azabenzotriazole tetramethyl uronium
HOAc: Acetic acid
HOBt: Hydroxy benzotriazole
KHMDS: Potassium bis(trimethylsilyl)amide
LCMS: Liquid chromatography-mass spectrometry
Mel: Methyl iodide
NaCNBH₃ or NaBH₃CN: Sodium cyanoborohydride
NaHMDS: Sodium bis(trimethylsilyl)amide
NaOAc: Sodium acetate
NMP: N-Methyl-2-pyrrolidone
PCy₃: Tricyclohexylphosphine
Pd(OAc)₂: Palladium(II) acetate
Pd₂(dba)₃: Tris(dibenzylideneacetone)dipalladium(0)
PE: Polyethylene
Ph₂O: Diphenyl ether
STAB: Sodium triacetoxyborohydride
t-BuNC: tert-Butyl isocyanide
t-BuOK: Potassium tert-butoxide
TEA: Triethylamine
TFA: Trifluoroacetic acid
THF: Tetrahydrofuran
TLC: Thin-layer chromatography
ZnCN₂: Zinc cyanide

Compounds described in the experimental were prepared from commercially available material. Purity of all final compounds were analyzed by HPLC with detection at 214 nM and 254 nM wavelength. All final compounds showed purity greater than 95%. All final compounds were characterized by LC/MS and H-NMR. The following are representative examples demonstrating how the claimed molecules can be made, however, a person of skill in the art would understand that the compounds could be prepared by other synthetic methods.

### Preparation of Example Compounds

### Example 1: Preparation of Synthesis of N-((1,3-trans)-3-((8-cyano-2-oxo-1,2-dihydroquinolin-5-yl)oxy)-2,2,4,4-tetramethylcyclobutyl)-4-(4-((4-(2-((S)-2,6-dioxopiperidin-3-yl)-1-oxoisoindolin-5-yl)piperazin-1-yl)methyl)piperidin-1-yl)-3-fluorobenzamide (Compound 16)

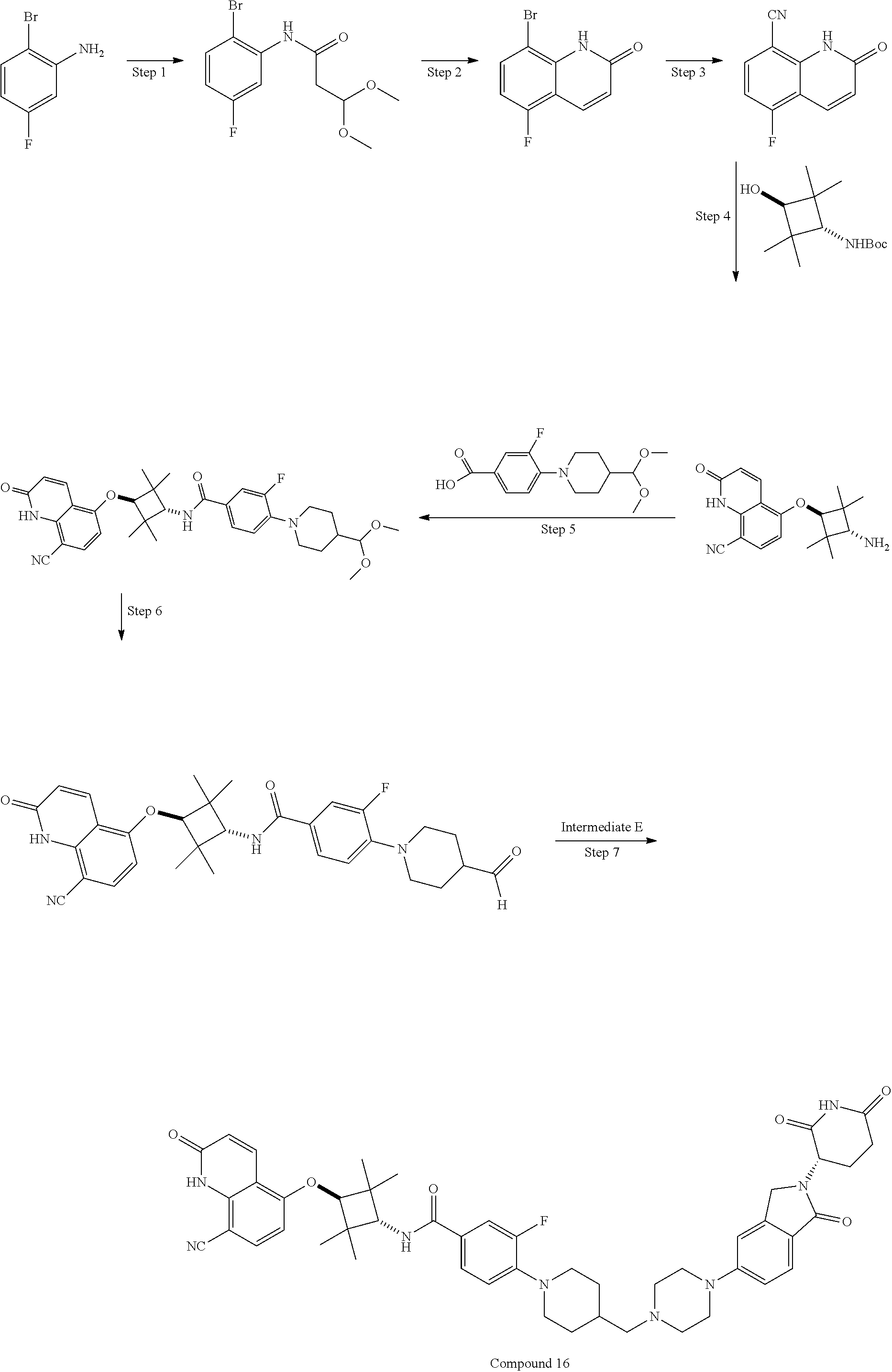

### Step 1: Preparation of N-(2-bromo-5-fluorophenyl)-3,3-dimethoxypropanamide

To a solution of 2-bromo-5-fluoroaniline (5 g, 26.3 mmol) in THF (100 mL) stirred at room temperature was added methyl 3,3-dimethoxypropanoate (4.7 g, 31.7 mmol). The mixture was cooled to 0° C. and NaHMDS (39.5 mL, 39.5 mmol) was added slowly. The reaction mixture was stirred at room temperature for 12 hours. The reaction was quenched with saturated NH₄Cl solution (200 mL) and then extracted with EA (200 mL×3). The combined organic layer was washed with brine, dried over Na₂SO₄, and concentrated in vacuum to obtain the desired product (8 g, 85% purity, 84.4% yield) as a yellow solid. LC/MS: 328.0 [M+Na]⁺; ¹HNMR (400 MHz, CDCl₃) δ=8.91 (s, 1H), 8.26 (dd, J=11.2, 2.8 Hz, 1H), 7.46 (dd, J=8.8, 5.6 Hz, 1H), 6.73-6.65 (m, 1H), 4.75 (t, J=4.8 Hz, 1H), 3.47 (s, 6H), 2.79 (d, J=5.2 Hz, 2H).

### Step 2: Preparation of 8-bromo-5-fluoroquinolin-2(1H)-one

A solution of N-(2-bromo-5-fluorophenyl)-3,3-dimethoxypropanamide (7 g, 22.9 mmol) in DCM (35 mL) was added to H₂SO₄ (21 mL) while stirred under argon at 0° C. The reaction mixture was stirred at 25° C. for 2 hours. The mixture was concentrated in vacuum and the residue was poured into ice-water. The solid was filtered and washed with H₂O, i-PrOH, and TBME. The solid was dried in vacuum to give the title compound (5.2 g, 93.8%) as a yellow solid. ¹HNMR (400 MHz, CDCl₃) δ 9.05 (brs, 1H), 7.96 (d, J=9.8 Hz, 1H), 7.65 (dd, J=8.7, 5.2 Hz, 1H), 6.84 (t, J=8.9 Hz, 1H), 6.70 (d, J=9.8 Hz, 1H).

### Step 3: Preparation of 5-fluoro-2-oxo-1,2-dihydroquinoline-8-carbonitrile

To a solution of 8-bromo-5-fluoroquinolin-2(1H)-one (5.7 g, 23.5 mmol) in DMF (100 mL) was added ZnCN₂ (5.5 g, 47 mmol) and RuPhos Pd G2 (1.8 g, 2.3 mmol) under nitrogen at 25° C. The reaction was stirred at 100° C. for 5 hours. The mixture was cooled to room temperature and the solid was filtrated. The solution was concentrated in vacuum and the residue was triturated with DCM. The solid was collected and purified via silica gel column chromatography with DCM/MeOH=20/1 to obtain the desired compound (2.6 g, 58.8%) as a yellow solid. LC/MS: 189.1 [M+H]⁺; ¹HNMR (400 MHz, DMSO) δ 12.03 (brs, 1H), 8.14 (d, J=5.9 Hz, 2H), 7.28 (t, J=8.9 Hz, 1H), 6.82 (s, 1H).

### Step 4: Preparation of 5-((1,3-trans)-3-amino-2,2,4,4-tetramethylcyclobutoxy)-2-oxo-1,2-dihydro quinoline-8-carbonitrile

To a solution of tert-butyl ((1,3-trans)-3-hydroxy-2,2,4,4-tetramethylcyclobutyl)carbamate (1.04 g, 4.25 mmol) in DMF (10 mL) was added 5-fluoro-2-oxo-1,2-dihydroquinoline-8-carbonitrile (400 mg, 2.13 mmol) and t-BuOK (2.39 g, 21.26 mmol). The reaction was stirred at 60° C. for 1 hour. The mixture was diluted with water and extracted with EA. The organic phase was washed with brine and dried over Na₂SO₄. The solvent was removed in vacuum and the residue was purified via silica gel column chromatography with DCM/MeOH=20/1 to obtain the desired compound (290 mg, 43.8%) as a yellow solid. LC/MS: 312.2 [M+H]⁺.

### Step 5: Preparation of N-((1,3-trans)-3-((8-cyano-2-oxo-1,2-dihydroquinolin-5-yl)oxy)-2,2,4,4-tetramethylcyclobutyl)-4-(4-(dimethoxymethyl)piperidin-1-yl)-3-fluorobenzamide

To a solution of 4-(4-(dimethoxymethyl)piperidin-1-yl)-3-fluorobenzoic acid (286 mg, 0.96 mmol) in DMF (6 mL) was added 5-((1,3-trans)-3-amino-2,2,4,4-tetramethylcyclobutoxy)-2-oxo-1,2-dihydroquinoline-8-carbonitrile (250 mg, 0.80 mmol), DIEA (311 mg, 2.41 mmol) and HATU (458 mg, 1.20 mmol). The reaction mixture was stirred at room temperature for 0.5 hour. The mixture was diluted with water and extracted with EA. The organic phase was washed with brine and dried over Na₂SO₄. The solvent was removed in vacuum and the residue was purified via silica gel column chromatography with DCM/MeOH=20/1 to afford the desired compound (100 mg, 21.1%) as a yellow solid. LC/MS: 591.3 [M+H]⁺.

### Step 6: Preparation of N-((1,3-trans)-3-((8-cyano-2-oxo-1,2-dihydroquinolin-5-yl)oxy)-2,2,4,4-tetramethylcyclobutyl)-3-fluoro-4-(4-formylpiperidin-1-yl)benzamide

A solution of N-((1,3-trans)-3-((8-cyano-2-oxo-1,2-dihydroquinolin-5-yl)oxy)-2,2,4,4-tetramethylcy-clobutyl)-4-(4-(dimethoxymethyl)piperidin-1-yl)-3-fluorobenzamide (100 mg, 0.17 mmol) in THF/HCl (2 N) (4 mL, 1/1) was stirred at room temperature for 1 hour. The mixture was concentrated in vacuum to give the desired compound (100 mg, crude) as a white solid. LC/MS: 545.3 [M+H]⁺.

### Step 7: Preparation of N-((1,3-trans)-3-((8-cyano-2-oxo-1,2-dihydroquinolin-5-yl)oxy)-2,2,4,4-tetramethylcyclobutyl)-4-(4-((4-(2-((S)-2,6-dioxopiperidin-3-yl)-1-oxoisoindolin-5-yl)piperazin-1-yl)methyl)piperidin-1-yl)-3-fluorobenzamide

To a solution of N-((1,3-trans)-3-((8-cyano-2-oxo-1,2-dihydroquinolin-5-yl)oxy)-2,2,4,4-tetramethylcyclobutyl)-3-fluoro-4-(4-formylpiperidin-1-yl)benzamide (100 mg, crude) in DCM/MeOH (6 mL, 1/1) stirred at room temperature was added (S)-3-(1-oxo-5-(piperazin-1-yl)isoindolin-2-yl)piperidine-2,6-dione benzene sulfate (83 mg, 0.17 mmol) and Et₃N (34 mg, 0.34 mmol). The mixture was stirred at room temperature for 30 minutes. Then HOAc (0.05 mL) and NaBH₃CN (21 mg, 0.34 mmol) were added. The reaction mixture was stirred at room temperature for 1 hour. The mixture was diluted with DCM (50 mL) and washed with brine. The organic layer was dried over Na₂SO₄ and concentrated in vacuum to give a crude product. The crude product was purified by Prep-TLC (DCM:MeOH=10:1) to obtain the desired product (40 mg, 27.4% for two steps). LC/MS: 857.3 [M+H]⁺; ¹HNMR (400 MHz, DMSO) δ 11.58 (brs, 1H), 10.92 (s, 1H), 8.21 (d, J=8.8 Hz, 1H), 7.94 (d, J=8.4 Hz, 1H), 7.69 (d, J=9.2 Hz, 1H), 7.65-7.58 (m, 2H), 7.49 (d, J=8.4 Hz, 1H), 7.09-7.00 (m, 3H), 6.72-6.61 (m, 2H), 5.01 (dd, J=13.2, 5.2 Hz, 1H), 4.41-4.25 (m, 2H), 4.22-4.06 (m, 2H), 3.45 (d, J=11.2 Hz, 2H), 3.27-3.22 (m, 5H), 2.93-2.81 (m, 1H), 2.71 (t, J=11.2 Hz, 2H), 2.66-2.51 (m, 2H), 2.39-2.27 (m, 2H), 2.21 (d, J=6.8 Hz, 2H), 1.96-1.89 (m, 1H), 1.80 (d, J=12.4 Hz, 2H), 1.75-1.65 (m, 1H), 1.32-1.24 (m, 2H), 1.22 (s, 6H), 1.19 (s, 1H), 1.16 (s, 6H).

### Example 2: Preparation of Synthesis of N-((1,3-trans)-3-((8-cyano-4-oxo-1,4-dihydroquinolin-5-yl)oxy)-2,2,4,4-tetramethylcyclobutyl)-4-(4-((4-(2-((S)-2,6-dioxopiperidin-3-yl)-1-oxoisoindolin-5-yl)piperazin-1-yl)methyl)piperidin-1-yl)-3-fluorobenzamide (Compound 29)

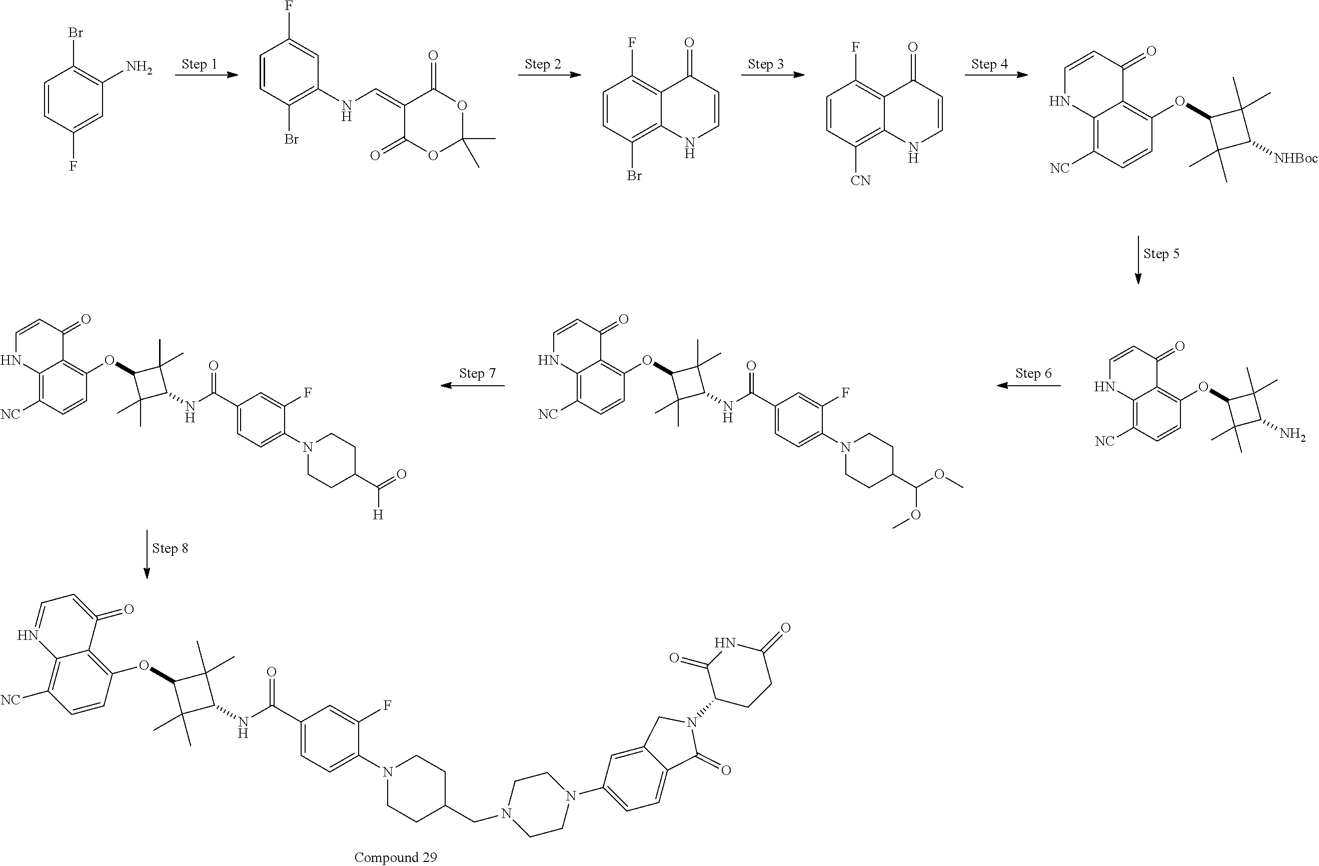

### Step 1: Preparation of 5-(((2-bromo-5-fluorophenyl)amino)methylene)-2,2-dimethyl-1,3-dioxane-4,6-dione

To a solution of 2-bromo-5-fluoroaniline (1 g, 5.3 mmol) in trimethoxymethane (20 mL) was added 2,2-dimethyl-1,3-dioxane-4,6-dione (0.76 g, 5.3 mmol) under nitrogen at 25° C. The reaction was stirred at 110° C. for 1 hour. The mixture was cooled to room temperature and triturated with EtOH. The solid was collected to give the desired product (1.2 g, 65.8%) as a yellow solid. ¹HNMR (400 MHz, CDCl₃) δ 11.60 (d, J=13.5 Hz, 1H), 8.55 (d, J=13.8 Hz, 1H), 7.61-7.56 (m, 1H), 7.14 (dd, J=9.4, 2.7 Hz, 1H), 6.89-6.83 (m, 1H), 1.74 (s, 6H).

### Step 2: Preparation of 8-bromo-5-fluoroquinolin-4(1H)-one

A mixture of 5-{[(2-bromo-5-fluorophenyl)amino]methylidene}-2,2-dimethyl-1,3-dioxane-4,6-dione (1.2 g, 3.4 mmol) and diphenylether (20 mL) was stirred at 200° C. for 30 minutes under nitrogen. The mixture was cooled to room temperature. The solid was filtered and washed with hexane to give the title compound (650 mg, 78.9%) as a yellow solid. LC/MS: 241.9 [M+H]⁺.

### Step 3: Preparation of 5-fluoro-4-oxo-1,4-dihydroquinoline-8-carbonitrile

To a solution of 8-bromo-5-fluoroquinolin-4(1H)-one (650 mg, 2.68 mmol) in DMF (10 mL) was added ZnCN₂ (630.6 mg, 5.37 mmol) and RuPhos Pd G2 (208.5 mg, 0.26 mmol) under nitrogen at 25° C. The reaction was stirred at 100° C. for 4 hours. The mixture was cooled to room temperature and the solid was filtrated off. The solution was concentrated in vacuum and the residue was triturated with DCM. The solid was collected to give the desired product (430 mg, 85.1%) as a yellow solid. LC/MS: 189.1 [M+H]⁺.

### Step 4: Preparation of tert-butyl ((1,3-trans)-3-((8-cyano-4-oxo-1,4-dihydroquinolin-5-yl)oxy)-2,2,4,4-tetramethylcyclobutyl)carbamate

To a solution of tert-butyl ((1,3-trans)-3-hydroxy-2,2,4,4-tetramethylcyclobutyl)carbamate (550 mg, 2.26 mmol) in THF (20 mL) was added NaH (452 mg, 60%, 11.3 mmol) and the mixture was stirred at 0° C. for 30 minutes. Then 5-fluoro-4-oxo-1,4-dihydroquinoline-8-carbonitrile (430 mg, 2.26 mmol) was added to the mixture at 50° C. The reaction was stirred at 50° C. for 6 hours. The mixture was poured into ice water and extracted with DCM. The organic phase was washed with brine and dried over Na₂SO₄. The solvent was removed in vacuum and the residue was purified by Prep-TLC with DCM:MeOH=10:1 to obtain the desired compound (360 mg, 38.3%) as a yellow solid. LC/MS: 412.2 [M+H]⁺.

### Step 5: Preparation of 5-((1,3-trans)-3-amino-2,2,4,4-tetramethylcyclobutoxy)-4-oxo-1,4-dihydro quinoline-8-carbonitrile

A mixture of tert-butyl((1,3-trans)-3-((8-cyano-4-oxo-1,4-dihydroquinolin-5-yl)oxy)-2,2,4,4-tetramethylcyclobutyl)carbamate (360 mg, 0.87 mmol) in DCM:TFA (8 mL, 3:1) was stirred at 25° C. for 1 hour under nitrogen. The solvent was removed in vacuum to give the title compound (350 mg, crude) as a yellow solid. LC/MS: 312.2 [M+H]⁺.

### Step 6: Preparation of N-((1,3-trans)-3-((8-cyano-4-oxo-1,4-dihydroquinolin-5-yl)oxy)-2,2,4,4-tetramethylcyclobutyl)-4-(4-(dimethoxymethyl)piperidin-1-yl)-3-fluorobenzamide

To a solution of 5-((1,3-trans)-3-amino-2,2,4,4-tetramethylcyclobutoxy)-4-oxo-1,4-dihydroquinoline-8-carbonitrile (350 mg, crude) in DMF (5 mL) was added 4-(4-(dimethoxymethyl)piperidin-1-yl)-3-fluorobenzoic acid (343.8 mg, 1.15 mmol), HOBT (234.3 mg, 1.73 mmol), EDCl (332.5 mg, 1.73 mmol) and DIEA (448.3 mg, 3.46 mmol). The mixture was stirred under nitrogen at 25° C. for 8 hours and then poured into water and extracted with DCM. The organic phase was washed with brine and dried over Na₂SO₄. The solvent was removed in vacuum and the residue was purified by Prep-TLC with DCM:MeOH=10:1 to obtain the desired compound (250 mg, 48.6% for two steps) as a yellow solid. LC/MS: 591.2 [M+H]⁺.

### Step 7: Preparation of N-((1,3-trans)-3-((8-cyano-4-oxo-1,4-dihydroquinolin-5-yl)oxy)-2,2,4,4-tetramethylcyclobutyl)-3-fluoro-4-(4-formylpiperidin-1-yl)benzamide

A mixture of N-((1,3-trans)-3-((8-cyano-4-oxo-1,4-dihydroquinolin-5-yl)oxy)-2,2,4,4-tetramethylcyclobutyl)-4-(4-(dimethoxymethyl)piperidin-1-yl)-3-fluorobenzamide (250 mg, 0.42 mmol) in THF/HCl (2N) (5 mL, 1/1) was stirred under nitrogen at 25° C. for 1 hour. The mixture was neutralized with saturated bicarbonate sodium solution and extracted with DCM. The organic phase was washed with brine and dried over Na₂SO₄. The solvent was removed in vacuum to obtain the desired compound (200 mg crude product, 87.4%) as a yellow solid. LC/MS: 545.2 [M+H]⁺.

### Step 8: Preparation of N-((1,3-trans)-3-((8-cyano-4-oxo-1,4-dihydroquinolin-5-yl)oxy)-2,2,4,4-tetramethylcyclobutyl)-4-(4-((4-(2-((S)-2,6-dioxopiperidin-3-yl)-1-oxoisoindolin-5-yl)piperazin-1-yl)methyl)piperidin-1-yl)-3-fluorobenzamide

To a solution of N-((1,3-trans)-3-((8-cyano-4-oxo-1,4-dihydroquinolin-5-yl)oxy)-2,2,4,4-tetramethylcyclobutyl)-3-fluoro-4-(4-formylpiperidin-1-yl)benzamide (200 mg crude product, 0.36 mmol) in DCM:MeOH (5 mL, 1:1) was added (S)-3-(1-oxo-5-(piperazin-1-yl)isoindolin-2-yl)piperidine-2,6-dione benzene sulfate (120.6 mg, 0.37 mmol) and DIEA (142.4 mg, 1.1 mmol). The mixture was stirred under nitrogen at 25° C. for 30 minutes. Then HOAc (0.05 mL) and sodium cyanoborohydride (45.4 mg, 0.72 mmol) were added. The mixture was stirred at 25° C. for 1 hour, then quenched with H₂O and extracted with DCM. The organic phase was washed with brine and dried over Na₂SO₄. The solvent was removed in vacuum and the residue was purified by Prep-HPLC with ACN—H₂O (0.1% TFA) to obtain the desired compound (40 mg, 12.7% for two steps) as a light green solid. LC/MS: 857.3 [M+H]⁺; ¹H NMR (400 MHz, DMSO) δ 10.98 (s, 1H), 9.43 (brs, 1H), 8.07 (d, J=8.4 Hz, 1H), 7.98 (brs, 1H), 7.74-7.66 (m, 3H), 7.61 (d, J=8.4 Hz, 1H), 7.21-7.15 (m, 2H), 7.12 (t, J=8.8 Hz, 1H), 6.70 (d, J=8.4 Hz, 1H), 6.33 (brs, 1H), 5.10-5.05 (m, 1H), 4.40-4.35 (m, 2H), 4.27-4.22 (m, 1H), 4.12 (d, J=9.2 Hz, 1H), 4.04-4.00 (m, 2H), 3.68-3.62 (m, 2H), 3.55-3.49 (m, 2H), 3.23-3.16 (m, 5H), 2.96-2.87 (m, 1H), 2.82-2.74 (m, 2H), 2.70-2.53 (m, 2H), 2.43-2.30 (m, 1H), 2.10-2.01 (m, 1H), 2.00-1.94 (m, 1H), 1.89 (d, J=12.4 Hz, 2H), 1.47-1.38 (m, 2H), 1.26 (s, 6H), 1.20 (s, 6H).

### Example 3: Preparation of N-((1,3-trans)-3-((8-cyano-2-oxo-1,2-dihydroquinolin-5-yl)oxy)-2,2,4,4-tetramethylcyclobutyl)-4-(4-((4-(2-((S)-2,6-dioxopiperidin-3-yl)-1-oxoisoindolin-5-yl)piperazin-1-yl)methyl)piperidin-1-yl)benzamide (Compound 18)

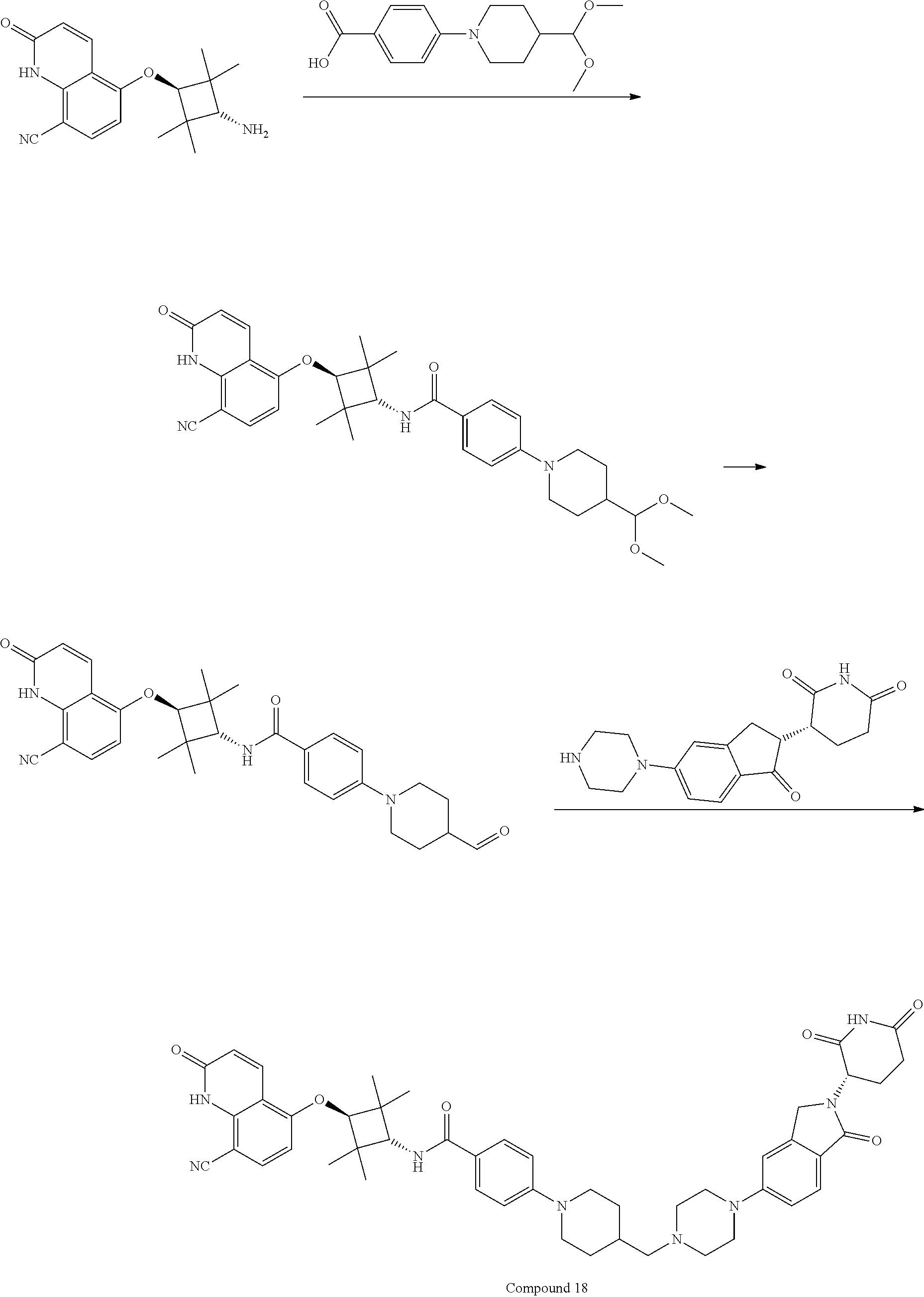

### Step 1: Preparation of N-((1,3-trans)-3-((8-cyano-2-oxo-1,2-dihydroquinolin-5-yl)oxy)-2,2,4,4-tetramethylcyclobutyl)-4-(4-(dimethoxymethyl)piperidin-1-yl)benzamide

To a solution of 4-[4-(dimethoxymethyl)piperidin-1-yl]benzoic acid (27 mg, 0.10 mmol) in DMF (1 mL) was added 5-((1,3-trans)-3-amino-2,2,4,4-tetramethylcyclobutoxy)-2-oxo-1,2-dihydroquino line-8-carbonitrile (30 mg, 0.10 mmol), TEA (58 mg, 0.58 mmol), HOBT (13 mg, 0.10 mmol) and EDCl (18 mg, 0.10 mmol). The reaction mixture was stirred at room temperature for 2 hours. The mixture was diluted with water and extracted with DCM. The organic phase was washed with brine and dried over Na₂SO₄. The solvent was removed in vacuum and the residue was purified via silica gel column chromatography with DCM/MeOH=20/1 to afford the desired compound (30 mg, 54%) as a yellow solid. LC/MS: 573.3 [M+H]+.

### Step 2: Preparation of N-((1,3-trans)-3-((8-cyano-2-oxo-1,2-dihydroquinolin-5-yl)oxy)-2,2,4,4-tetramethylcyclobutyl)-4-(4-formylpiperidin-1-yl)benzamide

A solution of N-((1,3-trans)-3-((8-cyano-2-oxo-1,2-dihydroquinolin-5-yl)oxy)-2,2,4,4-tetramethylcyclobutyl)-4-(4-(dimethoxymethyl)piperidin-1-yl)benzamide (30 mg, 0.05 mmol) in THF/HCl (2 N) (10 mL, 1/1) was stirred at room temperature for 0.5 hour. The mixture was concentrated in vacuum to give the desired compound (20 mg, 72%) as a white solid. LC/MS: 527.2 [M+H]+.

### Step 3: Preparation of N-((1,3-trans)-3-((8-cyano-2-oxo-1,2-dihydroquinolin-5-yl)oxy)-2,2,4,4-tetramethylcyclobutyl)-4-(4-((4-(2-((S)-2,6-dioxopiperidin-3-yl)-1-oxoisoindolin-5-yl)piperazin-1-yl)methyl)piperidin-1-yl)benzamide

To a solution of N-{3-[(8-cyano-2-oxo-1H-quinolin-5-yl)oxy]-2,2,4,4-tetramethylcyclobutyl}-4-(4-formylpiperidin-1-yl)benzamide (25 mg, 0.47 mmol) in DCM/MeOH (4 mL, 1/1) stirred at room temperature was added (S)-3-(1-oxo-5-(piperazin-1-yl)isoindolin-2-yl)piperidine-2,6-dione (28 mg, 0.06 mmol) and NaOAc (8 mg, 0.10 mmol). The mixture was stirred at room temperature for 30 minutes, and NaBH₃CN (6 mg, 0.10 mmol) was added. The reaction mixture was stirred at room temperature for 1 hour. The mixture was diluted with DCM (50 mL) and washed with brine. The organic layer was dried over Na₂SO₄ and concentrated in vacuum to give a crude product. The crude product was purified by Prep-HPLC with ACN—H₂O (0.1% TFA) to afford the desired product (8 mg, 19.3%). LC/MS: 839.1 [M+H]⁺.

1H NMR (400 MHz, DMSO) δ 11.59 (brs, 1H), 10.97 (s, 1H), 9.38 (brs, 1H), 8.38-8.12 (m, 1H), 8.05-7.90 (m, 1H), 7.77 (d, J=8.9 Hz, 2H), 7.61 (d, J=8.4 Hz, 1H), 7.55 (d, J=9.2 Hz, 1H), 7.22-7.15 (m, 2H), 7.00 (d, J=9.0 Hz, 2H), 6.68 (d, J=8.1 Hz, 1H), 5.10-5.05 (m, 1H), 4.42-4.35 (m, 2H), 4.25-4.20 (m, 1H), 4.15-4.10 (m, 1H), 4.05-4.00 (m, 2H), 3.70-3.60 (m, 1H), 3.25-3.10 (m, 6H), 2.95-2.90 (m, 1H), 2.85-2.80 (m, 2H), 2.70-2.60 (m, 2H), 2.42-2.32 (m, 1H), 2.15-2.03 (m, 1H), 1.99-1.87 (m, 1H), 1.86-1.82 (m, 2H), 1.36-1.29 (m, 2H), 1.28-1.21 (m, 7H), 1.20 (s, 6H).

### Example 4: Preparation of N-((1,3-trans)-3-((8-cyano-2-oxo-1,2-dihydroquinolin-5-yl)oxy)-2,2,4,4-tetramethylcyclobutyl)-4-(4-((4-(2-(2,6-dioxopiperidin-3-yl)-1,3-dioxoisoindolin-5-yl)piperazin-1-yl)methyl)piperidin-1-yl)benzamide (Compound 20)

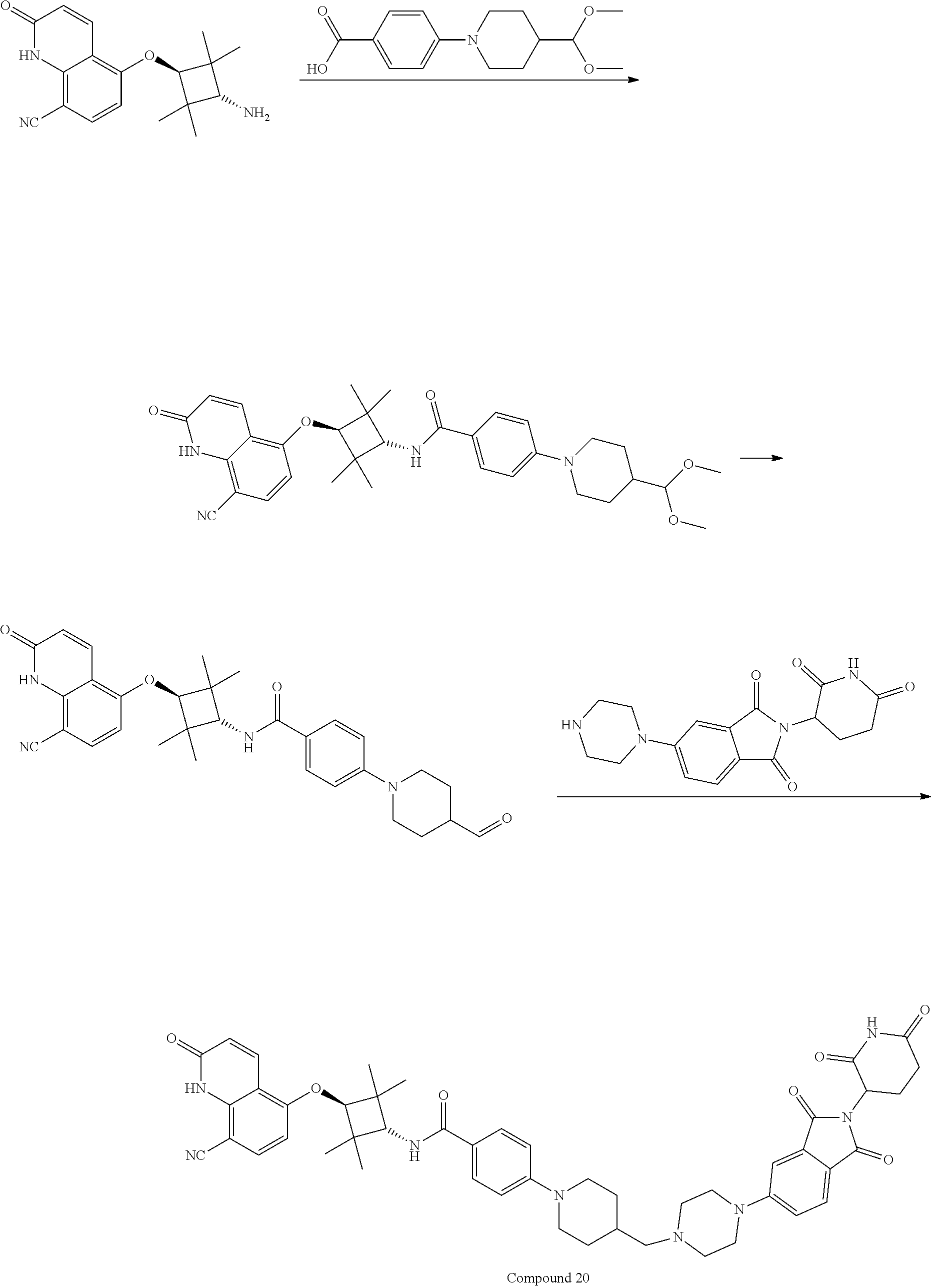

### Step 1: Preparation of N-((1,3-trans)-3-((8-cyano-2-oxo-1,2-dihydroquinolin-5-yl)oxy)-2,2,4,4-tetramethylcyclobutyl)-4-(4-(dimethoxymethyl)piperidin-1-yl)benzamide

To a solution of 5-((1,3-trans)-3-amino-2,2,4,4-tetramethylcyclobutoxy)-2-oxo-1,2-dihydroquinoline-8-carbonitrile (300 mg, 0.96 mmol) and 4-(4-(dimethoxymethyl) piperidin-1-yl)benzoic acid (269 mg, 0.96 mmol) in DMF (5 mL) was added EDCl (221 mg, 1.15 mmol), HOBt (156 mg, 1.15 mmol) and TEA (292 mg, 2.89 mmol). The mixture was stirred at 40° C. for 16 hours. The reaction was quenched with water (5 mL) and extracted with DCM (5 mL×2). The organic phase was concentrated in vacuum and the residue was purified by Flash Chromatography (PE:EA=100:1˜3:1) to give the title compound (350 mg, 60.2% yield) as a brown solid. LC/MS: 573.3[M+H]⁺.

### Step 2: Preparation of N-((1,3-trans)-3-((8-cyano-2-oxo-1,2-dihydroquinolin-5-yl)oxy)-2,2,4,4-tetramethylcyclobutyl)-4-(4-formylpiperidin-1-yl)benzamide

A solution of N-((1,3-trans)-3-((8-cyano-2-oxo-1,2-dihydroquinolin-5-yl)oxy)-2,2,4,4-tetramethylcyclobutyl)-4-(4-(dimethoxymethyl)piperidin-1-yl)benzamide (300 mg, 0.52 mmol) in HCl/dioxane (1 mL, 4 N) was stirred at room temperature for 1 hour. The mixture was concentrated in vacuum to give the title compound (300 mg, crude) as a colorless oil. LC/MS: 527.2 [M+H]⁺.

### Step 3: Preparation of N-((1,3-trans)-3-((8-cyano-2-oxo-1,2-dihydroquinolin-5-yl)oxy)-2,2,4,4-tetramethylcyclobutyl)-4-(4-((4-(2-(2,6-dioxopiperidin-3-yl)-1,3-dioxoisoindolin-5-yl)piperazin-1-yl)methyl)piperidin-1-yl)benzamide

To a solution of 2-(2,6-dioxopiperidin-3-yl)-5-(piperazin-1-yl)isoindoline-1,3-dione (45.5 mg, 0.13 mmol) and N-((1,3-trans)-3-((8-cyano-2-oxo-1,2-dihydroquinolin-5-yl)oxy)-2,2,4,4-tetramethylcyclobutyl)-4-(4-formylpiperidin-1-yl)benzamide (70 mg, 0.13 mmol) in DCM (2 mL) and MeOH (2 mL) was added DIEA (51.5 mg, 0.39 mmol) and HOAc (2 drops). The mixture was stirred at room temperature for 1 hour. NaBH₃CN (8.35 mg, 0.13 mmol) was added. The mixture was stirred at room temperature for 15 hours. The reaction was quenched with water (5 mL) and extracted with DCM (5 mL×2). The organic phase was concentrated in vacuum and the residue was purified by Prep-TLC (DCM:MeOH=10:1) to give the title compound (52.1 mg, 43.6% yield) as a white solid. LC/MS: 853.3 [M+H]⁺.

¹H NMR (400 MHz, DMSO) δ 11.63 (brs, 1H), 11.10 (s, 1H), 8.33-8.17 (m, 1H), 8.02-7.95 (m, 1H), 7.75 (d, J=8.7 Hz, 2H), 7.70 (dt, J=12.2, 2.9 Hz, 1H), 7.54 (d, J=9.2 Hz, 1H), 7.41-7.32 (m, 1H), 7.31-7.22 (m, 1H), 6.97 (d, J=8.8 Hz, 2H), 6.71 (d, J=8.8 Hz, 2H), 5.14-5.05 (dd, J=7.8, 8.2 Hz, 1H), 4.40 (s, 1H), 4.12 (d, J=9.0 Hz, 1H), 3.93-3.81 (m, 2H), 3.51-3.40 (m, 4H), 3.39-3.34 (m, 6H), 2.93-2.75 (m, 3H), 2.62-2.53 (m, 2H), 2.26-2.17 (m, 2H), 2.05-1.98 (m, 1H), 1.86-1.77 (m, 3H), 1.31-1.20 (m, 7H), 1.19 (s, 6H).

### Example 5: Preparation of N-((1,3-trans)-3-((8-cyano-4-oxo-1,4-dihydroquinolin-5-yl)oxy)-2,2,4,4-tetramethylcyclobutyl)-4-(4-((4-(2-((S)-2,6-dioxopiperidin-3-yl)-1-oxoisoindolin-5-yl)piperazin-1-yl)methyl)piperidin-1-yl)benzamide (Compound 30)

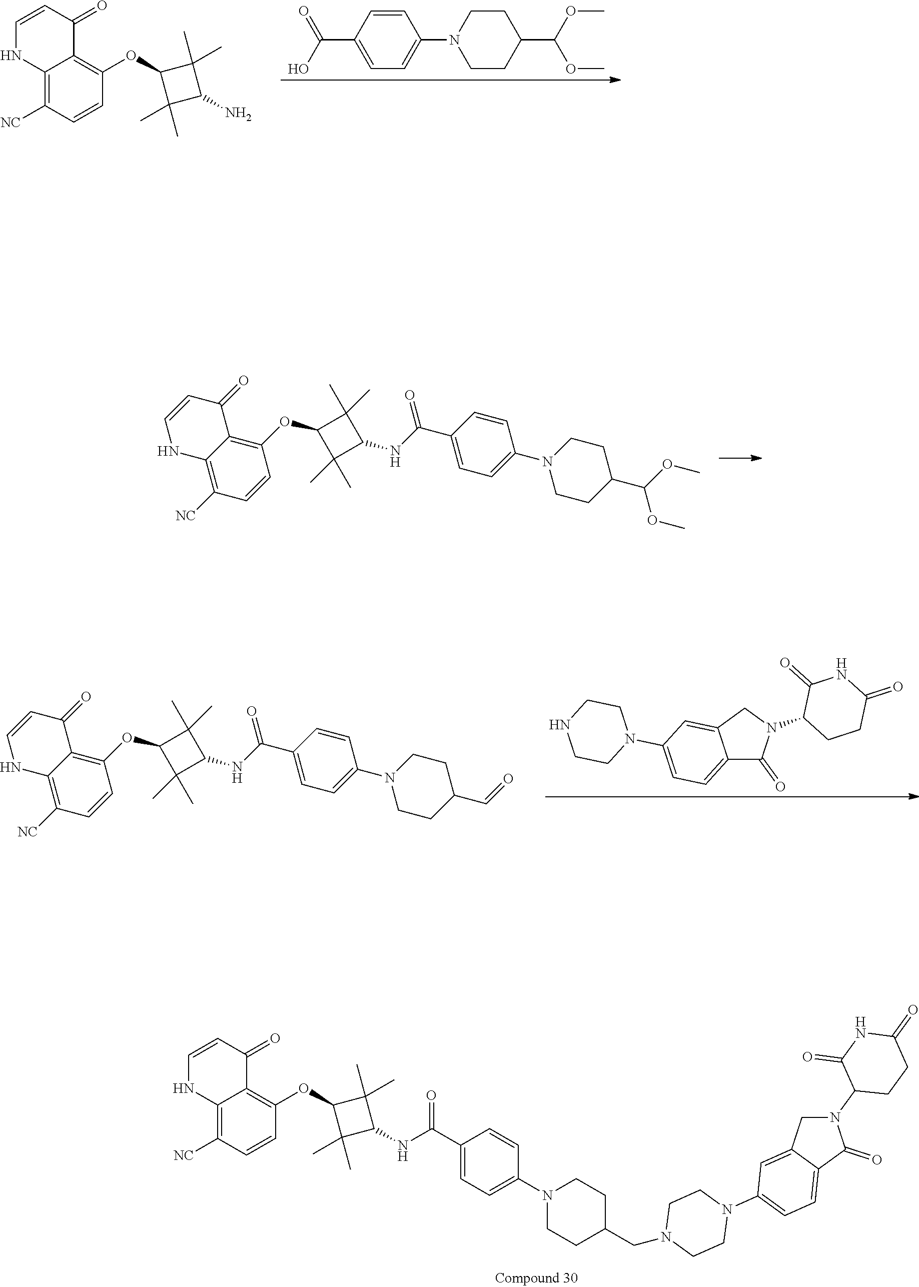

### Step 1: Preparation of N-((1,3-trans)-3-((8-cyano-4-oxo-1,4-dihydroquinolin-5-yl)oxy)-2,2,4,4-tetramethylcyclobutyl)-4-(4-(dimethoxymethyl)piperidin-1-yl)benzamide

To a solution of 5-((1,3-trans)-3-amino-2,2,4,4-tetramethylcyclobutoxy)-4-oxo-1,4-dihydroquinoline-8-carbonitrile (55 mg, 0.18 mmol) in DMF (5 mL) was added 4-(4-(dimethoxymethyl)piperidin-1-yl)benzoic acid (50 mg, 0.18 mmol), HOBT (36.3 mg, 0.27 mmol), EDCl (51.5 mg, 0.27 mmol) and DIEA (80.9 mg, 0.62 mmol). The reaction was stirred under nitrogen at 25° C. for 8 hours. The mixture was poured into water and extracted with DCM. The organic phase was washed with brine and dried over Na₂SO₄. The solvent was removed in vacuum and the residue was purified by Prep-TLC with DCM:MeOH=10:1 to afford the desired compound (60 mg, 58.2%) as a yellow solid. LC/MS: 573.3 [M+H]⁺.

### Step 2: Preparation of N-((1,3-trans)-3-((8-cyano-4-oxo-1,4-dihydroquinolin-5-yl)oxy)-2,2,4,4-tetra methylcyclobutyl)-4-(4-formylpiperidin-1-yl)benzamide

A mixture of N-((1,3-trans)-3-((8-cyano-4-oxo-1,4-dihydroquinolin-5-yl)oxy)-2,2,4,4-tetramethyl cyclobutyl)-4-(4-(dimethoxymethyl)piperidin-1-yl)benzamide (60 mg, 0.10 mmol) in THF/HCl (2N) (5 mL, 1/1) was stirred under nitrogen at 25° C. for 2 hours. The reaction mixture was neutralized with saturated bicarbonate sodium solution and extracted with DCM. The organic phase was washed with brine and dried over Na₂SO₄. The solvent was removed in vacuum to afford the desired compound (60 mg crude) as a yellow solid. LC/MS: 527.2 [M+H]⁺.

### Step 3: Preparation of N-((1,3-trans)-3-((8-cyano-4-oxo-1,4-dihydroquinolin-5-yl)oxy)-2,2,4,4-tetramethylcyclobutyl)-4-(4-((4-(2-((S)-2,6-dioxopiperidin-3-yl)-1-oxoisoindolin-5-yl)piperazin-1-yl)methyl)piperidin-1-yl)benzamide

To a solution of N-((1,3-trans)-3-((8-cyano-4-oxo-1,4-dihydroquinolin-5-yl)oxy)-2,2,4,4-tetramethylcyclobutyl)-4-(4-formylpiperidin-1-yl)benzamide (60 mg, crude) in DCM:MeOH (5 mL, 1:1) was added (S)-3-(1-oxo-5-(piperazin-1-yl)isoindolin-2-yl)piperidine-2,6-dione (41.1 mg, 0.12 mmol) and DIEA (44.2 mg, 0.34 mmol). The mixture was stirred under nitrogen at 25° C. for 30 minutes. HOAc (0.05 mL) and Sodium cyanoborohydride (14.3 mg, 0.22 mmol) were added. The reaction was stirred at 25° C. for 1 hour. The reaction mixture was quenched with H₂O and extracted with DCM. The organic phase was washed with brine and dried over Na₂SO₄. The solvent was removed in vacuum and the residue was purified by Prep-HPLC with ACN—H₂O (0.1% TFA) to afford the desired compound (20 mg, 20.9% for two steps) as a white solid. LC/MS: 839.4 [M+H]⁺.

¹H NMR (400 MHz, DMSO) δ 10.98 (s, 1H), 9.50 (brs, 1H), 8.07 (d, J=8.4 Hz, 1H), 7.99 (brs, 1H), 7.78 (d, J=8.8 Hz, 2H), 7.61 (d, J=8.4 Hz, 1H), 7.52 (d, J=9.6 Hz, 1H), 7.21-7.15 (m, 2H), 7.01 (d, J=9.2 Hz, 2H), 6.70 (d, J=8.8 Hz, 1H), 6.35 (brs, 1H), 5.10-5.05 (m, 1H), 4.41-4.34 (m, 2H), 4.28-4.21 (m, 1H), 4.11 (d, J=9.2 Hz, 1H), 4.07-3.98 (m, 2H), 3.91 (d, J=12.4 Hz, 2H), 3.70-3.60 (m, 2H), 3.23-3.18 (m, 3H), 3.15-3.11 (m, 2H), 2.99-2.89 (m, 1H), 2.84 (t, J=11.5 Hz, 2H), 2.69-2.55 (m, 2H), 2.45-2.32 (m, 1H), 2.16-2.04 (m, 1H), 2.03-1.92 (m, 1H), 1.85 (d, J=11.2 Hz, 2H), 1.37-1.29 (m, 2H), 1.26 (s, 6H), 1.20 (s, 6H).

### Example 6: Preparation of N-((1,3-trans)-3-((8-cyano-1-methyl-2-oxo-1,2-dihydroquinolin-5-yl)oxy)-2,2,4,4-tetramethylcyclobutyl)-4-(4-((4-(2-((S)-2,6-dioxopiperidin-3-yl)-1-oxoisoindolin-5-yl)piperazin-1-yl)methyl)piperidin-1-yl)benzamide (Compound 34)

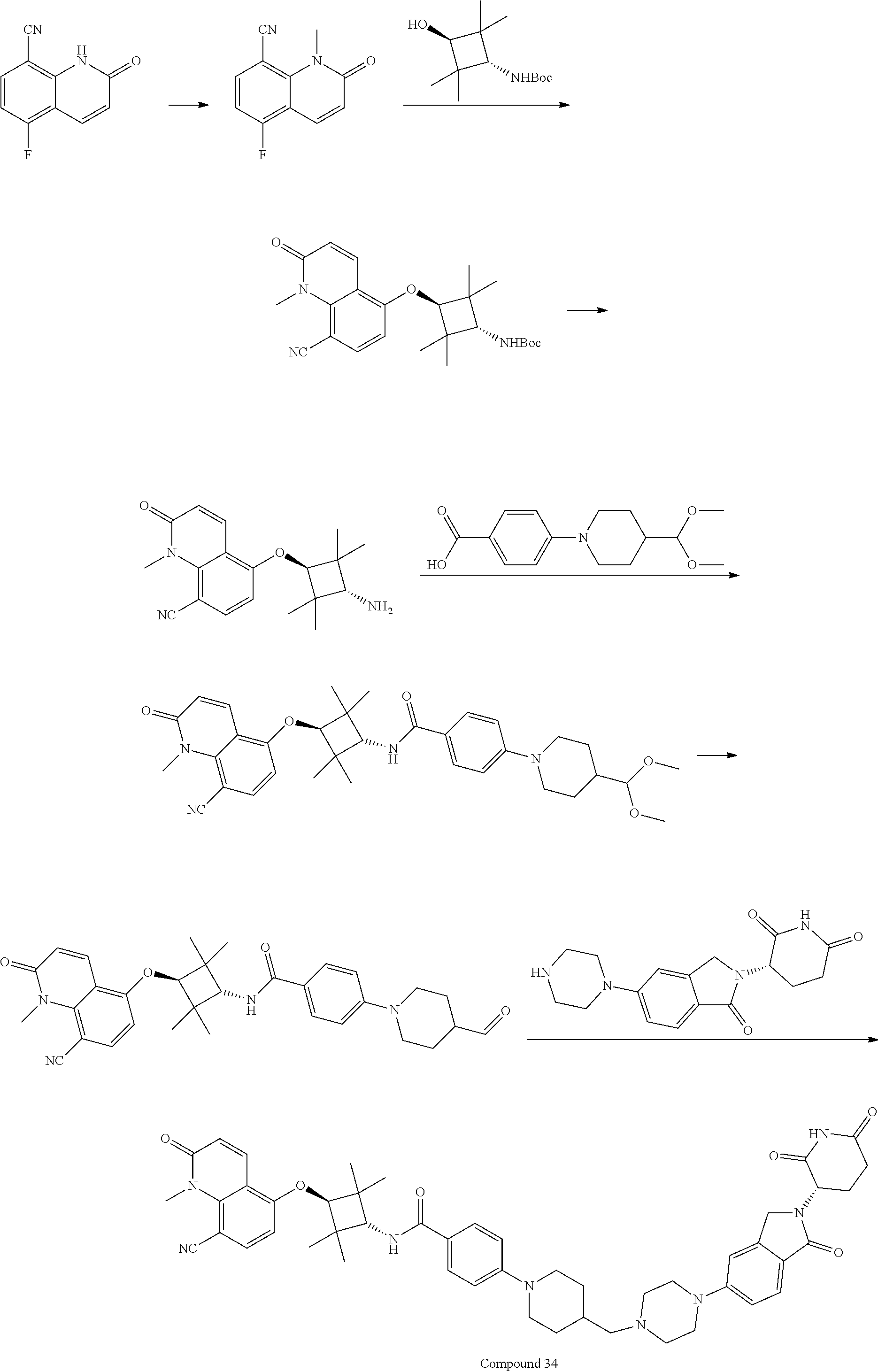

### Step 1: Preparation of 5-fluoro-1-methyl-2-oxo-1,2-dihydroquinoline-8-carbonitrile

To a solution of 5-fluoro-2-oxo-1,2-dihydroquinoline-8-carbonitrile (1.2 g, 6.37 mmol) and K₂CO₃ (1758 mg, 12.74 mmol) in DMSO (20 mL) was added Mel (1.1 g, 7.65 mmol). The mixture was stirred at 25° C. for 4 hours under N₂ atmosphere. The reaction was quenched with water (20 mL) and extracted with EA (20 mL×3). The organic phase was washed with brine and dried over Na₂SO₄. The solution was concentrated in vacuum and the residue was purified by flash chromatography (PE:EA=10:1) to give the desired compound (100 mg, 7.4% yield) as a white solid. LC/MS: 203.0 [M+H]⁺.

### Step 2: Preparation of tert-butyl ((1,3-trans)-3-((8-cyano-1-methyl-2-oxo-1,2-dihydroquinolin-5-yl)oxy)-2,2,4,4-tetramethylcyclobutyl)carbamate

To a solution of 5-fluoro-1-methyl-2-oxo-1,2-dihydroquinoline-8-carbonitrile (100 mg, 0.49 mmol) in THF (4 mL) was added NaH (39 mg, 60%, 1.0 mmol) at 0° C. The mixture was stirred for 30 min under N₂ atmosphere and then tert-butyl ((1,3-trans)-3-hydroxy-2,2,4,4-tetramethylcyclo butyl)carbamate (144 mg, 0.59 mmol) was added. The mixture was stirred at room temperature for 4 hours. The reaction was quenched with water (20 mL) and extracted with DCM (20 mL×3). The organic phase was washed with brine and dried over Na₂SO₄. The solution was concentrated in vacuum and the residue was purified by Prep-TLC (PE:EA=3:1) to give the desired compound (150 mg, 67.7% yield) as a white solid. LC/MS: 426.2 [M+H]⁺.

### Step 3: Preparation of 5-((1,3-trans)-3-amino-2,2,4,4-tetramethylcyclobutoxy)-1-methyl-2-oxo-1,2-dihydroquinoline-8-carbonitrile

To a solution of tert-butyl ((1,3-trans)-3-((8-cyano-1-methyl-2-oxo-1,2-dihydroquinolin-5-yl)oxy)-2,2,4,4-tetramethylcyclobutyl)carbamate (150 mg, 0.36 mmol) in DCM (5 mL) was added TFA (5 mL) at 0° C. The solution was stirred at 25° C. for 1 hour. The mixture was concentrated in vacuum to afford the desired compound (150 mg, crude) as a brown solid. LC/MS: 326.2 [M+H]⁺.

### Step 4: Preparation of N-((1,3-trans)-3-((8-cyano-1-methyl-2-oxo-1,2-dihydroquinolin-5-yl)oxy)-2,2,4,4-tetramethylcyclobutyl)-4-(4-(dimethoxymethyl)piperidin-1-yl)benzamide

To a solution of 5-((1,3-trans)-3-amino-2,2,4,4-tetramethylcyclobutoxy)-1-methyl-2-oxo-1,2-dihydroquinoline-8-carbonitrile (100 mg, 0.30 mmol) and 4-(4-(dimethoxymethyl) piperidin-1-yl)benzoic acid (103 mg, 0.37 mmol) in DMF (4 mL) was added DIEA (129 mg, 1.0 mmol), HOBT (49.8 mg, 0.37 mmol) and EDCl (70.7 mg, 0.37 mmol). The mixture was stirred at RT for 12 hours. The reaction was quenched with water (20 mL) and extracted with EA (20 mL×3). The organic phase was washed with brine and dried over Na₂SO₄. The solution was concentrated in vacuum and the residue was purified by Prep-TLC (DCM:MeOH=95:5) to give the desired compound (100 mg, 52.7% yield) as a white solid. LC/MS: 587.3 [M+H]⁺.

### Step 5: Preparation of N-((1,3-trans)-3-((8-cyano-1-methyl-2-oxo-1,2-dihydroquinolin-5-yl)oxy)-2,2,4,4-tetramethylcyclobutyl)-4-(4-formylpiperidin-1-yl)benzamide

A solution of N-((1,3-trans)-3-((8-cyano-1-methyl-2-oxo-1,2-dihydroquinolin-5-yl)oxy)-2,2,4,4-tetramethylcyclobutyl)-4-(4-(dimethoxymethyl)piperidin-1-yl)benzamide (100 mg, 0.17 mmol) in HCl/dioxane (4 M, 2 mL) was stirred at 25° C. for 1 hour. The mixture was concentrated in vacuum to afford the desired compound (60 mg, crude) as a white solid. LC/MS: 541.6 [M+H]⁺.

### Step 6: Preparation of N-((1,3-trans)-3-((8-cyano-1-methyl-2-oxo-1,2-dihydroquinolin-5-yl)oxy)-2,2,4,4-tetramethylcyclobutyl)-4-(4-((4-(2-((S)-2,6-dioxopiperidin-3-yl)-1-oxoisoindolin-5-yl)piperazin-1-yl)methyl)piperidin-1-yl)benzamide

To a solution of N-((1,3-trans)-3-((8-cyano-1-methyl-2-oxo-1,2-dihydroquinolin-5-yl)oxy)-2,2,4,4-tetramethylcyclobutyl)-4-(4-formylpiperidin-1-yl)benzamide (60 mg, 0.11 mmol) and (S)-3-(1-oxo-5-(piperazin-1-yl)isoindolin-2-yl)piperidine-2,6-dione (40 mg, 0.12 mmol) in DCM (4 mL) was added DIEA (28 mg, 0.22 mmol). The mixture was stirred at room temperature for 1 hour then STAB (70 mg, 0.33 mmol) was added. The solution was stirred for 4 hours. The mixture was poured into water (10 mL) and extracted with EA (20 mL×3). The combined organic layer was dried over Na₂SO₄ and concentrated in vacuum. The residue was purified by Prep-TLC (DCM:MeOH=95:5) to give the desired compound (13.8 mg, 21.7% yield) as a white solid. LC/MS: 853.3 [M+H]⁺.

¹H NMR (400 MHz, DMSO) δ 10.96 (s, 1H), 8.24 (d, J=9.8 Hz, 1H), 8.04 (d, J=8.7 Hz, 1H), 7.78-7.73 (m, 2H), 7.59-7.51 (m, 2H), 7.12-7.07 (m, 2H), 6.99-6.91 (m, 2H), 6.76 (dd, J=9.3, 6.9 Hz, 2H), 5.10-5.02 (m, 1H), 4.42 (s, 1H), 4.38-4.29 (m, 1H), 4.24-4.18 (m, 1H), 4.12 (d, J=9.1 Hz, 1H), 3.96 (s, 2H), 3.91-3.81 (m, 2H), 3.32-3.24 (m, 3H), 2.93-2.86 (m, 2H), 2.81-2.75 (m, 2H), 2.64-2.55 (m, 5H), 2.43-2.34 (m, 1H), 2.24-2.20 (m, 2H), 2.02-1.91 (m, 2H), 1.85-1.73 (m, 3H), 1.26 (s, 6H), 1.23-1.20 (m, 2H), 1.19 (s, 6H).

### Example 7: Preparation of N-((1,3-trans)-3-((8-cyano-1-methyl-4-oxo-1,4-dihydroquinolin-5-yl)oxy)-2,2,4,4-tetramethylcyclobutyl)-4-(4-((4-(2-((S)-2,6-dioxopiperidin-3-yl)-1-oxoisoindolin-5-yl)piperazin-1-yl)methyl)piperidin-1-yl)benzamide (Compound 39)

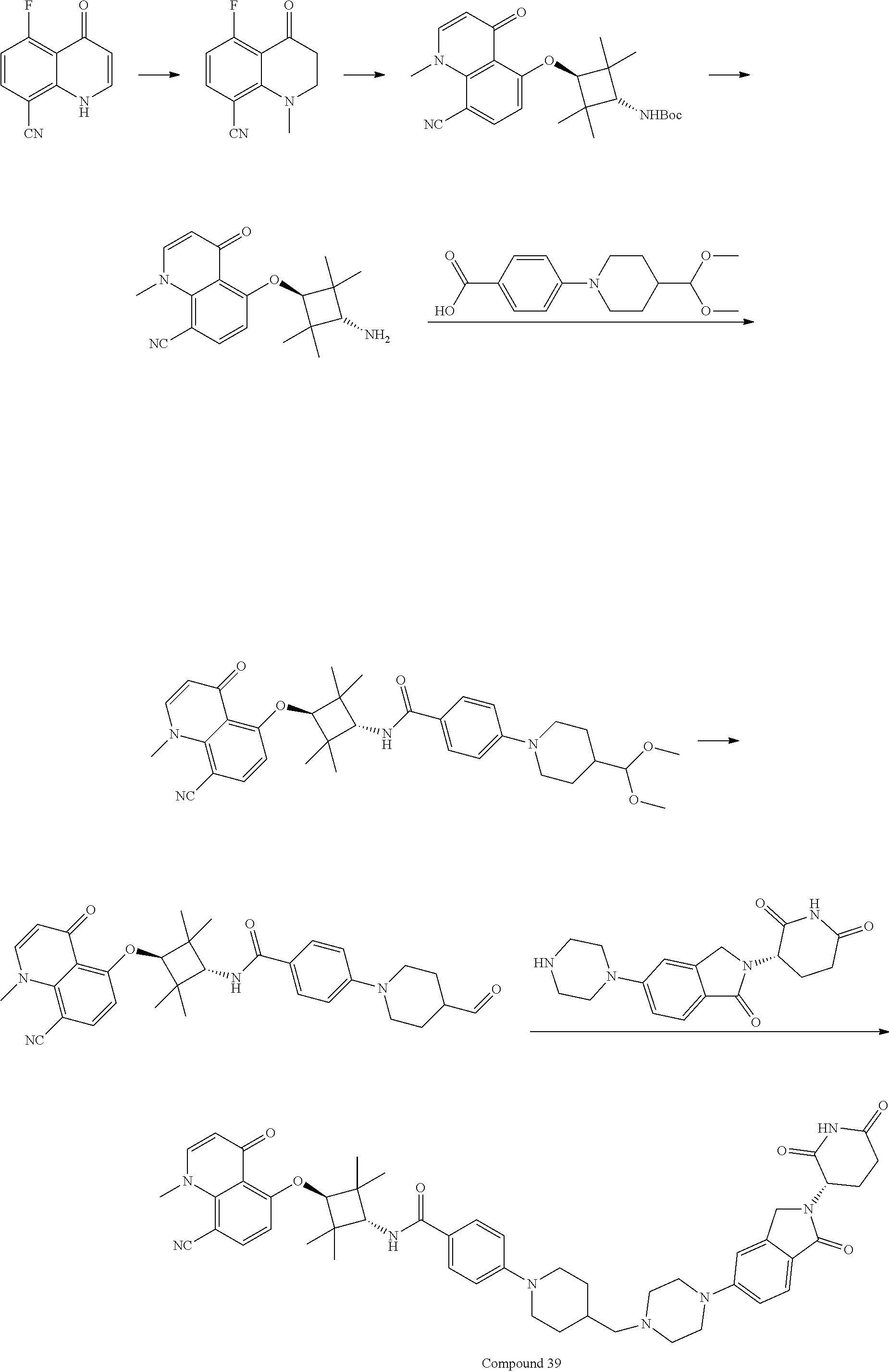

### Step 1: Preparation of 5-fluoro-1-methyl-4-oxo-1,4-dihydroquinoline-8-carbonitrile

To a solution of 5-fluoro-4-oxo-1,4-dihydroquinoline-8-carbonitrile (500 mg, 2.66 mmol) in DMF (10 mL) stirred at 0° C. was added NaH (318 mg, 60%, 7.97 mmol). The mixture was stirred at 0° C. for 30 minutes and then iodomethane (1.8 g, 13.25 mmol) was added. The reaction was stirred at 25° C. for 12 hours. The solvent was removed in vacuum and the residue was washed with EA. The combined organic phase was washed with brine and dried over Na₂SO₄. The solvent was removed in vacuum and the residue was purified by silica-gel column chromatography and eluted with 0-50% EA in PE to give the title compound (300 mg, 55.6% yield) as a white solid. LC/MS: 203.2 [M+H]⁺.

### Step 2: Preparation of tert-butyl((1,3-trans)-3-((8-cyano-1-methyl-4-oxo-1,4-dihydroquinolin-5-yl)oxy)-2,2,4,4-tetramethylcyclobutyl)carbamate

To a solution of 5-fluoro-1-methyl-4-oxo-1,4-dihydroquinoline-8-carbonitrile (300 mg, 1.48 mmol) in THF (20 mL) stirred at 0° C. was added NaH (296 mg, 60%, 7.4 mmol). The mixture was stirred at 0° C. for 30 minutes then tert-butyl((1,3-trans)-3-hydroxy-2,2,4,4-tetramethylcyclobutyl)carbamate (360 mg, 1.48 mmol) was added. The reaction mixture was stirred at 50° C. for 6 hours. The mixture was poured into ice water and extracted with DCM. The combined organic phase was washed with brine and dried over Na₂SO₄. The solvent was removed in vacuum and the residue was purified by Prep-TLC with DCM:MeOH=10:1 to afford the desired compound (400 mg, 63.5% yield) as a yellow solid. LC/MS: 426.3 [M+H]⁺.

### Step 3: Preparation of 5-((1,3-trans)-3-amino-2,2,4,4-tetramethylcyclobutoxy)-1-methyl-4-oxo-1,4-dihydroquinoline-8-carbonitrile

A mixture of tert-butyl ((1,3-trans)-3-((8-cyano-1-methyl-4-oxo-1,4-dihydroquinolin-5-yl)oxy)-2,2,4,4-tetramethylcyclobutyl)carbamate (400 mg, 0.94 mmol) in DCM:TFA (8 mL, 3:1) was stirred at room temperature for 1 hour. The solvent was removed in vacuum to give the title compound (230 mg, crude) as a yellow solid. LC/MS: 326.2 [M+H]⁺.

### Step 4: Preparation of N-((1,3-trans)-3-((8-cyano-1-methyl-4-oxo-1,4-dihydroquinolin-5-yl)oxy)-2,2,4,4-tetramethylcyclobutyl)-4-(4-(dimethoxymethyl)piperidin-1-yl)benzamide

To a solution of 5-((1,3-trans)-3-amino-2,2,4,4-tetramethylcyclobutoxy)-1-methyl-4-oxo-1,4-dihydroquinoline-8-carbonitrile (230 mg, 0.71 mmol) in DMF (5 mL) stirred at room temperature was added 4-(4-(dimethoxymethyl)piperidin-1-yl)benzoic acid (210.1 mg, 0.71 mmol), HOBT (143.3 mg, 1.06 mmol), EDCl (136.1 mg, 1.06 mmol) and DIEA (366.4 mg, 2.84 mmol). The reaction was stirred under nitrogen at room temperature for 8 hours. The mixture was poured into water and extracted with DCM. The organic phase was washed with brine and dried over Na₂SO₄. The solvent was removed in vacuum and the residue was purified by Prep-TLC with DCM:MeOH=10:1 to afford the desired compound (200 mg, 36.1% yield for two steps) as a yellow solid. LC/MS: 587.3 [M+H]⁺.

### Step 5: Preparation of N-((1,3-trans)-3-((8-cyano-1-methyl-4-oxo-1,4-dihydroquinolin-5-yl)oxy)-2,2,4,4-tetramethylcyclobutyl)-4-(4-formylpiperidin-1-yl)benzamide

A mixture of N-((1,3-trans)-3-((8-cyano-1-methyl-4-oxo-1,4-dihydroquinolin-5-yl)oxy)-2,2,4,4-tetramethylcyclobutyl)-4-(4-(dimethoxymethyl)piperidin-1-yl)benzamide (200 mg, 0.33 mmol) in THF/HCl (2 N) (5 mL, 1/1) was stirred under nitrogen at 25° C. for 1 hour. The mixture was neutralized with saturated bicarbonate sodium solution and extracted with DCM. The organic phase was washed with brine and dried over Na₂SO₄. The solvent was removed in vacuum to afford the desired compound (130 mg, 72.7% yield) as a yellow solid. LC/MS: 541.1 [M+H]⁺.

### Step 6: Preparation of N-((1,3-trans)-3-((8-cyano-1-methyl-4-oxo-1,4-dihydroquinolin-5-yl)oxy)-2,2,4,4-tetramethylcyclobutyl)-4-(4-((4-(2-((S)-2,6-dioxopiperidin-3-yl)-1-oxoisoindolin-5-yl)piperazin-1-yl)methyl)piperidin-1-yl)benzamide

To a solution of N-((1,3-trans)-3-((8-cyano-1-methyl-4-oxo-1,4-dihydroquinolin-5-yl)oxy)-2,2,4,4-tetramethylcyclobutyl)-4-(4-formylpiperidin-1-yl)benzamide (130 mg, 0.2 mmol) in DCM:MeOH (5 mL, 1:1) was added (S)-3-(1-oxo-5-(piperazin-1-yl)isoindolin-2-yl)piperidine-2,6-dione (75 mg, 0.22 mmol) and NaOAc (50 mg, 0.6 mmol). The mixture was stirred under nitrogen at 25° C. for 30 minutes and then AcOH (0.05 mL) and NaCNBH₃ (40 mg, 0.61 mmol) were added. The reaction was stirred at 25° C. for 2 hours. The reaction was quenched with H₂O and extracted with DCM. The organic phase was washed with brine and dried over Na₂SO₄. The solvent was removed in vacuum and the residue was purified by Prep-HPLC with ACN—H₂O (0.1% TFA) to afford the desired compound (35 mg, 20.5% yield) as a yellow solid. LC/MS: 853.4 [M+H]⁺.

¹H NMR (400 MHz, DMSO) δ 10.96 (s, 1H), 8.82 (d, J=5.6 Hz, 1H), 8.20 (d, J=8.4 Hz, 1H), 7.76 (d, J=8.8 Hz, 2H), 7.59-7.50 (m, 2H), 7.17 (d, J=5.6 Hz, 1H), 7.09 (s, 2H), 6.97 (d, J=9.2 Hz, 2H), 6.83 (d, J=8.8 Hz, 1H), 5.06 (dd, J=13.2, 5.2 Hz, 1H), 4.43 (s, 1H), 4.34 (d, J=17.2 Hz, 1H), 4.21 (d, J=17.2 Hz, 1H), 4.11 (d, J=9.2 Hz, 1H), 4.04 (s, 3H), 3.87 (d, J=12.0 Hz, 2H), 3.31-3.19 (m, 4H), 2.96-2.85 (m, 1H), 2.80 (t, J=12.0 Hz, 2H), 2.62-2.56 (m, 1H), 2.55-2.53 (m, 3H), 2.44-2.32 (m, 2H), 2.30-2.13 (m, 2H), 1.99-1.93 (m, 1H), 1.88-1.75 (m, 3H), 1.28 (s, 6H), 1.24-1.22 (m, 1H), 1.20 (s, 6H), 1.17 (d, J=7.2 Hz, 1H).

### Example 8: Preparation of 5-(4-((1-(4-(((1,3-trans)-3-((8-cyano-2-oxo-1,2-dihydroquinolin-5-yl)oxy)-2,2,4,4-tetramethylcyclobutyl)carbamoyl)phenyl)piperidin-4-yl)methyl)piperazin-1-yl)-N—((S)-2,6-dioxopiperidin-3-yl)picolinamide (Compound 43)

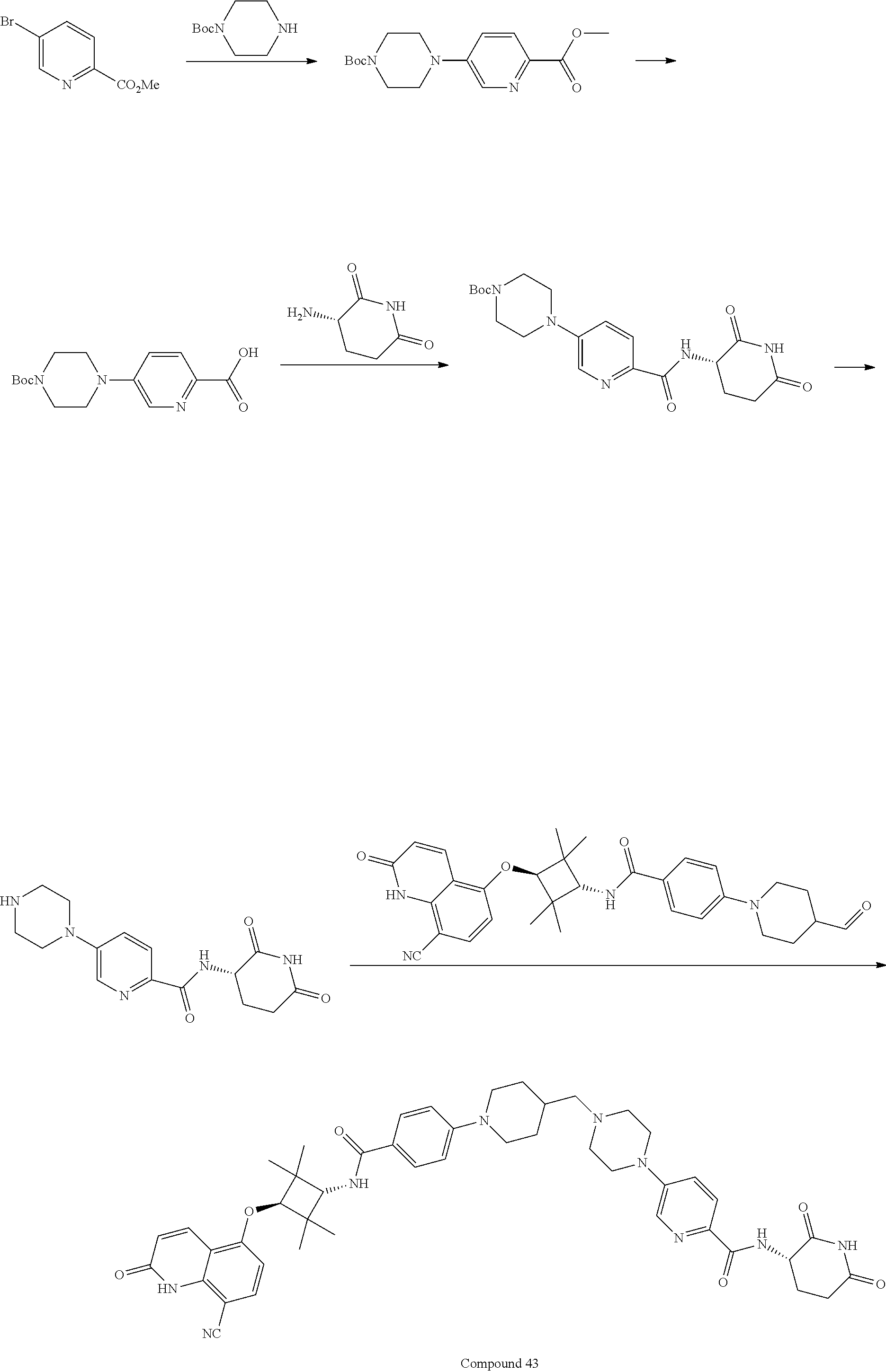

### Step 1: Preparation of tert-butyl 4-(6-(methoxycarbonyl)pyridin-3-yl)piperazine-1-carboxylate

To a solution of methyl 5-bromopyridine-2-carboxylate (2 g, 9.3 mmol) in toluene (20 mL) stirred under argon at room temperature was added tert-butyl piperazine-1-carboxylate (1.91 g, 10.2 mmol), BINAP (1.16 g, 1.8 mmol), Pd₂(dba)₃ (0.53 g, 0.93 mmol) and Cs₂CO₃ (9.09 g, 27.9 mmol). The reaction mixture was stirred at 100° C. temperature for 12 hours under argon atmosphere. The mixture was diluted with water and extracted with DCM. The organic phase was washed with brine and dried over Na₂SO₄. The solution was concentrated in vacuum. The residue was purified via silica gel column chromatography with DCM/MeOH=20/1 to afford the desired compound (1.9 g, 63.4% yield) as a yellow solid. LC/MS:322.2 [M+H]⁺.

### Step 2: Preparation of 5-(4-(tert-butoxycarbonyl)piperazin-1-yl)picolinic acid

To a solution of tert-butyl 4-[6-(methoxycarbonyl)pyridin-3-yl]piperazine-1-carboxylate (1.8 g, 5.6 mmol) in MeOH (20 mL) and H₂O (10 mL) was added NaOH (0.67 g, 16.8 mmol). The reaction mixture was stirred at room temperature for 2 hours. The mixture was acidified with HCl (2N) until pH=3 and extracted with DCM. The organic phase was washed with brine and dried over Na₂SO₄. The solution was concentrated in vacuum to give the desired compound (1.7 g, 98.2% yield) as a yellow solid. LC/MS: 308.2 [M+H]⁺.

### Step 3: Preparation of tert-butyl (S)-4-(6-((2,6-dioxopiperidin-3-yl)carbamoyl)pyridin-3-yl)piperazine-1-carboxylate

To a solution of 5-{4-[(tert-butoxy)carbonyl]piperazin-1-yl}pyridine-2-carboxylic acid (1.2 g, 3.9 mmol) in DMF (20 mL) was added (3S)-3-aminopiperidine-2,6-dione hydrochloride (640 mg, 3.9 mmol), DIEA (3 g, 23.4 mmol) and HATU (2.2 g, 5.9 mmol). The reaction mixture was stirred at room temperature for 2 hours. The mixture was diluted with water and extracted with DCM. The organic phase was washed with brine and dried over Na₂SO₄. The solution was concentrated under vacuum. The residue was purified by silica gel column chromatography with DCM/MeOH=20/1 to afford the desired compound (1.6 g, 98.1% yield) as a yellow solid. LC/MS:418.2 [M+H]⁺.

### Step 4: Preparation of (S)—N-(2,6-dioxopiperidin-3-yl)-5-(piperazin-1-yl)picolinamide

To a solution tert-butyl 4-(6-{[(3S)-2,6-dioxopiperidin-3-yl]carbamoyl}pyridin-3-yl)piperazine-1-carboxylate (1.6 g, 3.8 mmol) in DCM (10 mL) was added TFA (10 mL). The reaction was stirred at room temperature for 1 hour. The mixture was concentrated in vacuum to give the desired compound (2 g, crude) as a white solid. LC/MS: 318.1 [M+H]⁺.

### Step 5: Preparation of 5-(4-((1-(4-(((1,3-trans)-3-((8-cyano-2-oxo-1,2-dihydroquinolin-5-yl)oxy)-2,2,4,4-tetramethylcyclobutyl)carbamoyl)phenyl)piperidin-4-yl)methyl)piperazin-1-yl)-N—((S)-2,6-dioxopiperidin-3-yl)picolinamide

To a solution of (S)—N-(2,6-dioxopiperidin-3-yl)-5-(piperazin-1-yl)picolinamide (180 mg, crude) in DCM (2 mL) was added N-((1,3-trans)-3-((8-cyano-2-oxo-1,2-dihydroquinolin-5-yl)oxy)-2,2,4,4-tetramethylcyclobutyl)-4-(4-formylpiperidin-1-yl)benzamide (100 mg, 0.19 mmol), NaOAc (31 mg, 0.38 mmol) and NaBH₃CN (24 mg, 0.38 mmol). The mixture was stirred at room temperature for 16 hours. The reaction was diluted with water (10 mL) and extracted with DCM (10 mL×3). The organic phase was washed with brine and dried over Na₂SO₄. The solution was concentrated in vacuum and the residue was purified by flash chromatography on silica gel (5% MeOH in DCM) to give a crude product. The crude product was purified by Prep-HPLC (0.1% FA in H₂O/ACN=22˜25%) to give the desired product (25 mg, 15.9% yield) as a white solid. LC/MS: 828.3 [M+H]⁺.

¹H NMR (400 MHz, DMSO) δ 11.63 (brs, 1H), 10.86 (s, 1H), 8.74 (d, J=8.3 Hz, 1H), 8.33 (s, 1H), 8.25 (d, J=9.6 Hz, 1H), 7.98 (d, J=8.5 Hz, 1H), 7.87 (d, J=8.7 Hz, 1H), 7.75 (d, J=8.6 Hz, 2H), 7.54 (d, J=9.2 Hz, 1H), 7.47-7.40 (m, 1H), 6.97 (d, J=8.8 Hz, 2H), 6.75-6.67 (m, 2H), 4.79-4.72 (m, 1H), 4.40 (s, 1H), 4.12 (d, J=9.1 Hz, 1H), 3.87 (d, J=12.0 Hz, 2H), 3.35 (s, 4H), 2.80 (t, J=12.4 Hz, 3H), 2.58-2.52 (m, 5H), 2.28-2.11 (m, 3H), 2.04-1.98 (m, 1H), 1.84-1.76 (m, 3H), 1.26 (s, 6H), 1.24-1.21 (m, 1H), 1.20 (s, 6H).

### Example 9: Preparation of N-((1,3-trans)-3-((8-cyano-2-oxo-1,2-dihydroquinolin-5-yl)oxy)-2,2,4,4-tetramethylcyclobutyl)-4-(4-((4-(2-(2,6-dioxopiperidin-3-yl)-1-oxo-1,2-dihydrophthalazin-6-yl)piperazin-1-yl)methyl)piperidin-1-yl)benzamide (Compound 49)

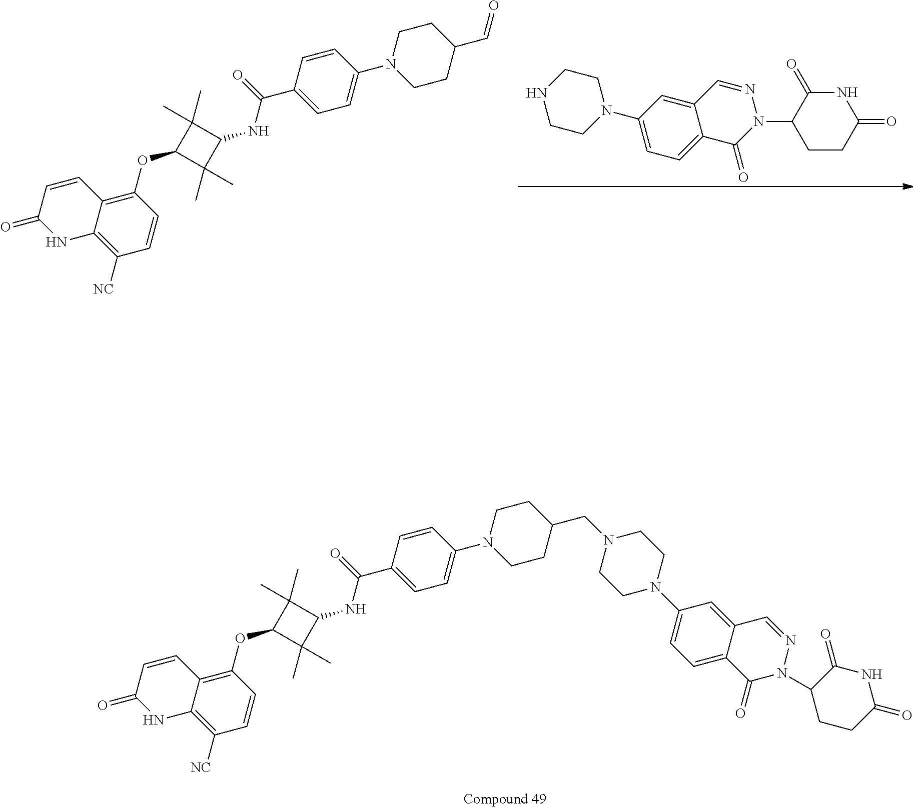

### Step 1: Preparation of N-((1,3-trans)-3-((8-cyano-2-oxo-1,2-dihydroquinolin-5-yl)oxy)-2,2,4,4-tetramethylcyclobutyl)-4-(4-((4-(2-(2,6-dioxopiperidin-3-yl)-1-oxo-1,2-dihydrophthalazin-6-yl)piperazin-1-yl)methyl)piperidin-1-yl)benzamide

To a solution of N-((1,3-trans)-3-((8-cyano-2-oxo-1,2-dihydroquinolin-5-yl)oxy)-2,2,4,4-tetramethylcyclobutyl)-4-(4-formylpiperidin-1-yl)benzamide (100 mg, 0.19 mmol) in DCM (2 mL) was added 3-[1-oxo-6-(piperazin-1-yl)phthalazin-2-yl]piperidine-2,6-dione prepared according to the procedure described in US20230131252 (page 35, intermediate 1-7) (72 mg, 0.19 mmol), NaOAc (31 mg, 0.38 mmol) and NaBH₃CN (24 mg, 0.38 mmol). The reaction was stirred at room temperature for 16 hours. The mixture was diluted with water (10 mL) and extracted with DCM (10 mL×3). The organic phase was washed with brine and dried over Na₂SO₄. The solution was concentrated in vacuum and the residue was purified by flash chromatography on silica gel (5% MeOH in DCM) to give a crude product. The crude product was purified by Prep-HPLC (0.1% FA in H₂O/ACN=22˜25%) to give the desired product (60 mg, 37.1% yield) as a white solid. LC/MS: 852.4 [M+H]⁺.

¹H NMR (400 MHz, DMSO) δ 11.01 (s, 1H), 8.30-8.20 (m, 2H), 8.04 (d, J=9.1 Hz, 1H), 7.97 (d, J=8.6 Hz, 1H), 7.74 (d, J=8.8 Hz, 2H), 7.56-7.48 (m, 2H), 7.26 (s, 1H), 6.97 (d, J=9.0 Hz, 2H), 6.77-6.64 (m, 2H), 5.80-5.71 (m, 1H), 4.40 (s, 1H), 4.12 (d, J=9.3 Hz, 1H), 3.87 (d, J=13.4 Hz, 2H), 3.46-3.40 (m, 4H), 2.97-2.87 (m, 1H), 2.80 (t, J=11.7 Hz, 2H), 2.65-2.55 (m, 2H), 2.54-2.52 (m, 5H), 2.23 (d, J=6.0 Hz, 2H), 2.11-2.06 (m, 2H), 1.87-1.83 (m, 1H), 1.82-1.78 (m, 2H), 1.26 (s, 6H), 1.24-1.21 (m, 2H), 1.20 (s, 6H).

Compounds in the following table (Table 4) were synthesized with the procedures described in Examples 1 to 9.

TABLE 4
Analytical Data of Exemplary Compounds of the Present Disclosure

| Compound # | Analytical Data |
|---|---|
| 3 | LCMS: ESI-MS m/z: 811.3 [M + H]⁺ |
| | 1H NMR (400 MHz, DMSO) δ 10.95 (s, 1H), 8.27-8.22 (m, 2H), |
| | 7.97 (d, J = 8.5 Hz, 1H), 7.75 (d, J = 8.4 Hz, 2H), 7.50 (dd, J = |
| | 18.4, 8.9 Hz, 2H), 7.12-7.02 (m, 2H), 6.74-6.65 (m, 2H), 6.44 (d, |
| | J = 8.5 Hz, 2H), 5.08-5.01 (m, 1H), 4.47-3.94 (m, 6H), 3.60- |
| | 3.54 (m, 2H), 3.45-3.20 (m, 3H), 3.09-2.81 (m, 3H), 2.71-2.58 |
| | (m, 3H), 2.41-2.33 (m,2H), 2.00-1.92 (m, 1H), 1.25 (s, 6H), 1.19 |
| | (s, 6H). |
| 4 | LCMS: ESI-MS m/z: 830.3 [M + H]⁺. |
| | 1H NMR (400 MHz, DMSO) δ 10.96 (s, 1H), 8.25 (d, J = 9.7 Hz, |
| | 1H), 8.15 (s, 1H), 7.98 (d, J = 8.6 Hz, 1H), 7.63-7.59 (m, 3H), |
| | 7.53 (d, J = 8.8 Hz, 1H), 7.10-7.05 (m, 3H), 6.72-6.67 (m, 2H), |
| | 6.60-6.55 (m, 1H), 5.08-5.03 (m, 1H), 4.40-4.34 (m, 2H), 4.31 |
| | (s, 1H), 4.23 (s, 1H), 4.14-4.11 (m, 2H), 3.70 (t, J = 6.0 Hz, 3H), |
| | 3.35-3.32 (m, 2H), 3.10-2.85 (m, 3H), 2.66 (d, J = 7.4 Hz, 3H), |
| | 2.56-2.53 (m, 3H), 2.40-2.33 (m, 2H), 1.98-1.94 (m, 1H), 1.25 |
| | (s, 6H), 1.19 (s, 6H). |
| 7 | LCMS: ESI-MS m/z: 837.4 [M + H]⁺. |
| | 1H NMR (400 MHz, DMSO) δ 11.51 (brs, 1H), 10.96 (s, 1H), 9.86 |
| | (brs, 1H), 8.22 (brs, 1H), 8.00-7.93 (m, 1H), 7.76 (d, J = 8.7 Hz, |
| | 2H), 7.60 (d, J = 8.4 Hz, 1H), 7.45 (d, J = 9.1 Hz, 1H), 7.20-7.14 |
| | (m, 2H), 6.68 (d, J = 8.5 Hz, 1H), 6.59 (d, J = 8.8 Hz, 2H), 5.07 |
| | (dd, J = 13.3, 5.1 Hz, 1H), 4.42-4.34 (m, 2H), 4.26-4.22 (m, |
| | 1H), 4.12 (d, J = 9.1 Hz, 2H), 4.05 (d, J = 8.7 Hz, 2H), 3.67 (d, J = |
| | 9.6 Hz, 4H), 3.31 (d, J = 7.7 Hz, 2H), 3.23-3.13(m, 6H), 2.95- |
| | 2.88 (m, 1H), 2.64-2.54 (m, 2H), 2.42-2.35 (m, 1H), 2.01-1.95 |
| | (m, 1H), 1.91 (s, 2H), 1.25 (s, 6H), 1.19 (s, 6H). |
| 10 | LC/MS: 855.3 [M + H]⁺ |
| | 1H NMR (400 MHz, DMSO) δ 11.51 (brs, 1H), 10.96 (s, 1H), |
| | 8.31-8.20 (m, 1H), 7.97 (d, J = 8.5 Hz, 1H), 7.64 (s, 1H), 7.65-7.61 |
| | (m, 2H), 7.60-7.57 (m, 1H), 7.09-7.05 (m, 2H), 6.75 (t, J = 9.1 |
| | Hz, 1H), 6.72-6.65 (m, 2H), 5.05 (dd, J = 13.0, 5.3 Hz, 1H), 4.39 |
| | (s, 1H), 4.27 (dd, J = 51.7, 17.0 Hz, 2H), 4.12 (d, J = 9.2 Hz, 1H), |
| | 3.75 (dd, J = 9.7, 3.1 Hz, 2H), 3.40 (d, J = 8.7 Hz, 2H), 3.30-3.27 |
| | (m, 3H), 2.95-2.86 (m, 1H), 2.64-2.56 (m, 5H), 2.36 (dd, J = 9.7, |
| | 3.1 Hz, 2H), 2.00-1.94 (s, 1H), 1.58 (s, 2H), 1.25 (s, 6H), 1.23 (s, |
| | 1H), 1.19 (s, 6H), 0.89-0.83 (m, 2H). |
| 12 | LC/MS: 825.3[M + H]⁺. |
| | 1H NMR (400 MHz, DMSO) δ 11.63 (brs, 1H), 10.96 (s, 1H), 8.26- |
| | 8.24 (m, 1H), 7.98 (d, J = 8.2 Hz, 1H), 7.75 (d, J = 8.7 Hz, 2H), |
| | 7.53 (d, J = 8.5 Hz, 1H), 7.43 (d, J = 9.1 Hz, 1H), 7.09-7.07 (m, |
| | 2H), 6.68 (d, J = 8.8 Hz, 2H), 6.55 (d, J = 8.9 Hz, 2H), 5.05 (dd, J = |
| | 13.1, 5.1 Hz, 1H), 4.40 (s, 1H), 4.27 (dd, J = 51.0, 16.8 Hz, 3H), |
| | 4.12 (d, J = 9.1 Hz, 1H), 3.46-3.43 (m, 1H), 3.36-3.34 (m, 2H), |
| | 3.33-3.30 (m, 2H), 3.06-3.04 (m, 1H), 2.90-2.88 (m, 1H), 2.60- |
| | 2.58 (m, 6H), 2.35-2.33 (m, 2H), 2.13-2.11 (m, 1H), 1.98-1.96 |
| | (m, 1H), 1.77-1.75 (m, 1H), 1.23-1.21 (m, 14H). |
| 13 | LC/MS: 825.4[M + H]⁺. |
| | 1H NMR (400 MHz,) δ 11.54 (brs, 0.5H), 10.97 (s, 1H), 10.08 |
| | (brs, 0.5H), 8.24 (brs, 1H), 7.98 (d, J = 8.4 Hz, 1H), 7.79 (d, J = |
| | 8.7 Hz, 2H), 7.60 (d, J = 8.4 Hz, 1H), 7.47 (d, J = 9.2 Hz, 1H), 7.20- |
| | 7.15 (m, 2H), 6.68 (d, J = 8.7 Hz, 2H), 6.57 (d, J = 8.8 Hz, 2H), |
| | 5.07 (dd, J = 13.3, 5.1 Hz, 1H), 4.41-4.39 (m, 1H), 4.37-4.20 (m, |
| | 2H), 4.13 (d, J = 9.1 Hz, 1H), 4.07-4.01 (m, 1H), 3.75-3.60 (m, |
| | 4H), 3.47-3.39 (m, 3H), 3.38-3.28 (m, 3H), 3.13-3.07 (m, 1H), |
| | 2.97-2.78 (m, 2H), 2.69-2.55 (m, 1H), 2.43-2.33 (m, 1H), 2.29- |
| | 2.22 (m, 1H), 2.01-1.93 (m, 1H), 1.87-1.75 (m, 1H), 1.29-1.14 |
| | (m, 14H). |
| 14 | LC/MS: 843.4[M + H]⁺ |
| | 1H NMR (400 MHz, DMSO) δ 11.64 (brs, 1H), 10.96 (s, 1H), 8.25 |
| | (d, J = 9.4 Hz, 1H), 7.98 (d, J = 8.5 Hz, 1H), 7.65-7.51 (m, 4H), |
| | 7.10-7.04 (m, 2H), 6.76-6.64 (m, 3H), 5.05 (dd, J = 13.2, 5.1 Hz, |
| | 1H), 4.39 (s, 1H), 4.27 (dd, J = 50.8, 17.0 Hz, 2H), 4.13 (d, J = 9.2 |
| | Hz, 1H), 3.60-3.55 (m, 1H), 3.51-3.44 (m, 2H), 3.32-3.28 (m, |
| | 4H), 3.23-3.20 (m, 1H), 2.96-2.84 (m, 1H), 2.61-2.53 (m, 5H), |
| | 2.44-2.31 (m, 3H), 2.10-2.05 (m, 1H), 2.00-1.91 (m, 1H), 1.71- |
| | 1.66 (m, 1H), 1.27-1.17 (m, 14H). |
| 15 | LC/MS: 843.3 [M + H]⁺. |
| | 1H NMR (400 MHz, DMSO) δ 11.69 (brs, 1H), 10.96 (s, 1H), 8.26 |
| | (s, 1H), 8.24 (s, 1H), 7.98 (d, J = 8.5 Hz, 1H), 7.67-7.49 (m, 4H), |
| | 7.10-7.04 (m, 2H), 6.77-6.63 (m, 3H), 5.05 (dd, J = 13.2, 5.1 Hz, |
| | 1H), 4.40 (s, 1H), 4.33 (d, J = 16.9 Hz, 1H), 4.21 (d, J = 16.5 Hz, |
| | 1H), 4.13 (d, J = 9.3 Hz, 1H), 3.63-3.53 (m, 1H), 3.52-3.44 (m, |
| | 2H), 3.26-3.17 (m, 2H), 2.97-2.84 (m, 1H), 2.63-2.52 (m, 7H), |
| | 2.44-2.33 (m, 4H), 2.14-2.03 (m, 1H), 2.00-1.92 (m, 1H), 1.73- |
| | 1.65 (m, 1H), 1.25 (s, 6H), 1.23-1.21 (m, 1H), 1.19 (s, 6H). |
| 19 | LC/MS: 871.3 [M + H]⁺ |
| | 1H NMR (400 MHz, DMSO) δ 11.55 (brs, 1H), 11.12 (s, 1H), 9.61- |
| | 9.43 (m, 1H), 8.33-8.15 (m, 1H), 7.98 (d, J = 7.2 Hz, 1H), 7.79 |
| | (d, J = 8.5 Hz, 1H), 7.77 (d, J = 12.0 Hz, 1H), 7.74-7.65 (m, 2H), |
| | 7.51 (s, 1H), 7.38 (d, J = 7.1 Hz, 1H), 7.15-7.05 (m, 1H), 6.69 (d, |
| | J = 8.7 Hz, 1H), 5.11 (dd, J = 12.9, 5.2 Hz, 1H), 4.41 (s, 1H), 4.23 |
| | (d, J = 12.4 Hz, 2H), 4.14 (d, J = 9.1 Hz, 1H), 3.65 (d, J = 11.4 Hz, |
| | 2H), 3.52 (d, J = 12.2 Hz, 2H), 3.34 (t, J = 11.8 Hz, 2H), 3.20- |
| | 3.12 (m, 4H), 2.95-2.84 (m, 1H), 2.79 (t, J =12.0 Hz, 2 H) 2.63- |
| | 2.52 (m, 2H), 2.09-2.00 (m, 2H), 1.89 (d, J = 11.5 Hz, 2H), 1.46- |
| | 1.35 (m, 2H), 1.26 (s, 6H), 1.20 (s, 6H). |
| 25 | LCMS: 811.3 [M + H]⁺ |
| | 1H NMR (400 MHz, DMSO) δ 10.98 (s, 1H), 9.88 (brs, 1H), 8.07 |
| | (d, J = 8.6 Hz, 1H), 7.78 (d, J = 8.6 Hz, 2H), 7.61 (d, J = 8.4 Hz, |
| | 1H), 7.48 (d, J = 9.2 Hz, 1H), 7.24-6.99 (m, 3H), 6.69 (d, J = 8.6 |
| | Hz, 1H), 6.47 (d, J = 8.6 Hz, 2H), 6.32 (brs, 1H), 5.07 (dd, J = |
| | 13.2, 4.8 Hz, 1H), 4.43-4.37 (m, 2H), 4.24-4.18 (m, 1H), 4.13- |
| | 4.06 (m, 4H), 3.71 (t, J = 12.0 Hz, 2H), 3.61-3.51 (m, 4H), 3.29- |
| | 3.09 (m, 6H), 2.95-2.88 (m, 1H), 2.67-2.57 (m, 1H), 2.41-2.33 |
| | (m, 1H), 2.03-1.94 (m, 1H), 1.25 (s, 6H), 1.19 (s, 6H). |
| 26 | LC/MS: 837.4[M + H]⁺. |
| | 1H NMR(400 MHz, ) δ 10.96 (s, 1H), 8.16-8.04 (m, 1H), 7.76- |
| | 7.72 (m, 2H), 7.54-7.51 (m, 1H), 7.44-7.35 (m, 1H), 7.09-7.05 |
| | (m, 2H), 6.77-6.61 (m, 1H), 6.58-6.55 (m, 2H), 5.05 (dd, J = |
| | 13.2, 5.2 Hz, 1H), 4.41-4.30 (m, 2H), 4.23-4.17 (m, 1H), 4.09 (d, |
| | J = 9.2 Hz, 1H), 3.61-3.57 (m, 2H), 3.31-3.27 (m, 6H), 2.96- |
| | 2.85 (m, 1H), 2.62-2.56 (m, 5H), 2.54-2.53 (m, 1H), 2.42-2.29 |
| | (m, 4H), 1.99-1.93 (m, 1H), 1.63-1.61 (m, 2H), 1.25 (s, 6H), |
| | 1.18 (s, 6H), 0.79-0.75 (m, 1H). |
| 32 | LCMS: 825.3 [M + H]⁺. |
| | 1HNMR (400 MHz, DMSO) δ 10.98 (s, 1H), 9.83 (brs, 1H), 8.07 |
| | (d, J = 8.4 Hz, 1H), 7.98 (brs, 1H), 7.79 (d, J = 8.4 Hz, 2H), 7.61 |
| | (d, J = 8.4 Hz, 1H), 7.43 (d, J = 9.2 Hz, 1H), 7.23-7.15 (m, 2H), |
| | 6.70 (d, J = 8.4 Hz, 1H), 6.58 (d, J = 8.8 Hz, 2H), 6.34 (brs, 1H), |
| | 5.08 (dd, J = 13.2, 5.2 Hz, 1H), 4.49-4.31 (m, 2H), 4.30-4.22 (m, |
| | 1H), 4.11 (d, J = 9.2 Hz, 1H), 4.12-3.96 (m, 2H), 3.75-3.68 (m, |
| | 1H), 3.63 (t, J = 8.8 Hz, 1H), 3.53-3.43 (m, 1H), 3.41-3.29 (m, |
| | 3H), 3.28-3.16 (m, 4H), 3.11 (t, J = 8.8 Hz, 1H), 3.00-2.78 (m, |
| | 2H), 2.68-2.52 (m, 1H), 2.44-2.32 (m, 1H), 2.31-2.21 (m, 1H), |
| | 2.04-1.92 (m, 1H), 1.90-1.75 (m, 1H), 1.30-1.22 (m, 7H), 1.19 |
| | (s, 6H). |
| 33 | LC/MS: 825.1 [M + H]⁺. |
| | 1H NMR (400 MHz, DMSO) δ 10.98 (s, 1H), 9.82 (brs, 1H), 8.07 |
| | (d, J = 8.4 Hz, 1H), 7.79 (d, J = 8.8 Hz, 2H), 7.61 (d, J = 8.4 Hz, |
| | 1H), 7.42 (d, J = 9.6 Hz, 1H), 7.22-7.15 (m, 2H), 6.69 (d, J = 8.8 |
| | Hz, 1H), 6.58 (d, J = 8.8 Hz, 2H), 5.07 (dd, J = 13.2, 5.2 Hz, 1H), |
| | 4.40-4.34 (m, 2H), 4.27-4.22 (m, 1H), 4.12-4.10 (m, 1H), 4.07- |
| | 4.03 (m, 2H), 3.66-3.60 (m, 4H), 3.48-3.41 (m, 2H), 3.39-3.32 |
| | (m, 3H), 3.26-3.19 (m, 4H), 3.14-3.10 (m, 1H), 2.94-2.84 (m, |
| | 2H), 2.68-2.62 (m, 1H), 2.59-2.55 (m, 1H), 2.41-2.32 (m, 1H), |
| | 2.03-1.93 (m, 1H), 1.88-1.77 (m, 1H), 1.26 (s, 6H), 1.19 (s, 6H). |
| 35 | LC/MS: 871.3[M + H]⁺. |
| | 1H NMR (400 MHz, DMSO) δ 10.96 (s, 1H), 8.24 (d, J = 9.6 Hz, |
| | 1H), 8.04 (d, J = 8.8 Hz, 1H), 7.74 (d, J = 9.2 Hz, 1H), 7.70-7.61 |
| | (m, 2H), 7.53 (d, J = 8.4 Hz, 1H), 7.14-7.02 (m, 3H), 6.76 (t, J = |
| | 9.2 Hz, 2H), 5.05 (dd, J = 13.2, 4.8 Hz, 1H), 4.41 (s, 1H), 4.39- |
| | 4.29 (m, 1H), 4.26-4.17 (m, 1H), 4.13 (d, J = 9.2 Hz, 1H), 3.96 (s, |
| | 3H), 3.58-3.44 (m, 4H), 3.29-3.26 (m, 2H), 2.99-2.85 (m, 1H), |
| | 2.82-2.71 (m, 2H), 2.69-2.54 (m, 3H), 2.40-2.32 (m, 1H), 2.29- |
| | 2.21 (m, 2H), 2.01-1.94 (m, 1H), 1.88-1.80 (m, 2H), 1.79-1.69 |
| | (m, 1H), 1.32-1.29 (m, 1H), 1.26 (s, 6H), 1.24-1.22 (m, 2H), |
| | 1.21 (s, 6H), 1.16-1.08 (m, 1H). |
| 40 | LCMS: 871.3 [M + H]⁺ |
| | 1H NMR (400 MHz, DMSO) δ 10.99 (s, 1H), 9.51 (brs, 1H), 8.83 |
| | (d, J = 5.4 Hz, 1H), 8.20 (d, J = 8.4 Hz, 1H), 7.77 (d, J = 9.4 Hz, |
| | 1H), 7.72-7.66 (m, 2H), 7.61 (d, J = 8.4 Hz, 1H), 7.23-7.15 (m, |
| | 3H), 6.83 (d, J = 8.6 Hz, 1H), 5.08 (dd, J = 13.2, 5.2 Hz, 1H), 4.43- |
| | 4.35 (m, 2H), 4.27-4.21 (m, 1H), 4.13 (d, J = 9.2 Hz, 1H), 4.04 |
| | (s, 3H), 3.66 (d, J = 8.0 Hz, 2H), 3.52 (d, J = 8.0 Hz, 2H), 3.30- |
| | 3.07 (m, 6H), 2.95-2.87 (m, 1H), 2.82-2.76 (m, 2H), 2.62 (t, J = |
| | 13.2 Hz, 1H), 2.42-2.38 (m, 2H), 2.13-2.04 (m, 1H), 2.01-1.87 |
| | (m, 1H), 1.89-1.75 (m, 2H), 1.46-1.38 (m, 2H), 1.29 (s, 6H), |
| | 1.23 (s, 1H), 1.21 (s, 6H). |
| 44 | LC/MS:846.3 [M + H]⁺. |
| | 1H NMR (400 MHz, DMSO) δ 10.88 (s, 1H), 9.54 (brs, 1H), 8.80 |
| | (d, J = 8.3 Hz, 1H), 8.43 (d, J = 2.4 Hz, 1H), 8.30-8.20 (m, 1H), |
| | 7.98 (d, J = 8.2 Hz, 1H), 7.94 (d, J = 8.8 Hz, 1H), 7.81-7.65 (m, |
| | 3H), 7.55 (dd, J = 8.9, 2.5 Hz, 1H), 7.15-7.05 (m, 1H), 6.80-6.65 |
| | (m, 2H), 4.83-4.69 (m, 1H), 4.41 (s, 1H), 4.20-4.05 (m, 3H), |
| | 3.67 (d, J = 10.4 Hz, 2H), 3.52 (d, J = 11.8 Hz, 2H), 3.35-3.10 (m, |
| | 6H), 2.85-2.75 (m, 3H), 2.60-2.52 (m, 1H), 2.25-2.15 (m, 1H), |
| | 2.11-1.97 (m, 2H), 1.89 (d, J = 12.1 Hz, 2H), 1.50-1.35 (m, 2H), |
| | 1.26 (s, 6H), 1.20 (s, 6H). |

### Example 10: Preparation of N-((1,3-trans)-3-((8-cyano-2-oxo-1,2-dihydroquinolin-5-yl)oxy)-2,2,4,4-tetramethylcyclobutyl)-4-(4-((4-(2-((S)-2,6-dioxopiperidin-3-yl)-1-oxoisoindolin-5-yl)piperazin-1-yl)methyl)-4-fluoropiperidin-1-yl)benzamide (Compound 84)

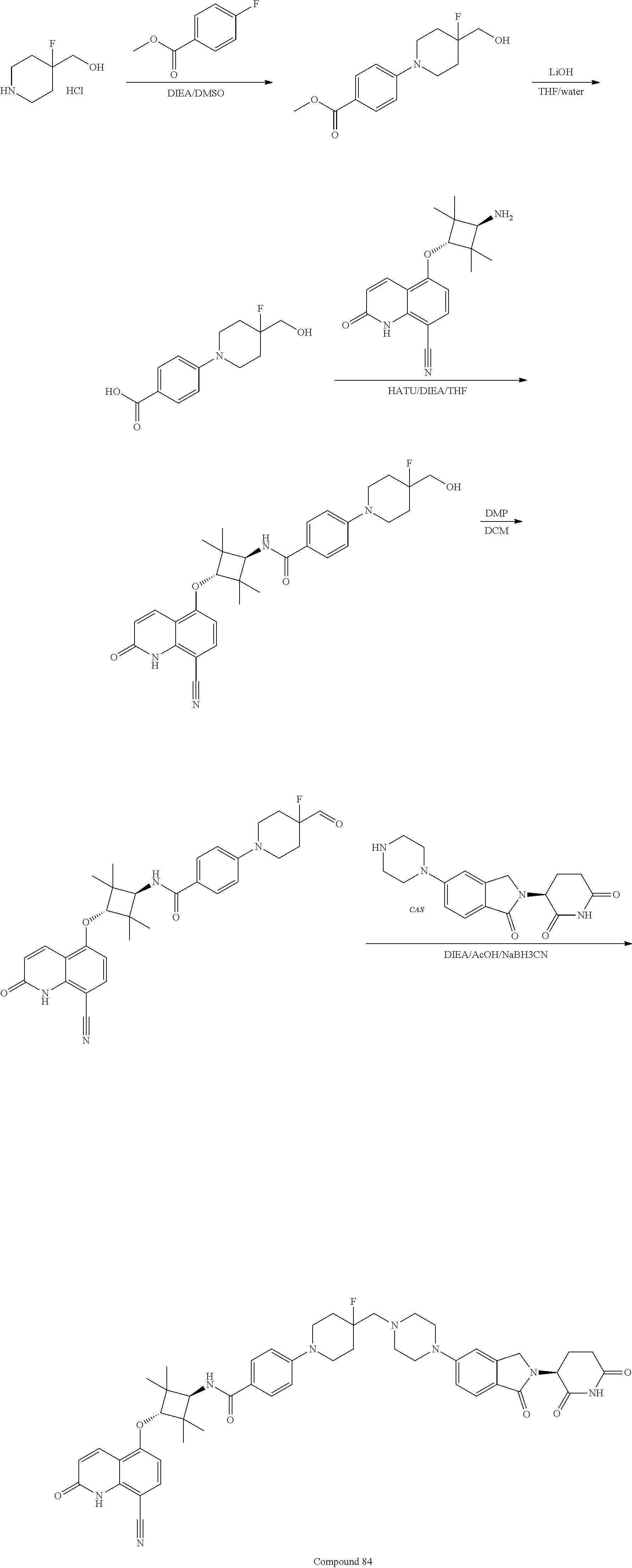

### Step 1: Preparation of methyl 4-(4-fluoro-4-(hydroxymethyl)piperidin-1-yl)benzoate

A mixture of (4-fluoropiperidin-4-yl)methanol hydrochloride (500.000 mg, 2.948 mmol, 1 eq), methyl 4-fluorobenzoate (545.210 mg, 3.537 mmol, 1.2 eq) and DIEA (1.524 g, 11.790 mmol, 4 eq) in DMSO (10 mL) was stirred at 120° C. for 16 hours. TLC showed the starting material was consumed completely. The mixture was poured into water, extracted with EA, washed with HCl (1 N). The combined organic layer was dried over anhydrous Na₂SO₄, filtered and concentrated to give methyl 4-(4-fluoro-4-(hydroxymethyl)piperidin-1-yl)benzoate (300.000 mg, yield 38.08%) as a pale solid which was used directly in next step without further purification.

### Step 2: Preparation of 4-(4-fluoro-4-(hydroxymethyl)piperidin-1-yl)benzoic acid

A mixture of methyl 4-(4-fluoro-4-(hydroxymethyl)piperidin-1-yl)benzoate (200.000 mg, 0.748 mmol, 1 eq), LiOH (125.582 mg, 2.993 mmol, 4 eq) in water (2 mL), THF (5 mL) and MeOH (5 mL) were added into a flask and the mixture was stirred at 45° C. for 2 hours. TLC showed the starting material was consumed completely. The mixture was concentrated under reduced pressure. The residue was dissolved in water, then adjusted to pH=3˜4, and filtered. The filter cake was collected to give 4-(4-fluoro-4-(hydroxymethyl)piperidin-1-yl)benzoic acid (150.000 mg, yield 79.15%) as a white solid. ¹H NMR (400 MHz, DMSO) δ 12.23 (br.s, 1H), 7.76 (d, J=6.8 Hz, 2H), 6.98 (d, J=9.2 Hz, 2H), 4.98 (s, 1H), 3.74 (d, J=13.2 Hz, 2H), 3.43 (d, J=20.0 Hz, 2H), 3.16-3.09 (m, 2H), 1.87-1.75 (m, 3H), 1.71-1.62 (m, 1H).

### Step 3: Preparation of N-((1,3-trans)-3-((8-cyano-2-oxo-1,2-dihydroquinolin-5-yl)oxy)-2,2,4,4-tetramethylcyclobutyl)-4-(4-fluoro-4-(hydroxymethyl)piperidin-1-yl)benzamide

HATU (202.673 mg, 0.533 mmol, 1.5 eq) was added to a mixture of 4-(4-fluoro-4-(hydroxymethyl)piperidin-1-yl)benzoic acid (90.000 mg, 0.355 mmol, 1 eq), 5-((1,3-trans)-3-amino-2,2,4,4-tetramethylcyclobutoxy)-2-oxo-1,2-dihydroquinoline-8-carbonitrile (148.327 mg, 0.426 mmol, 1.2 eq) and DIEA (183.717 mg, 1.421 mmol, 4 eq) in THF (5 mL). Then the mixture was stirred at room temperature for 14 hours. LCMS showed the starting material was consumed completely and the desired compound was detected. The mixture was concentrated and the residue was purified by column chromatography on silica gel to give N-((1,3-trans)-3-((8-cyano-2-oxo-1,2-dihydroquinolin-5-yl)oxy)-2,2,4,4-tetramethylcyclobutyl)-4-(4-fluoro-4-(hydroxymethyl)piperidin-1-yl)benzamide (150.000 mg, yield 77.22%) as a yellow solid.

### Step 4: Preparation of N-((1,3-trans)-3-((8-cyano-2-oxo-1,2-dihydroquinolin-5-yl)oxy)-2,2,4,4-tetramethylcyclobutyl)-4-(4-fluoro-4-formylpiperidin-1-yl)benzamide

Dess-Martin periodinane (174.578 mg, 0.412 mmol, 1.5 eq) was added to a mixture of N-((1,3-trans)-3-((8-cyano-2-oxo-1,2-dihydroquinolin-5-yl)oxy)-2,2,4,4-tetramethylcyclobutyl)-4-(4-fluoro-4-(hydroxymethyl)piperidin-1-yl)benzamide (150.000 mg, 0.274 mmol, 1 eq) in DCM (20 mL) at room temperature. The mixture was stirred at this temperature for 5 hours. LCMS showed minimal starting material remained. The mixture was filtered and the filtrate was concentrated to give N-((1,3-trans)-3-((8-cyano-2-oxo-1,2-dihydroquinolin-5-yl)oxy)-2,2,4,4-tetramethylcyclobutyl)-4-(4-fluoro-4-formylpiperidin-1-yl)benzamide (100.000 mg, yield 66.91%) (crude) as a yellow oil which was used directed in next step without further purification.

### Step 5: Preparation of N-((1,3-trans)-3-((8-cyano-2-oxo-1,2-dihydroquinolin-5-yl)oxy)-2,2,4,4-tetramethylcyclobutyl)-4-(4-((4-(2-((S)-2,6-dioxopiperidin-3-yl)-1-oxoisoindolin-5-yl)piperazin-1-yl)methyl)-4-fluoropiperidin-1-yl)benzamide (Compound 84)

A solution of DIEA (23.732 mg, 0.184 mmol, 1 eq) and (S)-3-(1-oxo-5-(piperazin-1-yl)isoindolin-2-yl)piperidine-2,6-dione (7,7-dimethyl-2-oxobicyclo[2.2.1]heptan-1-yl)methanesulfonate (113.242 mg, 0.202 mmol, 1.1 eq) in DCM (2 mL) and MeOH (2 mL) was stirred at room temperature for 5 mins. N-((1,3-trans)-3-((8-cyano-2-oxo-1,2-dihydroquinolin-5-yl)oxy)-2,2,4,4-tetramethylcyclobutyl)-4-(4-fluoro-4-formylpiperidin-1-yl)benzamide (100.000 mg, 0.184 mmol, 1 eq) and AcOH (33.078 mg, 0.551 mmol, 3 eq) were added subsequently. After 5 mins, NaBH₃CN (17.283 mg, 0.275 mmol, 1.5 eq) was added. The mixture was stirred at room temperature for another 0.5 hour. LCMS showed the starting material was consumed completely and desired compound was detected. The mixture was concentrated under reduced pressure and the residue was purified by Prep-HPLC to give N-((1,3-trans)-3-((8-cyano-2-oxo-1,2-dihydroquinolin-5-yl)oxy)-2,2,4,4-tetramethylcyclobutyl)-4-(4-((4-(2-((S)-2,6-dioxopiperidin-3-yl)-1-oxoisoindolin-5-yl)piperazin-1-yl)methyl)-4-fluoropiperidin-1-yl)benzamide (4.200 mg, yield 2.67%) as a white solid. LCMS: [M+H]⁺=857.10. ¹H NMR (400 MHz, DMSO) δ 11.58 (s, 1H), 10.95 (s, 1H), 8.25 (s, 1H), 7.97 (d, J=8.8 Hz, 1H), 7.77 (d, J=8.4 Hz, 2H), 7.56 (s, 1H), 7.52 (d, J=8.8 Hz, 1H), 7.07 (s, 1H), 7.05-7.00 (m, 3H), 6.68 (d, J=8.4 Hz, 2H), 5.05 (dd, J=13.2, 5.2 Hz, 1H), 4.40 (s, 1H), 4.33 (d, J=17.2 Hz, 1H), 4.20 (d, J=16.8 Hz, 1H), 4.12 (d, J=9.2 Hz, 1H), 3.66 (d, J=12.8 Hz, 2H), 3.33-3.26 (m, 4H), 3.14 (t, J=11.6 Hz, 2H), 2.95-2.85 (m, 1H), 2.66-2.54 (m, 6H), 2.44-2.32 (m, 1H), 1.99-1.91 (m, 3H), 1.85-1.68 (m, 2H), 1.25 (s, 6H), 1.24-1.23 (m, 1H), 1.19 (s, 6H).

### Example 11: Preparation of N-((1,3-trans)-3-((8-cyano-2-oxo-1,2-dihydroquinolin-5-yl)oxy)-2,2,4,4-tetramethylcyclobutyl)-4-(4-((4-(2-(2,6-dioxopiperidin-3-yl)-7-fluoro-1-oxoisoindolin-5-yl)piperazin-1-yl)methyl)piperidin-1-yl)-3-fluorobenzamide (Compound 109)

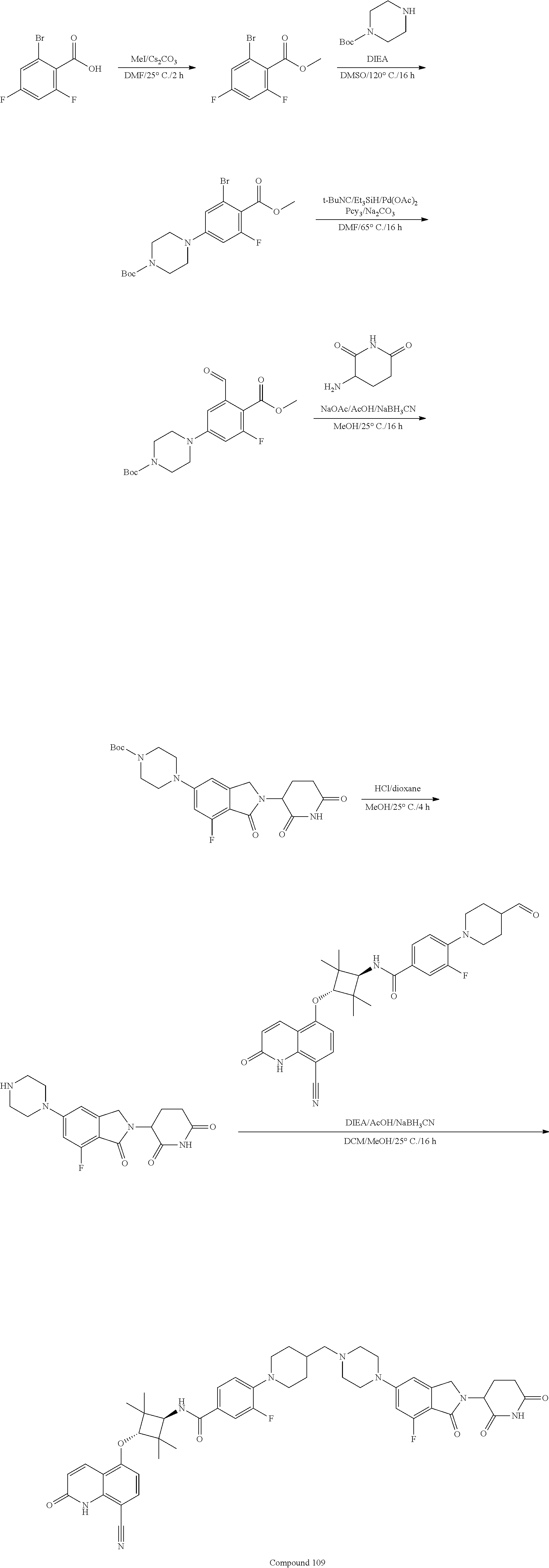

### Step 1: Preparation of methyl 2-bromo-4,6-difluorobenzoate

2-Bromo-4,6-difluorobenzoic acid (500 mg, 2.1 mmol, 1.0 eq), Mel (454 mg, 3.2 mmol, 1.5 eq) and Cs₂CO₃ (1.4 g, 4.2 mmol, 2.0 eq) were dissolved in DMF (10 mL) and the reaction mixture was stirred at 25° C. for 2 h. LCMS analysis indicated complete consumption of starting material and formation of product with desired mass. The reaction mixture was filtered and the filtrate was concentrated under reduced pressure to give methyl 2-bromo-4,6-difluorobenzoate (400 mg crude) as a yellow solid which was used directly in next step without further purification. LCMS: [M+H⁺]=251.

### Step 2: Preparation of tert-butyl 4-(3-bromo-5-fluoro-4-(methoxycarbonyl)phenyl)piperazine-1-carboxylate

Methyl 2-bromo-4,6-difluorobenzoate (400 mg, 1.6 mmol, 1.0 eq), tert-Butyl piperazine-1-carboxylate (298 mg, 1.6 mmol, 1.0 eq) and DIEA (413 mg, 3.2 mmol, 2.0 eq) were dissolved in DMSO (10 mL), and the reaction mixture was heated to 120° C. for 16 h. LC/MS analysis indicated complete consumption of starting material and formation of product with desired mass. The reaction mixture was extracted with ethyl acetate (10 mL×3). The organic layers were combined, washed with water and brine. The organic phase was dried over anhydrous sodium sulfate, concentrated under reduced pressure and the resulting crude residue was purified via column chromatography (PE:EA=5:1) to give tert-butyl 4-(3-bromo-5-fluoro-4-(methoxycarbonyl)phenyl)piperazine-1-carboxylate (220 mg, yield: 33%) as a white solid. LCMS: [M+H⁺]=417.

### Step 3: Preparation of tert-butyl 4-(3-fluoro-5-formyl-4-(methoxycarbonyl)phenyl)piperazine-1-carboxylate

4-(3-Bromo-5-fluoro-4-(methoxycarbonyl)phenyl)piperazine-1-carboxylate (220 mg, 0.5 mmol, 1.0 eq), tert-butylisocyanide (83 mg, 1.0 mmol, 2.0 eq), triethylsilane (174 mg, 1.5 mmol, 3.0 eq), Pd(OAc)₂ (11 mg, 0.05 mmol, 0.1 eq), tricyclohexyl phosphine (28 mg, 0.1 mmol, 0.2 eq) and Na₂CO₃ (106 mg, 1.0 mmol, 2.0 eq) were dissolved in DMF (5 mL). The reaction mixture was heated to 65° C. and allowed to stir for 16 h. LCMS analysis indicated complete consumption of starting material and formation of product with desired mass. The reaction mixture was extracted with ethyl acetate (10 mL×3). The organic layers were combined, washed with water and brine. The organic phase was dried over anhydrous sodium sulfate, concentrated under reduced pressure and the resulting crude residue was purified via column chromatography (PE:EA=5:1) to give tert-butyl 4-(3-fluoro-5-formyl-4-(methoxycarbonyl)phenyl)piperazine-1-carboxylate (180 mg, yield: 93%) as a yellow solid. LCMS: [M+H⁺]=367.

### Step 4: Preparation of tert-butyl 4-(2-(2,6-dioxopiperidin-3-yl)-7-fluoro-1-oxoisoindolin-5-yl)piperazine-1-carboxylate

tert-Butyl 4-(3-fluoro-5-formyl-4-(methoxycarbonyl)phenyl)piperazine-1-carboxylate (180 mg, 0.5 mmol, 1.0 eq), 3-aminopiperidine-2,6-dione (103 mg, 0.8 mmol, 1.5 eq) and NaOAc (136 mg, 1.0 mmol, 2.0 eq) were dissolved in MeOH (5 mL). The reaction mixture was stirred at 25° C. for 0.5 h. Then AcOH (150 mg, 2.5 mmol, 5.0 eq) was added into reaction mixture which was stirred for another 0.5 h. NaBH₃CN (38 mg, 0.6 mmol, 1.5 eq) was added into reaction mixture and the mixture was allowed to stir for 16 h. LCMS analysis indicated complete consumption of starting material and formation of product with desired mass. The reaction mixture was filtered and filter cake was washed by H₂O and MeOH to give 4-(2-(2,6-dioxopiperidin-3-yl)-7-fluoro-1-oxoisoindolin-5-yl)piperazine-1-carboxylate (40 mg, yield: 18%) as a light purple solid. LCMS: [M+H⁺]=447.

### Step 5: Preparation of 3-(7-fluoro-1-oxo-5-(piperazin-1-yl)isoindolin-2-yl)piperidine-2,6-dione

4-(2-(2,6-Dioxopiperidin-3-yl)-7-fluoro-1-oxoisoindolin-5-yl)piperazine-1-carboxylate (40 mg, 0.1 mmol, 1.0 eq) was dissolved in 4M HCl/dioxane (2 mL). The reaction mixture was stirred at 25° C. for 4 h. LCMS analysis indicated complete consumption of starting material and formation of product with desired mass. The reaction mixture was concentrated to give residue as 3-(7-fluoro-1-oxo-5-(piperazin-1-yl)isoindolin-2-yl)piperidine-2,6-dione (35 mg) as a HCl salt. LCMS: [M+H⁺]=347.

### Step 6: Preparation of N-((1,3-trans)-3-((8-cyano-2-oxo-1,2-dihydroquinolin-5-yl)oxy)-2,2,4,4-tetramethylcyclobutyl)-4-(4-((4-(2-(2,6-dioxopiperidin-3-yl)-7-fluoro-1-oxoisoindolin-5-yl)piperazin-1-yl)methyl)piperidin-1-yl)-3-fluorobenzamide (Compound 109)

3-(7-Fluoro-1-oxo-5-(piperazin-1-yl)isoindolin-2-yl)piperidine-2,6-dione (35 mg, 0.1 mmol, 1.0 eq), N-((1,3-trans)-3-((8-cyano-2-oxo-1,2-dihydroquinolin-5-yl)oxy)-2,2,4,4-tetramethylcyclobutyl)-3-fluoro-4-(4-formylpiperidin-1-yl)benzamide (54 mg, 0.1 mmol, 1.0 eq) and DIEA (13 mg, 0.1 mmol, 1.0 eq) were dissolved in DCM (2 mL) and the reaction mixture was stirred for 0.5 h. AcOH (6 mg, 0.1 mmol, 1.0 eq) was added into reaction mixture which was stirred for another 0.5 h. NaBH₃CN (8 mg, 0.12 mmol, 1.2 eq) was added into reaction mixture and allowed to stir 16 h. LC/MS analysis indicated complete consumption of starting material and formation of product with desired mass. The reaction mixture was concentrated under reduced pressure and the resulting crude residue was purified by Prep-HPLC to afford N-((1,3-trans)-3-((8-cyano-2-oxo-1,2-dihydroquinolin-5-yl)oxy)-2,2,4,4-tetramethylcyclobutyl)-4-(4-((4-(2-(2,6-dioxopiperidin-3-yl)-7-fluoro-1-oxoisoindolin-5-yl)piperazin-1-yl)methyl)piperidin-1-yl)-3-fluorobenzamide (21 mg, yield: 24%) as a white solid. LCMS: [M+H⁺]=875. ¹H NMR (400 MHz, DMSO) δ 11.60 (s, 1H), 10.95 (s, 1H), 8.25 (s, 1H), 7.98 (d, J=8.4 Hz, 1H), 7.75-7.62 (m, 3H), 7.17-6.88 (m, 3H), 6.69 (d, J=8.8 Hz, 2H), 5.01 (dd, J=13.2, 5.2 Hz, 1H), 4.44 (s, 1H), 4.37 (d, J=17.2 Hz, 1H), 4.22 (d, J=17.2 Hz, 1H), 4.13 (d, J=9.2 Hz, 1H), 3.53-3.47 (m, 2H), 3.42-3.35 (m, 2H), 3.30-3.10 (m, 3H), 3.06-2.83 (m, 2H), 2.77 (s, 2H), 2.65-2.51 (m, 2H), 2.37 (s, 1H), 2.25 (s, 1H), 2.01-1.91 (m, 2H), 1.89-1.81 (m, 2H), 1.45-1.35 (m, 2H), 1.31-1.27 (m, 2H), 1.26 (s, 6H), 1.20 (s, 6H).

### Example 12: Preparation of N-((1,3-trans)-3-((8-cyano-2-oxo-1,2-dihydroquinolin-5-yl)oxy)-2,2,4,4-tetramethylcyclobutyl)-4-(4-((4-(2-(2,6-dioxopiperidin-3-yl)-6-fluoro-1-oxoisoindolin-5-yl)piperazin-1-yl)methyl)piperidin-1-yl)-3-fluorobenzamide (Compound 108)

This compound was prepared using the same method as described in Example 11. LC/MS: [M+H⁺]=875.55; ¹H NMR (400 MHz, DMSO) δ 11.59 (br.s, 1H), 10.97 (s, 1H), 8.24 (d, J=9.6 Hz, 1H), 7.97 (d, J=8.8 Hz, 1H), 7.71 (d, J=8.8 Hz, 1H), 7.68 (s, 1H), 7.65 (d, J=5.2 Hz, 1H), 7.47 (d, J=11.2 Hz, 1H), 7.30 (d, J=7.2 Hz, 1H), 7.10 (t, J=8.8 Hz, 1H), 6.71 (d, J=9.6 Hz, 1H), 6.68 (d, J=8.8 Hz, 1H), 5.08 (dd, J=13.2, 5.2 Hz, 1H), 4.40 (s, 1H), 4.38 (d, J=15.6 Hz, 1H), 4.27 (d, J=17.2 Hz, 1H), 4.13 (d, J=9.2 Hz, 1H), 3.54-3.47 (m, 2H), 3.30-3.10 (m, 10H), 2.96-2.86 (m, 1H), 2.77 (t, J=11.6 Hz, 2H), 2.63-2.56 (m, 1H), 2.44-2.20 (m, 2H), 2.02-1.96 (m, 1H), 1.90-1.83 (m, 2H), 1.42-1.33 (m, 2H), 1.26 (s, 6H), 1.19 (s, 6H).

### Example 13: Preparation of N-((1,3-trans)-3-((8-cyano-2-oxo-1,2-dihydroquinolin-5-yl)oxy)-2,2,4,4-tetramethylcyclobutyl)-4-(4-((4-(2-((S)-2,6-dioxopiperidin-3-yl)-1-oxoisoindolin-5-yl)piperazin-1-yl)methyl)-4-fluoropiperidin-1-yl)-3-fluorobenzamide (Compound 107)

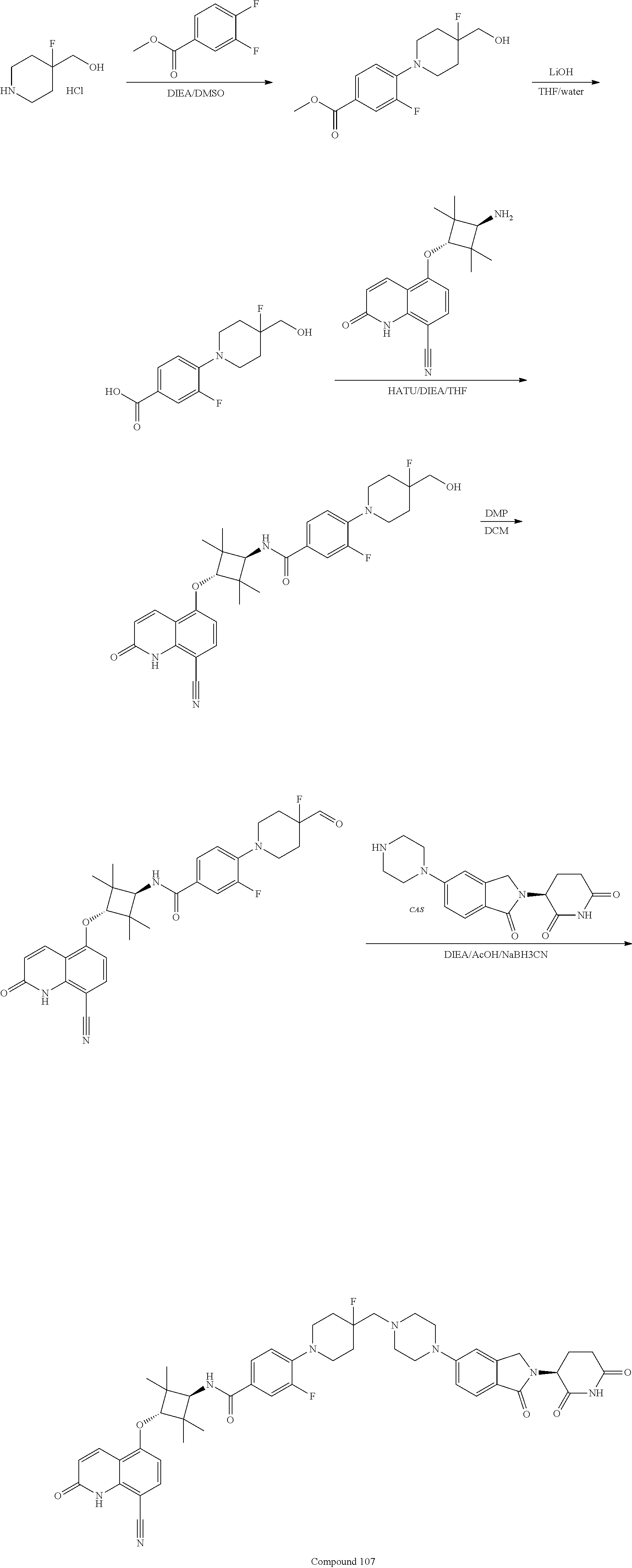

### Step 1: Preparation of methyl 3-fluoro-4-(4-fluoro-4-(hydroxymethyl)piperidin-1-yl)benzoate

A mixture of (4-fluoropiperidin-4-yl)methanol hydrochloride (500.000 mg, 2.948 mmol, 1 eq), methyl 3,4-difluorobenzoate (608.843 mg, 3.537 mmol, 1.2 eq) and DIEA (1.524 g, 11.790 mmol, 4 eq) in DMSO (10 mL) was stirred at 120° C. for 16 hours. TLC showed the starting material was consumed completely. The mixture was poured into water, extracted with EA, washed with HCl (1 N). The combined organic layer was dried over anhydrous Na₂SO₄, filtered and concentrated to give methyl 3-fluoro-4-(4-fluoro-4-(hydroxymethyl)piperidin-1-yl)benzoate (400.000 mg, yield 47.57%) as a white solid. LCMS: [M+H]⁺=286.10.

### Step 2: Preparation of 3-fluoro-4-(4-fluoro-4-(hydroxymethyl)piperidin-1-yl)benzoic acid

A mixture of methyl 3-fluoro-4-(4-fluoro-4-(hydroxymethyl)piperidin-1-yl)benzoate (200.000 mg, 0.701 mmol, 1 eq) and LiOH (29.416 mg, 0.701 mmol, 1 eq) in water (2 mL), THF (5 mL) and MeOH (5 mL) was stirred at 45° C. for 2 hours. TLC showed the starting material was consumed completely. The mixture was concentrated and the residue was dissolved in water, adjusted to pH 3˜4 with HCl (1 N), filtered and the filter cake was collected to give 3-fluoro-4-(4-fluoro-4-(hydroxymethyl)piperidin-1-yl)benzoic acid (180.000 mg, yield 94.65%) as a yellow solid. LCMS: [M+H]⁺=272.05. ¹H NMR (400 MHz, DMSO) δ 12.81 (br.s, 1H), 7.67 (dd, J=8.4, 2.0 Hz, 1H), 7.56 (dd, J=14.0, 2.0 Hz, 1H), 7.13 (t, J=8.4 Hz, 1H), 5.00 (br.s, 1H), 3.46 (d, J=20.0 Hz, 2H), 3.41-3.36 (m, 2H), 3.05-2.98 (m, 2H), 1.87-1.72 (m, 4H).

### Step 3: Preparation of N-((1,3-trans)-3-((8-cyano-2-oxo-1,2-dihydroquinolin-5-yl)oxy)-2,2,4,4-tetramethylcyclobutyl)-3-fluoro-4-(4-fluoro-4-(hydroxymethyl)piperidin-1-yl)benzamide

HATU (189.232 mg, 0.498 mmol, 1.5 eq) was added to a mixture of 3-fluoro-4-(4-fluoro-4-(hydroxymethyl)piperidin-1-yl)benzoic acid (90.000 mg, 0.332 mmol, 1 eq), 5-((1,3-trans)-3-amino-2,2,4,4-tetramethylcyclobutoxy)-2-oxo-1,2-dihydroquinoline-8-carbonitrile hydrochloride (115.408 mg, 0.332 mmol, 1 eq) and DIEA (128.650 mg, 0.995 mmol, 3 eq) in THF (10 mL) at room temperature, then the mixture was stirred at this temperature overnight. LC/MS showed the starting material was consumed completely. The mixture was concentrated and the residue was purified by column chromatography on silica gel to give N-((1,3-trans)-3-((8-cyano-2-oxo-1,2-dihydroquinolin-5-yl)oxy)-2,2,4,4-tetramethylcyclobutyl)-3-fluoro-4-(4-fluoro-4-(hydroxymethyl)piperidin-1-yl)benzamide (160.000 mg, purity 70%, yield 59.79%) as a yellow solid. LCMS: [M+H]⁺=565.20.

### Step 4: Preparation of N-((1,3-trans)-3-((8-cyano-2-oxo-1,2-dihydroquinolin-5-yl)oxy)-2,2,4,4-tetramethylcyclobutyl)-3-fluoro-4-(4-fluoro-4-formylpiperidin-1-yl)benzamide

Dess-Martin periodinane (126.199 mg, 0.298 mmol, 1.5 eq) was added to a mixture of N-((1,3-trans)-3-((8-cyano-2-oxo-1,2-dihydroquinolin-5-yl)oxy)-2,2,4,4-tetramethylcyclobutyl)-3-fluoro-4-(4-fluoro-4-(hydroxymethyl)piperidin-1-yl)benzamide (160.000 mg, 0.198 mmol, 1 eq, purity 70%) in DCM (10 mL) and THF (3 mL) at room temperature, then the mixture was stirred at this temperature for 14 hours. TLC showed the starting material was consumed completely. The mixture was concentrated and the residue was purified by column chromatography on silica gel to give N-((1,3-trans)-3-((8-cyano-2-oxo-1,2-dihydroquinolin-5-yl)oxy)-2,2,4,4-tetramethylcyclobutyl)-3-fluoro-4-(4-fluoro-4-formylpiperidin-1-yl)benzamide (100.000 mg, yield 89.61%) as a colorless oil.

### Step 5: Preparation of N-((1,3-trans)-3-((8-cyano-2-oxo-1,2-dihydroquinolin-5-yl)oxy)-2,2,4,4-tetramethylcyclobutyl)-4-(4-((4-(2-((S)-2,6-dioxopiperidin-3-yl)-1-oxoisoindolin-5-yl)piperazin-1-yl)methyl)-4-fluoropiperidin-1-yl)-3-fluorobenzamide (Compound 107)

A solution of (S)-3-(1-oxo-5-(piperazin-1-yl)isoindolin-2-yl)piperidine-2,6-dione (7,7-dimethyl-2-oxobicyclo[2.2.1]heptan-1-yl)methanesulfonate (camphor sulfonate, 109.621 mg, 0.196 mmol, 1.1 eq) and DIEA (22.973 mg, 0.178 mmol, 1 eq) in DCM (2 mL) and MeOH (2 mL) was stirred at room temperature for 5 mins. N-((1,3-trans)-3-((8-cyano-2-oxo-1,2-dihydroquinolin-5-yl)oxy)-2,2,4,4-tetramethylcyclobutyl)-3-fluoro-4-(4-fluoro-4-formylpiperidin-1-yl)benzamide (100.000 mg, 0.178 mmol, 1 eq) and AcOH (32.020 mg, 0.533 mmol, 3 eq) were subsequently. After 5 mins, NaBH₃CN (16.730 mg, 0.267 mmol, 1.5 eq) was added and the mixture was stirred at room temperature for another 0.5 hour. LC/MS showed the starting material was consumed completely. The mixture was concentrated and the residue was purified by Prep-HPLC to give N-((1,3-trans)-3-((8-cyano-2-oxo-1,2-dihydroquinolin-5-yl)oxy)-2,2,4,4-tetramethylcyclobutyl)-4-(4-((4-(2-((S)-2,6-dioxopiperidin-3-yl)-1-oxoisoindolin-5-yl)piperazin-1-yl)methyl)-4-fluoropiperidin-1-yl)-3-fluorobenzamide (2.700 mg, yield 1.74%) as a white solid. LC/MS: [M+H]⁺=875.05; 1H NMR (40 MHz, DMSO) δ 10.95 (s, 1H), 8.35 (s, 1H), 8.24 (d, J=9.6, 1H), 7.97 (d, J=8.4 Hz, 1H), 7.75 (d, J=9.2 Hz, 1H), 7.68 (s, 1H), 7.65 (d, J=3.2 Hz, 1H), 7.55-7.51 (m, 1H), 7.15 (t, J=8.4 Hz, 1H), 7.10-7.03 (m, 2H), 6.72-6.66 (m, 2H), 5.05 (dd, J=13.2, 5.2 Hz, 1H), 4.40 (s, 1H), 4.33 (d, J=17.2 Hz, 1H), 4.21 (d, J=16.8 Hz, 1H), 4.13 (d, J=9.2 Hz, 1H), 3.51-3.45 (m, 6H), 3.05-2.98 (m, 2H), 2.95-2.86 (m, 1H), 2.70-2.65 (m, 5H), 2.41-2.34 (m, 2H), 2.05-1.83 (m, 5H), 1.26 (s, 6H), 1.24-1.23 (m, 1H), 1.19 (s, 6H).

### Example 14: Preparation of N-((1,3-trans)-3-((8-cyano-2-oxo-1,2-dihydroquinolin-5-yl)oxy)-2,2,4,4-tetramethylcyclobutyl)-4-(4-((4-(2-((R)-2,6-dioxopiperidin-3-yl)-1-oxoisoindolin-5-yl)piperazin-1-yl)methyl)piperidin-1-yl)-3-fluorobenzamide (Compound 106)

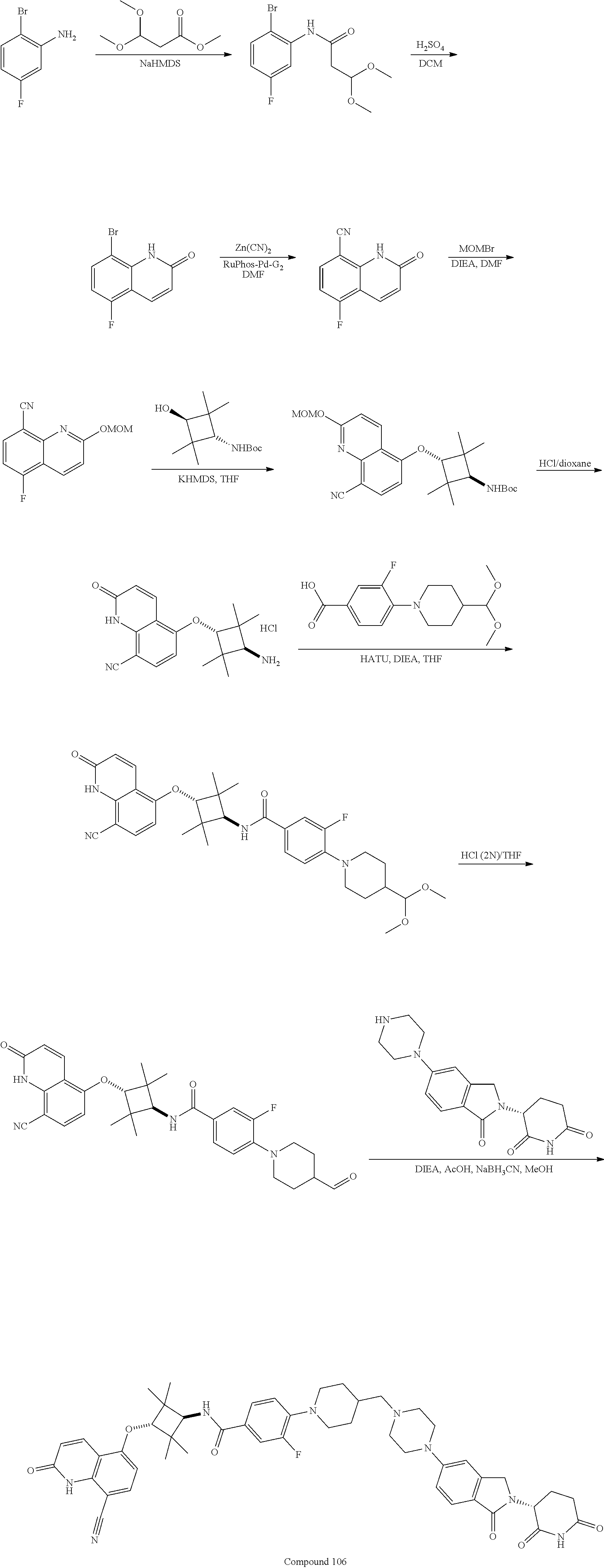

### Step 1: Preparation of N-(2-bromo-5-fluorophenyl)-3,3-dimethoxypropanamide

To a mixture of 2-bromo-5-fluoroaniline (60 g, 315.8 mmol, 1.0 eq) and methyl 3,3-dimethoxypropanoate (56 g, 378.9 mmol, 1.2 eq) in THF (1 L) was added NaHMDS (1 M, 474 mL) slowly at 0° C., then the mixture was stirred at 25° C. for 16 h. TLC showed the starting material was consumed completely. The reaction was quenched with saturated NH₄Cl solution and then extracted with EA. The organic layer was concentrated to give N-(2-bromo-5-fluorophenyl)-3,3-dimethoxypropanamide (80 g, crude) as a yellow solid which was used directly in the next step without further purification.

### Step 2: Preparation of 8-bromo-5-fluoroquinolin-2(1H)-one

To a solution of N-(2-bromo-5-fluorophenyl)-3,3-dimethoxypropanamide (80 g, 261.4 mmol) in DCM (420 mL) was added H₂SO₄ (250 mL, 98%) while stirred under N₂ atmosphere at 0° C. The reaction mixture was stirred at 25° C. for 16 h and then poured into ice water. The residue was filtered and washed with H₂O, i-PrOH and PE. Then the solid was dried to give 8-bromo-5-fluoroquinolin-2(1H)-one (60 g) as a yellow solid without further purification.

### Step 3: Preparation of 5-fluoro-2-oxo-1,2-dihydroquinoline-8-carbonitrile

To a solution of 8-bromo-5-fluoroquinolin-2(1H)-one (60 g, 247.9 mmol, 1.0 eq) in DMF (1.2 L) was added Zn(CN)₂ (58 g, 495.8 mmol, 2.0 eq) and Ruphos Pd G2 (19 g, 24.8 mmol, 0.1 eq). The reaction mixture was stirred at 120° C. under N₂ for 16 h. TLC analysis showed the starting material was consumed completely. The reaction mixture was filtered and the filtrate was concentrated. The crude residue was triturated with DCM and filtered to give 5-fluoro-2-oxo-1,2-dihydroquinoline-8-carbonitrile (30 g, yield: 64%) as a yellow solid.

### Step 4: Preparation of 5-fluoro-2-(methoxymethoxy)quinoline-8-carbonitrile

MOMBr (21.1 g, 143.5 mmol, 1.5 eq) was added dropwise to a solution of 5-fluoro-2-oxo-1,2-dihydroquinoline-8-carbonitrile (18 g, 95.7 mmol, 1.0 eq) and DIEA (24.7 g, 191.3 mol, 2 eq) in DMF (475 mL) at 0° C. under N₂ atmosphere. The mixture was stirred at 25° C. for 16 h. TLC showed the starting material was consumed completely. The reaction mixture was extracted with water and EA. The organic layers were combined and concentrated under reduced pressure. The crude material was crystallized in DCM/PE to give 5-fluoro-2-(methoxymethoxy)quinoline-8-carbonitrile (14 g, yield: 64%) as a yellow solid. ¹HNMR (500 MHz, 8.52 (d, J=9.0 Hz, 1H), 8.31 (dd, J=8.0, 5.5 Hz, 1H), 7.47 (t, J=9.0 Hz, 1H), 7.31 (d, J=9.0 Hz, 1H), 5.72 (s, 2H), 3.53 (s, 3H).

### Step 5: Preparation of tert-butyl ((1,3-trans)-3-((8-cyano-2-(methoxymethoxy)quinolin-5-yl)oxy)-2,2,4,4-tetramethylcyclobutyl)carbamate

To a solution of tert-butyl ((1,3-trans)-3-hydroxy-2,2,4,4-tetramethylcyclobutyl)carbamate (17.5 g, 71.7 mmol, 1.2eq) in THF (100 mL) was added KHMDS (1 M, 70 mL) at −10° C., then the mixture was stirred at this temperature for 0.5 h. A solution of 5-fluoro-2-(methoxymethoxy)quinoline-8-carbonitrile (14 g, 59.8 mmol, 1.0 eq) in THF (100 mL) was added into the reaction mixture slowly at −10° C. The reaction mixture was stirred for another 0.5 h and quenched with saturated NH₄Cl solution, then extracted with EA. The organic layers were combined and concentrated under reduced pressure. The crude residue was crystallized in DCM/PE to give tert-butyl ((1,3-trans)-3-((8-cyano-2-(methoxymethoxy)quinolin-5-yl)oxy)-2,2,4,4-tetramethylcyclobutyl)carbamate (18 g, yield: 66%) as a yellow solid.

### Step 6: Preparation of 5-((1,3-trans)-3-amino-2,2,4,4-tetramethylcyclobutoxy)-2-oxo-1,2-dihydroquinoline-8-carbonitrile hydrochloride

A solution of ((1,3-trans)-3-((8-cyano-2-(methoxymethoxy)quinolin-5-yl)oxy)-2,2,4,4-tetramethylcyclobutyl)carbamate (18 g, 39.5 mmol) in HCl/dioxane (4 M, 200 mL) was stirred at 25° C. for 16 h. The reaction mixture was filtered to give 5-((1,3-trans)-3-amino-2,2,4,4-tetramethylcyclobutoxy)-2-oxo-1,2-dihydroquinoline-8-carbonitrile hydrochloride (11 g, yield: 89%) as a yellow solid which was used directly in next step without further purification.

### Step 7: Preparation of N-((1,3-trans)-3-((8-cyano-2-oxo-1,2-dihydroquinolin-5-yl)oxy)-2,2,4,4-tetramethylcyclobutyl)-4-(4-(dimethoxymethyl)piperidin-1-yl)-3-fluorobenzamide

A mixture of 5-((1,3-trans)-3-amino-2,2,4,4-tetramethylcyclobutoxy)-2-oxo-1,2-dihydroquinoline-8-carbonitrile hydrochloride (5 g, 16.1 mmol, 1.0 eq), 4-(4-(dimethoxymethyl)piperidin-1-yl)-3-fluorobenzoic acid (4.8 g, 16.1 mmol, 1.0 eq), HATU (7.3 g, 19.3 mmol, 1.2 eq) and DIEA (6.2 g, 48.2 mmol, 3.0 eq) in THF (50 mL) was stirred at 25° C. for 2 h. The reaction mixture was concentrated and crude was purified by flash column (PE/EA=1/1) to give N-((1,3-trans)-3-((8-cyano-2-oxo-1,2-dihydroquinolin-5-yl)oxy)-2,2,4,4-tetramethylcyclobutyl)-4-(4-(dimethoxymethyl)piperidin-1-yl)-3-fluorobenzamide (6 g, yield: 63%) as a white solid.

### Step 8: Preparation of N-((1,3-trans)-3-((8-cyano-2-oxo-1,2-dihydroquinolin-5-yl)oxy)-2,2,4,4-tetramethylcyclobutyl)-3-fluoro-4-(4-formylpiperidin-1-yl)benzamide

A mixed solution of N-((1,3-trans)-3-((8-cyano-2-oxo-1,2-dihydroquinolin-5-yl)oxy)-2,2,4,4-tetramethylcyclobutyl)-4-(4-(dimethoxymethyl)piperidin-1-yl)-3-fluorobenzamide (6 g, 10.2 mmol) in THF/2M HCl (1/1, 17 mL) was stirred at 40° C. for 2 h. TLC analysis showed the starting material was consumed completely. The pH was adjusted to 8 and the reaction mixture was extracted with EA. The organic layer was concentrated to give N-((1,3-trans)-3-((8-cyano-2-oxo-1,2-dihydroquinolin-5-yl)oxy)-2,2,4,4-tetramethylcyclobutyl)-3-fluoro-4-(4-formylpiperidin-1-yl)benzamide (4.5 g, yield: 81%) as a white solid.

### Step 9: Preparation of N-((1,3-trans)-3-((8-cyano-2-oxo-1,2-dihydroquinolin-5-yl)oxy)-2,2,4,4-tetramethylcyclobutyl)-4-(4-((4-(2-((R)-2,6-dioxopiperidin-3-yl)-1-oxoisoindolin-5-yl)piperazin-1-yl)methyl)piperidin-1-yl)-3-fluorobenzamide (Compound 106)

A mixture of N-((1,3-trans)-3-((8-cyano-2-oxo-1,2-dihydroquinolin-5-yl)oxy)-2,2,4,4-tetramethylcyclobutyl)-3-fluoro-4-(4-formylpiperidin-1-yl)benzamide (2.1 g, 3.9 mmol, 1.0 eq), (R)-3-(1-oxo-5-(piperazin-1-yl)isoindolin-2-yl)piperidine-2,6-dione (1.3 g, 3.9 mmol, 1.0 eq), DIEA (0.75 g, 5.9 mmol, 1.5 eq) and AcOH (0.7 g, 11.7 mmol, 3.0 eq) in MeOH (20 mL) was stirred at 25° C. for 1 h. Then NaBH₃CN (0.3 g, 4.7 mmol, 1.2 eq) was added and the reaction mixture was stirred for another 2 h. The reaction mixture was concentrated and the crude was purified by flash column (DCM/MeOH=20/1) to give N-((1,3-trans)-3-((8-cyano-2-oxo-1,2-dihydroquinolin-5-yl)oxy)-2,2,4,4-tetramethylcyclobutyl)-4-(4-((4-(2-((R)-2,6-dioxopiperidin-3-yl)-1-oxoisoindolin-5-yl)piperazin-1-yl)methyl)piperidin-1-yl)-3-fluorobenzamide (2.3 g, yield: 70%) as an off-white solid. LCMS: [M+H]⁺=857.10. ¹H NMR (500 MHz, DMSO) δ 11.64 (br.s, 1H), 10.95 (s, 1H), 8.25 (d, J=8.0 Hz, 1H), 7.98 (d, J=8.5 Hz, 1H), 7.72 (d, J=9.5 Hz, 1H), 7.67 (s, 1H), 7.65 (d, J=6.5 Hz, 1H), 7.53 (d, J=8.5 Hz, 1H), 7.11-7.06 (m, 3H), 6.73-6.67 (m, 2H), 5.05 (dd, J=13.0, 5.5 Hz, 1H), 4.40 (s, 1H), 4.34 (d, J=16.5 Hz, 1H), 4.21 (d, J=17.0 Hz, 1H), 4.13 (d, J=9.0 Hz, 1H), 3.49 (d, J=11.5 Hz, 2H), 3.32-3.26 (m, 5H), 2.92-2.85 (m, 1H), 2.78-2.71 (m, 2H), 2.65-2.54 (m, 2H), 2.43-2.32 (m, 2H), 2.27-2.21 (m, 2H), 2.00-1.92 (m, 1H), 1.87-1.82 (m, 2H), 1.35-1.29 (m, 2H), 1.26 (s, 6H), 1.26-1.24 (m, 2H), 1.20 (s, 6H).

### Testing of Compounds for AR Degradation Activity

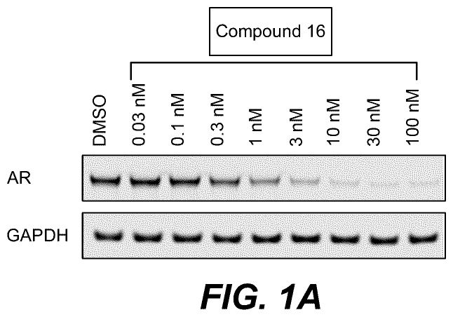

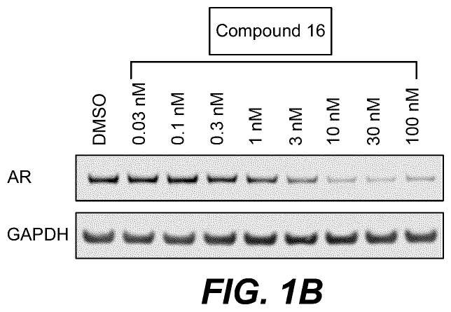

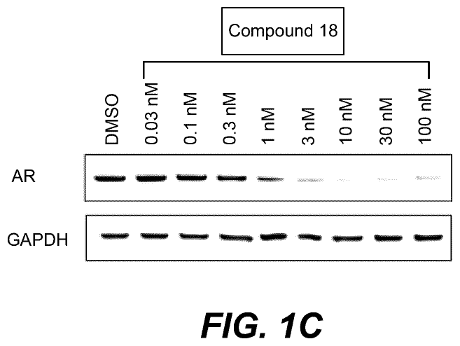

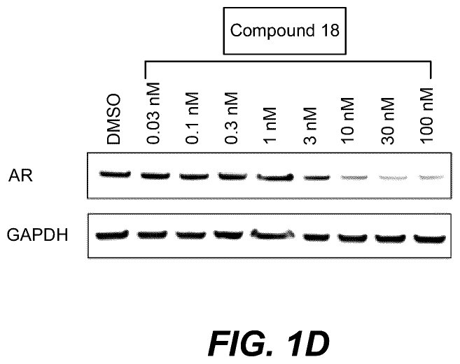

VCAP and LNCAP cells were obtained from American Type Culture Collection (ATCC). VCAP and LNCAP cells were plated in 24-well plates at 2×10E5 cells/well in the RPMI growth medium containing 10% FBS and 1% Penicillin Streptomycin, and then incubated at 37° C. overnight. The following day, the test compound was administered to the cells by using 1000× compound stock solution prepared in DMSO at various concentrations. After administration of the compound, the cells were then incubated at 37° C. for 24 hours. Upon completion, the cells were washed with PBS and protein was collected in Laemmli sample buffer (1×; VWR International). Proteins in cell lysate were separated by SDS-PAGE and transferred to Odyssey nitrocellulose membranes (Licor) with iblot® dry blotting transfer system (ThermoFisher). Nonspecific binding was blocked by incubating membranes with Intercept Blocking Buffer (Licor) for 1 hour at room temperature with gentle shaking. The membranes were then incubated overnight at 4° C. with Primary antibodies rabbit anti-AR (1:1,000, Cell Signaling, 5153) and mouse anti-GAPDH (1:5,000, Santa Cruz Biotechnology, sc-47724) diluted in Intercept Blocking Buffer containing 0.1% Tween 20. After washing 3 times with TBS-T, the membranes were incubated with IRDye® 800CW goat anti-mouse IgG (1:20,000, Licor) or IRDye® 800CW goat anti-rabbit IgG (1:20,000, Licor) for 1 hour. After TBS-T washes, membranes were rinsed in TBS and scanned on Odyssey@ CLx Imaging System (Licor). Results are shown in FIG. 1A-1D.

### Testing of Compounds for AR Degradation Activity Engineered PC3 Cells Overexpressing Wild Type AR

PC3 parental cells were obtained from American Type Culture Collection (ATCC) and maintained in the RPMI growth medium containing 10% FBS and 1% Penicillin Streptomycin. PC3 cells engineered with wildtype AR (PC3 AR-WT) were generated from the PC3 cell line by lentiviral transduction. First, full length AR wildtype was cloned into CD532A-2 lentivector (System Biosciences) through standard gene synthesis. Lentiviruses were subsequently produced using the lentiviral plasmid and MISSION Lentiviral Packaging Mix (Sigma) following the manufacturer's protocol. Next, PC3 cells were transduced with AR wildtype lentiviruses in the presence of 5 μg/ml polybrene for a day and then selected under 1 μg/ml puromycin in the fresh culture medium for 1 week.

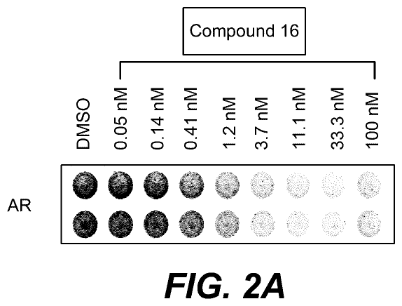

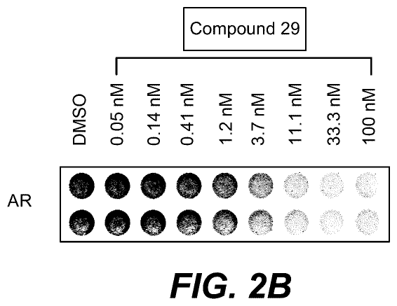

PC3 AR-WT cells were plated in 96-well plates (VWR #10062-900, or Corning #3904) in 90 uL culture medium at a density of 10,000 cells/well in the RPMI growth medium containing 10% FBS and 1% Penicillin Streptomycin, and then incubated at 37° C. overnight. The following day, the test compound was administered to the cells by using 1000× compound stock solution prepared in DMSO at various concentrations. 1000× compound stock solution was first diluted in culturing medium to 10×, then 10 uL compound medium was added to each well in the cell plates. After administration of the compound, the cells were then incubated at 37° C. for 24 hours. Upon completion, the cells were washed with PBS briefly. 150 uL/well of 4% formaldehyde was added and the plates were incubated at room temperature for 20 mins. The cells were washed with PBS briefly, and permeabilized with 150 uL/well of ice cold 100% methanol for 10 mins. The cells were washed with PBS briefly and blocked with 100 uL/well Licor blocking buffer for 1 hr at room temperature with gentle shaking. The cells were incubated overnight at 4° C. with 50 uL primary antibody rabbit anti-AR (1:500, Cell Signaling, #5153) diluted in Intercept Blocking Buffer (Licor, #927-60001) containing 0.1% Tween 20. The next day, the cells were washed with 200 uL PBS containing 0.1% Tween 20, 5×5 mins at room temperature with gentle shaking, and incubated with 50 uL secondary antibody, IRDye® 800CW Goat anti-Rabbit IgG (Licor, #926-32211), 1:1000, in Licor blocking buffer with 0.2% Tween 20 for 1 hr at room temperature with gentle shaking. The cells were washed with 200 uL PBS containing 0.1% Tween 20, 5×5 mins at room temperature with gentle shaking. The cells were washed with PBS for 5 mins. 100 uL fresh PBS was added to each well and the plates were imaged on a Li-Cor Odyssey CLX plate reader. The results are shown in FIGS. 2A and 2B, which illustrate the AR degradative activity of exemplary compounds 16 and 29 of the present disclosure.

The signal intensity of target proteins was calculated using ImageStudio software. AR fluorescent intensity (FLU) from DMSO (vehicle) treatment was set as 100%. Relative AR expression level was determined by the following formula:

$$AR Expression ( \% ) = \frac{{FLU}_{compound}}{{FLU}_{DMSO}} * 100$$

The percentage of AR expression was calculated using Microsoft Excel. The half-maximal degradation concentration (DC50) values were generated by GraphPad Prism (Version 9) using the dose-response equation of variable slope (four parameters). The quantitative results in PC3 AR-WT cells are shown in Table 5, which summarizes AR degradation DC50 of exemplary compounds.

### Testing of Compounds for Inhibition of R1881 Stimulated Cell Growth in LNCAP and VCAP Cells

LNCAP and VCAP cells were seeded in 96-well plates at 5000 (LNCAP) and 6000 (VCAP) cells/well in phenol red-free RPM11640 supplemented with 10% charcoal stripped serum (CSS), and then incubated at 37° C. overnight. The following day, the test compound was administered to the cells by using 1000× compound stock solution prepared in DMSO at various concentrations. 1000× compound stock solution was first diluted 1:100 in phenol red-free RPM11640 supplemented with 10% CSS and 1 nM R1881 to prepare the 10× treatment solutions containing serial dilutions of AC176 together in the presence of 1 nM, and then 10 uL compound medium was added to each well in the cell plates. After administration of the compound, the cells were then incubated at 37° C. for 6 days. Upon completion, the plates were equilibrated at room temperature for approximately 10 minutes. 100 ul of CellTiter-Glo® 3D Reagent (Promega) was added to each well. The plates were then incubated at room temperature for 30 minutes and luminescence was recorded by EnSpire plate reader (PerkinElmer). The results are shown in Table 5, which summarizes the growth inhibition (G150) in LNCAP and VCAP cells with exemplary compounds of the present disclosure.

TABLE 5
Biological Activities of Exemplified Compounds
in PC3 AR-WT cellular degradation assay, and LNCAP
and VCAP Cellular Growth Inhibition Assay.

| | PC3 AR WT | LNCAP, | VCAP, |
|---|---|---|---|
| | DC₅₀ (nM). | GI₅₀ (nM). | GI₅₀ (nM). |
| | A, ≤5 nM; | A, ≤50 nM; | A, ≤50 nM; |
| | B, >5 nM | B, >50 nM | B, >50 nM |
| | and ≤50 nM; | and ≤500 nM; | and ≤500 nM; |
| Compound # | C, >50 nM | C, >500 nM | C, >500 nM |
| 3 | A | A | B |
| 4 | A | A | A |
| 7 | B | C | B |
| 10 | A | A | A |
| 12 | A | B | A |
| 13 | B | C | B |
| 14 | A | B | A |
| 15 | A | C | A |
| 16 | A | A | A |
| 18 | A | A | A |
| 19 | B | B | B |
| 20 | B | C | B |
| 25 | A | A | B |
| 26 | A | C | B |
| 29 | A | N/A | N/A |
| 30 | A | A | A |
| 32 | A | B | B |
| 33 | B | B | B |
| 34 | A | A | A |
| 35 | N/A | A | A |
| 39 | N/A | A | A |
| 40 | N/A | B | A |
| 43 | B | C | B |
| 49 | B | C | B |

What is claimed is:

1. A compound of Formula 1A or Formula 1B, or a tautomer, stereoisomer or a mixture of stereoisomers, or a pharmaceutically acceptable salt, or hydrate, or deuterated derivative thereof: 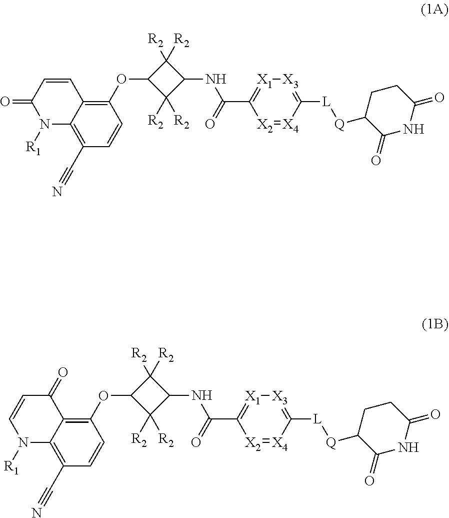
wherein:
R₁ is hydrogen, C₁-C₅ alkyl, C₁-C₅ halogenated alkyl, or C₃-C₅ cycloalkyl;
each R₂ is independently hydrogen, C₁-C₃ alkyl, or C₁-C₃ haloalkyl;
X₁ is CRₐ or N;
X₂ is CR_{b} or N;
X₃ is CR_{c} or N;
X₄ is CR_{d} or N;
each of Rₐ, R_{b}, R_{c}, and R_{d} is independently hydrogen, halogen, C₁-C₃ alkyl, C₁-C₃ haloalkyl, or C₁-C₃ alkoxy;
L is a linker of 1 to 15 carbon atoms in length, wherein one or more carbon atoms are optionally substituted with halogen, oxygen, nitrogen, alkylamino, hydroxyl, sulfur, sulfoxide, sulfone, or amide, and wherein one or more carbon atoms are optionally replaced by cycloalkyl, heterocycle, or heteroaryl, wherein the cycloalkyl, heterocycle, or heteroaryl are each independently substituted with 0, 1, or 2 R₃;
each R₃ is independently hydrogen, halogen, hydroxyl, C₁-C₄alkoxyl, C₁-C₄alkyl, or C₁-C₄haloalkyl;
Q is a 1 to 4 carbon atoms in length, wherein one or more carbon atoms are replaced by a heteroaryl, halogen substituted heteroaryl, alkoxy substituted heteroaryl, aryl, halogen substituted aryl, alkoxy substituted aryl, 6,5-membered fused heterocycle, 6,6-membered fused heterocycle, —C(═O), —C(═O)—NH—, or —C(═O)—NH—(C₁-C₃alkyl), wherein the 6,5-membered fused heterocycle or 6,6-membered fused heterocycle are each independently substituted with 0, 1, or 2 R₄; and
R₄ is independently hydrogen, halogen, hydroxyl, C₁-C₃alkyl, C₁-C₃haloalkyl, C₁-C₃alkoxy, or an oxo group.

2. The compound according to claim 1, wherein X₄ is N.

3. The compound according to claim 1, wherein X₃ and X₄ are each N.

4. The compound according to claim 1, wherein X₁ and X₄ are each N.

5. The compound according to claim 1, wherein X₂ and X₄ are each N.

6. The compound according to claim 1, wherein X₁ is CRₐ, X₂ is CR_{b}, X₃ is CR_{c}, and X₄ is CR_{d}.

7. The compound according to claim 6, wherein Rₐ, R_{b}, R_{c}, and R_{d} are each independently H, Cl, Br, F, or I.

8. The compound according to claim 1, wherein Rₐ, R_{b}, R_{c}, and R_{d} are each independently H or F.

9. The compound or pharmaceutically acceptable salt thereof according to claim 8, wherein Rₐ, R_{b}, R_{c}, and R_{d} are each H.

10. The compound according to claim 8, wherein Rₐ is F.

11. The compound according to claim 8, wherein R_{b} is F.

12. The compound according to claim 8, wherein R_{c} is F.

13. The compound according to claim 8, wherein R_{d} is F.

14. The compound according to claim 1, wherein R₁ is H.

15. The compound according to claim 1, wherein R₁ is —CH₃, C₁-C₅ alkyl, or a deuterated C₁-C₅ alkyl.

16. The compound according to claim 15, wherein R₁ is —CD₃.

17. The compound according to claim 1, wherein R₁ is —CH₂—CF₃, —CH₂CH₃, or —CH(CH₃)₂.

18. The compound according to claim 1, wherein R₁ is —CH₂—CH₃.

19. The compound according to claim 1, wherein R₁ is

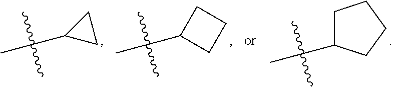

20. The compound according to any one of claims 1 to 19, wherein each R₂ is independently H, —CH₃, or —CH₂—CH₃.

21. The compound according to any one of claims 1 to 19, wherein each R₂ is independently H or —CH₃.

22. The compound according to any one of claims 1 to 21, wherein each R₂ is —CH₃.

23. The compound according to any one of claims 1 to 21, wherein each R₂ is identical.

24. The compound according to any one of claims 1 to 21, wherein each R₂ is different.

25. The compound according to any one of claims 1 to 24, wherein L is a linker of 1 to 10 carbon atoms in length, wherein one or more carbon atoms are optionally replaced by heterocycle, wherein the heterocycle is a monocyclic heterocycle, a fused heterocycle, a spiro heterocycle, or a bridged heterocycle, and wherein the monocyclic heterocycle, the fused heterocycle, the spiro heterocycle, or the bridged heterocycle are each independently substituted with 0 or 1 R₃.

26. The compound according to any one of claims 1 to 25, wherein L is:

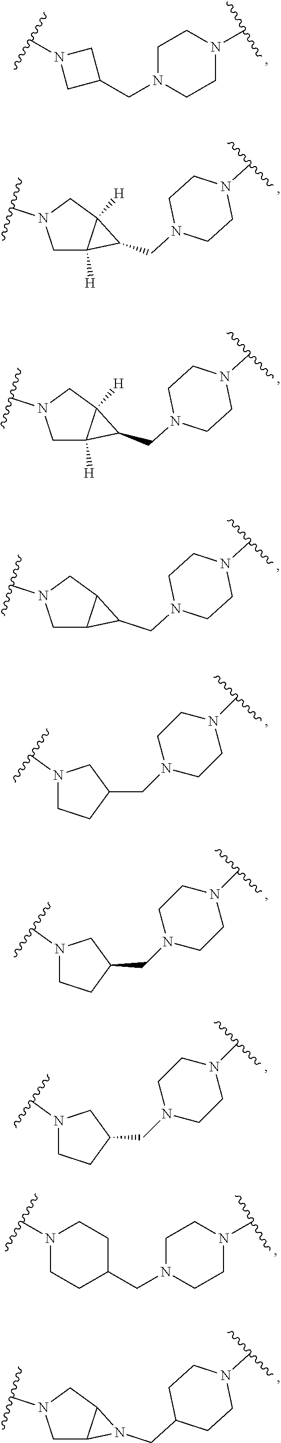

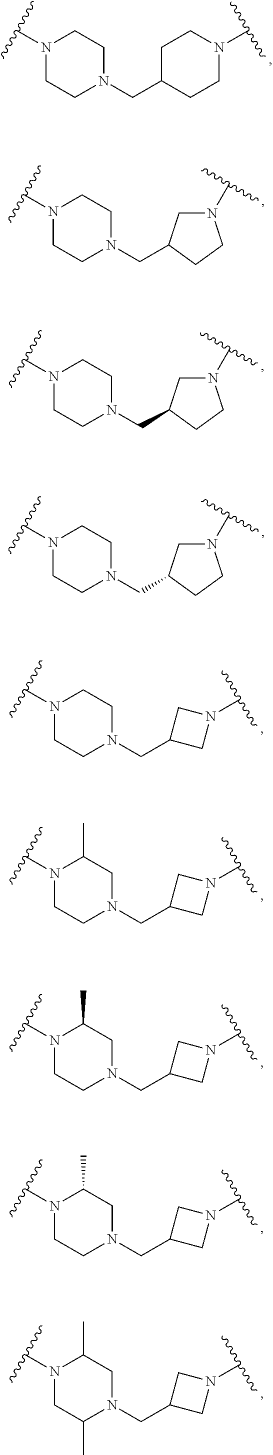

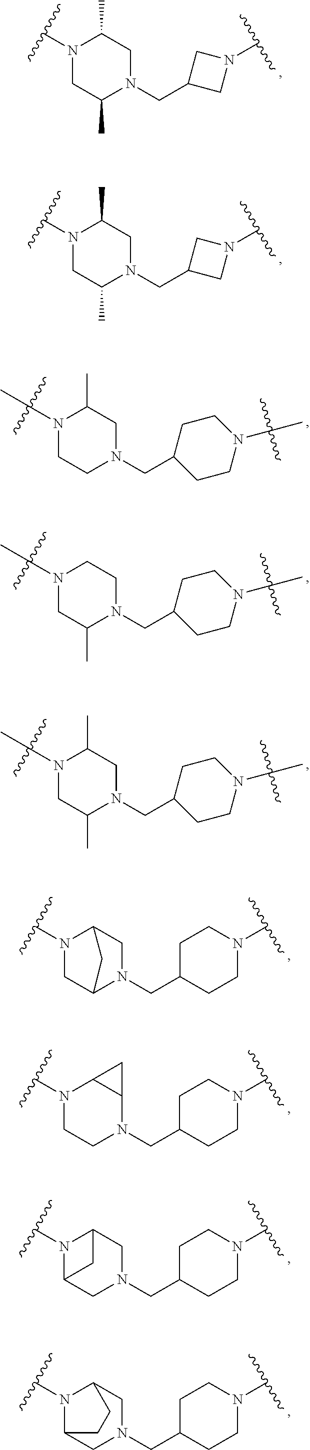

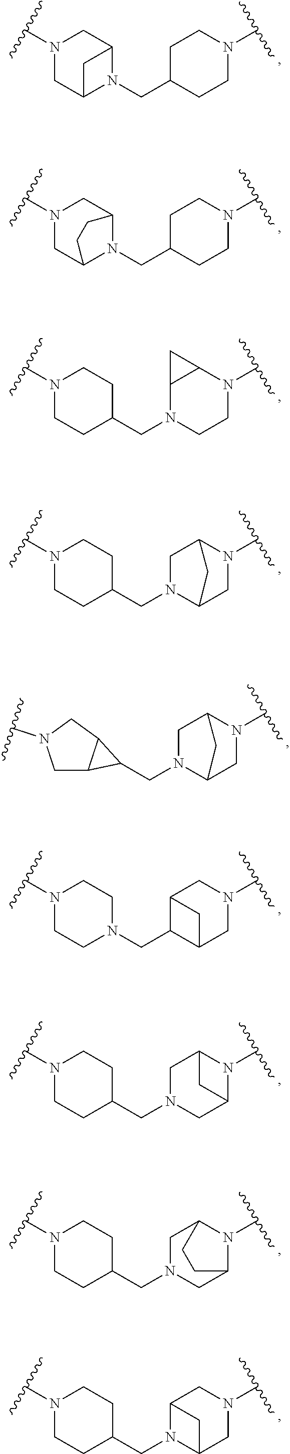

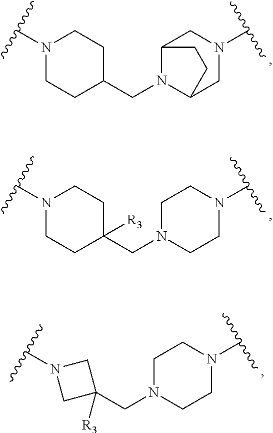

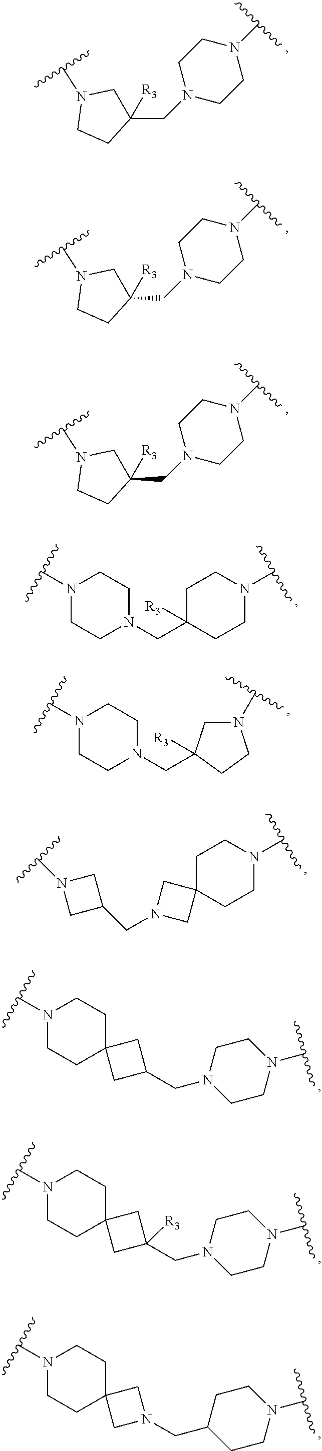

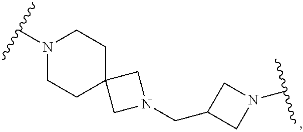 [SMILES: *N1CCC2(CC1)CN(CC1CN(*)C1)C2]

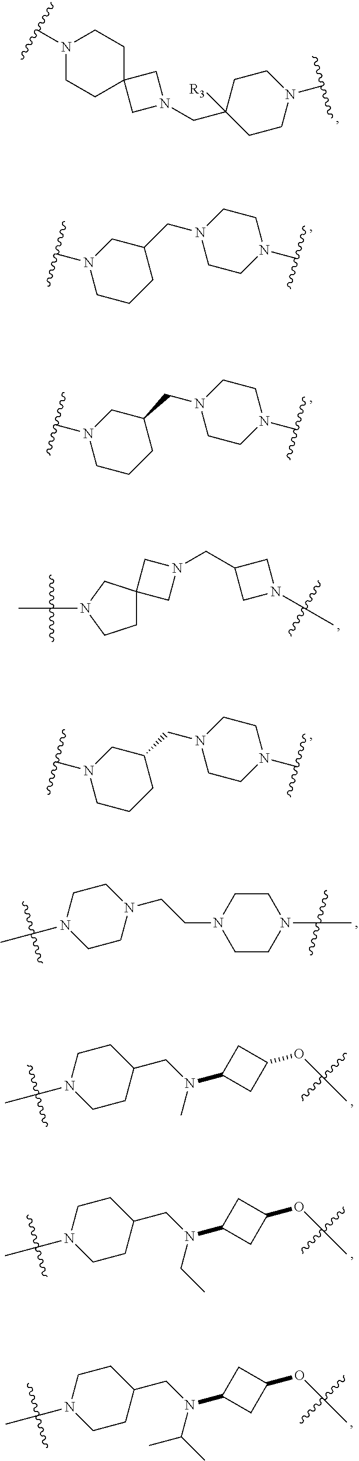

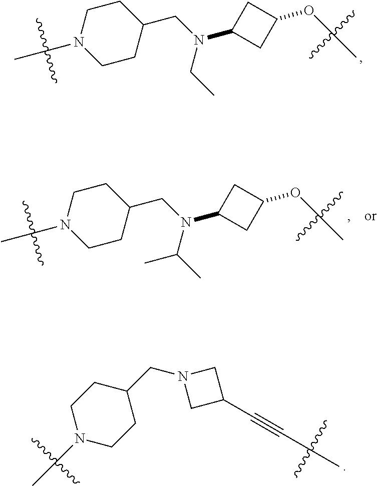 [SMILES: *O[C@H]1C[C@H](N(CC)CC2CCN(*)CC2)C1 ; *O[C@H]1C[C@H](N(CC2CCN(*)CC2)C(C)C)C1 ; *C#CC1CN(CC2CCN(*)CC2)C1]

27. The compound according to claim 26, wherein R₃ is independently hydrogen, halogen, hydroxyl, or C₁-C₃ alkoxyl.

28. The compound according to claim 27, wherein R₃ is 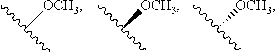
OH, or F.

29. The compound according to claim 27, wherein R₃ is F.

30. The compound according to any one of claims 1 to 29, wherein Q is:

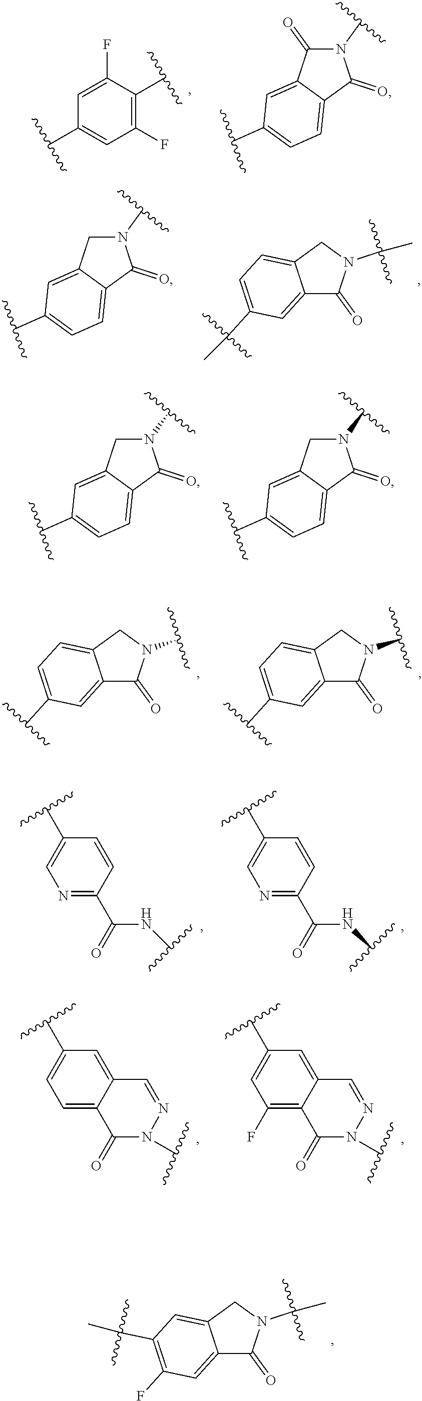

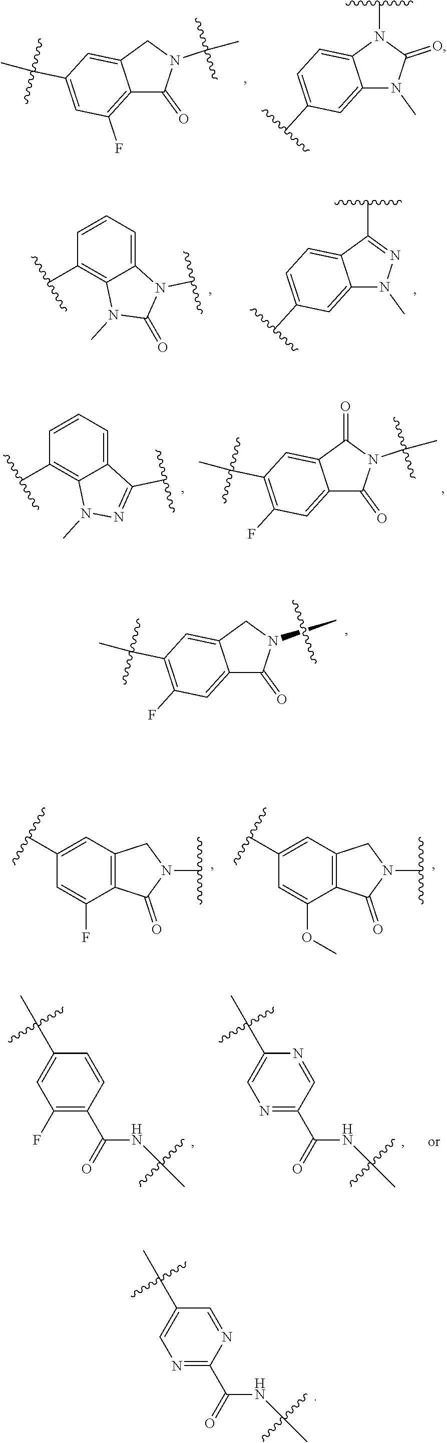

31. The compound according to claim 1, wherein the compound is:
N-((1,3-trans)-3-((8-cyano-2-oxo-1,2-dihydroquinolin-5-yl)oxy)-2,2,4,4-tetramethylcyclobutyl)-4-(3-((4-(2-(2,6-dioxopiperidin-3-yl)-1,3-dioxoisoindolin-5-yl)piperazin-1-yl)methyl)azetidin-1-yl)benzamide;
N-((1,3-trans)-3-((8-cyano-2-oxo-1,2-dihydroquinolin-5-yl)oxy)-2,2,4,4-tetramethylcyclobutyl)-4-(3-((4-(2-(2,6-dioxopiperidin-3-yl)-1-oxoisoindolin-5-yl)piperazin-1-yl)methyl)azetidin-1-yl)benzamide;
N-((1,3-trans)-3-((8-cyano-2-oxo-1,2-dihydroquinolin-5-yl)oxy)-2,2,4,4-tetramethylcyclobutyl)-4-(3-((4-(2-((S)-2,6-dioxopiperidin-3-yl)-1-oxoisoindolin-5-yl)piperazin-1-yl)methyl)azetidin-1-yl)benzamide;
N-((1,3-trans)-3-((8-cyano-2-oxo-1,2-dihydroquinolin-5-yl)oxy)-2,2,4,4-tetramethylcyclobutyl)-4-(3-((4-(2-((S)-2,6-dioxopiperidin-3-yl)-1-oxoisoindolin-5-yl)piperazin-1-yl)methyl)azetidin-1-yl)-3-fluorobenzamide;
N-((1,3-trans)-3-((8-cyano-2-oxo-1,2-dihydroquinolin-5-yl)oxy)-2,2,4,4-tetramethylcyclobutyl)-4-(3-((4-(2-(2,6-dioxopiperidin-3-yl)-1-oxoisoindolin-5-yl)piperazin-1-yl)methyl)azetidin-1-yl)-3-fluorobenzamide;
N-((1,3-trans)-3-((8-cyano-2-oxo-1,2-dihydroquinolin-5-yl)oxy)-2,2,4,4-tetramethylcyclobutyl)-4-(3-((4-(2-(2,6-dioxopiperidin-3-yl)-1,3-dioxoisoindolin-5-yl)piperazin-1-yl)methyl)azetidin-1-yl)-3-fluorobenzamide;
N-((1,3-trans)-3-((8-cyano-2-oxo-1,2-dihydroquinolin-5-yl)oxy)-2,2,4,4-tetramethylcyclobutyl)-4-((1α,5α,6α)-6-((4-(2-((S)-2,6-dioxopiperidin-3-yl)-1-oxoisoindolin-5-yl)piperazin-1-yl)methyl)-3-azabicyclo[3.1.0]hexan-3-yl)benzamide;
N-((1,3-trans)-3-((8-cyano-2-oxo-1,2-dihydroquinolin-5-yl)oxy)-2,2,4,4-tetramethylcyclobutyl)-4-((1α,5α,6α)-6-((4-(2-(2,6-dioxopiperidin-3-yl)-1-oxoisoindolin-5-yl)piperazin-1-yl)methyl)-3-azabicyclo[3.1.0]hexan-3-yl)benzamide;
N-((1,3-trans)-3-((8-cyano-2-oxo-1,2-dihydroquinolin-5-yl)oxy)-2,2,4,4-tetramethylcyclobutyl)-4-((1α,5α,6α)-6-((4-(2-(2,6-dioxopiperidin-3-yl)-1,3-dioxoisoindolin-5-yl)piperazin-1-yl)methyl)-3-azabicyclo[3.1.0]hexan-3-yl)benzamide;
N-((1,3-trans)-3-((8-cyano-2-oxo-1,2-dihydroquinolin-5-yl)oxy)-2,2,4,4-tetramethylcyclobutyl)-4-((1α,5α,6α)-6-((4-(2-((S)-2,6-dioxopiperidin-3-yl)-1-oxoisoindolin-5-yl)piperazin-1-yl)methyl)-3-azabicyclo[3.1.0]hexan-3-yl)-3-fluorobenzamide;
N-((1,3-trans)-3-((8-cyano-2-oxo-1,2-dihydroquinolin-5-yl)oxy)-2,2,4,4-tetramethylcyclobutyl)-6-((1α,5α,6α)-6-((4-(2-(2,6-dioxopiperidin-3-yl)-1,3-dioxoisoindolin-5-yl)piperazin-1-yl)methyl)-3-azabicyclo[3.1.0]hexan-3-yl)nicotinamide;
N-((1,3-trans)-3-((8-cyano-2-oxo-1,2-dihydroquinolin-5-yl)oxy)-2,2,4,4-tetramethylcyclobutyl)-4-((R)-3-((4-(2-((S)-2,6-dioxopiperidin-3-yl)-1-oxoisoindolin-5-yl)piperazin-1-yl)methyl)pyrrolidin-1-yl)benzamide;
N-((1,3-trans)-3-((8-cyano-2-oxo-1,2-dihydroquinolin-5-yl)oxy)-2,2,4,4-tetramethylcyclobutyl)-4-((S)-3-((4-(2-((S)-2,6-dioxopiperidin-3-yl)-1-oxoisoindolin-5-yl)piperazin-1-yl)methyl)pyrrolidin-1-yl)benzamide;
N-((1,3-trans)-3-((8-cyano-2-oxo-1,2-dihydroquinolin-5-yl)oxy)-2,2,4,4-tetramethylcyclobutyl)-4-((R)-3-((4-(2-((S)-2,6-dioxopiperidin-3-yl)-1-oxoisoindolin-5-yl)piperazin-1-yl)methyl)pyrrolidin-1-yl)-3-fluorobenzamide;
N-((1r,3S)-3-((8-cyano-2-oxo-1,2-dihydroquinolin-5-yl)oxy)-2,2,4,4-tetramethylcyclobutyl)-4-((S)-3-((4-(2-((S)-2,6-dioxopiperidin-3-yl)-1-oxoisoindolin-5-yl)piperazin-1-yl)methyl)pyrrolidin-1-yl)-3-fluorobenzamide;
N-((1,3-trans)-3-((8-cyano-2-oxo-1,2-dihydroquinolin-5-yl)oxy)-2,2,4,4-tetramethylcyclobutyl)-4-(4-((4-(2-((S)-2,6-dioxopiperidin-3-yl)-1-oxoisoindolin-5-yl)piperazin-1-yl)methyl)piperidin-1-yl)-3-fluorobenzamide;
N-((1,3-trans)-3-((8-cyano-2-oxo-1,2-dihydroquinolin-5-yl)oxy)-2,2,4,4-tetramethylcyclobutyl)-4-(6-((4-(2-((S)-2,6-dioxopiperidin-3-yl)-1-oxoisoindolin-5-yl)piperazin-1-yl)methyl)-3-azabicyclo[3.1.0]hexan-3-yl)benzamide;
N-((1,3-trans)-3-((8-cyano-2-oxo-1,2-dihydroquinolin-5-yl)oxy)-2,2,4,4-tetramethylcyclobutyl)-4-(4-((4-(2-((S)-2,6-dioxopiperidin-3-yl)-1-oxoisoindolin-5-yl)piperazin-1-yl)methyl)piperidin-1-yl)benzamide;
N-((1,3-trans)-3-((8-cyano-2-oxo-1,2-dihydroquinolin-5-yl)oxy)-2,2,4,4-tetramethylcyclobutyl)-4-(4-((4-(2-(2,6-dioxopiperidin-3-yl)-1,3-dioxoisoindolin-5-yl)piperazin-1-yl)methyl)piperidin-1-yl)-3-fluorobenzamide;
N-((1,3-trans)-3-((8-cyano-2-oxo-1,2-dihydroquinolin-5-yl)oxy)-2,2,4,4-tetramethylcyclobutyl)-4-(4-((4-(2-(2,6-dioxopiperidin-3-yl)-1,3-dioxoisoindolin-5-yl)piperazin-1-yl)methyl)piperidin-1-yl)benzamide;
N-((1,3-trans)-3-((8-cyano-2-oxo-1,2-dihydroquinolin-5-yl)oxy)-2,2,4,4-tetramethylcyclobutyl)-6-(4-((4-(2-(2,6-dioxopiperidin-3-yl)-1,3-dioxoisoindolin-5-yl)piperazin-1-yl)methyl)piperidin-1-yl)nicotinamide;
N-((1,3-trans)-3-((8-cyano-2-oxo-1,2-dihydroquinolin-5-yl)oxy)-2,2,4,4-tetramethylcyclobutyl)-2-(4-((4-(2-(2,6-dioxopiperidin-3-yl)-1,3-dioxoisoindolin-5-yl)piperazin-1-yl)methyl)piperidin-1-yl)pyrimidine-5-carboxamide;
N-((1,3-trans)-3-((8-cyano-2-oxo-1,2-dihydroquinolin-5-yl)oxy)-2,2,4,4-tetramethylcyclobutyl)-5-(4-((4-(2-(2,6-dioxopiperidin-3-yl)-1,3-dioxoisoindolin-5-yl)piperazin-1-yl)methyl)piperidin-1-yl)pyrazine-2-carboxamide;
N-((1,3-trans)-3-((8-cyano-2-oxo-1,2-dihydroquinolin-5-yl)oxy)-2,2,4,4-tetramethylcyclobutyl)-6-(4-((4-(2-(2,6-dioxopiperidin-3-yl)-1,3-dioxoisoindolin-5-yl)piperazin-1-yl)methyl)piperidin-1-yl)pyridazine-3-carboxamide;
N-((1,3-trans)-3-((8-cyano-1-methyl-2-oxo-1,2-dihydroquinolin-5-yl)oxy)-2,2,4,4-tetramethylcyclobutyl)-4-(4-((4-(2-((S)-2,6-dioxopiperidin-3-yl)-1-oxoisoindolin-5-yl)piperazin-1-yl)methyl)piperidin-1-yl)benzamide;
N-((1,3-trans)-3-((8-cyano-1-methyl-2-oxo-1,2-dihydroquinolin-5-yl)oxy)-2,2,4,4-tetramethylcyclobutyl)-4-(4-((4-(2-((S)-2,6-dioxopiperidin-3-yl)-1-oxoisoindolin-5-yl)piperazin-1-yl)methyl)piperidin-1-yl)-3-fluorobenzamide;
N-((1,3-trans)-3-((8-cyano-2-oxo-1,2-dihydroquinolin-5-yl)oxy)-2,2,4,4-tetramethylcyclobutyl)-4-(4-((4-(2-((S)-2,6-dioxopiperidin-3-yl)-3-oxoisoindolin-5-yl)piperazin-1-yl)methyl)piperidin-1-yl)benzamide;
N-((1,3-trans)-3-((8-cyano-2-oxo-1,2-dihydroquinolin-5-yl)oxy)-2,2,4,4-tetramethylcyclobutyl)-4-(4-((4-(2-((S)-2,6-dioxopiperidin-3-yl)-3-oxoisoindolin-5-yl)piperazin-1-yl)methyl)piperidin-1-yl)-3-fluorobenzamide;
N-((1,3-trans)-3-((8-cyano-1-methyl-2-oxo-1,2-dihydroquinolin-5-yl)oxy)-2,2,4,4-tetramethylcyclobutyl)-4-(4-((4-(2-((S)-2,6-dioxopiperidin-3-yl)-3-oxoisoindolin-5-yl)piperazin-1-yl)methyl)piperidin-1-yl)benzamide;
N-((1,3-trans)-3-((8-cyano-1-methyl-2-oxo-1,2-dihydroquinolin-5-yl)oxy)-2,2,4,4-tetramethylcyclobutyl)-4-(4-((4-(2-(2,6-dioxopiperidin-3-yl)-1,3-dioxoisoindolin-5-yl)piperazin-1-yl)methyl)piperidin-1-yl)benzamide;
5-(4-((1-(4-(((1,3-trans)-3-((8-cyano-2-oxo-1,2-dihydroquinolin-5-yl)oxy)-2,2,4,4-tetramethylcyclobutyl)carbamoyl)phenyl)piperidin-4-yl)methyl)piperazin-1-yl)-N—((S)-2,6-dioxopiperidin-3-yl)picolinamide;
5-(4-((1-(4-(((1,3-trans)-3-((8-cyano-2-oxo-1,2-dihydroquinolin-5-yl)oxy)-2,2,4,4-tetramethylcyclobutyl)carbamoyl)-2-fluorophenyl)piperidin-4-yl)methyl)piperazin-1-yl)-N—((S)-2,6-dioxopiperidin-3-yl)picolinamide;
5-(4-((1-(4-(((1,3-trans)-3-((8-cyano-1-methyl-2-oxo-1,2-dihydroquinolin-5-yl)oxy)-2,2,4,4-tetramethylcyclobutyl)carbamoyl)phenyl)piperidin-4-yl)methyl)piperazin-1-yl)-N—((S)-2,6-dioxopiperidin-3-yl)picolinamide;
N-((1,3-trans)-3-((8-cyano-2-oxo-1,2-dihydroquinolin-5-yl)oxy)-2,2,4,4-tetramethylcyclobutyl)-4-(4-((4-(2-(2,6-dioxopiperidin-3-yl)-1-oxo-1,2-dihydrophthalazin-6-yl)piperazin-1-yl)methyl)piperidin-1-yl)benzamide;
N-((1,3-trans)-3-((8-cyano-1-methyl-2-oxo-1,2-dihydroquinolin-5-yl)oxy)-2,2,4,4-tetramethylcyclobutyl)-4-(4-((4-(2-(2,6-dioxopiperidin-3-yl)-1-oxo-1,2-dihydrophthalazin-6-yl)piperazin-1-yl)methyl)piperidin-1-yl)benzamide;
N-((1,3-trans)-3-((8-cyano-2-oxo-1,2-dihydroquinolin-5-yl)oxy)cyclobutyl)-4-(4-((4-(2-((S)-2,6-dioxopiperidin-3-yl)-1-oxoisoindolin-5-yl)piperazin-1-yl)methyl)piperidin-1-yl)benzamide;
N-((1,3-trans)-3-((8-cyano-1-methyl-2-oxo-1,2-dihydroquinolin-5-yl)oxy)cyclobutyl)-4-(4-((4-(2-((S)-2,6-dioxopiperidin-3-yl)-1-oxoisoindolin-5-yl)piperazin-1-yl)methyl)piperidin-1-yl)benzamide;
N-((1,3-trans)-3-((8-cyano-1-(methyl-d3)-2-oxo-1,2-dihydroquinolin-5-yl)oxy)-2,2,4,4-tetramethylcyclobutyl)-4-(4-((4-(2-(2,6-dioxopiperidin-3-yl)-1,3-dioxoisoindolin-5-yl)piperazin-1-yl)methyl)piperidin-1-yl)benzamide;
N-((1,3-trans)-3-((8-cyano-1-(methyl-d3)-2-oxo-1,2-dihydroquinolin-5-yl)oxy)-2,2,4,4-tetramethylcyclobutyl)-4-(4-((4-(2-(2,6-dioxopiperidin-3-yl)-3-oxoisoindolin-5-yl)piperazin-1-yl)methyl)piperidin-1-yl)benzamide;
N-((1,3-trans)-3-((8-cyano-2-oxo-1,2-dihydroquinolin-5-yl)oxy)-2,2,4,4-tetramethylcyclobutyl)-4-(4-((1-(2-(2,6-dioxopiperidin-3-yl)-1-oxoisoindolin-5-yl)piperidin-4-yl)methyl)piperazin-1-yl)benzamide;
N-((1,3-trans)-3-((8-cyano-1-methyl-2-oxo-1,2-dihydroquinolin-5-yl)oxy)-2,2,4,4-tetramethylcyclobutyl)-4-(4-((1-(2-(2,6-dioxopiperidin-3-yl)-1-oxoisoindolin-5-yl)piperidin-4-yl)methyl)piperazin-1-yl)benzamide;
N-((1,3-trans)-3-((8-cyano-2-oxo-1,2-dihydroquinolin-5-yl)oxy)-2,2,4,4-tetramethylcyclobutyl)-4-(4-(((3S)-1-(2-(2,6-dioxopiperidin-3-yl)-1-oxoisoindolin-5-yl)pyrrolidin-3-yl)methyl)piperazin-1-yl)benzamide;
N-((1,3-trans)-3-((8-cyano-2-oxo-1,2-dihydroquinolin-5-yl)oxy)-2,2,4,4-tetramethylcyclobutyl)-4-(4-(((3R)-1-(2-(2,6-dioxopiperidin-3-yl)-1-oxoisoindolin-5-yl)pyrrolidin-3-yl)methyl)piperazin-1-yl)benzamide;
N-((1,3-trans)-3-((8-cyano-2-oxo-1,2-dihydroquinolin-5-yl)oxy)-2,2,4,4-tetramethylcyclobutyl)-4-(4-((1-(2-(2,6-dioxopiperidin-3-yl)-1-oxoisoindolin-5-yl)azetidin-3-yl)methyl)piperazin-1-yl)benzamide;
N-((1,3-trans)-3-((8-cyano-2-oxo-1,2-dihydroquinolin-5-yl)oxy)-2,2,4,4-tetramethylcyclobutyl)-4-((2S)-4-((1-(2-(2,6-dioxopiperidin-3-yl)-1-oxoisoindolin-5-yl)azetidin-3-yl)methyl)-2-methylpiperazin-1-yl)benzamide;
N-((1,3-trans)-3-((8-cyano-2-oxo-1,2-dihydroquinolin-5-yl)oxy)-2,2,4,4-tetramethylcyclobutyl)-4-((2R)-4-((1-(2-(2,6-dioxopiperidin-3-yl)-1-oxoisoindolin-5-yl)azetidin-3-yl)methyl)-2-methylpiperazin-1-yl)benzamide;
N-((1,3-trans)-3-((8-cyano-2-oxo-1,2-dihydroquinolin-5-yl)oxy)-2,2,4,4-tetramethylcyclobutyl)-4-((2R,5S)-4-((1-(2-(2,6-dioxopiperidin-3-yl)-1-oxoisoindolin-5-yl)azetidin-3-yl)methyl)-2,5-dimethylpiperazin-1-yl)benzamide;
N-((1,3-trans)-3-((8-cyano-2-oxo-1,2-dihydroquinolin-5-yl)oxy)-2,2,4,4-tetramethylcyclobutyl)-4-((2S,5R)-4-((1-(2-(2,6-dioxopiperidin-3-yl)-1-oxoisoindolin-5-yl)azetidin-3-yl)methyl)-2,5-dimethylpiperazin-1-yl)benzamide;
N-((1,3-trans)-3-((8-cyano-2-oxo-1,2-dihydroquinolin-5-yl)oxy)-2,2,4,4-tetramethylcyclobutyl)-4-(5-((1-(2-(2,6-dioxopiperidin-3-yl)-1-oxoisoindolin-5-yl)piperidin-4-yl)methyl)-2,5-diazabicyclo[2.2.1]heptan-2-yl)benzamide;
N-((1,3-trans)-3-((8-cyano-2-oxo-1,2-dihydroquinolin-5-yl)oxy)-2,2,4,4-tetramethylcyclobutyl)-4-(5-((1-(2-(2,6-dioxopiperidin-3-yl)-1-oxoisoindolin-5-yl)piperidin-4-yl)methyl)-2,5-diazabicyclo[4.1.0]heptan-2-yl)benzamide;
N-((1,3-trans)-3-((8-cyano-2-oxo-1,2-dihydroquinolin-5-yl)oxy)-2,2,4,4-tetramethylcyclobutyl)-4-(3-((1-(2-(2,6-dioxopiperidin-3-yl)-1-oxoisoindolin-5-yl)piperidin-4-yl)methyl)-3,6-diazabicyclo[3.1.1]heptan-6-yl)benzamide;
N-((1,3-trans)-3-((8-cyano-2-oxo-1,2-dihydroquinolin-5-yl)oxy)-2,2,4,4-tetramethylcyclobutyl)-4-(3-((1-(2-(2,6-dioxopiperidin-3-yl)-1-oxoisoindolin-5-yl)piperidin-4-yl)methyl)-3,8-diazabicyclo[3.2.1]octan-8-yl)benzamide;
N-((1,3-trans)-3-((8-cyano-2-oxo-1,2-dihydroquinolin-5-yl)oxy)-2,2,4,4-tetramethylcyclobutyl)-4-(6-((1-(2-(2,6-dioxopiperidin-3-yl)-1-oxoisoindolin-5-yl)piperidin-4-yl)methyl)-3,6-diazabicyclo[3.1.1]heptan-3-yl)benzamide;
N-((1,3-trans)-3-((8-cyano-2-oxo-1,2-dihydroquinolin-5-yl)oxy)-2,2,4,4-tetramethylcyclobutyl)-4-(8-((1-(2-(2,6-dioxopiperidin-3-yl)-1-oxoisoindolin-5-yl)piperidin-4-yl)methyl)-3,8-diazabicyclo[3.2.1]octan-3-yl)benzamide;
N-((1,3-trans)-3-((8-cyano-2-oxo-1,2-dihydroquinolin-5-yl)oxy)-2,2,4,4-tetramethylcyclobutyl)-4-(4-((5-(2-((S)-2,6-dioxopiperidin-3-yl)-1-oxoisoindolin-5-yl)-2,5-diazabicyclo[4.1.0]heptan-2-yl)methyl)piperidin-1-yl)benzamide;
N-((1,3-trans)-3-((8-cyano-2-oxo-1,2-dihydroquinolin-5-yl)oxy)-2,2,4,4-tetramethylcyclobutyl)-4-(4-((5-(2-((S)-2,6-dioxopiperidin-3-yl)-1-oxoisoindolin-5-yl)-2,5-diazabicyclo[2.2.1]heptan-2-yl)methyl)piperidin-1-yl)benzamide;
N-((1,3-trans)-3-((8-cyano-2-oxo-1,2-dihydroquinolin-5-yl)oxy)-2,2,4,4-tetramethylcyclobutyl)-4-(6-((5-(2-((S)-2,6-dioxopiperidin-3-yl)-1-oxoisoindolin-5-yl)-2,5-diazabicyclo[2.2.1]heptan-2-yl)methyl)-3-azabicyclo[3.1.0]hexan-3-yl)benzamide;
N-((1,3-trans)-3-((8-cyano-2-oxo-1,2-dihydroquinolin-5-yl)oxy)-2,2,4,4-tetramethylcyclobutyl)-4-(4-((3-(2-(2,6-dioxopiperidin-3-yl)-1-oxoisoindolin-5-yl)-3-azabicyclo[3.1.1]heptan-6-yl)methyl)piperazin-1-yl)benzamide;
N-((1,3-trans)-3-((8-cyano-2-oxo-1,2-dihydroquinolin-5-yl)oxy)-2,2,4,4-tetramethylcyclobutyl)-4-(4-((6-(2-((S)-2,6-dioxopiperidin-3-yl)-1-oxoisoindolin-5-yl)-3,6-diazabicyclo[3.1.1]heptan-3-yl)methyl)piperidin-1-yl)benzamide;
N-((1,3-trans)-3-((8-cyano-2-oxo-1,2-dihydroquinolin-5-yl)oxy)-2,2,4,4-tetramethylcyclobutyl)-4-(4-((8-(2-((S)-2,6-dioxopiperidin-3-yl)-1-oxoisoindolin-5-yl)-3,8-diazabicyclo[3.2.1]octan-3-yl)methyl)piperidin-1-yl)benzamide;
N-((1,3-trans)-3-((8-cyano-2-oxo-1,2-dihydroquinolin-5-yl)oxy)-2,2,4,4-tetramethylcyclobutyl)-4-(4-((3-(2-((S)-2,6-dioxopiperidin-3-yl)-1-oxoisoindolin-5-yl)-3,6-diazabicyclo[3.1.1]heptan-6-yl)methyl)piperidin-1-yl)benzamide;
N-((1,3-trans)-3-((8-cyano-2-oxo-1,2-dihydroquinolin-5-yl)oxy)-2,2,4,4-tetramethylcyclobutyl)-4-(4-((3-(2-((S)-2,6-dioxopiperidin-3-yl)-1-oxoisoindolin-5-yl)-3,8-diazabicyclo[3.2.1]octan-8-yl)methyl)piperidin-1-yl)benzamide;
N-((1,3-trans)-3-((8-cyano-2-oxo-1,2-dihydroquinolin-5-yl)oxy)-2,2,4,4-tetramethylcyclobutyl)-4-(4-((4-(2-((S)-2,6-dioxopiperidin-3-yl)-1-oxoisoindolin-5-yl)piperazin-1-yl)methyl)-4-fluoropiperidin-1-yl)benzamide;
N-((1,3-trans)-3-((8-cyano-2-oxo-1,2-dihydroquinolin-5-yl)oxy)-2,2,4,4-tetramethylcyclobutyl)-4-(3-((4-(2-((S)-2,6-dioxopiperidin-3-yl)-1-oxoisoindolin-5-yl)piperazin-1-yl)methyl)-3-fluoroazetidin-1-yl)benzamide;
N-((1,3-trans)-3-((8-cyano-2-oxo-1,2-dihydroquinolin-5-yl)oxy)-2,2,4,4-tetramethylcyclobutyl)-4-((R)-3-((4-(2-((S)-2,6-dioxopiperidin-3-yl)-1-oxoisoindolin-5-yl)piperazin-1-yl)methyl)-3-fluoropyrrolidin-1-yl)benzamide;
N-((1,3-trans)-3-((8-cyano-2-oxo-1,2-dihydroquinolin-5-yl)oxy)-2,2,4,4-tetramethylcyclobutyl)-4-((S)-3-((4-(2-((S)-2,6-dioxopiperidin-3-yl)-1-oxoisoindolin-5-yl)piperazin-1-yl)methyl)-3-fluoropyrrolidin-1-yl)benzamide;
N-((1,3-trans)-3-((8-cyano-2-oxo-1,2-dihydroquinolin-5-yl)oxy)-2,2,4,4-tetramethylcyclobutyl)-4-((S)-3-((4-(2-((S)-2,6-dioxopiperidin-3-yl)-1-oxoisoindolin-5-yl)piperazin-1-yl)methyl)-3-methylpyrrolidin-1-yl)benzamide;
N-((1,3-trans)-3-((8-cyano-2-oxo-1,2-dihydroquinolin-5-yl)oxy)-2,2,4,4-tetramethylcyclobutyl)-4-((R)-3-((4-(2-((S)-2,6-dioxopiperidin-3-yl)-1-oxoisoindolin-5-yl)piperazin-1-yl)methyl)-3-methylpyrrolidin-1-yl)benzamide;
N-((1,3-trans)-3-((8-cyano-2-oxo-1,2-dihydroquinolin-5-yl)oxy)-2,2,4,4-tetramethylcyclobutyl)-4-(3-((4-(2-((S)-2,6-dioxopiperidin-3-yl)-1-oxoisoindolin-5-yl)piperazin-1-yl)methyl)-3-methylazetidin-1-yl)benzamide;
N-((1,3-trans)-3-((8-cyano-2-oxo-1,2-dihydroquinolin-5-yl)oxy)-2,2,4,4-tetramethylcyclobutyl)-4-(4-((4-(2-((S)-2,6-dioxopiperidin-3-yl)-1-oxoisoindolin-5-yl)piperazin-1-yl)methyl)-4-methylpiperidin-1-yl)benzamide;
N-((1,3-trans)-3-((8-cyano-2-oxo-1,2-dihydroquinolin-5-yl)oxy)-2,2,4,4-tetramethylcyclobutyl)-4-(4-((1-(2-(2,6-dioxopiperidin-3-yl)-1-oxoisoindolin-5-yl)-4-methylpiperidin-4-yl)methyl)piperazin-1-yl)benzamide;
N-((1,3-trans)-3-((8-cyano-2-oxo-1,2-dihydroquinolin-5-yl)oxy)-2,2,4,4-tetramethylcyclobutyl)-4-(4-((1-(2-(2,6-dioxopiperidin-3-yl)-1-oxoisoindolin-5-yl)-4-fluoropiperidin-4-yl)methyl)piperazin-1-yl)benzamide;
N-((1,3-trans)-3-((8-cyano-2-oxo-1,2-dihydroquinolin-5-yl)oxy)-2,2,4,4-tetramethylcyclobutyl)-4-(4-((1-(2-(2,6-dioxopiperidin-3-yl)-1-oxoisoindolin-5-yl)-3-methylpyrrolidin-3-yl)methyl)piperazin-1-yl)benzamide;
N-((1,3-trans)-3-((8-cyano-2-oxo-1,2-dihydroquinolin-5-yl)oxy)-2,2,4,4-tetramethylcyclobutyl)-4-(4-((1-(2-(2,6-dioxopiperidin-3-yl)-1-oxoisoindolin-5-yl)-3-fluoropyrrolidin-3-yl)methyl)piperazin-1-yl)benzamide;
N-((1,3-trans)-3-((8-cyano-2-oxo-1,2-dihydroquinolin-5-yl)oxy)-2,2,4,4-tetramethylcyclobutyl)-4-(3-((7-(2-(2,6-dioxopiperidin-3-yl)-1-oxoisoindolin-5-yl)-2,7-diazaspiro[3.5]nonan-2-yl)methyl)azetidin-1-yl)benzamide;
N-((1,3-trans)-3-((8-cyano-2-oxo-1,2-dihydroquinolin-5-yl)oxy)-2,2,4,4-tetramethylcyclobutyl)-4-(2-((4-(2-((S)-2,6-dioxopiperidin-3-yl)-1-oxoisoindolin-5-yl)piperazin-1-yl)methyl)-7-azaspiro[3.5]nonan-7-yl)benzamide;
N-((1,3-trans)-3-((8-cyano-2-oxo-1,2-dihydroquinolin-5-yl)oxy)-2,2,4,4-tetramethylcyclobutyl)-4-(2-((4-(2-((S)-2,6-dioxopiperidin-3-yl)-1-oxoisoindolin-5-yl)piperazin-1-yl)methyl)-7-azaspiro[3.5]nonan-7-yl)-3-fluorobenzamide;
N-((1,3-trans)-3-((8-cyano-2-oxo-1,2-dihydroquinolin-5-yl)oxy)-2,2,4,4-tetramethylcyclobutyl)-4-((R)-3-((4-(2-((S)-2,6-dioxopiperidin-3-yl)-1-oxoisoindolin-5-yl)piperazin-1-yl)methyl)piperidin-1-yl)benzamide;
N-((1,3-trans)-3-((8-cyano-2-oxo-1,2-dihydroquinolin-5-yl)oxy)-2,2,4,4-tetramethylcyclobutyl)-4-((R)-3-((4-(2-((S)-2,6-dioxopiperidin-3-yl)-1-oxoisoindolin-5-yl)piperazin-1-yl)methyl)piperidin-1-yl)-3-fluorobenzamide;
N-((1,3-trans)-3-((8-cyano-2-oxo-1,2-dihydroquinolin-5-yl)oxy)-2,2,4,4-tetramethylcyclobutyl)-4-((S)-3-((4-(2-((S)-2,6-dioxopiperidin-3-yl)-1-oxoisoindolin-5-yl)piperazin-1-yl)methyl)piperidin-1-yl)benzamide;
N-((1,3-trans)-3-((8-cyano-2-oxo-1,2-dihydroquinolin-5-yl)oxy)-2,2,4,4-tetramethylcyclobutyl)-4-((S)-3-((4-(2-((S)-2,6-dioxopiperidin-3-yl)-1-oxoisoindolin-5-yl)piperazin-1-yl)methyl)piperidin-1-yl)-3-fluorobenzamide;
N-((1,3-trans)-3-((8-cyano-4-oxo-1,4-dihydroquinolin-5-yl)oxy)-2,2,4,4-tetramethylcyclobutyl)-4-(3-((4-(2-((S)-2,6-dioxopiperidin-3-yl)-1-oxoisoindolin-5-yl)piperazin-1-yl)methyl)azetidin-1-yl)benzamide;
N-((1,3-trans)-3-((8-cyano-4-oxo-1,4-dihydroquinolin-5-yl)oxy)-2,2,4,4-tetramethylcyclobutyl)-4-((1α,5α,6α)-6-((4-(2-((S)-2,6-dioxopiperidin-3-yl)-1-oxoisoindolin-5-yl)piperazin-1-yl)methyl)-3-azabicyclo[3.1.0]hexan-3-yl)benzamide;
N-((1,3-trans)-3-((8-cyano-4-oxo-1,4-dihydroquinolin-5-yl)oxy)-2,2,4,4-tetramethylcyclobutyl)-4-(6-((4-(2-(2,6-dioxopiperidin-3-yl)-1-oxoisoindolin-5-yl)piperazin-1-yl)methyl)-3-azabicyclo[3.1.0]hexan-3-yl)benzamide;
N-((1,3-trans)-3-((8-cyano-4-oxo-1,4-dihydroquinolin-5-yl)oxy)-2,2,4,4-tetramethylcyclobutyl)-6-(6-((4-(2-(2,6-dioxopiperidin-3-yl)-1,3-dioxoisoindolin-5-yl)piperazin-1-yl)methyl)-3-azabicyclo[3.1.0]hexan-3-yl)nicotinamide;
N-((1,3-trans)-3-((8-cyano-4-oxo-1,4-dihydroquinolin-5-yl)oxy)-2,2,4,4-tetramethylcyclobutyl)-4-(4-((4-(2-((S)-2,6-dioxopiperidin-3-yl)-1-oxoisoindolin-5-yl)piperazin-1-yl)methyl)piperidin-1-yl)-3-fluorobenzamide;
N-((1,3-trans)-3-((8-cyano-4-oxo-1,4-dihydroquinolin-5-yl)oxy)-2,2,4,4-tetramethylcyclobutyl)-4-(4-((4-(2-((S)-2,6-dioxopiperidin-3-yl)-1-oxoisoindolin-5-yl)piperazin-1-yl)methyl)piperidin-1-yl)benzamide;
N-((1,3-trans)-3-((8-cyano-4-oxo-1,4-dihydroquinolin-5-yl)oxy)-2,2,4,4-tetramethylcyclobutyl)-4-(4-((4-(2-((S)-2,6-dioxopiperidin-3-yl)-6-fluoro-1-oxoisoindolin-5-yl)piperazin-1-yl)methyl)piperidin-1-yl)benzamide;
N-((1,3-trans)-3-((8-cyano-4-oxo-1,4-dihydroquinolin-5-yl)oxy)-2,2,4,4-tetramethylcyclobutyl)-4-((R)-3-((4-(2-((S)-2,6-dioxopiperidin-3-yl)-1-oxoisoindolin-5-yl)piperazin-1-yl)methyl)pyrrolidin-1-yl)benzamide;
N-((1,3-trans)-3-((8-cyano-4-oxo-1,4-dihydroquinolin-5-yl)oxy)-2,2,4,4-tetramethylcyclobutyl)-4-((S)-3-((4-(2-((S)-2,6-dioxopiperidin-3-yl)-1-oxoisoindolin-5-yl)piperazin-1-yl)methyl)pyrrolidin-1-yl)benzamide;
N-((1,3-trans)-3-((8-cyano-1-methyl-4-oxo-1,4-dihydroquinolin-5-yl)oxy)-2,2,4,4-tetramethylcyclobutyl)-4-(4-((4-(2-((S)-2,6-dioxopiperidin-3-yl)-1-oxoisoindolin-5-yl)piperazin-1-yl)methyl)piperidin-1-yl)benzamide;
N-((1,3-trans)-3-((8-cyano-1-methyl-4-oxo-1,4-dihydroquinolin-5-yl)oxy)-2,2,4,4-tetramethylcyclobutyl)-4-(4-((4-(2-((S)-2,6-dioxopiperidin-3-yl)-1-oxoisoindolin-5-yl)piperazin-1-yl)methyl)piperidin-1-yl)-3-fluorobenzamide;
N-((1,3-trans)-3-((8-cyano-1-methyl-4-oxo-1,4-dihydroquinolin-5-yl)oxy)-2,2,4,4-tetramethylcyclobutyl)-4-(4-((4-(2-(2,6-dioxopiperidin-3-yl)-1,3-dioxoisoindolin-5-yl)piperazin-1-yl)methyl)piperidin-1-yl)benzamide;
5-(4-((1-(4-(((1,3-trans)-3-((8-cyano-4-oxo-1,4-dihydroquinolin-5-yl)oxy)-2,2,4,4-tetramethylcyclobutyl)carbamoyl)-2-fluorophenyl)piperidin-4-yl)methyl)piperazin-1-yl)-N—((S)-2,6-dioxopiperidin-3-yl)picolinamide;
5-(4-((1-(4-(((1,3-trans)-3-((8-cyano-4-oxo-1,4-dihydroquinolin-5-yl)oxy)-2,2,4,4-tetramethylcyclobutyl)carbamoyl)phenyl)piperidin-4-yl)methyl)piperazin-1-yl)-N—((S)-2,6-dioxopiperidin-3-yl)picolinamide;
5-(4-((1-(4-(((1,3-trans)-3-((8-cyano-1-methyl-4-oxo-1,4-dihydroquinolin-5-yl)oxy)-2,2,4,4-tetramethylcyclobutyl)carbamoyl)phenyl)piperidin-4-yl)methyl)piperazin-1-yl)-N—((S)-2,6-dioxopiperidin-3-yl)picolinamide;
N-((1,3-trans)-3-((8-cyano-4-oxo-1,4-dihydroquinolin-5-yl)oxy)-2,2,4,4-tetramethylcyclobutyl)-4-(4-((4-(2-(2,6-dioxopiperidin-3-yl)-1-oxo-1,2-dihydrophthalazin-6-yl)piperazin-1-yl)methyl)piperidin-1-yl)benzamide;
N-((1,3-trans)-3-((8-cyano-1-methyl-4-oxo-1,4-dihydroquinolin-5-yl)oxy)-2,2,4,4-tetramethylcyclobutyl)-4-(4-((4-(2-(2,6-dioxopiperidin-3-yl)-1-oxo-1,2-dihydrophthalazin-6-yl)piperazin-1-yl)methyl)piperidin-1-yl)benzamide;
N-((1,3-trans)-3-((8-cyano-4-oxo-1,4-dihydroquinolin-5-yl)oxy)cyclobutyl)-4-(4-((4-(2-((S)-2,6-dioxopiperidin-3-yl)-1-oxoisoindolin-5-yl)piperazin-1-yl)methyl)piperidin-1-yl)benzamide;
N-((1,3-trans)-3-((8-cyano-1-methyl-4-oxo-1,4-dihydroquinolin-5-yl)oxy)cyclobutyl)-4-(4-((4-(2-((S)-2,6-dioxopiperidin-3-yl)-1-oxoisoindolin-5-yl)piperazin-1-yl)methyl)piperidin-1-yl)benzamide;
N-((1,3-trans)-3-((8-cyano-1-(methyl-d3)-4-oxo-1,4-dihydroquinolin-5-yl)oxy)-2,2,4,4-tetramethylcyclobutyl)-4-(4-((4-(2-(2,6-dioxopiperidin-3-yl)-1,3-dioxoisoindolin-5-yl)piperazin-1-yl)methyl)piperidin-1-yl)benzamide;
N-((1,3-trans)-3-((8-cyano-1-(methyl-d3)-4-oxo-1,4-dihydroquinolin-5-yl)oxy)-2,2,4,4-tetramethylcyclobutyl)-4-(4-((4-(2-(2,6-dioxopiperidin-3-yl)-3-oxoisoindolin-5-yl)piperazin-1-yl)methyl)piperidin-1-yl)benzamide;
4-((1,3-trans)-3-(((1-(4-(((1,3-trans)-3-((8-cyano-2-oxo-1,2-dihydroquinolin-5-yl)oxy)-2,2,4,4-tetramethylcyclobutyl)carbamoyl)phenyl)piperidin-4-yl)methyl)(isopropyl)amino)cyclobutoxy)-N-(2,6-dioxopiperidin-3-yl)-2-fluorobenzamide;
4-((1-((1-(4-(((1,3-trans)-3-((8-cyano-2-oxo-1,2-dihydroquinolin-5-yl)oxy)-2,2,4,4-tetramethylcyclobutyl)carbamoyl)phenyl)piperidin-4-yl)methyl)azetidin-3-yl)ethynyl)-N-(2,6-dioxopiperidin-3-yl)-2-fluorobenzamide;
5-((1,3-trans)-3-(((1-(4-(((1,3-trans)-3-((8-cyano-2-oxo-1,2-dihydroquinolin-5-yl)oxy)-2,2,4,4-tetramethylcyclobutyl)carbamoyl)phenyl)piperidin-4-yl)methyl)(isopropyl)amino)cyclobutoxy)-N-(2,6-dioxopiperidin-3-yl)picolinamide;
N-((1,3-trans)-3-((8-cyano-2-oxo-1,2-dihydroquinolin-5-yl)oxy)-2,2,4,4-tetramethylcyclobutyl)-4-(4-((4-(2-((R)-2,6-dioxopiperidin-3-yl)-1-oxoisoindolin-5-yl)piperazin-1-yl)methyl)piperidin-1-yl)-3-fluorobenzamide;
N-((1,3-trans)-3-((8-cyano-2-oxo-1,2-dihydroquinolin-5-yl)oxy)-2,2,4,4-tetramethylcyclobutyl)-4-(4-((4-(2-((S)-2,6-dioxopiperidin-3-yl)-1-oxoisoindolin-5-yl)piperazin-1-yl)methyl)-4-fluoropiperidin-1-yl)-3-fluorobenzamide;
N-((1,3-trans)-3-((8-cyano-2-oxo-1,2-dihydroquinolin-5-yl)oxy)-2,2,4,4-tetramethylcyclobutyl)-4-(4-((4-(2-(2,6-dioxopiperidin-3-yl)-6-fluoro-1-oxoisoindolin-5-yl)piperazin-1-yl)methyl)piperidin-1-yl)-3-fluorobenzamide;
N-((1,3-trans)-3-((8-cyano-2-oxo-1,2-dihydroquinolin-5-yl)oxy)-2,2,4,4-tetramethylcyclobutyl)-4-(4-((4-(2-(2,6-dioxopiperidin-3-yl)-7-fluoro-1-oxoisoindolin-5-yl)piperazin-1-yl)methyl)piperidin-1-yl)-3-fluorobenzamide;
N-((1,3-trans)-3-((8-cyano-2-oxo-1,2-dihydroquinolin-5-yl)oxy)-2,2,4,4-tetramethylcyclobutyl)-4-(4-((4-(1-(2,6-dioxopiperidin-3-yl)-3-methyl-2-oxo-2,3-dihydro-1H-benzo[d]imidazol-5-yl)piperazin-1-yl)methyl)piperidin-1-yl)-3-fluorobenzamide;
N-((1,3-trans)-3-((8-cyano-2-oxo-1,2-dihydroquinolin-5-yl)oxy)-2,2,4,4-tetramethylcyclobutyl)-4-(4-((4-(3-(2,6-dioxopiperidin-3-yl)-1-methyl-1H-indazol-6-yl)piperazin-1-yl)methyl)piperidin-1-yl)-3-fluorobenzamide;
N-((1,3-trans)-3-((8-cyano-2-oxo-1,2-dihydroquinolin-5-yl)oxy)-2,2,4,4-tetramethylcyclobutyl)-4-(4-((4-(1-(2,6-dioxopiperidin-3-yl)-3-methyl-2-oxo-2,3-dihydro-1H-benzo[d]imidazol-4-yl)piperazin-1-yl)methyl)piperidin-1-yl)-3-fluorobenzamide;
N-((1,3-trans)-3-((8-cyano-2-oxo-1,2-dihydroquinolin-5-yl)oxy)-2,2,4,4-tetramethylcyclobutyl)-4-(4-((4-(3-(2,6-dioxopiperidin-3-yl)-1-methyl-1H-indazol-7-yl)piperazin-1-yl)methyl)piperidin-1-yl)-3-fluorobenzamide;
N-((1,3-trans)-3-((8-cyano-2-oxo-1,2-dihydroquinolin-5-yl)oxy)-2,2,4,4-tetramethylcyclobutyl)-4-(4-((4-(4-(2,6-dioxopiperidin-3-yl)-3,5-difluorophenyl)piperazin-1-yl)methyl)piperidin-1-yl)-3-fluorobenzamide;
N-((1,3-trans)-3-((8-cyano-2-oxo-1,2-dihydroquinolin-5-yl)oxy)-2,2,4,4-tetramethylcyclobutyl)-4-(4-((4-(2-(2,6-dioxopiperidin-3-yl)-7-methyl-1-oxoisoindolin-5-yl)piperazin-1-yl)methyl)piperidin-1-yl)-3-fluorobenzamide;
N-((1,3-trans)-3-((8-cyano-2-oxo-1,2-dihydroquinolin-5-yl)oxy)-2,2,4,4-tetramethylcyclobutyl)-4-(4-((4-(2-(2,6-dioxopiperidin-3-yl)-7-methyl-1-oxoisoindolin-5-yl)piperazin-1-yl)methyl)piperidin-1-yl)benzamide;
N-((1,3-trans)-3-((8-cyano-2-oxo-1,2-dihydroquinolin-5-yl)oxy)-2,2,4,4-tetramethylcyclobutyl)-4-(4-((4-(2-(2,6-dioxopiperidin-3-yl)-7-methoxy-1-oxoisoindolin-5-yl)piperazin-1-yl)methyl)piperidin-1-yl)-3-fluorobenzamide;
N-((1,3-trans)-3-((8-cyano-2-oxo-1,2-dihydroquinolin-5-yl)oxy)-2,2,4,4-tetramethylcyclobutyl)-4-(4-((4-(2-(2,6-dioxopiperidin-3-yl)-7-methoxy-1-oxoisoindolin-5-yl)piperazin-1-yl)methyl)piperidin-1-yl)benzamide;
4-(4-((1-(4-(((1,3-trans)-3-((8-cyano-2-oxo-1,2-dihydroquinolin-5-yl)oxy)-2,2,4,4-tetramethylcyclobutyl)carbamoyl)phenyl)piperidin-4-yl)methyl)piperazin-1-yl)-N—((S)-2,6-dioxopiperidin-3-yl)-2-fluorobenzamide;
4-(4-((1-(4-(((1,3-trans)-3-((8-cyano-2-oxo-1,2-dihydroquinolin-5-yl)oxy)-2,2,4,4-tetramethylcyclobutyl)carbamoyl)-2-fluorophenyl)piperidin-4-yl)methyl)piperazin-1-yl)-N—((S)-2,6-dioxopiperidin-3-yl)-2-fluorobenzamide;
4-(4-((1-(4-(((1,3-trans)-3-((8-cyano-1-methyl-2-oxo-1,2-dihydroquinolin-5-yl)oxy)-2,2,4,4-tetramethylcyclobutyl)carbamoyl)phenyl)piperidin-4-yl)methyl)piperazin-1-yl)-N—((S)-2,6-dioxopiperidin-3-yl)-2-fluorobenzamide;
4-(4-((1-(4-(((1,3-trans)-3-((8-cyano-1-methyl-2-oxo-1,2-dihydroquinolin-5-yl)oxy)-2,2,4,4-tetramethylcyclobutyl)carbamoyl)-2-fluorophenyl)piperidin-4-yl)methyl)piperazin-1-yl)-N—((S)-2,6-dioxopiperidin-3-yl)-2-fluorobenzamide;
N-((1,3-trans)-3-((8-cyano-2-oxo-1,2-dihydroquinolin-5-yl)oxy)-2,2,4,4-tetramethylcyclobutyl)-4-(3-((7-(4-(2,6-dioxopiperidin-3-yl)-3,5-difluorophenyl)-2,7-diazaspiro[3.5]nonan-2-yl)methyl)azetidin-1-yl)benzamide;
4-(2-((1-(4-(((1,3-trans)-3-((8-cyano-2-oxo-1,2-dihydroquinolin-5-yl)oxy)-2,2,4,4-tetramethylcyclobutyl)carbamoyl)phenyl)azetidin-3-yl)methyl)-2,7-diazaspiro[3.5]nonan-7-yl)-N—((S)-2,6-dioxopiperidin-3-yl)-2-fluorobenzamide;
4-(3-((6-(4-(((1,3-trans)-3-((8-cyano-2-oxo-1,2-dihydroquinolin-5-yl)oxy)-2,2,4,4-tetramethylcyclobutyl)carbamoyl)phenyl)-2,6-diazaspiro[3.4]octan-2-yl)methyl)azetidin-1-yl)-N—((S)-2,6-dioxopiperidin-3-yl)-2-fluorobenzamide;
N-((1,3-trans)-3-((8-cyano-2-oxo-1,2-dihydroquinolin-5-yl)oxy)-2,2,4,4-tetramethylcyclobutyl)-4-(2-((1-(4-(2,6-dioxopiperidin-3-yl)-3,5-difluorophenyl)azetidin-3-yl)methyl)-2,6-diazaspiro[3.4]octan-6-yl)benzamide;
4-(3-((7-(4-(((1,3-trans)-3-((8-cyano-2-oxo-1,2-dihydroquinolin-5-yl)oxy)-2,2,4,4-tetramethylcyclobutyl)carbamoyl)phenyl)-2,7-diazaspiro[3.5]nonan-2-yl)methyl)azetidin-1-yl)-N—((S)-2,6-dioxopiperidin-3-yl)-2-fluorobenzamide; or
N-((1,3-trans)-3-((8-cyano-2-oxo-1,2-dihydroquinolin-5-yl)oxy)-2,2,4,4-tetramethylcyclobutyl)-4-(2-((1-(4-(2,6-dioxopiperidin-3-yl)-3,5-difluorophenyl)azetidin-3-yl)methyl)-2,7-diazaspiro[3.5]nonan-7-yl)benzamide;
or a pharmaceutically acceptable salt thereof,
wherein one or more hydrogen atoms is independently and optionally replaced by a deuterium atom.

32. A pharmaceutical composition comprising at least one compound or pharmaceutically acceptable salt thereof according to any one of claims 1 to 31 and a pharmaceutically acceptable carrier.

33. The pharmaceutical composition according to claim 32, wherein the compound or pharmaceutically acceptable salt thereof is present in a therapeutically effective amount.

34. A method of treating cancer in a subject in need thereof, comprising administering to said subject at least one compound or pharmaceutically acceptable salt thereof according to any one of claims 1 to 31 or a pharmaceutical composition according to claim 32 or 33.

35. The method according to claim 34, wherein the cancer is prostate cancer, head and neck cancer, skin cancer, sarcoma, renal cell carcinoma, adrenocortical carcinoma, bladder cancer, lung cancer, gastric carcinoma, esophageal carcinoma, pancreatic adenocarcinoma, colorectal cancer, connective tissue cancer, glioblastoma multiforme, cervical cancer, uterine cancer, ovarian cancer, or breast cancer.

36. The method according to claim 34 or 35, wherein the cancer is prostate cancer.

37. The method according to any one of claims 34 to 36, wherein the cancer is androgen receptor positive.

38. The method according to any one of claims 34 to 37, wherein the subject has been previously treated with an anti-cancer agent.

39. The method according to claim 38, wherein the anti-cancer agent is enzalutamide, apalutamide, bicalutamide, darolutamide, flutamide, abiraterone, or a combination of any of the foregoing.

40. The method according to claim 39, wherein the anti-cancer agent is enzalutamide, apalutamide, or darolutamide.

41. A use of a compound or pharmaceutically acceptable salt thereof according to any one of claims 1 to 31 or a pharmaceutical composition according to claim 32 or 33 for treating cancer.

42. The use according to claim 41, wherein the cancer is prostate cancer, head and neck cancer, skin cancer, sarcoma, renal cell carcinoma, adrenocortical carcinoma, bladder cancer, lung cancer, gastric carcinoma, esophageal carcinoma, pancreatic adenocarcinoma, colorectal cancer, connective tissue cancer, glioblastoma multiforme, cervical cancer, uterine cancer, ovarian cancer, or breast cancer.

43. The use according to claim 41 or 42, wherein the cancer is prostate cancer.

44. The use according to any one of claims 41 to 43, wherein the cancer is androgen receptor positive.

45. A use of a compound or pharmaceutically acceptable salt thereof according to any one of claims 1 to 31 or a pharmaceutical composition according to claim 32 or 33 in the manufacture of a medicament.

46. The use according to claim 45, wherein the medicament is for the treatment of a cancer, wherein the cancer is prostate cancer, head and neck cancer, skin cancer, sarcoma, renal cell carcinoma, adrenocortical carcinoma, bladder cancer, lung cancer, gastric carcinoma, esophageal carcinoma, pancreatic adenocarcinoma, colorectal cancer, connective tissue cancer, glioblastoma multiforme, cervical cancer, uterine cancer, ovarian cancer, or breast cancer.

47. The use according to claim 46, wherein the cancer is prostate cancer.

48. The use according to claim 46 or 47, wherein the cancer is androgen receptor positive.

49. A method of inhibiting cell growth, comprising contacting a cell with at least one compound or pharmaceutically acceptable salt thereof according to any one of claims 1 to 31 or a pharmaceutical composition according to claim 32 or 33.

50. The method according to claim 49, wherein the cell is a cancer cell.

51. The method according to claim 50, wherein the cancer cell is a prostate cancer cell.

52. The method according to any one of claims 49 to 51, wherein the cell is androgen receptor positive.
